SEQUENCE LISTING (200 sequences)

SEQ ID NO: 1 - DNA, 1149 bp, Chlamydomonas reinhardtii
atggccgcgggatctgttggcgtcttcgccaccgatgagaagattggcagcctgctggaccagtccatcacgcgccactttctgtcgactgtgaccgaccagcagggcaagatctgtgccgagtatgtgtggatcggcggctccatgcacgacgtgcgctccaagtcgcgcaccctgtccaccatccccacgaagcccgaggacctgccccactggaactacgacggctcctccaccggccaggcccccggccacgactcagaggtctatctcattccccgctccatcttcaaggaccccttccgcggcggcgacaacatcctggtcatgtgcgactgctacgagccgcccaaggtcaaccccgacggcaccctggccgcgcccaagccgatccccacgaacacccgctttgcctgcgccgaggtgatggagaaggccaagaaggaggagccctggttcggcattgagcaggagtacacgctgctcaacgccatcaccaagtggccgctgggctggcccaagggcggctaccccgccccccagggcccctactactgctcggccggcgccggcgtggccatcggccgcgacgtggcggaggtgcactaccgcctgtgcctggccgcgggcgttaacatcagcggcgtgaacgccgaggtgctgcccagccagtgggagtaccaggtgggcccgtgcgagggcatcaccatgggcgaccacatgtggatgagccgctatatcatgtaccgcgtgtgcgagatgttcaacgtggaggtctcgttcgaccccaagcccatccccggcgactggaacggctccggcggccacaccaactactccactaaggccacccgcaccgcgcccgacggctggaaggtcatccaggagcactgcgccaagctggaggcgcgccacgccgtgcacatcgccgcctacggcgagggcaacgagcgccgcctgaccggcaagcacgagaccagcagcatgagcgacttcagctggggcgtggccaaccgcggctgctccatccgcgtgggccgcatggtgccggtggagaagtcgggctactatgaggaccgccggcctgcctccaacctggacgcctacgtcgtcacccgcctcatcgtggagaccaccatccttctgtaa

SEQ ID NO: 2 - protein, 382 aa, Chlamydomonas reinhardtii
MAAGSVGVFATDEKIGSLLDQSITRHFLSTVTDQQGKICAEYVWIGGSMHDVRSKSRTLSTIPTKPEDLPHWNYDGSSTGQAPGHDSEVYLIPRSIFKDPFRGGDNILVMCDCYEPPKVNPDGTLAAPKPIPTNTRFACAEVMEKAKKEEPWFGIEQEYTLLNAITKWPLGWPKGGYPAPQGPYYCSAGAGVAIGRDVAEVHYRLCLAAGVNISGVNAEVLPSQWEYQVGPCEGITMGDHMWMSRYIMYRVCEMFNVEVSFDPKPIPGDWNGSGGHTNYSTKATRTAPDGWKVIQEHCAKLEARHAVHIAAYGEGNERRLTGKHETSSMSDFSWGVANRGCSIRVGRMVPVEKSGYYEDRRPASNLDAYVVTRLIVETTILL

SEQ ID NO: 3 - protein, 15 aa, Artificial sequence
  motif 1
GYYEDRRPAANVDPY

SEQ ID NO: 4 - protein, 13 aa, Artificial sequence
  motif 2
DPIRGAPHVLVLC

SEQ ID NO: 5 - protein, 8 aa, Artificial sequence
  motif 3
GAHTNFST

SEQ ID NO: 6 - DNA, 1179 bp, Oryza sativa
ttgcagttgtgaccaagtaagctgagcatgcccttaacttcacctagaaaaaagtatacttggcttaactgctagtaagacatttcagaactgagactggtgtacgcatttcatgcaagccattaccactttacctgacattttggacagagattagaaatagtttcgtactacctgcaagttgcaacttgaaaagtgaaatttgttccttgctaatatattggcgtgtaattcttttatgcgttagcgtaaaaagttgaaatttgggtcaagttactggtcagattaaccagtaactggttaaagttgaaagatggtcttttagtaatggagggagtactacactatcctcagctgatttaaatcttattccgtcggtggtgatttcgtcaatctcccaacttagtttttcaatatattcataggatagagtgtgcatatgtgtgtttatagggatgagtctacgcgccttatgaacacctacttttgtactgtatttgtcaatgaaaagaaaatcttaccaatgctgcgatgctgacaccaagaagaggcgatgaaaagtgcaacggatatcgtgccacgtcggttgccaagtcagcacagacccaatgggcctttcctacgtgtctcggccacagccagtcgtttaccgcacgttcacatgggcacgaactcgcgtcatcttcccacgcaaaacgacagatctgccctatctggtcccacccatcagtggcccacacctcccatgctgcattatttgcgactcccatcccgtcctccacgcccaaacaccgcacacgggtcgcgatagccacgacccaatcacacaacgccacgtcaccatatgttacgggcagccatgcgcagaagatcccgcgacgtcgctgtcccccgtgtcggttacgaaaaaatatcccaccacgtgtcgctttcacaggacaatatctcgaaggaaaaaaatcgtagcggaaaatccgaggcacgagctgcgattggctgggaggcgtccagcgtggtggggggcccacccccttatccttagcccgtggcgctcctcgctcctcgggtccgtgtataaataccctccggaactcactcttgctggtcaccaacacgaagtaaaaggacaccagaaacatagtacacttgagctcactccaaactcaaacactcacacca

SEQ ID NO: 7 - DNA, 53 bp, Artificial sequence
  primer prm08458
ggggacaagtttgtacaaaaaagcaggcttaaacaatggccgcgggatctgtt

SEQ ID NO: 8 - DNA, 50 bp, Artificial sequence
  primer prm08459
ggggaccactttgtacaagaaagctgggtgctgctcctgcgcttacagaa

SEQ ID NO: 9 - protein, 357 aa, Aureococcus anophagefferens
MASMDQAVLGKYMGLDTGDDCQVEYVFLDKDQVARSKCRTLPLKKVQGPVDAYPKWNYDGSSTGQAPGDDSEVMIVPRAKYPDPFRGGNHVLVLCDTYEPDGTPLPTNTRAPAVARFESGGAKEQVPWYGLEQEYTLFNLDGVTPLGWPVGGFPKPQGPYYCGAGADRAFGRAVSEAHYRACLYAGLEVSGTNAEVMPGQWEYQIGPSIGIDAADQLTISRYILSRVCEDLGVIVTIDPKPIAGDWNGAGMHINFSTESTRKEGGLAVIEAMCEKLGAKHTEHIAAYGEGNERRLTGDCETASIDQFSYGVADRGCSIRIPRDTAADKKGYLEDRRPASNVDPYVATSLIFATCTSA

SEQ ID NO: 10 - protein, 380 aa, Chlamydomonas reinhardtii
MAFALRGVTAKASGRTAGARSSGRTLTVRVQAYGMKAEYIWADGNEGKPEKGMIFNEMRSKTKCFEAPLGLDASEYPDWSFDGSSTGQAEGNNSDCILRPVRVVTDPIRGAPHVLVMCEVFAPDGKPHSTNTRAKLREIIDDKVTAEDCWYGFEQEYTMLAKTSGHIYGWPAGGFPAPQGPFYCGVGAESAFGRPLAEAHMEACMKAGLVISGINAEVMPGQWEYQIGPVGPLALGDEVMLSRWLLHRLGEDFGIVSTFNPKPVRTGDWNGTGAHTNFSTKGMRVPGGMKVIEEAVEKLSKTHIEHITQYGIGNEARLTGKHETCDINTFKHGVADRGSSIRIPLPVMLKGYGYLEDRRPAANVDPYTVARLLIKTVLKG

SEQ ID NO: 11 - protein, 375 aa, Chlamydomonas reinhardtii
MRLNTQVSGRATGAPRQGRRLTVRVQAYGMKAEYIWADGNEGKAEKGMIFNEMRSKTKCFEAPLGLDASEYPDWSFDGSSTGQAEGNNSDCILRPVRVVTDPIRGAPHVLVMCEVFAPDGKPHSTNTRAKLREIIDDKVTAEDCWYGFEQEYTMLAKTSGHIYGWPAGGFPAPQGPFYCGVGAESAFGRPLAEAHMEACMKAGLVISGINAEVMPGQWEYQIGPVGPLALGDEVMLSRWLLHRLGEDFGIVSTFNPKPVRTGDWNGTGAHTNFSTKGMRVPGGMKVIEEAVEKLSKTHIEHITQYGIGNEARLTGKHETCDINTFKHGVADRGSSIRIPLPVMLKGYGYLEDRRPAANVDPYTVARLLIKTVLKG

SEQ ID NO: 12 - protein, 577 aa, Chlamydomonas reinhardtii
MDLATALGLGIAPPPPADDSSHHSTTEACTLPAYLRAPEVTAQVMAEYIWLMGGTGQLRSKTKVLDAKPSCAEEAPIMIVESNPDGQLAEPNHELFLKPRKIFRDPFRGGDHILVLCDTFIVAQVVAEAGAAPSTVLQPSETNSRVACENVLRVAEQQEPVFAVEQEYAIIHPAYPTKVPLGPRRPSTSRASSCHSGSRRSSYVSSGSARGGIGKNSSHHGGKQSHAAAAAAAAAVAGIPWPSPDACEQTAQEASAARQKASRQLADSHLRCCLFAGVRVTGADVHSLDGLHSYKIGPSPGVDLGDDLWTSRYLLQRVAEQHSASVSWEPDSMPSERPLGCHFKYSTASTRQAPHGLNAIEQQLVRLQATHVQHQVAYNDGRLDRLSSPEASTFTHAVGSANASVVVPSLTFLQQGGYFTDRRPPSDADPYKVTLLLAATTLDIPLPKLPASSSAGNTAANCSGGMSAGPSSCPAAAALPFGSPMQSYLLAAAAAQRQQQQQHLMFDTESEECDSVDEDDAMTEDSAALLAKMDDDGGAAEASSCDSDFEDQDDASSSPITGTWADNDCTHMLGAGI

SEQ ID NO: 13 - protein, 386 aa, Helicosporidum sp.
MSPPTGEKYSLPPVFGTQGQITQLLDPIMAERFKDLSQHGKVMAEYVWIGGTGSDLRCKTRVLDSVPNSVEDLPVWNYDGSSTGQAPGDDSEVFLIPRAIYRDPFRGGDNILVLADTYEPPRVLPNGKVSPPVPLPTNSRHACAEAMDKAAAHEPWFGIEQEYTVLDARTKWPLGWPSNGFPGPQGPYYCAAGAGCAIGRDLIEAHLKACLFAGINVSGVNAEVMPSQWEYQVGPCTGIESGDQMWMSRYILIRCAELYNVEVSFDPKPVPGDWNGAGGHVNYSNKATRTAETGWAAIQQQVEKLGKRHAVHIAAYGEGNERRLTGKHETSSMNDFSWGVANRGASVRVGRLVPVEKCGYYEDRRPASNLDPYVVTRLLVETTLLM

SEQ ID NO: 14 - protein, 416 aa, Thalassiosira pseudonana
MKLSIALLSMAATATAFAPSLTTPSRTTSLSMVNPLEIRTGKAQLDHSVIDRFNALPYPADKVLAEYVWVDAKGECRSKTRTLPVARTTAVDNLPRWNFDGSSTGQAPGDDSEVILRPCRIFKDPFRPRNDGVDNILVMCDTYTPAGEALPTNTRAIAAKAFEGKEDEEIWFGLEQEFTLFNLDQRTPLGWPKGGVPARAQGPYYCSVGPENSFGRAITDTMYRACLYAGIEISGTNGEVMPGQQEYQVGPCVGIDAGDQLQMSRYILQRVCEEFQVYCTLHPKPIVEGDWNGAGMHTNVSTKSMREEGGLEVIKKAIYKLGAKHQEHIAVYGEGNELRLTGKHETASIDQFSFGVANRGASVRIGRDTEAEGKGYFEDRRPSSNADPYLVTGKIMATIMEDVDVPEISALDRAEA

SEQ ID NO: 15 - protein, 379 aa, Volvox carterii
MATMRMSTKAQGRVGIVRNTRTLTVRVRAYGMKAEYIWADGNEGRPEKGMIFNEMRSKTKVFDEALPLEAGQYPDWSFDGSSTGQAAGNNSDCILRPVRVIKDPIRGEPHVLVMCEVFAPDGTPHPTNTRAKLRDIIDDKVLAEDCWYGLEQEYTMLQKTTGQIYGWPSGGYPAPQGPFYCGVGAESAFGRPLAEAHMEACMKAGLKISGINAEVMPGQWEYQIGPVGPLEMGDEVMLSRWLLHRLGEDFGIVCTFNPKPVRTGDWNGTGAHTNFSTKSMRQPGGMKVIEDAVEKLSKTHIEHITQYGLGNEARLTGKHETCDINTFKHGVADRGSSIRIPLPVMLKGYGYLEDRRPAANVDPYTVARLLIKSILKGPQ

SEQ ID NO: 16 - protein, 382 aa, Volvox carterii
MAAGSIGVFATDEKIGSLLDQSITRHFLTNVTDQCGKITAEYVWIGGSMQDLRSKSRTLTSVPTKPEDLPHWNYDGSSTGQAPGHDSEVYLIPRRIFRDPFRGGDNILVMCDCYEPPKANADGILQPPKPIPTNTRYACAEAMEKAKDEEPWFGIEQEYTLLNAITKWPLGWPKGGYPAPQGPYYCSAGAGVAIGRDVAEVHYRLCLYAGVNISGVNAEVLPSQWEYQVGPCEGIEMGDHMWMSRYIMYRVCEMFNVEVSFDPKPIPGDWNGSGGHTNYSTKATRTAPNGWKAIQEHCQKLEARHAVHIAAYGEGNERRLTGKHETSSMNDFSWGVANRGCSIRVGRMVPVEKCGYYEDRRPASNLDPYVVTKLIVETTVLL

SEQ ID NO: 17 - protein, 356 aa, Arabidopsis thaliana
MSLLADLVNLDISDNSEKIIAEYIWVGGSGMDMRSKARTLPGPVTDPSKLPKWNYDGSSTGQAPGQDSEVILYPQAIFKDPFRRGNNILVMCDAYTPAGEPIPTNKRHAAAEIFANPDVIAEVPWYGIEQEYTLLQKDVNWPLGWPIGGFPGPQGPYYCSIGADKSFGRDIVDAHYKASLYAGINISGINGEVMPGQWEFQVGPSVGISAADEIWIARYILERITEIAGVVVSFDPKPIPGDWNGAGAHTNYSTKSMREEGGYEIIKKAIEKLGLRHKEHISAYGEGNERRLTGHHETADINTFLWGVANRGASIRVGRDTEKEGKGYFEDRRPASNMDPYVVTSMIAETTLLWNP

SEQ ID NO: 18 - protein, 430 aa, Arabidopsis thaliana
MAQILAASPTCQMRVPKHSSVIASSSKLWSSVVLKQKKQSNNKVRGFRVLALQSDNSTVNRVETLLNLDTKPYSDRIIAEYIWIGGSGIDLRSKSRTIEKPVEDPSELPKWNYDGSSTGQAPGEDSEVILYPQAIFRDPFRGGNNILVICDTWTPAGEPIPTNKRAKAAEIFSNKKVSGEVPWFGIEQEYTLLQQNVKWPLGWPVGAFPGPQGPYYCGVGADKIWGRDISDAHYKACLYAGINISGTNGEVMPGQWEFQVGPSVGIDAGDHVWCARYLLERITEQAGVVLTLDPKPIEGDWNGAGCHTNYSTKSMREEGGFEVIKKAILNLSLRHKEHISAYGEGNERRLTGKHETASIDQFSWGVANRGCSIRVGRDTEAKGKGYLEDRRPASNMDPYIVTSLLAETTLLWEPTLEAEALAAQKLSLNV

SEQ ID NO: 19 - protein, 356 aa, Brassica napus
MSLLTDLVNLDLSDNTEKIIAEYIWVGGSGMDMRSKARTLPGPVTDPSKLPKWNYDGSSTGQAPGEDSEVILYPQAIFKDPFRRGNNILVMCDTYTPAGEPIPTNKRHAAAQIFSNPDVVAEVPWYGIEQEYTLLQKDVNWPVGWPIGGFPGPQGPYYCSVGADKSFGRDIVDAHYKACLYAGINISGINGEVMPGQWEFQVGPSVGISAADEVWIARYILERITEIAGVVVSFDPKPIPGDWNGAGAHTNYSTKSMREEGGYEIIKKAIDKLGLRHKEHISAYGEGNERRLTGHHETADINTFKWGVANRGASIRVGRDTEKEGKGYFEDRRPASNMDPYTVTSMIAETTLLWNP

SEQ ID NO: 20 - protein, 428 aa, Brassica napus
MAQILAASPTCQMRLTKPSSIASSKLWNSVVLKQKKQSSSKVRSFKVMALQSDNSTINRVESLLNLDTKPFTDRIIAEYIWIGGSGIDLRSKSRTLEKPVEDPSELPKWNYDGSSTGQAPGEDSEVILYPQAIFRDPFRGGNNILVICDTYTPAGEPIPTNKRARAAEIFSNKKVNEEIPWFGIEQEYTLLQPNVNWPLGWPVGAYPGPQGPYYCGVGAEKSWGRDISDAHYKACLYAGINISGTNGEVMPGQWEFQVGPSVGIEAGDHVWCARYLLERITEQAGVVLTLDPKPIEGDWNGAGCHTNYSTKSMREDGGFEVIKKAILNLSLRHMEHISAYGEGNERRLTGKHETASIDQFSWGVANRGCSIRVGRDTEKKGKGYLEDRRPASNMDPYIVTSLLAETTLLWEPTLEAEALAAQKLSLKV

SEQ ID NO: 21 - protein, 364 aa, Hordeum vulgare
MAAATTNVSYTTNLLKYMGLDQKGSAMAEYIWIDAVGGVRSKSKTLTSIPPSGEFTVDDLPEWNFDGSSTGQAPGDNSDVYLRPVAVFPDPFRGAPNILVITECWDPDGTPNKYNHRHEAAKLMEAHKAQKPWFGLEQEYTLLDMHDRPYGWPAGGFPGPQGPYYCGVGSGKVYCRDIVEAHYKACLFAGVKISGTNAEVMPAQWEFQVGPCEGIELGDQLWLARFLLHRIAEEFGAKISFHPKPIPGDWNGAGLHSNFSSEEMRKPGGMKAIEAAMKKLEARHKEHIAVYGEDNTMRLTGRHETGNIDSFTYGVANRGTSIRIPREVSQKGFGYFEDRRPASNADPYQITGIMVETIFGGLDK

SEQ ID NO: 22 - protein, 356 aa, Oryza sativa
MASLTDLVNLNLSDTTEKIIAEYIWIGGSGMDLRSKARTLSGPVTDPSKLPKWNYDGSSTGQAPGEDSEVILYPQAIFKDPFRKGNNILVMCDCYTPAGEPIPTNKRHNAAKIFSSPEVASEEPWYGIEQEYTLLQKDINWPLGWPVGGFPGPQGPYYCGIGADKSFGRDIVDSHYKACLYAGINISGINGEVMPGQWEFQVGPSVGISAGDQVWVARYILERITEIAGVVVSFDPKPIPGDWNGAGAHTNYSTKSMRNDGGYEIIKSAIEKLKLRHKEHISAYGEGNERRLTGRHETADINTFSWGVANRGASVRVGRETEQNGKGYFEDRRPASNMDPYIVTSMIAETTIIWKP

SEQ ID NO: 23 - protein, 428 aa, Oryza sativa
MAQAVVPAMQCQVGAVRARPAAAAAAAGGRVWGVRRTGRGTSGFRVMAVSTETTGVVTRMEQLLNMDTTPFTDKIIAEYIWVGGTGIDLRSKSRTISKPVEDPSELPKWNYDGSSTGQAPGEDSEVILYPQAIFKDPFRGGNNILVMCDTYTPAGEPIPTNKRNRAAQVFSDPKVVSQVPWFGIEQEYTLLQRDVNWPLGWPVGGYPGPQGPYYCAVGSDKSFGRDISDAHYKACLYAGINISGTNGEVMPGQWEYQVGPSVGIEAGDHIWISRYILERITEQAGVVLTLDPKPIQGDWNGAGCHTNYSTKSMREDGGFEVIKKAILNLSLRHDLHISAYGEGNERRLTGLHETASIDNFSWGVANRGCSIRVGRDTEAKGKGYLEDRRPASNMDPYVVTALLAETTILWEPTLEAEVLAAKKLALKV

SEQ ID NO: 24 - protein, 346 aa, Physcomitrella patens
MALAQKAEYIWMDGQEGQKGIRFNEMRSKTKVIQEPIKAGSLDFPKWSFDGSSTGQAEGRFSDCILNPVFSCLDPIRGDNHVLVLCEVLNPDSTPHETNTRRKIEELLTPDVLAEETLFGFEQEYTMFNKAGKVYGWPEGGFPHPQGPFYCGVGLEAVYGRPLVEAHMDACIKAGLKISGINAEVMPGQWEFQIGPAGPLEVGDHVMIARWLLHRLGEDFGITCTFEPKPMEGDWNGAGAHTNYSTKSMRVDGGIKAIHAAIEKLSKKHVEHISSYGLGNERRLTGKHETANINTFKSGVADRGASIRIPLGVSLDGKGYLEDRRPAANVDPYVVARMLIQTTLKN

SEQ ID NO: 25 - protein, 346 aa, Physcomitrella patens
MALAQKAEYIWMDGQEGQKGIRFNEMRSKTKVIQEPIKAGSLDFPKWSFDGSSTGQAEGRFSDCILNPVFSCPDPIRGDNHVLVLCEVLNPDSTPHETNTRRKIEELLTPDVLAEETLFGFEQEYTMFNKAAKVYGWPEGGFPHPQGPFYCGVGLEAVYGRPLVEAHMDACIKAGLKISGINAEVMPGQWEFQIGPAGPLEVGDHVMVARWLLHRLGEDFGITCTFEPKPMEGDWNGAGAHTNYSTKSMRVDGGIKAIHAAIEKLSKKHAEHISSYGLGNERRLTGKHETANINTFKSGVADRGASIRIPLGVSLEGKGYLEDRRPAANVDPYVVARMLIQTTLKN

SEQ ID NO: 26 - protein, 371 aa, Pinus taeda
MATPITSRTETLQKYLKLDQKGMIMAEYVWVDADGGTRSKSRTLPEKEYKPEDLPVWNFDGSSTNQAPGDNSDVYLRPCAVYPDPFRGSPNIIVLAECWNADGTPNKYNFRHDCVKVMDTYADDEPWFGLEQEYTLLGSDNRPYGWPAGGFPAPQGEYYCGVGTGKVVQRDIVEAHYKACLYAGIQISGTNAEVMPAQWEYQVGPCTGIAMGDQLWISRFFLHRVAEEFGAKVSLHPKPIAGDWNGALSFPGLCFISVILISLQGLHSNFSTKAMREEGGMKVIEEALKKLEPHHVECIAEYGEDNELRLTGRHETGSIDSFSWGVANRGTSIRVPRETAAKGYGYFEDRRPASNADPYRVTKVLLQFSMA

SEQ ID NO: 27 - protein, 354 aa, Pinus taeda
MAYAYRPELLAPYLSLPQGEKVQAEYVWVDGDGGLRSKTCTVDKKVTDIGQLRVWDFDGSSTNQAPGGNSDVYLRPAAIFKDPFRGGDNILVLAECYNNDGTPNKTNHRHHAAKVMELAKDQKPWFGLEQEYTLFDVDGTPFGWPKGGFPGPQGPYYCGAGAGKVYARDLIEAHYRVCLYAGIKISGVNAEVMPAQWEFQVGPCEGIEMGDHLWMARYLLIRLAEQWGIKVSFHPKPLAGDWNGSGCHTNYSTAPMREEGGMKHIEAAIEKLAQKHDEHIAVYGDDNDMRLTGRHETGHIGTFSSGVANRGASIRIPRHVAAKGYGYLEDRRPASNVDPYRVTSIIVETTVTNA

SEQ ID NO: 28 - protein, 416 aa, Phaedactylum tricornutum
MKLNIAAIALFAASASAFAPRFASPRSHATVLSAVLEERTGQSQLDPAVIERYAALPYPDDTVLAEYVWVDAVGNTRSKTRTLPAKKAASVEALPKWNFDGSSTDQAPGDDSEVILRPCRIFKDPFRPRNDGLDNVLVMCDCYTPNGEAIPTNHRAKAMESFESREDEEIWFGLEQEFTLFNLDKRTPLGWPEGGMPNRPQGPYYCSVGPENNFGRHITESMYRACLYAGINISGTNGEVMPGQQEYQVGPCVGIDAGDQLMMSRYILQRVCEDFQVYCTLHPKPIVDGDWNGAGMHTNVSTKSMREEGGLEVIKKAIYKLGAKHLEHIAVYGEGNELRLTGKHETASMDKFCYGVANRGASIRIGRDTEAEGKGYFEDRRPSSNADPYIVTGKIMNTIMEDVEVPDIAPMDKAVA

SEQ ID NO: 29 - protein, 423 aa, Zea mays
MAQAVVPAMQCRVGVKAAAGRVWSAGRTRTGRGGASPGFKVMAVSTGSTGVVPRLEQLLNMDTTPYTDKVIAEYIWVGGSGIDIRSKSRTISKPVEDPSELPKWNYDGSSTGQAPGEDSEVILYPQAIFKDPFRGGNNVLVICDTYTPQGEPLPTNKRHRAAQIFSDPKVAEQVPWFGIEQEYTLLQKDVNWPLGWPVGGFPGPQGPYYCAVGADKSFGRDISDAHYKACLYAGINISGTNGEVMPGQWEYQVGPSVGIEAGDHIWISRYILERITEQAGVVLTLDPKPIQGDWNGAGCHTNYSTKTMREDGGFEEIKRAILNLSLRHDLHISAYGEGNERRLTGKHETASIGTFSWGVANRGCSIRVGRDTEAKGKGYLEDRRPASNMDPYIVTGLLAETTILWQPSLEAEALAAKKLALKV

SEQ ID NO: 30 - protein, 356 aa, Zea mays
MACLTDLVNLNLSDTTEKIIAEYIWIGGSGMDLRSKARTLPGPVTDPSKLPKWNYDGSSTGQAPGEDSEVILYPQAIFKDPFRRGNNILVMCDCYTPAGEPIPTNKRYSAAKIFSSLEVAAEEPWYGIEQEYTLLQKDTNWPLGWPIGGFPGPQGPYYCGIGAEKSFGRDIVDAHYKACLYAGINISGINGEVMPGQWEFQVGPSVGISSGDQVWVARYILERITEIAGVVVTFDPKPIPGDWNGAGAHTNYSTESMRKEGGYEVIKAAIEKLKLRHKEHIAAYGEGNERRLTGRHETADINTFSWGVANRGASVRVGRETEQNGKGYFEDRRPASNMDPYVVTSMIAETTIVWKP

SEQ ID NO: 31 - DNA, 1074 bp, Aureococcus anophagefferens
atggcgtccatggaccaggccgtgctcggcaagtacatgggcctcgacacgggcgacgactgccaggtcgagtacgtcttcctcgacaaggaccaggtcgcgcggtccaagtgccgcacgctgcccctcaagaaggtccagggccccgtggacgcgtaccccaagtggaactacgacggctcgtcgacgggacaggcgcccggcgacgactccgaggtcatgatcgtgccccgcgccaagtaccccgaccccttccgcggcgggaaccacgtcctcgtgctctgcgacacctacgagcccgacgggacgcctctaccgacgaacacgcgcgcgcccgccgtcgcccgcttcgagtcgggcggcgcgaaggagcaggtgccctggtacggcctcgagcaggagtacacgctcttcaacctcgacggcgtcacgcccctgggctggcccgtcggcggcttccccaagccccagggcccctactactgcggcgcgggcgcggaccgcgcgttcggccgcgccgtgtccgaggcgcactaccgcgcgtgcctctacgcgggcctcgaggtctcgggcacgaacgccgaggtcatgcccggccagtgggagtaccagatcggcccctccatcggcatcgacgccgcggaccagctcacgatctcgcgctacatcctcagccgcgtctgcgaggacctcggcgtcatcgtcaccatcgaccccaagcccatcgccggcgactggaacggcgcgggcatgcacatcaacttctccaccgagtccacgcgcaaggagggcggcctcgcggtcatcgaggccatgtgcgagaagctcggcgcgaagcacacggagcacatcgccgcgtacggcgagggcaacgagcgccgcctcacgggcgactgcgagacggcctccatcgaccagttctcctacggcgtcgccgaccgcggctgctccatccgcatcccccgcgacaccgcggccgacaagaagggctacctcgaggaccgccgccccgcgtccaacgtggatccctacgtcgcgacgtcgctcatcttcgcgacctgcacgtccgcctag

SEQ ID NO: 32 - DNA, 2031 bp, Chlamydomonas reinhardtii
ctcacacacgcacaattctttactctgctgcctgtccactcgcctgtccaactactaccagtcggggatttcttctcctgaaggtctaaccatggccgcgggatctgttggcgtcttcgccaccgatgagaagattggcagcctgctggaccagtccatcacgcgccactttctgtcgactgtgaccgaccagcagggcaagatctgtgccgagtatgtgtggatcggcggctccatgcacgacgtgcgctccaagtcgcgcaccctgtccaccatccccacgaagcccgaggacctgccccactggaactacgacggctcctccaccggccaggcccccggccacgactcagaggtctatctcattccccgctccatcttcaaggaccccttccgcggcggcgacaacatcctggtcatgtgcgactgctacgagccgcccaaggtcaaccccgacggcaccctggccgcgcccaagccgatccccacgaacacccgctttgcctgcgccgaggtgatggagaaggccaagaaggaggagccctggttcggcattgagcaggagtacacgctgctcaacgccatcaccaagtggccgctgggctggcccaagggcggctaccccgccccccagggcccctactactgctcggccggcgccggcgtggccatcggccgcgacgtggcggaggtgcactaccgcctgtgcctggccgcgggcgttaacatcagcggcgtgaacgccgaggtgctgcccagccagtgggagtaccaggtgggcccgtgcgagggcatcaccatgggcgaccacatgtggatgagccgctatatcatgtaccgcgtgtgcgagatgttcaacgtggaggtctcgttcgaccccaagcccatccccggcgactggaacggctccggcggccacaccaactactccactaaggccacccgcaccgcgcccgacggctggaaggtcatccaggagcactgcgccaagctggaggcgcgccacgccgtgcacatcgccgcctacggcgagggcaacgagcgccgcctgaccggcaagcacgagaccagcagcatgagcgacttcagctggggcgtggccaaccgcggctgctccatccgcgtgggccgcatggtgccggtggagaagtcgggctactatgaggaccgccggcctgcctccaacctggacgcctacgtcgtcacccgcctcatcgtggagaccaccatccttctgtaagcgcaggagcagcggcacgcaggagcagcagtgggcgatggtggtggtggcgtttgtgctggcctgagcgaggggggggccacggaagggcgatcgtggccaaggcggagggaagcggcggtcgagccgcgtggtgatcaaggtgaggcgtggtgcgcgtgtttgcattgacatgcgggctcgtttggcgccgtggcttgaagctggagcaattccaactgcatttggtttgccgggacgtgtagcggttcaggaaagatggggtgacggcagcgaggacccgctgtgtgttctggtccagtctgccaaagggacttcggacgcaggatgctgcatcatctgtggcgcagtcaactgatctctacgaagagccgcagtgccataccatttgtcgtgtgcgtttcgagcctggctgtgtggacgccggcgcagaggtcgcctggttgtgtgcaagtgtatgccgtcggcgacggaagggagcgtacaccgtgcggccaagcgacacggcgctctgtacgtgcccgtcgtcaagtgcatgagcggaggaccccgcgcagcgcggggtgcgtggcatgacgtgagctcttattggctgtgcgcgacgcatgcgttccctcatgtaggggaggcgttgcatacaggaacggtcgcggccgtgtttggtgttcaaactgtgttttgtcttggtattgtgtcctggttccaacagtggttgggtgattgtgcacttgaaacattctttgtgtgggtcggacccactctgtcgttctgtaacacgggaaagcatacgg

SEQ ID NO: 33 - DNA, 3248 bp, Chlamydomonas reinhardtii
cctgttacagcaacatacagttctagccaagtagcaacgcacaacttcaagccttgatcaatcagtatggacctcgccaccgcgcttggactgggcatagcccccccgccgcccgcggacgactcctcccaccacagcaccacggaagcatgcactctgccggcgtatctgcgcgcgccggaggtgacggcccaggttatggccgagtacatctggctgatgggtgggaccggccagctgcgcagcaagactaaggtgctggacgccaagccgtcttgtgccgaggaggcccctatcatgattgtggagagcaacccagacggccagctcgccgagccgaaccatgagcttttcctcaagccccgcaagatcttccgggaccccttccgcggcggcgaccacattctggtcctctgcgacacattcatcgtcgcccaggttgtcgcggaggctggtgcggctccctcgaccgtgctgcagcccagcgagaccaacagccgcgttgcgtgcgagaacgtcctgcgcgttgccgagcagcaggagcccgtgtttgcggtggagcaggagtacgccatcatccacccggcgtaccccacgaaggttccgctgggacctcggcgcccttcgacctcgcgcgccagcagctgccacagcggctcgcgccgcagcagctacgtgtccagtggctcagcgcgcggcgggatcggcaagaacagcagccaccacggcggcaagcagtcgcacgccgctgccgccgccgctgcggcggcggtcgccggcatcccttggcccagcccggacgcatgtgagcagacggcccaagaagcgagcgcagcgaggcagaaggcgtcgagacagcttgcggactcgcacctgcgctgttgcctctttgcgggcgtgagggtgacgggcgcggacgtgcactcgcttgacggtctgcactcgtacaagatcgggccgtcgccgggggtggacctcggcgatgacctctggaccagcagatacctgctacagcgggtcgcagagcagcacagcgcatcggtgtcgtgggaacccgactcaatgccgtcggaacggccgctgggctgccacttcaaatacagtacggcgtcgacgcggcaggcgccacacggcttgaacgcgatagaacagcagctcgtgcggctgcaggctacgcacgttcagcaccaggtggcctacaacgacggcaggctggaccggctgtcctcgccggaggcctccacgtttacgcacgcggtcggctcggccaacgcctccgtcgtagtgcccagcctaaccttcctgcagcagggcggctacttcacggaccgccgcccgccgtcggatgccgacccctacaaggtgaccctgctcctggcagcgaccacgctggacatccccctgcccaagctgcccgcgtcctcgtccgccggcaacacggcggccaactgcagtggcggcatgtcggcgggcccgtcctcgtgtcccgctgctgctgccctgcctttcggcagtcccatgcagagctacctgctggccgctgcggccgcccaacggcagcagcagcagcagcacctgatgttcgacacggagagcgaggagtgcgactccgtcgacgaagatgatgcgatgactgaagactcggcagctctgctggccaagatggacgacgatggcggcgctgcagaggcgtcgtcgtgcgactcggacttcgaggaccaggacgatgccagctccagccctatcaccggcacctgggcggacaacgactgcacccacatgctgggtgctggcatttaagactactagactgagaatggaaccttgtttgcctcttgtattgcttcgtgcagttcaaagtgtgcatgtccgggtgctcgagtgtgtgcgcgttcccataatgcgcgtgttccagtagtacgtgtgcccatgttccagtagtagttctcgctgcagggttattgttgacaagctttgtctgatgcctttctcgtgcgttttttcctcgtgcacatggacgcgagatgttctggtctgatggatccgagattttgagcggcatgcaatcacgccggagcgcggccgcacccctctcactgctatatcgataccctggtcagggtttacgcgcgtcatcccgtagatggagtgggagcgaaagagacttgtgcaagctgtacaccgcaattggcgctttggctgattgttccgtgccagttctgcatgccgtgacggtatcgaaatgaatgtgtccaagcatttggctgggtggcgattgaaggatcgggatggacctgatgggcatcacctggtgcatgtgcgctcaagccgttcaatggaaagaatggcaagatgggtttgcagtgtgcacatgcctaatgctacctagtgaacacgtgtgcctgccgtgaatgtgtgtgtgtgtgtgtttaggttcaccctgtttaccgagctatccggggcagacatccctccgattatcatcataaatgcatggctggcatggggagctgactacaaccgggggtttcaagatttacacaaccgccagccgacttgcggtgctggcggatcggactgacgtagtgggctcatccttgaggcgtgtagagtgtgcagtactgactggtggcagcgctgtagtagcggcgtacgagccgcatatcaagcattacgggtgaccatttccaaatgattacaatctggtgcggcggcggaggtgcggcttgggttctgcagcccattctatcactggcgcggaggtcatcaagccggagccgacctgacacgggcccgtaagggatgcacggtcaacaggccaggaaagcaggatggcacaggccgtgtgtcgtgtgacgcgatccatgtcacggtggctggatgaagttagcagtatcaatggcatgactgcgagcatggtcgctgtgtggcgccaggccaaacatagacggtcaagcagtatcatgcaggtgaaccccgtgaagggatgtgcacgcatgagcagtatcaatggcatgactgcgagcatggtcgctgtgtggcgccaggccaaacatagacggtcaagcagtatcatgcaggtgaaccccgtgaagggatgtgcacgcatgaactctaatgttgattagcaagtgtacggttgttctgtatgtcgtggggcgtctgttcgcgggtggtgcatgggtgcattgacctggctgtgagtattcatgtaaacgttttgggattctgtacatctccagaacccg

SEQ ID NO: 34 - DNA, 1593 bp, Chlamydomonas reinhardtii
ctttacctcgttgcaaagatggcgttcgctctgcgtggtgttaccgctaaggcctcgggccgcactgctggcgcccgctcgtcgggccgcaccctgacggtgcgcgtccaggcctatggcatgaaggctgagtacatctgggcggatggcaacgagggcaagcctgagaagggcatgatcttcaacgagatgcgctcgaagaccaagtgcttcgaggcccccctgggcctggacgcctcggagtaccccgactggtcgttcgatggctcgtccaccggccaggctgagggcaacaactcggactgcatcctgcgccccgtgcgcgtggtgaccgaccccatccgcggtgccccccacgtgctggtgatgtgcgaggtgttcgcccccgatggcaagccccactccaccaacacccgcgccaagctccgcgagatcattgacgacaaggtcactgccgaggactgctggtacggcttcgagcaggagtacaccatgctggccaagacctctggccacatctacggctggcccgctggcggcttccctgctccccagggccccttctactgcggtgtgggcgctgagtccgccttcggccgccccctggctgaggcccacatggaggcctgcatgaaggccggtctggtcatctccggcatcaacgccgaggtgatgcccggccagtgggagtaccagatcggccccgtcggccctctggccctgggcgacgaggtgatgctgtcccgctggctgctgcaccgcctgggcgaggacttcggcattgtgtcgaccttcaaccccaagcccgtgcgcaccggtgactggaacggcactggcgcccacactaacttctcgaccaagggcatgcgcgtgcccggcggcatgaaggtgatcgaggaggccgtggagaagctgtccaagacccacatcgagcacatcacccagtacggcattggcaacgaggcgcgcctgaccggcaagcacgagacctgcgacatcaacaccttcaagcacggtgtggctgaccgcggctcttccatccgcattcccctgcccgtcatgctcaagggctacggctacctggaggaccgccgccccgctgccaacgtcgacccctacaccgtggcgcgcctgctgatcaagaccgtgctcaagggctaaatgcccagcatgcgccagctaataagggcagcgatgaggcggaggggtgcgtgactcggatgtgagctgtgatgagggggttgcttctatcggctaagggtgtgtgtgtgtgtgtctgtctatgctgggccgggtatgtggaccggcgacctgacgtttggaatgcgtgcgtgtgcacactgcccggttgcagtgtctgcgcatgtatttcctggcaactccaaagcctacggttgagcaagtgacctgtctttggttggacgattgttctgacacgtcgattgctgctaggttaacgggaggttgcggcgtgagccctgcgacgagctgcgtaatactatttccttgtacttcttcctcgcgcgccctcctgggtgctgacgcattgtcaggtttgctcaggtcgccacatgtaatcgaacacgtcaacag

SEQ ID NO: 35 - DNA, 1328 bp, Helicosporidum sp.
catttcttattcctttggagctgtgctccttttggttttgtgcagttgtgtactgccggcactccttcgccttcggtgctttctgcgtagagctcaagcatgtctcctcccactggcgaaaagtactctctgccccccgtcttcgggacgcaggggcagatcacccagctgcttgaccctatcatggctgagcgcttcaaggacctctctcagcacggcaaagtgatggcggagtacgtctggattggcggcacgggcagcgacctgcggtgcaagacccgcgttctggactcggtccccaacagcgtcgaggatctgccggtgtggaactacgacggctcctccacaggccaggcccccggcgacgattcggaagtattcctcatcccccgcgccatctaccgcgatcctttccgcggcggggacaacatcctggtgctggcggacacgtacgagcccccacgcgtgctccccaacggcaaggtttccccccccgtgccgctgcccaccaactcccgccacgcctgcgccgaggccatggacaaggctgcggcgcacgagccctggttcgggatcgagcaggagtacacggtgctggacgcccgcaccaagtggcccctgggctggccctccaacggcttccccggtccccaaggcccttactactgcgcggctggcgcggggtgtgccatcggccgagacctgatcgaggcgcatctcaaggcgtgcctgttcgcgggcatcaacgtctcgggcgtgaacgccgaggtgatgcccagccagtgggagtaccaggtgggtccctgcaccggcatcgaaagcggagaccagatgtggatgagccggtacattctcatccggtgcgccgagctctacaacgtggaggtttctttcgaccccaagcccgtgcctggcgactggaacggcgccggcgggcacgtcaactactccaacaaggccacccgcacggccgagacgggctgggcggccatccagcagcaagtcgagaagctgggcaagcgccatgccgtgcacatcgccgcttacggcgagggcaacgagcgccgcctcacgggcaagcacgagaccagctccatgaacgacttctcgtggggcgtggccaaccgcggcgcctcggtgcgggtgggccgtctcgtgccggtggagaagtgcggctactacgaagaccgacgcccggcctccaacctggacccttacgtggtcacgcgcctgctggtggagaccacgctgctcatgtagatatgcaggggggtgggtgggagatggcaacggctgtgacttgcgtggatgtagatagttttcgggtg

SEQ ID NO: 36 - DNA, 1470 bp, Thalassiosira pseudonana
caaaatcaaccaaccatgaagctctccatcgccctcctctccatggccgcgacggccacagccttcgccccatccctcaccaccccctcccgcaccacctccctctccatggtaaaccccctcgagatcagaaccggaaaagcccaactagaccactccgtcatcgaccgcttcaacgcacttccctaccccgctgacaaagtactggccgaatacgtctgggtcgacgccaagggagagtgccgttcaaagacgcgtactcttcccgtggctcgtaccacggctgtggacaatttgcctcgttggaactttgatggaagttcgacaggtcaggctcctggtgatgatagtgaggttatcttgagaccgtgtaggatcttcaaggatcctttcaggccacgtaatgacggtgtggacaacatcttggtgatgtgtgatacttatactcctgccggagaggctttgcctacgaatacgagggcgattgccgcaaaagcctttgaaggaaaggaagacgaagaaatctggttcggcctcgaacaagaattcaccctcttcaacctcgaccaacgcacccccctcggctggcccaagggaggcgtccccgcccgcgcccaaggcccctactactgctccgtcggacccgagaactccttcggacgtgccatcaccgacaccatgtaccgtgcctgtctctacgccggtattgagatcagcggtaccaatggagaggtcatgcccggtcagcaagagtatcaggttggaccatgtgtaggaattgatgctggtgatcagcttcagatgtcacgatacattcttcaacgtgtgtgtgaggagttccaggtctactgtactctacaccccaagcctattgtggagggagattggaacggagccggtatgcacaccaatgtctccaccaaatccatgcgtgaggagggaggacttgaggtcatcaaaaaggcaatttacaaacttggagccaagcatcaagagcacatcgctgtttacggagagggcaatgagttgcgtttgactggaaaacacgagactgcaagtattgatcagttctcgtttggagttgcaaataggggagctagtgtgaggattggaagggataccgaggctgagggtaagggatactttgaggacaggaggcctagttcgaatgctgatccttatttggttactggaaagattatggctaccatcatggaggacgttgacgttccagaaatcagtgcccttgaccgtgccgaggcctaagcacttcttctttcttcccaaacacactccttctttctctttggagaacttttgaacatgacgagtaggaatacgacactgattgcacaattcaaatgagtttggcaagtgtacagtcttctttgttgagagaatgtctcatttttcatgccgaggctacgataattgactaatgctactaaaggagaatagtttgctgaattg

SEQ ID NO: 37 - DNA, 2446 bp, Volvox carterii
caaaatctgtaatcatggctaccatgcgcatgtccacgaaggctcagggccgcgtcgggattgtccgcaacacgcggaccctgacagtgcgcgtacgtgcgtatggtatgaaggccgaatatatctgggccgatggaaatgagggccggcccgagaagggcatgatctttaacgagatgcgctcgaagacgaaggtctttgatgaggctctacccctggaagctggccagtaccccgactggtccttcgatggctcttcgaccggccaggccgccggcaacaactccgactgcatcctcaggcccgtccgcgtcatcaaggaccccatccgcggtgagccgcacgtgctggtgatgtgcgaggtgttcgcccctgatggcaccccgcaccctaccaacactcgtgccaagctgcgcgacatcattgacgacaaggtccttgccgaggactgctggtacggtctggagcaggagtacaccatgcttcaaaagaccaccggccagatctacggctggcccagcggcggttaccctgcaccccagggccccttctactgcggtgtcggtgcggagtcggcgttcggccggcccctggctgaggctcacatggaggcttgcatgaaggctggtcttaagatctctggcatcaacgccgaggtgatgccaggccagtgggagtaccagattggcccggtgggtcccttggagatgggcgatgaggtgatgctgtcgcgctggctgctgcaccgtctgggcgaggatttcggcattgtctgcaccttcaaccccaagcctgtccgcaccggcgactggaacggcactggcgcgcacaccaacttctcgaccaagtccatgcgccagcctggcggcatgaaggtgattgaggacgccgtggagaagctctccaagacccacattgagcacatcacccagtacggtctgggcaatgaggctcgtctgaccggcaagcacgagacgtgcgacatcaacaccttcaagcacggtgttgcggaccgcggctcgtccattcgcatcccgttgccggtcatgctgaagggctacggctacctggaggaccgtcgcccggctgccaacgttgacccgtacactgtcgcccgcctgctcatcaaatccatcctcaagggcccgcagtaaatgatccctcgtactgagccacttcggtcattccgacgcacccataggcaacttacggttacctagtctcggacgttcttgtgaatgggttggcctcatttgcaggatggcatgatgggacaggtgtaagatgttctagaggctctggagtgggcttggggctggagataccccggtgcatgtttgtagctgtgggttggctggtacgatgtgacaagaaccgtccggaactattaagaagttcattggatcaatggacaatatatttattgcggaaatgtctttttgcgcgttgacaagtggctagctgctactgatcctactattatctgccatacttacgcagtttaattttcggcatcagtgcacacgttctcctgtaatggttaggaaacatgtgctattgaggagacgtgcgtgtgactgatatcctgacacgcctaggtatcggagtgtacgttgagttccagttcacgggttcatgcggctagcgggcatgccttggcgacggctgcaattgcaccgagttgccgaggggtgcatgtgcatagcgggttgtcgcatacggagataatgctctttgtgtttggggctttttttcctgtgtgtagctctcttctacagcagctaagcgagcttaattcgtgaggtacagagagtttcatctactgtatagattactttatttccttccgggtattgaacgattgcatgccgtacctgggcatgtagtcttcgacgtacgtgtgctaagcttctgcggttctcatgaagtggagatgccgcatttgtatcatgattgcacaaatataacgatcttggtgtgtcaggcccgggcaagctgctgtgcaatcacctgatactgtctcgattgatactgtcctaaaacgattgttatcattactgcgtgacatcgtacgacgcagcgtaacttttcttcaagcacaactgtcattgacatactgtcttaacgacaggcacaaatcatcaagtgtttaaaacggcaggccgttccgcgctgacctggtgctggcatggctcctcatccagcgaatggcaatcaaagtgggtaggaaacccaacttgatttaaacacttgtttggtatagtacggcaaaagtcaacgacccccgaacttggctgtacagcatgggtggtgattttcttcagggacacttgaaactttgtatacacatgccggaatacagtcaacaatatttattaaagcaattgattacagaagtcttaacagtgataggacactcatttagttggcagttgtaaaatgttat

SEQ ID NO: 38 - DNA, 2269 bp, Volvox carterii
aaggactttcgtcggcaactcaccgcgtcgcacacagttctgcttcaggccagcttgagataaatggctgctggatcaattggcgtttttgcaactgatgagaagattggaagccttctggaccagtccattacccgccacttcctgaccaatgtaacggatcagtgtggcaagatcaccgcggagtatgtgtggattggcgggagcatgcaggacttgaggtctaagtcccgcaccctgacttctgttcccacaaaacccgaggaccttccgcattggaactacgacggttcgtccacgggccaagcgccgggccacgactcagaggtgtacctcatcccccgccgcattttccgggatccgtttcgggggggtgacaacatccttgtcatgtgcgattgctacgagccgcccaaggccaacgcggacggtattctgcaaccgcccaagcccatcccaaccaacactcgctacgcgtgcgccgaggctatggagaaagccaaggatgaggagccatggttcggcattgagcaggagtacacgctgctgaacgcgattaccaagtggccgcttggctggcccaagggcggttaccccgcaccgcagggcccgtactactgctctgccggtgcaggtgtggctataggccgcgacgttgccgaggttcactacaggttgtgtctgtacgctggggtcaacatcagcggcgtgaacgctgaggtgctgccatcgcaatgggagtaccaggtgggcccatgcgagggcattgagatgggcgaccacatgtggatgtcccgttacatcatgtaccgcgtatgtgagatgttcaacgtggaggtgtcgttcgaccccaagcccattcccggcgactggaacggctcaggtggccacaccaactactccaccaaggccacacgcactgcgcctaacggctggaaggccatccaagagcactgccagaagctggaagcgcgccacgcggttcacattgccgcctatggtgagggcaacgagcgccgcctgacgggaaagcacgagacgtcgtccatgaacgacttctcatggggcgtcgcgaaccgcggctgctccatccgcgttggccgcatggtgcccgtggagaagtgcggctattacgaggatcgccgccccgcctccaacctggacccgtacgtggtcaccaagctcatcgttgagaccacggtcctcctgtaatggcgtgggtcagcaaaatggtgggtcggcatgttcattaggtgtagttgtaacggcaatccgggtggatagtgctcagtcgcggcgtgtttgtggacgttatcatcagcgtgctatagtgatgggcggctgagaccgtatgagactcgcgcgcaatggcggttgtggcaaggtttttaagtgtccccgccatcttattccatgccccggctttcggaggctgctgctgaatgaagcgtccggggttggcctaccccactggggctgctgtcggcaaaacaaggtgcaacgccagacggtgtaggctgttggatctgggtgcttcgatgtgccgggcactggaggacacaatctaagcaagggccgagcggtttcatcgttaggaaactgatttgacgttggctgtatacaggaacggagatttatgactcgcgtccatgctcattgcaggggcatgctggtacaagggtaatgtgtcctttggctgtgtgaaccgctcgccatgcaggattgtgctggcgagtccgggattgcgtcgcacttggctaattgtagcactaaaacgctttttacagtaaaatacgaccacctggacgactgacacgactacactggtttgatggactgcaggcagaggccgtctgcagatgttattgtgcatcctcgtggatatgggtgttttgttgttcggatgatgtaggcgtccggatgaggtgatggctcgtggggacagattacaaatgtcgttggtgcatattttttagtatcgcgatgatggtttggagcgaaacgtattgtcgccagtgcaatatatacacgcgagccaccgcgtaagtagtgaggatcctcggccatacctttcttatatcgaacccctccattgtgtcatcaccttttggccacgaaatacacagatttccatattttggtgctatctatatgatgagttaagtccctgatgccgtctttttgacgtccgaggagttggtacgtgacgggcaagtgacagctatcaaaaactttcgatggtagcttttgtaatcaccggtcgccgcac

SEQ ID NO: 39 - DNA, 1341 bp, Arabidopsis thaliana
ctctataaacacacactctcaggagagaagttgtattgatcgtcttctctttccctaaacacactgattattttctctccgacgccgccatgtctctgctctcagatctcgttaacctcaacctcaccgatgccaccgggaaaatcatcgccgaatacatatggatcggtggatctggaatggatatcagaagcaaagccaggacactaccaggaccagtgactgatccatcaaagcttcccaagtggaactacgacggatccagcaccggtcaggctgctggagaagacagtgaagtcattctataccctcaggcaatattcaaggatcccttcaggaaaggcaacaacatcctggtgatgtgtgatgcttacacaccagctggtgatcctattccaaccaacaagaggcacaacgctgctaagatcttcagccaccccgacgttgccaaggaggagccttggtatgggattgagcaagaatacactttgatgcaaaaggatgtgaactggccaattggttggcctgttggtggctaccctggccctcagggaccttactactgtggtgtgggagctgacaaagccattggtcgtgacattgtggatgctcactacaaggcctgtctttacgccggtattggtatttctggtatcaatggagaagtcatgccaggccagtgggagttccaagtcggccctgttgagggtattagttctggtgatcaagtctgggttgctcgataccttctcgagaggatcactgagatctctggtgtaattgtcagcttcgacccgaaaccagtcccgggtgactggaatggagctggagctcactgcaactacagcactaagacaatgagaaacgatggaggattagaagtgatcaagaaagcgatagggaagcttcagctgaaacacaaagaacacattgctgcttacggtgaaggaaacgagcgtcgtctcactggaaagcacgaaaccgcagacatcaacacattctcttggggagtcgcgaaccgtggagcgtcagtgagagtgggacgtgacacagagaaggaaggtaaagggtacttcgaagacagaaggccagcttctaacatggatccttacgttgtcacctccatgatcgctgagacgaccatactcggttgatgacacatttcatgatttgatttctctccaatttggttttttttttttcccttttgattgcacttttcgataataaaaaaataattcttattatgggcgtattgttgtgacattttgtgttttgtttcgaataattaaataagcgcttcttaaggtgaaaataaataataattagtgatttttaatc

SEQ ID NO: 40 - DNA, 1494 bp, Arabidopsis thaliana
tgtggagagccaaaaagtctccaaagtcttcacgtcaccctcttcctcaatctctgcacccacccctcctccttctataagtactactcttcatatctctctctaccaaaatatcaaaacacgagacagatttgattccatttttattactgttactatcatccaaacccttggtatttgtagccatgagtcttgtttcagatctcatcaaccttaacctctcagactccactgacaaaatcattgctgaatacatatgggttggtggttctggaatggacatgagaagcaaagccaggactctacctggaccagtgactgacccttcgcagctaccaaagtggaactatgatggttcaagcacaggccaagctcctggtgaagacagtgaagtcatcttataccctcaagccatattcaaggatcctttccgtagaggaaacaacattcttgtcatgtgcgatgcgtacactcccgcgggtgaaccaatcccgactaacaaaagacacgctgcggctaaggtctttagcaaccctgatgttgcagctgaagtgccatggtatggtattgagcaagaatacactttactccagaaagatgtgaagtggcctgttggttggcctattggtggttatcccggccctcagggaccgtactattgcggtattggagcagacaaatcttttggcagagatgttgttgattctcactacaaggcctgcttatacgctgggatcaacattagtggcatcaatggagaagtcatgccgggtcagtgggagttccaggtcggtccagctgttggtatctcggctgctgatgaaatttgggtcgctcgttacattttggagaggatcacagagattgctggtgtagtggtatcttttgacccgaaaccgattcccggtgactggaacggtgctggtgctcactgcaactacagtaccaagtcaatgagggaagaaggcggttacgagatcatcaagaaagcaatcgataaattgggactgagacacaaagaacacattgctgcttacggtgaaggcaatgagcgtcgtctcacaggacaccacgagactgctgacatcaacactttcctttggggtgttgcgaaccgtggagcatcgatccgagtaggacgtgatacggagaaagaagggaaaggatactttgaggacaggaggccagcttcgaacatggatccttacattgtcacttccatgattgcagagactacaatcctctggaatccttgatgatcatcagatcaagaaaaaatcttgaatgtcactcaaatttgtgtttcttgcaagattcaaagtttgtgttctctatcaagcaatgtcttaggataagtcaaagatttgctctgcttattctgctttttatttacttcacatcctattgaaaacatttctgtgtattatttatgaataaacattatcttaaaagggctgatttatttactaatgcatgcattcaccacttaagatc

SEQ ID NO: 41 - DNA, 1317 bp, Brassica napus
ggctcacctcagactgattattataactcgatcgtcatcttcttcggcttgatggaaacagaaaaaatgtctccactctcagatctcctaaacctcaacctcgacaccaagcaaatcatcgctgaatacatatggatcggtgggtctggaatggacattagaagcaaaggcaggacattaccaggaccagtaagtgatccatcaaagcttccgaaatggaactacgatggatccagcaccaatcaagccgccggagatgacagtgaagtcattctatatcctcaggcgatttttaaagacccgttcaggaaagggaataacattctcgtgatgtgtgatgcttacacaccgaaaggagatccaatcccgaccaacaataggcacaaagccgtgaaaatcttcgatcatcccaatgtgaaggctgaagagccttggtttgggatagagcaagaatacacattacttaagaaagacgtcaagtggccattgggttggccccttggtggctttcctggtcctcagggaccgtactattgtgcggtcggtgcagacaaagcctttgggcgtgacattgtggatggtcactacaaagcttgtctttacgctggtttaagcataggtggtgccaatggtgaagtcatgcctggtcaatgggagtttcaaatcagccctactgttggtattggtgcaggtgatcagttatgggttgctcgctacatactcgagaggattactgagatatgcggcgtgattgtctcatttgatcccaaaccaatcgagggtgattggaacggagcagctgctcatacaaacttcagtacaaaatcaatgaggaaagaaggaggattggacttgataaaaaaagcaatagggaagcttgaagtgaagcataaacaacacattgctgcttatggtgaaggcaatgagaggcgcctcactgggaagcatgaaaccgcagacatcaacaagttctcttggggagttgcggatcgtggagcatcggtgagagtgggaagagatacggagaaagaagggaaaggttattttgaagatcgaagaccttcgtctaatatggatccttatcttgttacctccatgatagctgaaaccaccatcctcggctaagctttcttttgaagttgttgcatacgttcttttgtttcttcatgtttcggtttaatttcggtttgagacttttttttttggtgctaataattcatgggatggtcttgatcctattgtttgtttatcctggttcagttgttagtgttaaacaaaattgaattgggaaaataaaggttcttagttcttactttt

SEQ ID NO: 42 - DNA, 1555 bp, Brassica napus
ttcatatttgtcaactcttcctttgccatttgttgcaaacactcaagtctcctgatatcagagttagagtcttcttcaagttccagggataaaaatggcgcagatcttggcagcttctccaacatgtcaaatgagattgactaaacccagctccattgcatcgtcaaagttatggaactcggttgtgttgaaacagaagaaacagagcagcagcaaagtcagaagcttcaaagtgatggctctccaatctgataacagcacaatcaacagagttgagagtcttctcaatctagacaccaaacctttcactgaccggatcatcgctgagtacatctggattggcggatctggaattgaccttaggagcaagtcaaggacgcttgaaaagcccgtggaagatccttctgaacttcccaagtggaactatgatggttcaagtaccggtcaagcacctggtgaagatagtgaagtgattctctatccgcaagctatcttcagggatcctttccgtggaggcaataacatattggttatctgtgatacctacacaccagctggtgagccaattccaacaaacaaacgtgcaagagctgctgagattttcagcaacaagaaggtcaatgaagagattccatggtttggcattgaacaagagtacactttacttcagccaaacgtgaactggcctttgggttggcccgttggagcgtatcctggtccccagggtccttactactgtggagttggagctgaaaagtcttggggccgtgacatttcagatgctcattacaaagcttgtttgtatgctggaattaacatcagtggtactaatggtgaagttatgccaggacagtgggaattccaagttggcccgagcgtaggaatcgaagcaggtgatcacgtttggtgtgctagataccttcttgagagaatcacagaacaagctggtgttgtcctaacacttgatcccaaaccgattgagggtgactggaacggtgctggttgccataccaattacagcacaaagagcatgagagaggacggaggatttgaggtgattaaaaaggcaatcttgaacctctcgcttcgtcacatggagcacatcagtgcctacggtgaaggcaatgagagaaggttgactggaaagcacgagacagccagtatcgaccaattctcatggggagtggctaaccgtggatgctcaattcgtgtgggacgtgataccgagaagaaaggaaaaggttacttggaagatcggcgtccagcgtctaacatggacccatacattgtgacttcactgttggcagagaccacacttctctgggagccaacccttgaggctgaagcacttgctgctcagaagctttctttaaaagtttaatttattaatgaacacacatgtctgtttatgtggtcttcccgggatcatcagtcttgtttagaacacgtgttcggattacgacattcttgtctcttttttttcatttgcattgtttaaaaaacccagaatttcgtggacaatgttcatccttttctattggttgtttatggtctt

SEQ ID NO: 43 - DNA, 1456 bp, Hordeum vulgare
  misc_feature (1237)..(1237): n is a, c, g, or t
gaattccctccctccctgccctcagtcgtccagccgggttcctccatccctcccgccatggcgctcctcaccgatctcctcaacctcgacctctccggctccacggagaagatcatcgccgagtacatatggatcggcggatctggcatggatctcaggagcaaggccaggcacctccccggcccggtcacccaccccagcaagctgcccaagtggaactacgacggctccagcaccggccaggccccgggcgaggacagcgaggtcatcctgtacccacaggccatcctcaaggacccgttcagggagggaaacaacatccttgtcatgtgcgattgctacaccccacgtggagagccaatccccaccaacaagagatacaacgctgctaagatccttagcaaccccgatgttgccaaggaggagccatggtacggtattgagcaggagtacaccctcctacagaaggacatcaactggcctctcggctggcctgttggtggcttccctggtcctcagggtccctactactgtggtattggtgctgacaagtcgtttgggcgtgacatagttgactcccactacaaggcttgcctctttggcggcgtcaacatcagtggcatcaacggcgaggtcatgcccggacagtgggagttccaagttggcccgactgttggcatttctgctggtgaccaagtgtgggtcgctcgctacattcttgagaggatcaccgagatcgccggagttgtcgtcacgtttgaccccaagcccatcccaggcgactggaacggtgctggtgctcacacgaactacagtaccgagtcgatgaggaatgacggtgggttcaaggtcatcgtggacgcggtcgagaagctcaagctgaagcacaaggagcacatcgcggcctacggcgagggcaacgagcgccgtctgaccggcaagcacgagacggccgacatcaacacctccagctggggtgtggcaaaccgtggcgcgtcggtgcgcgtgggccgggagacggagcagaacggcaagggctacttcgaggaccgccggccggcgtccaacatggacccctacgtggtcacctccatgatcgcccagaccaccatcctgtggaagccctgaagctccgatcgccgtgtgatggaccgtcggtgatggggtccggtggtggccattggaggattcgtgccttgggcgaaaattcttccagcattttccttttacgtgtggntgnatactactcctagtccgcttaggtaggtcacatcatcatggtcatctcatcagggtgtctggtctctcttctcgctctcgtctntgggtgggtggtgggtgatgggtggcaaggggcgtgtcaaagcagattgatatggtaataaaacaagattactacagtatntgggtgattgttaacccttgccgtctggatgctatggtctcgtgtaatctc

SEQ ID NO: 44 - DNA, 1495 bp, Oryza sativa
attgatagcctgtgcgtctccaagaagaggcttgccgctgccgccattggagccctctcgtttctgctcgagctctgcatttcttcagtaggaggaggaggaggaagagttggagtcgccatgtcgtcgtccctgctcactgacctcgttaacctcgacctgtcggagagcacggacaaggtcatcgccgagtacatatgggttggtggtactgggatggatgtgaggagcaaagccagaacgttgtctggacctgttgatgacccaagcaagcttccaaagtggaactttgatggctccagcaccggtcaggctaccggtgacgacagtgaagtcatcctccaccctcaagccatcttcagagacccattcaggaaggggaagaacatcctggtcatgtgtgactgttatgcgccgaatggcgagccgattccgacgaacaaccggtacaatgcagcaaggatcttcagtcatcctgatgtcaaggctgaagagccatggtatgggattgagcaggagtacacccttcttcagaagcacatcaactggcctcttggctggccactaggtggctatccaggccctcagggtccgtactactgtgcggcgggagccgataaatcgtacgggcgcgacatcgttgatgcccactacaaggcctgcctgtttgccggcatcaacatcagcgggatcaacgcagaagtcatgccggggcagtgggagttccagattggccctgtcgttggcgtctccgcaggggatcatgtctgggtggcacgctacattcttgagaggatcactgagattgctggcgtcgtcgtgtccttcgaccccaagcccattccgggagactggaatggcgccggtgctcacaccaactacagcaccaagtcgatgaggagcaatggcggctacgaggtgatcaagaaagcgatcaagaagcttggcatgcgccaccgtgagcacatcgccgcctacggcgacggcaacgagcgccgcctcaccggccgccacgagaccgccgacatcaacaacttcgtctggggcgtagcgaaccgcggcgcgtcggtgcgtgtcggccgggacaccgagaaggacggcaaaggttacttcgaggacaggaggccggcgtccaacatggacccgtacctggtgaccgccatgatcgccgagaccaccatcctctgggagcccagccacggccacggccacggccaatccaacggcaagtgaggaggagtcgcctcgcccgggttgatgaactgctttctcgcgttctgggtttcatggaaatctgtgtgtgtgtgttctctgacgctggtgctgttagaaacttccaataattcagaaataactgcgatgtgctctcaaatttctcatgaggccatcacctgcagcatctcatgaaatagatctattgcaatgacaataccaatggcaacgcaaaattttatggtacctccagataccatctactctcctcaataatgacaat

SEQ ID NO: 45 - DNA, 1677 bp, Oryza sativa
atcgacgtcgcctcctctcctcctcctcctcgtcgctgcattccggttgagtgagttggtgattatctgtagggggtgaaaatggcgcaggcggtggtgccggcgatgcagtgccaggtcggggccgtgcgggcgaggccggcggcggctgcggcggcggcgggggggagggtgtggggagtcaggaggaccgggcgcggcacgtcggggttcagggtgatggccgtgagcacggagaccaccggggtggtgacgcggatggagcagctgctcaacatggacaccacccccttcaccgacaagatcatcgccgagtacatctgggttggaggaactggaattgacctcagaagcaaatcaaggacaatatcaaaaccagtggaggacccctcggagctaccaaaatggaactacgatggatcaagcacagggcaagctccaggagaagatagtgaagtcatcttatacccacaggctatattcaaggacccatttcgaggtggcaacaacatattggttatgtgtgatacctacacaccagctggggaacccatccctactaacaaacgtaacagggctgcacaagtattcagtgatccaaaggttgtcagccaagtgccatggtttggaatagaacaggagtacactttgctccagagagacgtaaactggcctcttggctggcccgttggaggctaccctgggccccagggtccatactactgcgctgtaggatcggacaaatcgtttggccgtgacatatcagatgctcactacaaggcatgtctttatgctggaattaacattagtggaacaaatggagaggtcatgcctggtcagtgggagtaccaggttggacctagtgtcggtattgaagctggagaccacatatggatttcaagatatattcttgagagaataacggagcaggctggtgtagtgcttacccttgaccccaaaccaattcagggagactggaatggagctgggtgccacacaaactacagcaccaagagtatgcgtgaagatggaggatttgaggtgatcaagaaggcaatcctaaacctatcacttcgccatgacttgcatataagtgcatatggtgaaggaaatgaaaggaggttgacaggtttacacgagacagctagcattgacaatttctcatggggtgtggcaaaccgtggatgctctattcgggtggggcgagacaccgaggcgaagggaaaaggctacttggaagaccgtcgcccggcatcaaacatggacccgtacgtcgtgacagcgctattggctgaaaccacaattctttgggagccaaccctcgaagcggaggttcttgctgctaagaagttggccctgaaggtatgaagaacttggacgatgaatcggggcaaataaatcccagcaaaatttgtttgctgcccaccagtcttgatcttgtatttcttctgtctggggattggtctgtacaaatctgcagtttctagaaaaccacgccaccttccattcgccagttaacattttggttgaacaccacacttgatctgggtctgtattttgagtccatttgtgagtgacagaacggatgatgaaacacatcagggacacttttaagtttcttcagtcctgcgtccttccctcgaaataaaaatgtttccttgttttttatcccgggct

SEQ ID NO: 46 - DNA, 1041 bp, Physcomitrella patens
atggccttggcacagaaggcagagtacatctggatggatggacaggagggtcagaaagggatccgcttcaacgaaatgcgatccaagaccaaggtgatccaggagcccatcaaggccggatctttggacttccccaagtggtcattcgacggttccagcactgggcaagcagaggggcgattctccgactgtatcctgaaccccgtgtttagctgccttgaccccatccgcggggacaaccacgtgctggttctgtgtgaggtgttgaaccccgacagcacaccccatgaaaccaacacccggcgcaagatcgaggaattgttgaccccggatgtgctggcagaggagacactgttcggatttgagcaggagtatacgatgttcaacaaggccggaaaggtatacgggtggccagaaggaggtttcccacacccacagggccccttctactgtggagtgggtctggaggcggtttacgggcgacctctggtggaggcgcacatggatgcgtgcatcaaggctgggctgaagatcagtggtatcaatgccgaggtcatgccgggacagtgggagttccagatcggccccgctggacctttggaagtgggtgaccacgtcatgatcgcacgttggttgcttcaccgcttgggtgaggacttcggcattacttgcacgttcgagcccaagcccatggaaggtgactggaatggtgctggagctcacaccaactactcgacgaagtcaatgagggtggacggcggtatcaaggccatccacgccgccattgagaagttgtccaagaagcacgtggagcacatctcctcatacgggttgggcaatgagcgtcgtctgactggaaagcacgagactgccaacatcaacactttcaaatcgggggtcgcagacagaggtgcatcgatccgtatccctcttggagtgtctcttgacggcaagggttatttggaggatcgcagacccgcggcgaatgtggacccttacgtggtggcacgcatgctgatccagacgactttgaagaactag

SEQ ID NO: 47 - DNA, 1041 bp, Physcomitrella patens
atggccttggcacagaaggcagagtacatctggatggatggacaggagggtcagaaagggatccgctttaacgaaatgcgatccaagaccaaggtgatccaggagcccatcaaggccggatctttggacttccccaagtggtctttcgatggttctagcactgggcaagcagaagggcgattctccgactgcattctgaaccccgtgttcagctgccccgaccccatccgcggggacaaccacgtgctggttctgtgcgaggtgttgaaccccgacagcacaccccatgaaaccaacacccggcgcaagatcgaggaactattgaccccggatgtgctggcagaggagacactgttcggatttgagcaggagtacaccatgttcaacaaggccgcgaaggtgtacgggtggccagagggaggtttcccacacccacaagggcccttttactgtggagtgggtcttgaggcggtttacgggcgacctctggtggaggcgcacatggatgcgtgcatcaaggccgggctgaagatcagtggtattaatgccgaggtgatgccgggacagtgggagttccagatcggccccgctggacctctggaggtgggtgaccacgtcatggtcgcgcgttggctgcttcaccgcttgggtgaggactttggcattacttgcactttcgagcccaagcccatggaaggagactggaacggtgctggagctcacaccaactactcgacgaagtcgatgagggtggacggcggtatcaaggccatccacgcggccattgagaagctgtccaagaagcacgcggagcacatctcctcatacgggttgggcaatgagcgtcgtctgacaggcaagcacgagaccgccaacatcaacacattcaagtcgggagttgcggacagaggtgcgtcgatccgtattccgcttggagtgtccctggagggcaaaggttacttggaagaccgtaggccagcggcgaacgtggacccttacgtagtggcccgcatgcttatccaaacgactttgaagaactag

SEQ ID NO: 48 - DNA, 1584 bp, Pinus taeda
ttcctttgccttaaaaaatagaggtttcttaataccccgtcttcgttcattggtttctataaattcttcctcaggttggggttgctctttgcatcaattgctataaattcttatttcagtggcctttatttcgaaatagcagatcaaaggccttcactgcttgcagaattatacttgtgcgggagcctgtgattttgtggtacatccaagatgtctctactgacggatttgatcaacttggatctctctgatgtcactgagaagatcatcgctgagtacatatggatcggaggctctggcatggatatccgcagcaaggccaggaccttatctcacccagttacggaccccaaagatctacccaagtggaattatgatggatccagtactggacaggctcctggaaaggacagtgaagtcatcctttaccctcaggctatcttcagggatccattccgcaggggtaacaacatcttggtgatttgtgatacatataccccagctggagaacctattcctactaacaagagagcaaatgctgctaaaatatttagccatccagatgttgttgccgaggaaccatggtacgggattgaacaagaatacactcttctgcaaaaggatgtgaattggccgcttggatggcccgtaggtggttaccctggtcctcagggtccttattattgtggaactggagcagacaaagcctacggccgtgatattgtcgatgcccactataaggcttgcctgtatgcaggaatcaacattagtggcatcaatggagaagtcatgcccggtcaatgggaatttcaagttggcccgacggttggtatttcagctggtgatcaagtctgggctgcacgttaccttcttgagagaatcacagaagtggctggtgttgtcctctcatttgaccccaaacccattcagggtgattggaatggtgctggtgctcacactaactacagtacgaaatcaatgagggaagaagggggaattaaagtgatcaaaacggccattgaaaagttagggttgaggcataaggaacacattgctgcctatggagagggcaacgagaggcgtttgactggccgacatgagacagcagacataaacacattttcatggggagttgcaaatcgtggagcttctattcgagttggacgtgacacggaacgtgaaggcaaagggtacttcgaagaccgcaggccagcttccaacatggacccctatatagtaacatctatgattgctgagacaaccatccttttgaagtgagagtaacattgtttactgaatgaataaagatgccgatacgattgaagtgttcttgatgctagtcaaattgcgaagggatccccaattgtttgtggggcatattctcatttgaatttctttatgtgcctaaagtatttcccctatttctgttaataagaacattctggaaataggacttgagatttagggtgctttatattcagtgtctaatttgtctttcagattttcattgttccatgactctgatatgattggtgtgcaattgaatttaatgaattcagaagttcttttattgcttgtgaaaaa

SEQ ID NO: 49 - DNA, 1304 bp, Pinus taeda
tttgtatctcgtttcgtatttcctcactcgcaatccatcttatccccgtatcacaaccacattcacaatggctactcctatcacctcacggacggagactctccagaagtatctcaagcttgatcagaagggtatgatcatggctgagtacgtctgggttgatgccgatggtggcactcgttccaagtctcgcacattgcccgagaaagaatacaagcccgaggatcttcccgtttggaacttcgatggttcttccactaaccaggcccctggtgacaactccgatgtctacctccgtccctgcgccgtctaccctgatcccttccgcggctctcccaacatcattgttcttgctgagtgctggaacgccgatggcactcccaacaaatacaacttccgtcacgattgcgtgaaggtcatggacacctacgccgacgacgagccttggtttggcctcgagcaggagtataccctcctcggctctgacaaccgaccctatggctggcccgccggtggtttccctgctccccaaggcgagtactactgtggtgtgggcactggaaaggttgtccagcgcgatatcgtcgaggcccattataaagcctgtttgtacgccggcatccagatctctggaacgaacgccgaggtcatgcctgctcagtgggaatatcaggtcggcccctgcactggcattgcaatgggcgaccaactctggatttcgcgattctttttacatcgagtcgctgaggaattcggtgcaaaggtttctttgcaccccaagcccattgctggcgattggaacggagctttaagtttccctggtctctgtttcatatccgtgatactaatatctttacagggtttgcactccaacttctccacgaaagcaatgcgcgaggagggtggtatgaaggttattgaggaggccctgaagaagcttgaacctcaccacgtcgagtgtatcgcagagtatggtgaggataacgaattgcgtttgaccggccgtcacgagacgggatccatcgacagcttttcttggggtgtcgccaaccgtggcacaagcatccgcgtgccacgcgaaacggctgctaagggctatggctactttgaggaccgccgtcctgcttccaacgccgatccctaccgcgttaccaaggttctcctccaattttctatggcttagagcgagttttagagtttttgctttctgatgacatggtctacggcgtgaaggtttgggaaactattgattacatagatagcatgaaagcttgtcctgaaggacagtaatgacaaccaatcag

SEQ ID NO: 50 - DNA, 1251 bp, Phaedactylum tricornutum
atgaaattaaacattgctgctattgcgctatttgctgcatcggcttcggcctttgctcctcgatttgcgtcgcctcgctcccacgctaccgtactgtccgcggtcctcgaagaacgaacggggcagtctcagctcgaccctgccgtcatcgagcgatacgctgcgcttccctacccggatgataccgttcttgccgaatatgtatgggtcgatgccgtgggtaacacgcgctccaagacacgcacgcttcctgccaagaaggctgcatctgtcgaggctcttcccaagtggaactttgatggctcttcgacggaccaggctcccggagacgactcggaagttattctacgtccttgccgtatcttcaaagatcctttccgacctcgtaacgatggtctcgacaatgttctcgtcatgtgcgattgctacacaccgaacggcgaagcaattcccacgaaccaccgtgccaaggctatggaatcttttgaatccagggaagacgaagagatctggttcgggctcgaacaggaatttacgctgttcaacttggacaagcgtacccctctcggctggccagaaggcggcatgcccaatcgccctcaaggaccttactattgtagtgttggacccgaaaataacttcggacgtcacattacggaatccatgtaccgggcttgtctctacgcaggcatcaacatttcgggaacgaatggagaagtcatgcccggacaacaggaataccaggttggaccctgcgtgggaattgacgcaggggatcagctcatgatgagccgatacattcttcagcgtgtctgcgaggatttccaggtatattgtacactccatcccaagcccatcgttgacggtgactggaacggcgccggcatgcacaccaatgtttctactaaatccatgcgcgaggaaggtggccttgaagttatcaaaaaggcgatttacaagttgggggccaagcaccttgagcacatcgctgtgtacggtgaaggtaacgaacttcgcctgacaggcaagcacgaaacggccagcatggacaagttttgctacggtgttgccaaccgtggagcgtccattcgaattggtcgcgacaccgaagccgaggggaagggatacttcgaggatcgtcgtccgtcatctaacgccgatccttacattgttacgggaaagatcatgaatacaattatggaagatgtggaagtccccgatattgctccaatggacaaggccgtggcctaa

SEQ ID NO: 51 - DNA, 1768 bp, Zea mays
caacgacagcgagccctatcccctcagcaaaagccagatgcctgttgccgtcgcggccactggatgccaagtactttttatatacgccgtccgcgcccacgacccccgagacccgcctcccctcgtcgtctcgtctcgcctcgcgtcgtctgcgctcgcggctcgtcacaggtgaggtctcggcgggagaggggcggcggccggtccgtgtccgtgtccgtcgacggttggttcgggaatggcgcaggcggtggtgccggcgatgcagtgccgggtcggagtgaaggcggcggcggggagggtgtggagcgccggcaggactaggaccggccgcggcggcgcctcgccggggttcaaggtcatggccgtcagcacgggcagcaccggggtggtgccgcgcctcgagcagctgctcaacatggacaccacgccctacaccgacaaggtcatcgccgagtacatctgggtcggaggatctggaatcgacatccgaagcaaatcaaggacgatttcgaaacccgtggaggatccctcggaactaccaaaatggaactacgatggatctagcacaggacaagccccgggagaagacagtgaagtcattctatacccccaggctatcttcaaggacccattccgaggtggcaacaacgttttggttatctgtgacacctacacgccacagggggaaccccttccaactaacaaacgccacagggctgcgcaaattttcagtgacccaaaggtcgctgaacaagtgccatggtttggcatagagcaagagtacactttgctccagaaagatgtaaattggcctcttggttggcctgttggaggcttccctggtccccagggtccatactactgtgccgtaggagccgacaaatcatttggccgtgacatatcagatgctcactacaaggcatgcctctacgctggaatcaacattagtggaacaaacggggaggtcatgcctggtcagtgggagtaccaagttggacctagtgttggtattgaagcaggagatcacatatggatttcgagatacattctcgagagaatcacagagcaagctggggttgtccttacccttgatccaaaaccaattcagggtgactggaacggagctggctgccacacaaattacagcacaaagaccatgcgcgaagacggcgggtttgaagagatcaagagagcaatcctgaacctttctctgcgccatgatctgcatattagtgcatacggagaaggaaatgaaagaagactgactgggaaacatgagactgcgagcatcggaacgttctcatggggtgtggcaaaccgcggctgctctatccgtgtggggcgggataccgaggcaaaagggaaaggttacctggaagaccgtcggccggcatcaaacatggacccgtacattgtgacggggctactggccgagaccacgatcctctggcagccatccctcgaggcggaggctcttgccgccaagaagctggcgctgaaggtgtgaagcagctgaaggatggttcaggcaccaatataaaccggtccgcgacaagattgatctttgtgtccatggcgtgggtcttgcgactctctgctcggcggtgccactctgtacaaaatcacggctgtctttgattcatcggatattcggatacgtttgtttgttactttttgcttggacacccaccatgtttggaacttttttgggctccgtttgggggctgaacgatggtcagtggaaattttaaaaattcgtcgtctc

SEQ ID NO: 52 - DNA, 1531 bp, Zea mays
cacgccacatcctcccctccttcctccttgggttcccagcccgtgcgcccgcctgtcgcagtcgcaccgcagccgccggccatggcctgcctcaccgacctcgtcaacctcaacctctcggacaccacagagaagatcatcgccgagtacatatggatcggtggatctggcatggatctcaggagcaaagccaggaccctcccgggcccggtgaccgatcccagcaagctgcccaagtggaactacgacggctccagcaccggccaggcccccggcgaggacagcgaggtcatcctgtacccgcaggccatcttcaaggacccattcaggaggggcaacaacatccttgtcatgtgcgattgctacaccccagctggcgagccaattcccaccaacaagaggtacagcgccgccaagatcttcagcagccttgaggtcgctgccgaggagccctggtatggtatcgagcaggagtacaccctccttcagaaggacaccaactggcccctcgggtggcctattggcggcttccctggccctcagggtccttactactgtggaatcggcgcggagaaatcgttcgggcgtgacatagtcgacgcccactacaaggcctgcctgtacgcaggcatcaacatcagtggcatcaacggggaggtcatgccggggcagtgggagttccaggtcggaccgtccgtcggcatctcttcgggcgatcaggtgtgggttgctcgctacattcttgagaggatcaccgagatcgccggcgtggtggtgacgttcgacccgaagccgatcccgggcgactggaacggcgcgggcgcccacaccaactacagcaccgagtccatgaggaaggagggcgggtacgaggtgatcaaggcggccatcgagaagctgaagctgcggcacaaggagcacatcgcggcctacggcgagggcaacgagcgccggctcaccggcaggcacgagaccgccgacatcaacaccttcagctggggagtcgccaaccgtggcgcgtcggtgcgcgtgggccgcgagacggagcagaacggcaagggctacttcgaggaccgccggccggcgtccaacatggacccctacgtggtcacctccatgatcgccgagaccaccatcgtctggaagccctgaggcaccccgtggccgtgtcgtgtcggtttgctccgcgtacggcgctggccgttgcatcgcagggcccagcggttgcgcaactattttcccttccccgttctgtttgcttgtactactactctaccgctagtcctgcatagcattttagctagaacacaacaacagccaaaaaaaagtattgttgcttgcttcgacgcttgccaccacttccattccatgccgtccgtccgcttccttcctgtgtaatcctcctccaataatagacgtgccatgttgcatcctctattcctctgcattgtataaaagtggtgtaattcttttgctacgcctccaatgtctgggcttttagctgctgatgcgatgtcagattctgtcacgg

SEQ ID NO: 53 - protein, 354 aa, Hordeum vulgare
MASLADLVNLNLSDCTDKVIVEYLWVGGSGIDIRSKARTVNGPITDASQLPKWNYDGSSTGQAPGEDSEVILYPQAIFKDPFRRGDNILVMCDCYTPQGVPIPTNKRHNAAKIFNSAKVAAEETWYGIEQEYTLLQKDVNWPLGWPIGGYPGPQGPYYCAAGADKAFGRDIVDAHYKACLYAGINISGINGEVMPGQWEFQVGPSVGIAASDQLWVARYILERITEVAGVVLSLDPKPIPGDWNGAGAHTNYSTKSMRQAGGYEVIKKAIEKLGKRHMQHIAAYGEGNERRLTGHHETADINTFKWGVADRGASIRVGRDTEKDGKGYFEDRRPASNMDPYVVTSMIAETTLLL

SEQ ID NO: 54 - protein, 427 aa, Hordeum vulgare
MAQAVVQAMQCQVGVRGRTAVPARQPAGRVWGVRRAARATSGFKVLALGPETTGVIQRMQQLLDMDTTPFTDKIIAEYIWVGGSGIDLRSKSRTISKPVEDPSELPKWNYDGSSTGQAPGEDSEVILYPQAIFKDPFRGGNNILVICDTYTPQGEPIPTNKRHMAAQIFSDPKVTSQVPWFGIEQEYTLMQRDVNWPLGWPVGGYPGPQGPYYCAVGSDKSFGRDISDAHYKACLYAGIEISGTNGEVMPGQWEYQVGPSVGIDAGDHIWASRYILERITEQAGVVLTLDPKPIQGDWNGAGCHTNYSTLSMREDGGFDVIKKAILNLSLRHDLHIAAYGEGNERRLTGLHETASISDFSWGVANRGCSIRVGRDTEAKGKGYLEDRRPASNMDPYTVTALLAETTILWEPTLEAEALAAKKLALKV

SEQ ID NO: 55 - protein, 1455 aa, Hordeum vulgare
GCTCGAGCTGCACACCTCATCTCATCATCGTCTTCCCCCCATTGCCATCGACCTCCCTCCCTGCGAGCAGCAGCAGCAGCAATGGCCAGCCTCGCCGACCTCGTTAATCTCAACCTCAGCGACTGCACGGACAAGGTCATCGTCGAGTACCTCTGGGTTGGAGGATCTGGTATCGACATCAGGAGCAAAGCAAGGACGGTGAACGGACCCATCACCGACGCGAGCCAGCTGCCCAAGTGGAACTACGACGGCTCCAGCACCGGCCAGGCTCCCGGAGAGGACAGCGAAGTCATCCTCTACCCCCAGGCCATTTTCAAGGACCCGTTCAGGAGGGGTGACAACATCCTTGTTATGTGCGACTGCTACACACCACAAGGTGTGCCAATTCCCACTAACAAGAGGCACAATGCTGCCAAGATCTTCAACAGCGCTAAGGTTGCAGCTGAGGAGACATGGTATGGTATTGAGCAGGAGTACACACTCCTCCAGAAGGATGTGAACTGGCCTCTTGGCTGGCCAATTGGTGGCTACCCTGGTCCTCAGGGACCATACTACTGCGCCGCCGGTGCCGACAAGGCGTTCGGGCGTGACATCGTGGACGCCCACTACAAGGCGTGCCTCTACGCCGGGATCAACATCAGCGGCATCAACGGGGAGGTCATGCCCGGCCAGTGGGAGTTCCAAGTTGGGCCGTCCGTCGGGATCGCCGCCTCCGACCAGCTGTGGGTGGCGCGCTACATCCTCGAGAGGATCACAGAGGTTGCCGGGGTGGTGCTGTCCCTGGACCCGAAGCCGATCCCGGGTGACTGGAACGGCGCGGGCGCGCACACCAACTACAGCACCAAGTCCATGAGGCAGGCCGGCGGCTACGAGGTGATCAAGAAGGCCATCGAGAAGCTTGGCAAGCGCCACATGCAGCACATCGCCGCCTACGGCGAGGGCAACGAGCGCCGCCTCACCGGCCACCACGAGACCGCCGACATCAACACCTTCAAATGGGGCGTGGCGGACCGCGGCGCGTCCATCCGCGTGGGGCGCGACACGGAGAAGGACGGCAAGGGCTACTTCGAGGACCGCAGGCCGGCCTCCAACATGGACCCCTACGTCGTCACCTCCATGATCGCCGAGACCACGCTTCTCCTCTGAGCACACGGCCGGCAATGCCTACTCCACCGCCAGATGACACTTTGGGCAGGCTCTCGTCTCGACTCTCTCGATCGAGGGTGGTGATTGATTTCTGCAAAACATTTCCCGTTTCCGTTTCTTTTGCAATTGCAAGGTCTAGTCTGTTTTTGGGGCGTGCCTTTGGTATCTTTCATAGTAGTACGTCTACTGCTCTTCAGGATAAGAAGAGTCTTCAGTGTACTCTGAAAATAATGTTGTTTCCGCATTCTGATAAAATGGAATCATGGAACCGGTTGTGATTCTGTCTGTTCAAAAAAAAAAAAAAAAAAA

SEQ ID NO: 56 - DNA, 1775 bp, Hordeum vulgare
  misc_feature (1724)..(1724): n is a, c, g, or t
tcgcccctctcctccctcgccccctcgcctcgctcctctcgcccgcgtcgctgtctctggtttcggggcggcggagtcgctgtacgtaagtaagtaagtacgtagagacgacgatggcgcaggcggttgtgcaggcgatgcagtgccaggtgggggtgaggggcaggacggccgtcccggcgaggcagcccgcgggcagggtgtggggcgtcaggagggccgcccgcgccacctccgggttcaaggtgctggcgctcggcccggagaccaccggggtcatccagaggatgcagcagctgctcgacatggacaccacgcccttcaccgacaagatcatcgccgagtacatctgggttggaggatctggaattgacctcagaagcaaatcaaggacgatttcgaagccagtggaggacccgtcagagctgccgaaatggaactacgacggatcgagcacggggcaggctcctggggaagacagtgaagtcatcctatacccacaggccatattcaaggacccattccgaggaggcaacaacatactggttatctgtgacacctacacaccacagggggaacccatccctactaacaaacgccacatggctgcacaaatcttcagtgaccccaaggtcacttcacaagtgccatggttcggaatcgaacaggagtacactctgatgcagagggatgtgaactggcctcttggctggcctgttggagggtaccctggcccccagggtccatactactgcgccgtaggatcagacaagtcatttggccgtgacatatcagatgctcactacaaggcgtgcctttacgctggaattgaaatcagtggaacaaacggggaggtcatgcctggtcagtgggagtaccaggttggacccagcgttggtattgatgcaggagaccacatatgggcttccagatacattctcgagagaatcacggagcaagctggtgtggtgctcacccttgacccaaaaccaatccagggtgactggaacggagctggctgccacacaaactacagcacattgagcatgcgcgaggatggaggtttcgacgtgatcaagaaggcaatcctgaacctttcacttcgccatgacttgcacatagccgcatatggtgaaggaaacgagcggaggttgacagggctacacgagacagctagcatatcagacttctcatggggtgtggcgaaccgtggctgctctattcgtgtggggcgagacaccgaggcgaagggcaaaggatacctggaggaccgtcgcccggcctccaacatggacccgtacaccgtgacggcgctgctggccgagaccacgatcctgtgggagccgaccctcgaggcggaggccctcgctgccaagaagctggcgctgaaggtatgaaggacctgaaaaaaggacgaattcttcttccggggaaaagaaaataaatcggcgagcggcgagaccgttggccgtccattcttgttgatcctgtggttccgtcggggcactgcctgtacaaaatcctcacagtttgtagaaccactcccgcgtgtgtttttccgcttgaactgagtccatttgatctgttgggactgtacactcactgtacctgagtccatatggagaactacgttattataaaacgataatgaatcgcaaaaaaaaaaaaaaaaaaagtcacaaaacagaaaaaaaaaaactcaagggggggcccgggccccagtnccgcctatcggaggtctgtgtacatccattggccccccctgcacaaccccc

SEQ ID NO: 57 - DNA, 1428 bp, Arabidopsis thaliana
atggagcattctagtgatttgactgttgaagctatgatgcttgactctaaagcttctgatcttgacaaagaagaacgtcctgaggtactctctttaatcccaccatatgaagggaaatctgtgcttgaacttggagctggtattggtcgtttcactggtgaattggctcaaaaggctggtgaagttatcgctcttgacatcatcgaaagcgcgattcagaagaatgaaagtgttaatgggcattacaagaacatcaagtttatgtgtgctgatgtaacatctccagacttgaaaatcaaagatggatctatcgacttgattttctcaaactggttgctcatgtatctctctgataaagaggtggaactaatggcagagagaatgattggatgggtcaagccagggggatacattttcttcagagaatcttgcttccatcaatctggggacagcaagcgaaagtcaaaccccactcactaccgtgaacccagattctacacaaaggttttccaggaatgtcagacacgtgatgcttctggcaattcatttgagctctctatggttggctgcaaatgcattggggcttatgtgaagaacaagaagaatcagaatcagatttgctggatatggcaaaaagtcagcgtggagagtgacaaggatttccagcgtgtcttggacaatgttcaatacaagtctagtgggatcttgcgctatgagcgtgtctttggggaaggatatgtgagcactggtggatttgagacaactaaagaatttgtggcgaagatggaccttaaaccgggacagaaagtcctagatgttggttgtggtatcggtggaggtgacttctacatggctgagaatttcgatgttcatgttgttggaatcgatctgtcggtcaacatgatctctttcgcactggagcgggccattggactcaaatgctcagtcgagtttgaagtcgctgattgcaccaccaaaacatatcccgataattcctttgatgtcatttacagccgtgacactattctgcacatccaagacaagccagctctattcaggacattcttcaagtggcttaaaccagggggtaaagttctcatcactgactattgtagaagtgctgaaactccgtctcctgaattcgcagagtacataaaacaaagaggatatgatctacatgatgttcaagcttacggacagatgctgaaagacgcaggctttgacgacgttatcgctgaggaccgtactgatcagtttgtacaagtcctcaggcgtgaattagaaaaagtggagaaagaaaaggaagaattcatcagcgacttctcagaagaggattacaatgacattgttggaggatggtcggcaaagcttgaaaggactgcatctggtgaacagaaatggggattattcatagccgacaagaagtaa

SEQ ID NO: 58 - protein, 475 aa, Arabidopsis thaliana
MEHSSDLTVEAMMLDSKASDLDKEERPEVLSLIPPYEGKSVLELGAGIGRFTGELAQKAGEVIALDIIESAIQKNESVNGHYKNIKFMCADVTSPDLKIKDGSIDLIFSNWLLMYLSDKEVELMAERMIGWVKPGGYIFFRESCFHQSGDSKRKSNPTHYREPRFYTKVFQECQTRDASGNSFELSMVGCKCIGAYVKNKKNQNQICWIWQKVSVESDKDFQRVLDNVQYKSSGILRYERVFGEGYVSTGGFETTKEFVAKMDLKPGQKVLDVGCGIGGGDFYMAENFDVHVVGIDLSVNMISFALERAIGLKCSVEFEVADCTTKTYPDNSFDVIYSRDTILHIQDKPALFRTFFKWLKPGGKVLITDYCRSAETPSPEFAEYIKQRGYDLHDVQAYGQMLKDAGFDDVIAEDRTDQFVQVLRRELEKVEKEKEEFISDFSEEDYNDIVGGWSAKLERTASGEQKWGLFIADKK

SEQ ID NO: 59 - DNA, 1428 bp, Arabidopsis thaliana
atggagcattctagtgatttgactgttgaagctatgatgcttgactctaaagcttctgatcttgacaaagaagaacgtcctgaggtactctctttaatcccaccatatgaagggaaatctgtgcttgaacttggagctggtattggtcgtttcactggtgaattggctcaaaaggctggtgaagttatcgctcttgacttcatcgaaagcgcgattcagaagaatgaaagtgttaatgggcattacaagaacatcaagtttatgtgtgctgatgtaacatctccagacttgaaaatcaaagatggatctatcgacttgattttctcaaactggttgctcatgtatctctctgataaagaggtggaactaatggcagagagaatgattggatgggtcaagccagggggatacattttcttcagagaatcttgcttccatcaatctggggacagcaagcgaaagtcaaaccccactcactaccgtgaacccagattctacacaaaggttttccaggaatgtcagacacgtgatgcttctggcaattcatttgagctctctatggttggctgcaaatgcattggggcttatgtgaagaacaagaagaatcagaatcagatttgctggatatggcaaaaagtcagcgtggagaatgacaaggatttccagcgtttcttggacaatgttcaatacaagtctagtgggatcttgcgctatgagcgtgtctttggggaaggatatgtgagcactggtggatttgagacaactaaagaatttgtggcgaagatggaccttaaaccgggacagaaagtcctagatgttggttgtggtatcggtggaggtgacttctacatggctgagaatttcgatgttcatgttgttggaatcgatctgtcggtcaacatgatctctttcgcactggagcgggccattggactcaaatgctcagtcgagtttgaagtcgctgattgcaccaccaaaacatatcccgataattcctttgatgtcatttacagccgtgacactattctgcacatccaagacaagccagctctattcaggacattcttcaagtggcttaaaccagggggtaaagttctcatcactgactattgcagaagtgctgaaactccgtctcctgaattcgcagagtacataaaacaaagaggatatgatctacatgatgttcaagcttacggacagatgctgaaagacgcaggctttgacgacgttatcgctgaggaccgtactgatcagtttgtacaagtcctcaggcgtgaattagaaaaagtggagaaagaaaaggaagaattcatcagcgacttctcagaagaggattacaatgacattgttggaggatggtcggcaaagcttgaaaggactgcatctggtgaacagaaatggggattattcatagccgacaagaagtaa

SEQ ID NO: 60 - protein, 475 aa, Arabidopsis thaliana
MEHSSDLTVEAMMLDSKASDLDKEERPEVLSLIPPYEGKSVLELGAGIGRFTGELAQKAGEVIALDFIESAIQKNESVNGHYKNIKFMCADVTSPDLKIKDGSIDLIFSNWLLMYLSDKEVELMAERMIGWVKPGGYIFFRESCFHQSGDSKRKSNPTHYREPRFYTKVFQECQTRDASGNSFELSMVGCKCIGAYVKNKKNQNQICWIWQKVSVENDKDFQRFLDNVQYKSSGILRYERVFGEGYVSTGGFETTKEFVAKMDLKPGQKVLDVGCGIGGGDFYMAENFDVHVVGIDLSVNMISFALERAIGLKCSVEFEVADCTTKTYPDNSFDVIYSRDTILHIQDKPALFRTFFKWLKPGGKVLITDYCRSAETPSPEFAEYIKQRGYDLHDVQAYGQMLKDAGFDDVIAEDRTDQFVQVLRRELEKVEKEKEEFISDFSEEDYNDIVGGWSAKLERTASGEQKWGLFIADKK

SEQ ID NO: 61 - DNA, 1668 bp, Arabidopsis thaliana
atggtcggcgaagaagatagcgagagagctcagtccagtaagatggagatcgagcgagaatcgaatttgggatctgcgagtgtgctgatgcagtcgaaggtcatctccgtctcgaatttcttctctattcatagatttcattaccctcgtgaaaaaatcgtctcttttttgtttcctagtgtgttctcaaggataatggcttcgtatggcgaggagcgtgaaatccaaaagaattactggaaagagcattcagtgggattgagtgttgaagctatgatgcttgattccaaagcttctgacctcgacaaagaagaacgtcctgagatacttgcgtttcttccacctattgaagggacaacagtgctagagtttggtgctggaattggtcgttttactactgaattagctcagaaggccggccaggtcattgcggttgacttcattgaaagtgttatcaaaaagaatgagaacattaacggtcactacaagaacgtcaaatttctgtgcgctgatgtcacatcaccaaatatgaactttccaaatgagtctatggatctgatattctccaactggctgctaatgtatctctctgatcaagaggttgaagatttggcgaaaaagatgttacaatggacaaaggttggcgggtatattttctttcgggagtcttgtttccatcagtctggtgataacaagcggaagtacaacccaacacactaccgtgaacctaaattttacacaaagcttttcaaagaatgccatatgaatgacgaagatgggaattcgtatgaactctctttggttagctgtaaatgcattggagcttatgtgagaaacaaaaagaaccagaaccagatatgctggctttggcagaaagtcagttcggataatgataggggcttccaacgcttcttggacaatgtccagtataagtctagtggtatcttacgctatgagcgtgtctttggagaagggtttgttagcacagggggactcgagacaacaaaggaattcgtggatatgctggatctgaaacctggccaaaaagttctagacgttgggtgcggaataggaggaggggacttctacatggctgagaactttgacgtggatgttgtgggcattgatctatctgtaaacatgatctcttttgcgcttgaacacgcaataggactcaaatgctctgtagaattcgaagtagctgattgcaccaagaaggagtatcctgataacacctttgatgttatttatagcagagacaccattctacatatccaagacaagccagcattgttcagaagattctacaaatggttgaagccgggagggaaagttctcatcactgattactgcagaagccccaaaaccccatctccagactttgcaatctacatcaagaaacgaggttatgatcttcatgatgtacaagcatacggtcagatgctgagagatgctggtttcgaggaggtaatcgcggaggatagaaccgatcagttcatgaaagtcctgaaacgggaactggatgcagtggagaaggagaaggaagaattcatcagtgacttctcgaaagaggattacgaggatattataggcgggtggaagtcaaagctacttaggagctcaagtggtgagcagaagtggggtttgttcatcgccaagagaaactga

SEQ ID NO: 62 - protein, 555 aa, Arabidopsis thaliana
MVGEEDSERAQSSKMEIERESNLGSASVLMQSKVISVSNFFSIHRFHYPREKIVSFLFPSVFSRIMASYGEEREIQKNYWKEHSVGLSVEAMMLDSKASDLDKEERPEILAFLPPIEGTTVLEFGAGIGRFTTELAQKAGQVIAVDFIESVIKKNENINGHYKNVKFLCADVTSPNMNFPNESMDLIFSNWLLMYLSDQEVEDLAKKMLQWTKVGGYIFFRESCFHQSGDNKRKYNPTHYREPKFYTKLFKECHMNDEDGNSYELSLVSCKCIGAYVRNKKNQNQICWLWQKVSSDNDRGFQRFLDNVQYKSSGILRYERVFGEGFVSTGGLETTKEFVDMLDLKPGQKVLDVGCGIGGGDFYMAENFDVDVVGIDLSVNMISFALEHAIGLKCSVEFEVADCTKKEYPDNTFDVIYSRDTILHIQDKPALFRRFYKWLKPGGKVLITDYCRSPKTPSPDFAIYIKKRGYDLHDVQAYGQMLRDAGFEEVIAEDRTDQFMKVLKRELDAVEKEKEEFISDFSKEDYEDIIGGWKSKLLRSSSGEQKWGLFIAKRN

SEQ ID NO: 63 - DNA, 1476 bp, Arabidopsis thaliana
atggctgcatcgtacgaagaagagcgtgatattcagaagaattactggatagagcattccgctgatctgactgttgaagctatgatgcttgactcgagagcttctgatctcgacaaggaagaacgtcctgaggtactctctttgctccctccatatgaaggcaaatcagtgttggaacttggagctggtattggtcgtttcactggtgaattagctcaaaaggctggtgaactcattgctcttgacttcattgataacgttatcaagaagaatgaaagtatcaatgggcattacaagaatgtcaagtttatgtgtgctgatgttacatcccctgacctcaagatcactgatggatctcttgacttgattttctccaactggctgctcatgtatctttctgacaaagaggtggagcttttggcagaaaggatggtcggttggatcaaggttggaggatacattttcttccgtgaatcttgcttccaccaatcaggggacagtaagcggaaatccaaccccactcactaccgtgaaccccgtttctattccaaggtctttcaagagtgtcagactcgggatgctgctggaaattcatttgagctctctatgatcggatgcaagtgcattggagcttatgtcaagaacaagaagaatcagaatcagatttgttggatatggcagaaggtcagctcagaaaatgacagaggcttccaacgtttcttggacaatgtccaatacaaatccagtggaatcctacgctatgagcgtgtctttggccaagggtttgtgagcactggtggacttgagacaaccaaagaatttgtggagaaaatgaatctgaaaccaggacagaaagtcttagatgttgggtgtggcattggtggaggtgacttctacatggctgagaagtttgatgttcacgttgttggtatcgatctttctgtcaacatgatctctttcgcattggaacgtgctattggactcagctgctcggttgagtttgaggttgctgattgcaccacaaaacactacccagataattcgtttgatgtcatttacagccgtgacactattctgcacatccaagacaaaccagccttgtttaggactttcttcaaatggcttaaaccgggaggtaaagttctcatcagcgactactgtagaagccccaaaactccatctgctgagttttcagagtacatcaaacagagaggatatgatctccatgacgttcaagcttatggacagatgctaaaagacgctggcttcactgatgtgatcgcagaggaccgtactgatcagtttatgcaagtcctgaaacgtgaattagacagggtggagaaagaaaaggaaaaattcatctccgacttctccaaagaggattacgatgacattgttggaggatggaagtcaaagctggagaggtgtgcatcggatgagcagaaatggggacttttcatcgccaacaagaattaa

SEQ ID NO: 64 - protein, 491 aa, Arabidopsis thaliana
MAASYEEERDIQKNYWIEHSADLTVEAMMLDSRASDLDKEERPEVLSLLPPYEGKSVLELGAGIGRFTGELAQKAGELIALDFIDNVIKKNESINGHYKNVKFMCADVTSPDLKITDGSLDLIFSNWLLMYLSDKEVELLAERMVGWIKVGGYIFFRESCFHQSGDSKRKSNPTHYREPRFYSKVFQECQTRDAAGNSFELSMIGCKCIGAYVKNKKNQNQICWIWQKVSSENDRGFQRFLDNVQYKSSGILRYERVFGQGFVSTGGLETTKEFVEKMNLKPGQKVLDVGCGIGGGDFYMAEKFDVHVVGIDLSVNMISFALERAIGLSCSVEFEVADCTTKHYPDNSFDVIYSRDTILHIQDKPALFRTFFKWLKPGGKVLISDYCRSPKTPSAEFSEYIKQRGYDLHDVQAYGQMLKDAGFTDVIAEDRTDQFMQVLKRELDRVEKEKEKFISDFSKEDYDDIVGGWKSKLERCASDEQKWGLFIANKN

SEQ ID NO: 65 - DNA, 1500 bp, Oryza sativa
atggacgccgcggccgccaccgctgttaatggagtgcttgaggtggaggagaggaaggcgcagaagagctactgggaggagcactccaaggacctcaccgtcgaggccatgatgctcgactcccgcgccgccgatctcgacaaggaggagcgccccgagatattgtctttacttcctccttacgaaggaaaatcagtactggaacttggtgctggaataggtcgcttcactggagaactagtgaaaacagctgggcatgttcttgcaatggatttcattgaaagtgtgattaagaagaatgaaagcataaacggtcaccacaagaatgcatcctttatgtgtgcggatgtcacatgtccagacctgatgattgaggataactccattgatctgatattttcaaactggttactgatgtatctttcagacgaggaggttgagaagctagtaaagagaatggtaagatggctaaaggttggcggctatatcttctttagggaatcttgtttccatcagtctggagattcaaaaaggaaagtgaatcctacacattaccgggagccaaggttttacactaaggtgtttaaagagtgtcaagctcttgatcaagatgggaattcctttgaactctctgtacttacttgcaagtgtgttggagcttacgtgaaaagcaagaaaaatcaaaaccagatatgttggctatggcaaaaggttgattcaacagaagatcgggggtttcaaagatttttggacaatgtgcagtacaaagccagtggaatattacgctatgaacgcatctttggagaaggctttgtgagcactggtggaattgaaactacaaaagaatttgtggacaggctggatctcaaacctggccagaacgttcttgatgttggatgtggaattgggggcggtgatttttatatggctgacaagtatgatgttcatgttgttggtattgatctttcgataaacatggtttcttttgcacttgagcgtgctattgggcgtaagtgctcagttgagtttgaagtcgctgattgcaccaaaaagacatacccagacaacacgtttgacgtcatctacagtcgtgatactatccttcacatacaagataaaccctcactatttaaaagtttcttcaagtggctcaaacctgggggtaaggtcctaattagtgattactgcaagtgccctgggaaaccttcagaagagttcgcagcttacattaagcaaaggggttatgaccttcacgacgtcagggcttacggacagatgcttgagaatgctggtttccatgatgtcattgctgaagaccgcaccgatcagttcctcgatgttctagagagggagcttgctaaagttgaaaagaacaaaaacgagttcgtctctgatttcagccaggaggactacgacgccattgtgaatggatggaaggcaaaacttcaaaggagttctgctggtgagcagaggtgggggctgttcatcgcgaccaagtga

SEQ ID NO: 66 - protein, 499 aa, Oryza sativa
MDAAAATAVNGVLEVEERKAQKSYWEEHSKDLTVEAMMLDSRAADLDKEERPEILSLLPPYEGKSVLELGAGIGRFTGELVKTAGHVLAMDFIESVIKKNESINGHHKNASFMCADVTCPDLMIEDNSIDLIFSNWLLMYLSDEEVEKLVKRMVRWLKVGGYIFFRESCFHQSGDSKRKVNPTHYREPRFYTKVFKECQALDQDGNSFELSVLTCKCVGAYVKSKKNQNQICWLWQKVDSTEDRGFQRFLDNVQYKASGILRYERIFGEGFVSTGGIETTKEFVDRLDLKPGQNVLDVGCGIGGGDFYMADKYDVHVVGIDLSINMVSFALERAIGRKCSVEFEVADCTKKTYPDNTFDVIYSRDTILHIQDKPSLFKSFFKWLKPGGKVLISDYCKCPGKPSEEFAAYIKQRGYDLHDVRAYGQMLENAGFHDVIAEDRTDQFLDVLERELAKVEKNKNEFVSDFSQEDYDAIVNGWKAKLQRSSAGEQRWGLFIATK

SEQ ID NO: 67 - DNA, 1476 bp, Oryza sativa
atgcgtgcagggatcggggaggtggagaggaaggcgcagcggagctactgggaggagcactccaaggacctcaccgtcgaggccatgatgctcgactcccgcgccgccgacctcgacaaggaggagcgccccgaggtcctgtctgtactcccttcttacaaagggaaatcagtactggagcttggtgctggaataggacgctttactggggaactggcaaaagaagctggccatgttttagccctagacttcattgaaagtgtgattaagaagaatgagaacataaatgggcatcacaagaacataacctttatgtgcgctgatgtcacgtctccggacctgacgatcgaagataactctattgatctcatattctcaaactggctactaatgtacctttcagatgaggaggtcgagaagctagtaggaagaatggtgaaatggctgaaggtaggtggccatatattctttagggagtcatgctttcaccaatctggagattccaaaaggaaggtgaatccaacacattaccgggagccaaggttctatacaaagatatttaaagaatgccattcctatgataaagatgggggttcttatgaactttctctagaaacatgcaagtgcattggggcttatgtgaaaagcaagaaaaatcaaaatcagttatgttggctatgggaaaaggttaagtcaacagaagacagaggattccaaagattcctggacaatgtgcagtacaaaaccactggaatcttacgctatgagcgtgtcttcggagagggttatgtcagcactggtggaattgaaaccacaaaggaatttgtggataagctggatcttaaacctggacagaaagtgcttgatgttgggtgcggaattggaggcggcgacttctatatggctgaaaactacgatgcccatgttcttggtattgatctttcaatcaacatggtttcatttgcaatcgaacgtgccattggacgcaagtgttcggttgagtttgaagtagctgattgcaccacaaagacctacgcaccaaatacatttgatgtgatctacagccgtgacaccattcttcacatacatgataaacctgctttgttcagaagtttcttcaagtggctgaaacctgggggcaaagtcctcatcagtgattactgtaggaatcctgggaaaccatcagaagaatttgctgcttacattaagcagagaggctatgacctccacgatgtgaagacttacggaaagatgcttgaggatgctggtttccatcatgtcattgctgaagaccgcacggaccagttcctgcgtgttcttcaaagggagcttgctgaagttgagaagaacaaagaagccttcatggcagacttcacccaggaggactacgatgacattgtgaacggctggaacgcgaagctgaagcggagctctgccggtgagcagaggtgggggctgttcattgcaaccaaatga

SEQ ID NO: 68 - protein, 491 aa, Oryza sativa
MRAGIGEVERKAQRSYWEEHSKDLTVEAMMLDSRAADLDKEERPEVLSVLPSYKGKSVLELGAGIGRFTGELAKEAGHVLALDFIESVIKKNENINGHHKNITFMCADVTSPDLTIEDNSIDLIFSNWLLMYLSDEEVEKLVGRMVKWLKVGGHIFFRESCFHQSGDSKRKVNPTHYREPRFYTKIFKECHSYDKDGGSYELSLETCKCIGAYVKSKKNQNQLCWLWEKVKSTEDRGFQRFLDNVQYKTTGILRYERVFGEGYVSTGGIETTKEFVDKLDLKPGQKVLDVGCGIGGGDFYMAENYDAHVLGIDLSINMVSFAIERAIGRKCSVEFEVADCTTKTYAPNTFDVIYSRDTILHIHDKPALFRSFFKWLKPGGKVLISDYCRNPGKPSEEFAAYIKQRGYDLHDVKTYGKMLEDAGFHHVIAEDRTDQFLRVLQRELAEVEKNKEAFMADFTQEDYDDIVNGWNAKLKRSSAGEQRWGLFIATK

SEQ ID NO: 69 - DNA, 1488 bp, Oryza sativa
atggacgccgtcgcggcgaatgggatcggggaggtggagaggaaggcgcagcggagctactgggaggagcactccaaggacctcaccgtcgaggccatgatgctcgactcccgcgccgccgacctcgacaaggaggagcgccccgaggtcctgtctgtactcccttcttacaaagggaaatcagtactggagcttggtgctggaataggacgctttactggggaactggcaaaagaagctggccatgttttagccctagacttcattgaaagtgtgattaagaagaatgagaacataaatgggcatcacaagaacataacctttatgtgcgctgatgtcacgtctccggacctgacgatcgaagataactctattgatctcatattctcaaactggctactaatgtacctttcagatgaggaggtcgagaagctagtaggaagaatggtgaaatggctgaaggtaggtggccatatattctttagggagtcatgctttcaccaatctggagattccaaaaggaaggtgaatccaacacattaccgggagccaaggttctatacaaagatatttaaagaatgccattcctatgataaagatgggggttcttatgaactttctctagaaacatgcaagtgcattggggcttatgtgaaaagcaagaaaaatcaaaatcagttatgttggctatgggaaaaggttaagtcaacagaagacagaggattccaaagattcctggacaatgtgcagtacaaaaccactggaatcttacgctatgagcgtgtcttcggagagggttatgtcagcactggtggaattgaaaccacaaaggaatttgtggataagctggatcttaaacctggacagaaagtgcttgatgttgggtgcggaattggaggcggcgacttctatatggctgaaaactacgatgcccatgttcttggtattgatctttcaatcaacatggtttcatttgcaatcgaacgtgccattggacgcaagtgttcggttgagtttgaagtagctgattgcaccacaaagacctacgcaccaaatacatttgatgtgatctacagccgtgacaccattcttcacatacatgataaacctgctttgttcagaagtttcttcaagtggctgaaacctgggggcaaagtcctcatcagtgattactgtaggaatcctgggaaaccatcagaagaatttgctgcttacattaagcagagaggctatgacctccacgatgtgaagacttacggaaagatgcttgaggatgctggtttccatcatgtcattgctgaagaccgcacggaccagttcctgcgtgttcttcaaagggagcttgctgaagttgagaagaacaaagaagccttcatggcagacttcacccaggaggactacgatgacattgtgaacggctggaacgcgaagctgaagcggagctctgccggtgagcagaggtgggggctgttcattgcaaccaaatga

SEQ ID NO: 70 - protein, 495 aa, Oryza sativa
MDAVAANGIGEVERKAQRSYWEEHSKDLTVEAMMLDSRAADLDKEERPEVLSVLPSYKGKSVLELGAGIGRFTGELAKEAGHVLALDFIESVIKKNENINGHHKNITFMCADVTSPDLTIEDNSIDLIFSNWLLMYLSDEEVEKLVGRMVKWLKVGGHIFFRESCFHQSGDSKRKVNPTHYREPRFYTKIFKECHSYDKDGGSYELSLETCKCIGAYVKSKKNQNQLCWLWEKVKSTEDRGFQRFLDNVQYKTTGILRYERVFGEGYVSTGGIETTKEFVDKLDLKPGQKVLDVGCGIGGGDFYMAENYDAHVLGIDLSINMVSFAIERAIGRKCSVEFEVADCTTKTYAPNTFDVIYSRDTILHIHDKPALFRSFFKWLKPGGKVLISDYCRNPGKPSEEFAAYIKQRGYDLHDVKTYGKMLEDAGFHHVIAEDRTDQFLRVLQRELAEVEKNKEAFMADFTQEDYDDIVNGWNAKLKRSSAGEQRWGLFIATK

SEQ ID NO: 71 - DNA, 1164 bp, Oryza sativa
atgtgcgctgatgtcacgtctccggacctgacgatcgaagataactctattgatctcatattctcaaactggctactaatgtacctttcagatgaggaggtcgagaagctagtaggaagaatggtgaaatggctgaaggtaggtggccatatattctttagggagtcatgctttcaccaatctggagattccaaaaggaaggtgaatccaacacattaccgggagccaaggttctatacaaagatatttaaagaatgccattcctatgataaagatgggggttcttatgaactttctctagaaacatgcaagtgcattggggcttatgtgaaaagcaagaaaaatcaaaatcagttatgttggctatgggaaaaggttaagtcaacagaagacagaggattccaaagattcctggacaatgtgcagtacaaaaccactggaatcttacgctatgagcgtgtcttcggagagggttatgtcagcactggtggaattgaaaccacaaaggaatttgtggataagctggatcttaaacctggacagaaagtgcttgatgttgggtgcggaattggaggcggcgacttctatatggctgaaaactacgatgcccatgttcttggtattgatctttcaatcaacatggtttcatttgcaatcgaacgtgccattggacgcaagtgttcggttgagtttgaagtagctgattgcaccacaaagacctacgcaccaaatacatttgatgtgatctacagccgtgacaccattcttcacatacatgataaacctgctttgttcagaagtttcttcaagtggctgaaacctgggggcaaagtcctcatcagtgattactgtaggaatcctgggaaaccatcagaagaatttgctgcttacattaagcagagaggctatgacctccacgatgtgaagacttacggaaagatgcttgaggatgctggtttccatcatgtcattgctgaagaccgcacggaccagttcctgcgtgttcttcaaagggagcttgctgaagttgagaagaacaaagaagccttcatggcagacttcacccaggaggactacgatgacattgtgaacggctggaacgcgaagctgaagcggagctctgccggtgagcagaggtgggggctgttcattgcaaccaaatga

SEQ ID NO: 72 - protein, 387 aa, Oryza sativa
MCADVTSPDLTIEDNSIDLIFSNWLLMYLSDEEVEKLVGRMVKWLKVGGHIFFRESCFHQSGDSKRKVNPTHYREPRFYTKIFKECHSYDKDGGSYELSLETCKCIGAYVKSKKNQNQLCWLWEKVKSTEDRGFQRFLDNVQYKTTGILRYERVFGEGYVSTGGIETTKEFVDKLDLKPGQKVLDVGCGIGGGDFYMAENYDAHVLGIDLSINMVSFAIERAIGRKCSVEFEVADCTTKTYAPNTFDVIYSRDTILHIHDKPALFRSFFKWLKPGGKVLISDYCRNPGKPSEEFAAYIKQRGYDLHDVKTYGKMLEDAGFHHVIAEDRTDQFLRVLQRELAEVEKNKEAFMADFTQEDYDDIVNGWNAKLKRSSAGEQRWGLFIATK

SEQ ID NO: 73 - DNA, 1446 bp, Populus trichocarpa
atggctactcatgtggaagaacgcgatattcagaagaagtattggatggataacatttccgatttgagtgtgaatgcaatgatgcttgactcgaaagcatccgaacttgacaaggaagaacgacctgagatactttctctgcttccaccttatgaaggaaaaacagttttggaactcggagctggtattggccgtttcacaggggaattagcacagaaggctggccaagtagtggctttggacttcattgagagtgcaataaaaaagaatgaaaatatcaacggacactataagaatgtcaagtttatgtgcgctgatgtgacatccccagatctgaatatttcagaggggtcggtggatttgatattctcaaattggcttctcatgtatctctctgacaaagaggtggagaatctggtagaaaggatggtcaaatgggtgaaggttgatgggtttattttcttcagagagtcttgttttcatcaatctggagattctaagcgaaaatacaacccaacccattaccgggaacccagattctacacgaaggtgtttaaagaatgccatacgcgtgatgggtctggagattctttcgaactctctcttgttggctgcaaatgcatctcagcttatatttgttggatatggcagaaagttagttcatatgaggataaggggttccagcgattcttagataatgttcagtataaatccaatggcatattacgttatgagcgtgtctttggacaaggttatgtgagtacaggaggaattgaaacaactaaagaatttgtgggaaaactggatcttaagcctggccagaaagtcctagatgttggctgtgggattgggggaggtgacttttacatggctgagaactttgatgtggaggttgtaggcattgacctctccataaatatgatttcgtttgcccttgaacgtgccattgggctcaaatgttctgtggagtttgaagttgctgattgtactacaaagacatatcctgacaacacatttgatgttatctacagccgtgacaccattttgcacattcaagacaaacctgcattatttagatctttcttcaagtggttgaagcctggaggtaaagtacttatcagtgattactgcaagtgtgatggaactccatcaccagaattcgccgagtacattaaacagagaggatatgatcttcatgatgtaaaagcatatggccagatgcttagggatgctggttttgatgaggtcgttgcagaggaccgaactgatcagttcaacaaagttctgcaaagggagttaaatgctatagagaaggacaaggatgagttcatccacgacttttccgaaggggactataatgatatagttggtggatggaaggcaaagctgatcaggagttcatctggggagcagcgatggggcctgttcatcgccaagaaaaaatga

SEQ ID NO: 74 - protein, 481 aa, Populus trichocarpa
MATHVEERDIQKKYWMDNISDLSVNAMMLDSKASELDKEERPEILSLLPPYEGKTVLELGAGIGRFTGELAQKAGQVVALDFIESAIKKNENINGHYKNVKFMCADVTSPDLNISEGSVDLIFSNWLLMYLSDKEVENLVERMVKWVKVDGFIFFRESCFHQSGDSKRKYNPTHYREPRFYTKVFKECHTRDGSGDSFELSLVGCKCISAYICWIWQKVSSYEDKGFQRFLDNVQYKSNGILRYERVFGQGYVSTGGIETTKEFVGKLDLKPGQKVLDVGCGIGGGDFYMAENFDVEVVGIDLSINMISFALERAIGLKCSVEFEVADCTTKTYPDNTFDVIYSRDTILHIQDKPALFRSFFKWLKPGGKVLISDYCKCDGTPSPEFAEYIKQRGYDLHDVKAYGQMLRDAGFDEVVAEDRTDQFNKVLQRELNAIEKDKDEFIHDFSEGDYNDIVGGWKAKLIRSSSGEQRWGLFIAKKK

SEQ ID NO: 75 - DNA, 1038 bp, Populus trichocarpa
atgacttatgttgtgttgaaaggatatctatatgatccgattgattgcgtaaggcacgcggtagcaacggaaccggggaaagtggagaatctggttgaaaggatggtcaaatggctaaaggttggggggttcattttctttagagagtcttgttttcatcaatctggagattccaagcgaaaatacaacccaacccactaccgtgaacccagattctacacaaagatttgttggatatggcagaaagtcagttcaaatgatgataaggggttccagcgattcttagataatgtccaatataaatctaatggcatattacgttatgagcgcgtctttggtcaaggttttgtgagcacaggaggaatggagacaactaaagaatttgtggaaaagctggatcttaagcctggccagaaagtcctagatgttggctgtgggattgggggaggtgacttttacatggctgagaactttgaagtggaggttgtaggcattgacctctccgtaaatatgatttcatttgctctcgaacgtgccattggactcaaatgctctgttgagtttgaagttgctgattgcactacgaagacatatcctgacaatacttttgatgttatctacagccgggacaccattttgcacattcaagacaaacctgcattatttagatctttcttcaagtggctgaagcctggaggtaaagtacttatcagtgattactgcaagtgtgctggaactccatcaccagaatttgcagagtacattaaacagagaggatatgatcttcatgatgtgaaagcatatggccagatgcttagggatgctggttttgatgaggtcattgcagaagaccgaactgatcagttcaaccaagttctgctaagggaattaaaagctatagaaaaggagaaggatgaatttatccatgacttctctgaagaagactataatgatatagttggtggatggaaggcaaagctgatcaggagttcatctggcgagcagcgatggggcctgttcattgccaagaaaaaatga

SEQ ID NO: 76 - protein, 345 aa, Populus trichocarpa
MTYVVLKGYLYDPIDCVRHAVATEPGKVENLVERMVKWLKVGGFIFFRESCFHQSGDSKRKYNPTHYREPRFYTKICWIWQKVSSNDDKGFQRFLDNVQYKSNGILRYERVFGQGFVSTGGMETTKEFVEKLDLKPGQKVLDVGCGIGGGDFYMAENFEVEVVGIDLSVNMISFALERAIGLKCSVEFEVADCTTKTYPDNTFDVIYSRDTILHIQDKPALFRSFFKWLKPGGKVLISDYCKCAGTPSPEFAEYIKQRGYDLHDVKAYGQMLRDAGFDEVIAEDRTDQFNQVLLRELKAIEKEKDEFIHDFSEEDYNDIVGGWKAKLIRSSSGEQRWGLFIAKKK

SEQ ID NO: 77 - DNA, 1506 bp, Zea Mays
atggacaccgtcggcgtccccgtggtggccgttgcgaatgggatcggggaggtggagcgcaaggtgcagaagagctactgggaggagcactccaagtgcctcactgtcgagtccatgatgctcgactcccgcgccgccgacctcgacaaggaagagcgacccgagatcctgtctttgcttccctcttacaaagggaaatcagttctagaactcggtgctggaattggacgctttactggagatctggcaaaagaagctgggcacgttctggcactagactttattgaaagtgtgattaagaagaaccaaagcataaatgggcatcacaagaacataaccttcaggtgtgccgatgtgacatctaacgacttgaagattgaagataactctgttgatctgatattttcaaactggctattaatgtatctttcagatgaggaggtccaaaagcttgtggggaaaatggtaaaatggttaaaggtcggaggccatattttctttagagaatcatgttttcaccaatctggagattccaaaaggaaggtgaacccaacacactatcgagaaccaaggttttataccaaggtatttaaagagggccattcatttgatcaagatggaggttcgtttgaactttctctagtgacctgtaaatgcattggggcttatgtcaaaaacaagaagaatcaaaaccagatatgctggttatgggaaaaggtaaaatcaacagaagacagagattttcaaagattcctggacaacgtgcaatacaaaacaagtgggatattacgttacgagcgtgtctttggtgaaggttttgtgagcactggtggaatcgagacaacaaaggaatttgtgggcatgctcgatcttaaaccgggccagaaagtacttgatgtcggatgtggaattggaggcggcgacttttacatggctgcaaactatgatgtccatgttcttggtattgatctttcggtgaacatggtttcatttgcaattgaacgtgccattggacgcaagtgctctgttgaatttgaagttgctgattgcaccacaaaggattacccagaaaatagttttgacgtcatctacagccgtgacaccatccttcacatacaagacaagcctgctctgttcagaagcttcttcaaatggctaaagcccggtggcaaagtcctaatcagcgactactgtaagaatcctggaaaaccatcagaagaatttgctgcgtacattaagcagagaggctatgaccttcacgacgtgaaggcttatggacagatgctgaaggatgctggttttcataatgtcatcgcggaagatcgcactgagcagttcttgaatgttctacagagggagctaggtgaagttgaaaagaacaaagacgctttcctggcagacttcacccaggaggactatgacgacattgtgaatggctggaacgcgaagctgaaacggagctctgccggcgagcagaggtgggggttgttcattgccaccaagtga

SEQ ID NO: 78 - protein, 501 aa, Zea Mays
MDTVGVPVVAVANGIGEVERKVQKSYWEEHSKCLTVESMMLDSRAADLDKEERPEILSLLPSYKGKSVLELGAGIGRFTGDLAKEAGHVLALDFIESVIKKNQSINGHHKNITFRCADVTSNDLKIEDNSVDLIFSNWLLMYLSDEEVQKLVGKMVKWLKVGGHIFFRESCFHQSGDSKRKVNPTHYREPRFYTKVFKEGHSFDQDGGSFELSLVTCKCIGAYVKNKKNQNQICWLWEKVKSTEDRDFQRFLDNVQYKTSGILRYERVFGEGFVSTGGIETTKEFVGMLDLKPGQKVLDVGCGIGGGDFYMAANYDVHVLGIDLSVNMVSFAIERAIGRKCSVEFEVADCTTKDYPENSFDVIYSRDTILHIQDKPALFRSFFKWLKPGGKVLISDYCKNPGKPSEEFAAYIKQRGYDLHDVKAYGQMLKDAGFHNVIAEDRTEQFLNVLQRELGEVEKNKDAFLADFTQEDYDDIVNGWNAKLKRSSAGEQRWGLFIATK

SEQ ID NO: 79 - DNA, 1488 bp, Zea Mays
atggccgccgccgtgaatgggagcctagacgtgcatgagaggaaggcgcagaagagctactgggaggagcactccggggagctcaacctcgaggccattatgctcgactcccgtgccgccgaactcgacaaggaggagcgccccgaggttctgtctttacttccttcatatgaagggaaatctatactggagctgggagctggaataggccgctttactggtgaactggctaaaacatctgggcatgtttttgcagtggatttcgttgaaagtgtgattaaaaagaatggaagtataaatgatcactatggcaacacatcctttatgtgtgctgatgttacatccccggacctgatgattgaagcaaactccattgatctgatattttcaaactggttgctgatgtatctttcagatgaggagattgacaagttggtagaaagaatggtaaaatggttgaaggtcggtggttatatcttctttagggaatcttgcttccatcaatccggagatacagaaaggaaatttaatccaacacactatcgagaaccaaggttttataccaaggtatttaaagaatgccaaacctttaatcaggatggcacttccttcaaactttctttgattacattcaaatgcattggagcttatgtaaacatcaagaaagatcaaaaccagatatgttggctatggaaaaaagtaaactcatcagaagatgggggatttcaaagttttttggacaatgtgcagtacaaagccactggaatactacgctatgaacgtatctttggagatggctacgtgagtactggtggagctgagactacaaaagaatttgtggagaaactgaatcttaagcctgggcagaaggtgcttgatgttggatgtggaattgggggaggtgacttttatatggctgagaagtatggtacacatgtcgttggtattgacctttccattaacatgataatgtttgcccttgagcgttccattgggtgtaagtgcttagttgagtttgaagttgctgattgcaccacaaagacatacccagaccacatgtttgatgtcatctacagtcgtgacactatccttcatatacaagataaaccctccttgtttaaaagtttcttcaaatggctgaaacctgggggaaaggttctaatcagtgattactgcaagagtcctggaaaaccatcagaagagtttgcaacatacattaagcagaggggttatgatctccatgacgtggaggcttatggacagatgctgaaggatgctggttttcataatgtcatcgcggaagatcgcactgagcagttcttgaatgttctacagagggagataggtgaagttgaaaagaacaaagacgctttcctggcagacttcacccaggaggactatgacgacattgtgaacggctggaacgcgaagctgaaacggagctctggcggtgagcagaggtgggggttgttcattgccaccaagtga

SEQ ID NO: 80 - protein, 495 aa, Zea Mays
MAAAVNGSLDVHERKAQKSYWEEHSGELNLEAIMLDSRAAELDKEERPEVLSLLPSYEGKSILELGAGIGRFTGELAKTSGHVFAVDFVESVIKKNGSINDHYGNTSFMCADVTSPDLMIEANSIDLIFSNWLLMYLSDEEIDKLVERMVKWLKVGGYIFFRESCFHQSGDTERKFNPTHYREPRFYTKVFKECQTFNQDGTSFKLSLITFKCIGAYVNIKKDQNQICWLWKKVNSSEDGGFQSFLDNVQYKATGILRYERIFGDGYVSTGGAETTKEFVEKLNLKPGQKVLDVGCGIGGGDFYMAEKYGTHVVGIDLSINMIMFALERSIGCKCLVEFEVADCTTKTYPDHMFDVIYSRDTILHIQDKPSLFKSFFKWLKPGGKVLISDYCKSPGKPSEEFATYIKQRGYDLHDVEAYGQMLKDAGFHNVIAEDRTEQFLNVLQREIGEVEKNKDAFLADFTQEDYDDIVNGWNAKLKRSSGGEQRWGLFIATK

SEQ ID NO: 81 - DNA, 1086 bp, Zea Mays
atgtatctttcagatgaagaggttgaacagctagttcagagaatggtaaaatggttgaaggttggtggctatatcttctttagggaatcttgcttccatcaatctggagattcaaaaaggaaagttaatccgacacactatagggaaccaagtttttatactaaggttttcaaagaatgccatacctttgatcaagatgggaattctttcgaactttctctggttacttgcaagtgtattggtgcttatgttaaaaacaagaaaaaccaaaaccagatatgttggctatggcaaaaggtccattctacagaagataaaggatttcaaagatttttggacaatgtgcagtacaaagccagtggaatattacgttacgagcgcatttttggagaaggttatgtgagcactggtggagttgagactacaaaagaatttgtggacaagctggatctcaaacctggacataaggtgcttgatgttggatgtggaattgggggaggtgacttttatatggccgaaaaatatgatgctcatgttgttggtattgatctttccataaacatggtatcatttgcacttgagcgtgccattgggcgcagttgctcagtggagtttgaagttgctgattgcactacgaagacatacccagacaacacatttgatgtcatatacagccgtgatactatccttcacatacatgacaaaccctctttgttcaaaagttttttcaagtggctgaagcctgggggcaaggtccttatcagtgactactgcaggagtcctgggaaaccatcagaggaatttgcagcgtacattaagcagagaggttatgacctacatgctgtggaggcttatggacagatgttgaagagtgctggttttcgtgatgtcattgctgaggatcgaactgatcagttccttggtgttttagataaggagttagctgaatttgaaaagaacaaggacgatttcctgtctgacttcacccaggaggactacgatgatatcgtgaacggttggaaggcaaaactgcagaggagttctgctggtgaacagaggtgggggctgttcatcgccaccaaatga

SEQ ID NO: 82 - protein, 361 aa, Zea Mays
MYLSDEEVEQLVQRMVKWLKVGGYIFFRESCFHQSGDSKRKVNPTHYREPSFYTKVFKECHTFDQDGNSFELSLVTCKCIGAYVKNKKNQNQICWLWQKVHSTEDKGFQRFLDNVQYKASGILRYERIFGEGYVSTGGVETTKEFVDKLDLKPGHKVLDVGCGIGGGDFYMAEKYDAHVVGIDLSINMVSFALERAIGRSCSVEFEVADCTTKTYPDNTFDVIYSRDTILHIHDKPSLFKSFFKWLKPGGKVLISDYCRSPGKPSEEFAAYIKQRGYDLHAVEAYGQMLKSAGFRDVIAEDRTDQFLGVLDKELAEFEKNKDDFLSDFTQEDYDDIVNGWKAKLQRSSAGEQRWGLFIATK

SEQ ID NO: 83 - DNA, 56 bp, Artificial sequence
  primer 1
ggggacaagtttgtacaaaaaagcaggcttaaacaatggagcattctagtgatttg

SEQ ID NO: 84 - DNA, 50 bp, Artificial sequence
  primer 2
ggggaccactttgtacaagaaagctgggtcagagttttgggataaaaaca

SEQ ID NO: 85 - DNA, 2194 bp, Oryza sativa
aatccgaaaagtttctgcaccgttttcaccccctaactaacaatatagggaacgtgtgctaaatataaaatgagaccttatatatgtagcgctgataactagaactatgcaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagagattttttttaaaaaaatagaatgaagatattctgaacgtattggcaaagatttaaacatataattatataattttatagtttgtgcattcgtcatatcgcacatcattaaggacatgtcttactccatcccaatttttatttagtaattaaagacaattgacttatttttattatttatcttttttcgattagatgcaaggtacttacgcacacactttgtgctcatgtgcatgtgtgagtgcacctcctcaatacacgttcaactagcaacacatctctaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagaggaggagaggcaaagaaaaccaagcatcctccttctcccatctataaattcctccccccttttcccctctctatataggaggcatccaagccaagaagagggagagcaccaaggacacgcgactagcagaagccgagcgaccgccttctcgatccatatcttccggtcgagttcttggtcgatctcttccctcctccacctcctcctcacagggtatgtgcctcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggatcggaatcttgcgattttgtgagtaccttttgtttgaggtaaaatcagagcaccggtgattttgcttggtgtaataaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttattagctctcaccccttcattattctgagctgaaagtctggcatgaactgtcctcaattttgttttcaaattcacatcgattatctatgcattatcctcttgtatctacctgtagaagtttctttttggttattccttgactgcttgattacagaaagaaatttatgaagctgtaatcgggatagttatactgcttgttcttatgattcatttcctttgtgcagttcttggtgtagcttgccactttcaccagcaaagttc

SEQ ID NO: 86 - protein, 110 aa, Artificial sequence
  Methyltransferase type 11 domain
PPYEGKSVLELGAGIGRFTGELAQKAGEVIALDIIESAIQKNESVNGHYKNIKFMCADVTSPDLKIKDGSIDLIFSNWLLMYLSDKEVELMAERMIGWVKPGGYIFFRES

SEQ ID NO: 87 - protein, 108 aa, Artificial sequence
  Methyltransferase type 11 domain
DLKPGQKVLDVGCGIGGGDFYMAENFDVHVVGIDLSVNMISFALERAIGLKCSVEFEVADCTTKTYPDNSFDVIYSRDTILHIQDKPALFRTFFKWLKPGGKVLITDY

SEQ ID NO: 88 - protein, 180 aa, Artificial sequence
  ubiE/COQ5 methyltransferase domain
ERVFGEGYVSTGGFETTKEFVAKMDLKPGQKVLDVGCGIGGGDFYMAENFDVHVVGIDLSVNMISFALERAIGLKCSVEFEVADCTTKTYPDNSFDVIYSRDTILHIQDKPALFRTFFKWLKPGGKVLITDYCRSAETPSPEFAEYIKQRGYDLHDVQAYGQMLKDAGFDDVIAEDRTDQ

SEQ ID NO: 89 - protein, 13 aa, Artificial sequence
  motif 5
IFFRESCFHQSGD

SEQ ID NO: 90 - protein, 6 aa, Artificial sequence
  motif 6
EYIKQR

SEQ ID NO: 91 - protein, 6 aa, Artificial sequence
  motif 7
WGLFIA

SEQ ID NO: 92 - DNA, 1239 bp, Arabidopsis thaliana
atggtggccacctctgctacgtcgtcattctttcctgtaccatcttcttcacttgatcctaatggaaaaggcaataagattgggtctacgaatcttgctggactcaattctgcacctaactctggtaggatgaaggttaaaccaaacgctcaggctccacctaagattaatgggaaaaaggttggtttgcctggttctgtagatattgtaaggactgataccgagacctcatcacaccctgcgccgagaactttcatcaaccagttacctgactggagcatgcttcttgctgctataactacgattttcttagcggctgagaaacagtggatgatgcttgattggaaacctaggcgttctgacatgctggtggatccttttggtatagggagaattgttcaggatggccttgtgttccgtcagaatttttctattaggtcatatgaaataggtgctgatcgctctgcatctatagaaaccgtcatgaatcatctgcaggaaacggcgcttaatcatgttaagactgctggattgcttggagatgggtttggctctacacctgagatgtttaagaagaacttgatatgggttgtcactcgtatgcaggttgtggttgataaatatcctacttggggagatgttgttgaagtagacacctgggtcagtcagtctggaaagaatggtatgcgtcgtgattggctagttcgggactgtaatactggagaaaccttaacacgagcatcaagtgtgtgggtgatgatgaataaactgacaaggagattgtcaaagattcctgaagaggttcgaggggaaatagagccttattttgtgaattctgatcctgtccttgccgaggacagcagaaagttaacaaaaattgatgacaagactgctgactatgttcgatctggtctcactcctcgatggagtgacctagatgttaaccagcatgtgaataatgtaaagtacattgggtggatcctggagagtgctccagtgggaataatggagaggcagaagctgaaaagcatgactctggagtatcggagggaatgcgggagagacagtgtgcttcagtccctcactgcagttacgggttgcgatatcggtaacctggcaacagcgggggatgtggaatgtcagcatttgctccgactccaggatggagcggaagtggtgagaggaagaacagagtggagtagtaaaacaccaacaacaacttggggaactgcaccgtaa

SEQ ID NO: 93 - protein, 412 aa, Arabidopsis thaliana
MVATSATSSFFPVPSSSLDPNGKGNKIGSTNLAGLNSAPNSGRMKVKPNAQAPPKINGKKVGLPGSVDIVRTDTETSSHPAPRTFINQLPDWSMLLAAITTIFLAAEKQWMMLDWKPRRSDMLVDPFGIGRIVQDGLVFRQNFSIRSYEIGADRSASIETVMNHLQETALNHVKTAGLLGDGFGSTPEMFKKNLIWVVTRMQVVVDKYPTWGDVVEVDTWVSQSGKNGMRRDWLVRDCNTGETLTRASSVWVMMNKLTRRLSKIPEEVRGEIEPYFVNSDPVLAEDSRKLTKIDDKTADYVRSGLTPRWSDLDVNQHVNNVKYIGWILESAPVGIMERQKLKSMTLEYRRECGRDSVLQSLTAVTGCDIGNLATAGDVECQHLLRLQDGAEVVRGRTEWSSKTPTTTWGTAP

SEQ ID NO: 94 - DNA, 1245 bp, Aquilegia formosa x Aquilegia pubescens
atggtcgcatccgccgctaccgcagcattctttcccgttactaaagcttcttctacaaaggcttcacttgtgcctggtggaggatcagataatttggacactcgaggaatcaattcgtcgaaacctacttcttctggaggtttgaaagttaaggctaatgcacaagcaactcctaaaattaatggaacttctattcattacccaccatcatctgaacgtttgaagaattccgatgaaacttcaattgcacctgccagaacatttatcaatcaattgcctgattggagtgttcttcttaccgccatcaccgcaatgttcttagcagctgagaaacagtggacacttcttgattggaaaccgaggagatccgacatgcttgttgatccttttggtttagggaagattgttcaggatgggcttgtttttcaacagaatttctcaattagatcgtatgaaataggtgttgatgggacgacgtctatagaatcatttatgaaccatttgcaggaaactgctcttaaccatgctaagactgtggggcttcttggcgatggcttcggttcaactgaagctatgagcaaaagaaacttgatctgggtggtagctaggatgcagattcttgtgaatagatatcctacgtggggtgatactgttcaggtagatacttgggttgctgcaaatgggaagaatggtatgcgtcgtgattggcttgttcgtgacgggaattctggggaaacccttgcaagagcttcaagcaagtgggtgatgatgaatacaagtacgcggaaactatctaaaatgccagatgatgttagggttgaaatagagccttattttatggattgtgctcctattgttgaggaagatggcagaaagctgccaaagcttgatgaaagcacatcagattatgttcgaaatggcctaacgcctcgatggaatgatctggatctcaatcagcatgtgaacaatgtcaagtacataggctggattcttgagagttctatctcaatgttggagaatcatgagcttgcaggcatcactctagagtatcggaaggagtgtcggaaggacaatgtgctgcaatccttgactgctgtcagcaaagatgccaaaggctggcctgagtgtgttcacttgcttcgtcttgacagtggggctgaggttgtcaggggaagcactatgtggaggccgaagcgcatcaacaactttggatctgtgggccgaattcctaccgatggcatgtag

SEQ ID NO: 95 - protein, 414 aa, Aquilegia formosa x Aquilegia pubescens
MVASAATAAFFPVTKASSTKASLVPGGGSDNLDTRGINSSKPTSSGGLKVKANAQATPKINGTSIHYPPSSERLKNSDETSIAPARTFINQLPDWSVLLTAITAMFLAAEKQWTLLDWKPRRSDMLVDPFGLGKIVQDGLVFQQNFSIRSYEIGVDGTTSIESFMNHLQETALNHAKTVGLLGDGFGSTEAMSKRNLIWVVARMQILVNRYPTWGDTVQVDTWVAANGKNGMRRDWLVRDGNSGETLARASSKWVMMNTSTRKLSKMPDDVRVEIEPYFMDCAPIVEEDGRKLPKLDESTSDYVRNGLTPRWNDLDLNQHVNNVKYIGWILESSISMLENHELAGITLEYRKECRKDNVLQSLTAVSKDAKGWPECVHLLRLDSGAEVVRGSTMWRPKRINNFGSVGRIPTDGM

SEQ ID NO: 96 - DNA, 1242 bp, Arachis hypogaea
atggcaactgctgctactgcttccattttccctgttccttcaccctcaccagatgcaggtgcagatggcaacaaacttgttggtggctctgttaaacttcaagggctcaaatctaaacatgcatcttctggtggcttgcaagttaaagctcatgcccaagctccacccaagattaatggaagcacagtagaaagcttgaagcatgatgatgatttgccttcccctccccccaggacttttattaaccagttacctgattggagcatgcttcttgctgctataactacaattttcctggcagcagaaaagcagtggatgatgcttgattggaaaccaaggcgatctgacatgcttattgatccctttggaataggaagaattgttcaagatggtctagtgttccgtcaaaacttttctattagatcatatgaaattggtgccgatcgaacagcatctatagagacagtaatgaaccatctgcaggaaactgcacttaatcatgtcaagactgctggacttcttggtgatggctttggttccacaccagaaatgtgcaaaaagaacttgatatgggtagtcacacggatgcaggttgtggttgatcgttatcctacatggggtgatgttgttcaagtagatacttgggtatctgcatctgggaagaatggcatgcgtcgtgattggcttctgcgtgactgcaaaactggtgaagtattgacgagagcctccagtgtttgggtcatgatgaataaactaacaaggaggctatctaaaattccagaagaagtcagagcggagatagcatcttattttgtgaattccgctccaattctggaagaggataacagaaaactatctaaacttgatgacaataccgctgattacattcgcacgggtcttagtcctagatggaatgatctagatgtcaatcagcatgttaacaatgtgaagtacattggctggattctggagagtgctccgcagccaatcttggagagtcatgagctttctgcaatgactttggagtataggagggagtgtggtagggacagtgtgctgcagtccctcactgctgtgtctgctgccgacgtcggcaatcttgctcacagggggcaactcgagtgcaagcatttgcttcgacttgaagatggtgctgaaattgtgaggggtaggactgagtggaggcccaaacctgtgagcaactttgacattgtgaatcaggttccagccgaaagcatctaa

SEQ ID NO: 97 - protein, 413 aa, Arachis hypogaea
MATAATASIFPVPSPSPDAGADGNKLVGGSVKLQGLKSKHASSGGLQVKAHAQAPPKINGSTVESLKHDDDLPSPPPRTFINQLPDWSMLLAAITTIFLAAEKQWMMLDWKPRRSDMLIDPFGIGRIVQDGLVFRQNFSIRSYEIGADRTASIETVMNHLQETALNHVKTAGLLGDGFGSTPEMCKKNLIWVVTRMQVVVDRYPTWGDVVQVDTWVSASGKNGMRRDWLLRDCKTGEVLTRASSVWVMMNKLTRRLSKIPEEVRAEIASYFVNSAPILEEDNRKLSKLDDNTADYIRTGLSPRWNDLDVNQHVNNVKYIGWILESAPQPILESHELSAMTLEYRRECGRDSVLQSLTAVSAADVGNLAHRGQLECKHLLRLEDGAEIVRGRTEWRPKPVSNFDIVNQVPAESI

SEQ ID NO: 98 - DNA, 1236 bp, Brassica juncea
atggtggccacctctgctacgtccttattctttcctctcccatcttcctccctcgaccccaacgyaaaaaccaacaacagagtcacctccaccaacttcgccggactcggtccaacgccaaactctggcggcaggatgaaggttaaaccaaacgcccaggctccrcccaagatcaacggsaagaaagttggtctccctggctcggtagagatcgagacctcacaacaacaacaacccgcaccgaggacgttcatcaaccagctgcctgactggagcatgcttctcgccgccattacgaccgtcttcctagcggctgagaaacagtggatgatgcttgactggaaaccgaggcgttccgacatgattatggaaccgtttggtctagggagaatcgttcaggatgggcttgtgttccgtcagaatttttctattaggtcttatgagataggtgctgatcgctctgcatctatagaaacggttatgaatcatttacaggaaacggccctaaacyatgttaagactgctggactgctgggggatgggtttggttctacccctgagatggttaagaagascttgatatgggtcgttactcgtatgcaggttgttgttgatacctatcctacttggggagatgttgttgaagtagatacatgggtcagcaagtctggaaagaatggtatgcgtcgtgattggctagtccgggatggcaatactggacaaattttaacaagagcatcaagtgtatgggtgatgatgaataaactgacgagaagattatcaaagattcctgaagaggttcgaggggagatagagccttactttgtggattttgaccctgtccttgccgaggacagcaggaagttaacaaaactggatgacaaaactgctgactatgtccgttctggtctcactccgcgttggagtgacttagatgttaaccagcatgttaacaatgtaaagtacatagggtggatactggagagtgctccagtggggatgatggagagtcagaagctgaaaagcatgactctggagtatcgcagggagtgcgggagggacagtgtgcttcagtccctcaccgcggtttcgggctgcgatatcggtaacctcgggacagctggtgaagttgaatgtcagcatctgctccgactccaggatggagctgaagtggtgagaggaagaacagagtggagttccaaaacaccaacaacaacttgggacattacaccgtga

SEQ ID NO: 99 - protein, 411 aa, Brassica juncea
  UNSURE (22)..(22): Unknown amino acid
MVATSATSLFFPLPSSSLDPNXKTNNRVTSTNFAGLGPTPNSGGRMKVKPNAQAPPKINGKKVGLPGSVEIETSQQQQPAPRTFINQLPDWSMLLAAITTVFLAAEKQWMMLDWKPRRSDMIMEPFGLGRIVQDGLVFRQNFSIRSYEIGADRSASIETVMNHLQETALNXVKTAGLLGDGFGSTPEMVKKXLIWVVTRMQVVVDTYPTWGDVVEVDTWVSKSGKNGMRRDWLVRDGNTGQILTRASSVWVMMNKLTRRLSKIPEEVRGEIEPYFVDFDPVLAEDSRKLTKLDDKTADYVRSGLTPRWSDLDVNQHVNNVKYIGWILESAPVGMMESQKLKSMTLEYRRECGRDSVLQSLTAVSGCDIGNLGTAGEVECQHLLRLQDGAEVVRGRTEWSSKTPTTTWDITP

SEQ ID NO: 100 - DNA, 1287 bp, Brachypodium sylvaticum
atggcagggtcccttgccgcctcggcgttcttccccagcccaggatcttcaccagctgcattggctaaaagctccaagaacacgtccggtgaattacctgagactttgagtgtccgtggaattgtcgcaaagcctaacacgcctcctgcgtccatgcaagtgaaaactaaggcccaagcgctccccaaggttaatggcaccaaggttaatctcaagacttcaagctctgacaaggaagacacagtgccgtacagttcttcaaagacattctataaccaactgccagattggagcatgctgcttgcagctgtcacgaccatcttcctggccgcagagaagcagtggacaatgcttgattggaaaccgaagaggcctgacatgcttgtcgacacatttggctttggcagaatcatccaggatgggatggtttttaggcagaactttttgattagatcctacgagattggtgctgatcgtacagcttctatagagacattaatgaatcatttacaggaaacagctcttaaccatgtcaagactgctggtctccttggagatggctttggtgctactcaggagatgagtaaacggaacttgatctgggttgtcagcaaaattcagcttcttgtagagcgatatccatcgtgggaagatatggttcaagtcgatacatgggtagcttcttctggaaaaaatggcatgcgtcgagattggcatatccgtgactacaattcggggcaaacgatcttgagagctacaagtgtttgggttacgatgaataagaacactagaaaactttcaaaaatgcctgatgaagttagggctgaaataggcccgcacttcaacaatgaccgttccgctttaacagaggagcatagtgacaagttagctaagccagggaggaaaggtggtgaccctgctaccaaacagttcataaggaaggggcttaccccaaaatggggtgaccttgatgtcaaccaacatgtgaacaatgtgaagtatattgggtggattcttgagagtgctccaatttcaatactggaaaagcatgagcttgcaagcatgacactggaatacaggaaggagtgtggccgtgacagcgtgctgcagtctcttaccaatgtcataggtgagtgcaccgacggcagcccagagtctgctatccagtgcagccatctgctccagctggagtctggaactgacatcgtgaaggctcacacaaagtggcgaccgaagagagcgcagggcgaaggaaacacagggttgttcccagcttcgagtgcataa

SEQ ID NO: 101 - protein, 428 aa, Brachypodium sylvaticum
MAGSLAASAFFPSPGSSPAALAKSSKNTSGELPETLSVRGIVAKPNTPPASMQVKTKAQALPKVNGTKVNLKTSSSDKEDTVPYSSSKTFYNQLPDWSMLLAAVTTIFLAAEKQWTMLDWKPKRPDMLVDTFGFGRIIQDGMVFRQNFLIRSYEIGADRTASIETLMNHLQETALNHVKTAGLLGDGFGATQEMSKRNLIWVVSKIQLLVERYPSWEDMVQVDTWVASSGKNGMRRDWHIRDYNSGQTILRATSVWVTMNKNTRKLSKMPDEVRAEIGPHFNNDRSALTEEHSDKLAKPGRKGGDPATKQFIRKGLTPKWGDLDVNQHVNNVKYIGWILESAPISILEKHELASMTLEYRKECGRDSVLQSLTNVIGECTDGSPESAIQCSHLLQLESGTDIVKAHTKWRPKRAQGEGNTGLFPASSA

SEQ ID NO: 102 - DNA, 1257 bp, Citrus sinensis
atggttgctactgccgcagcttctgcgttcttcccagtttcctcaccatctggggattctgttgcaaagaccaaaaatctcggatctgctaatctgggaggtattaagtcaaaatcctcttctgggagtttgcaggttaaggctaatgcgcaagcaccttccaagataaatggtacttcagttggtttgacaacaccagcagaaagtttgaagaatggtgatatctccacgtcatcacctcctcctaggacttttattaaccagttacctgactggagtatgcttcttgctgctataacaacaatcttcttggcagcagagaagcagtggatgatgcttgattggaaaccaaggcgatctgacatgcttgtggacccatttgggattgggaaaatagttcaggatggtttcattttccggcaaaatttctcaattagatcatatgagataggtgctgatggtactgcatctatagagacattaatgaatcatttacaggaaacagcgcttaatcatgttatgactgctggtcttctagatgctggctttggtgcaaccccagcgatggctaaaaagaacctgatatgggtggttactcggatgcaggttgttgtagaccgctatcccacttggaatgatgttgtaaatgtagaaacttgggttagtgcatctggaaaaaatggtatgcggcgtgattggctcattcgcaatgctaagacaggtgaaacattaacaagagcaaccagtctgtgggtaatgatgaataaactgactaggaggttgtccaaaatgcccgatgaagttcgtcaggaaattgaaccgtattttctgaattctgaccctgttgtcgatgaggatagcaggaaattaccaaaacttggcgacagtactgcagattatgttcgtagaggtttaactcctaggtggagtgatttagatgtcaaccagcatgtcaataatgtgaagtacattggctggatcctagagagtgctcctcagcagatcttggagagtcatcagctggcatctgtgaccctggagtataggagggagtgcggaagggacagtgtgttgcagtccctgactgctgtctcagacaaggacattggcaatttggtgaacttgggcagtgtggagtgccagcacttgctccgactagaggaaggtgctgaagttttgagagcaaggactgaatggaggccaaaggatgcccacaactttgggaatgttggtccaatccctgcagaaagcacttaa

SEQ ID NO: 103 - protein, 418 aa, Citrus sinensis
MVATAAASAFFPVSSPSGDSVAKTKNLGSANLGGIKSKSSSGSLQVKANAQAPSKINGTSVGLTTPAESLKNGDISTSSPPPRTFINQLPDWSMLLAAITTIFLAAEKQWMMLDWKPRRSDMLVDPFGIGKIVQDGFIFRQNFSIRSYEIGADGTASIETLMNHLQETALNHVMTAGLLDAGFGATPAMAKKNLIWVVTRMQVVVDRYPTWNDVVNVETWVSASGKNGMRRDWLIRNAKTGETLTRATSLWVMMNKLTRRLSKMPDEVRQEIEPYFLNSDPVVDEDSRKLPKLGDSTADYVRRGLTPRWSDLDVNQHVNNVKYIGWILESAPQQILESHQLASVTLEYRRECGRDSVLQSLTAVSDKDIGNLVNLGSVECQHLLRLEEGAEVLRARTEWRPKDAHNFGNVGPIPAEST

SEQ ID NO: 104 - DNA, 1254 bp, Elaeis guineensis
atggttgcttcgattgtcgcttgggcctttttccccacaccatctttctcccccacggcatcagcaaaagcttcgaagaccattggtgaaggctccgagaatttgaatgttcggggtatcatagccaaacccacttcttcttcggcggctaagcagggtaaggtgatggcccaagccgtccccaagatcaatggcgcgaaggttggcctgaaagctgaatcccaaaaggccgaggaagatgctgccccttcctcagccccgaggacattctataatcaactacctgactggagcgtgctccttgccgccgtaacaacgatctttttggctgccgagaagcagtggacccttcttgattggaagccacggcgtcccgacatgcttactggtgcatttagccttgggaagattgtgcaggatggactagttttcaggcagaacttttccatcaggtcatatgagattggggctgatcggacggcttctatagaaacgttaatgaaccatttacaggaaacagcacttaatcatgtgaggaatgctgggcttctgggcgatggttttggtgccacaccagagatgagtaaaagaaatttgatttgggttgtcactaaaatgcaggtcctgattgagcactatccttcctggggggatgttgttgaagtagatacatgggttggtgcatctggtaaaaatgggatgcgtcgtgattggcatgttcgtgactaccgaacaggccaaactatattgagagccaccagtatctgggtgatgatggataaacacactaggaagttgtctaaaatgcccgaagaagtcagagcagagatagggccttactttatggaacatgctgctattgtggacgaggacagcagaaagcttccaaagcttgatgatgatactgcagattatattaaatggggcctgactcctcgatggagtgatttagatgtgaatcagcatgtgaacaatgtcaaatatataggctggattcttgagagcgctccaatatcaatcctggagaatcacgagctggcgagtatgactctggaatataggagggagtgtgggagggacagcgttctgcaatccctcaccgcagtcgctaatgactgcactggtggccttccagaagctagcatcgagtgccagcatctgctgcagctggaatgcggggccgagattgttaggggacggacacagtggaggcccaggcgtgcctccggtcccacttcagctggaagtgcttga

SEQ ID NO: 105 - protein, 417 aa, Elaeis guineensis
MVASIVAWAFFPTPSFSPTASAKASKTIGEGSENLNVRGIIAKPTSSSAAKQGKVMAQAVPKINGAKVGLKAESQKAEEDAAPSSAPRTFYNQLPDWSVLLAAVTTIFLAAEKQWTLLDWKPRRPDMLTGAFSLGKIVQDGLVFRQNFSIRSYEIGADRTASIETLMNHLQETALNHVRNAGLLGDGFGATPEMSKRNLIWVVTKMQVLIEHYPSWGDVVEVDTWVGASGKNGMRRDWHVRDYRTGQTILRATSIWVMMDKHTRKLSKMPEEVRAEIGPYFMEHAAIVDEDSRKLPKLDDDTADYIKWGLTPRWSDLDVNQHVNNVKYIGWILESAPISILENHELASMTLEYRRECGRDSVLQSLTAVANDCTGGLPEASIECQHLLQLECGAEIVRGRTQWRPRRASGPTSAGSA

SEQ ID NO: 106 - DNA, 1221 bp, Garcinia mangostana
atggttgctactgccgccacgtcatcattctttccgttgacttccccttctggggatgccaaatcgggcaatcccggaaaagggtcggtgagttttgggtcaatgaagtcgaaatccgcggcttcctcgaggggtttacaagtgaaggccaatgcacaggcacccactaagatcaatggatccacggatgatgctcaattgcctgccccgaggacttttattaaccagttgcctgattggagcatgcttcttgctgctattactaccgtgtttttggcagccgagaagcagtggatgatgttggattggaagcctaggaggcccgacatgcttattgacacgtttggtttggggaggattgtgcaggatggtcttgtttttcgacagaatttctcgattaggtcctatgaaattggtgctgatcgtactgcgtctatagagacggttatgaatcatctgcaagaaactgccctcaatcatgttaagactgcaggacttctgggtgatggattcggttcaacaccagagatgtctaaaaggaatctcatatgggttgttactaagatgcaggtcgaagtcgatcggtatcctacatggggtgacgttgttcaggtagatacttgggtgagtgcatcaggaaagaatggaatgcgtcgagattggcttcttcgtgatggtaatactggggagacattaaccagagcttcaagtgtgtgggtgatgatgaataaactgacaaggagattgtctaaaattcccgaagaagttcgggaggaaataggatcttactttgtgaattctgatcctgttgtggaggaggatggtagaaaggtgacaaaacttgatgacaacactgcagattttgttcgcaaagggttaactcctaaatggaatgacttggacatcaatcagcatgtgaataatgtgaagtatattggctggatccttgagagcgctccacagccaatcctggaaacccgtgagctctcagcggtgactttggagtataggagggagtgtggaagggacagtgtgctgcggtctctgaccgccgtttctggcggtggcgttggtgatttaggacacgctggtaacgtcgagtgccagcacgtgcttcgcttggaggatggagctgagattgttcgtggaaggaccgagtggaggcccaaatacattaacaacttcagtatcatgggccagattccgacagatgcttcttag

SEQ ID NO: 107 - protein, 406 aa, Garcinia mangostana
MVATAATSSFFPLTSPSGDAKSGNPGKGSVSFGSMKSKSAASSRGLQVKANAQAPTKINGSTDDAQLPAPRTFINQLPDWSMLLAAITTVFLAAEKQWMMLDWKPRRPDMLIDTFGLGRIVQDGLVFRQNFSIRSYEIGADRTASIETVMNHLQETALNHVKTAGLLGDGFGSTPEMSKRNLIWVVTKMQVEVDRYPTWGDVVQVDTWVSASGKNGMRRDWLLRDGNTGETLTRASSVWVMMNKLTRRLSKIPEEVREEIGSYFVNSDPVVEEDGRKVTKLDDNTADFVRKGLTPKWNDLDINQHVNNVKYIGWILESAPQPILETRELSAVTLEYRRECGRDSVLRSLTAVSGGGVGDLGHAGNVECQHVLRLEDGAEIVRGRTEWRPKYINNFSIMGQIPTDAS

SEQ ID NO: 108 - DNA, 1251 bp, Glycine max
atggtggcaacagctgctacttcatcatttttccctgttacttcaccctcgccggactctggtggagcaggcagcaaacttggtggtgggcctgcaaaccttggaggactaaaatccaaatctgcgtcttctggtggcttgaaggcaaaggcgcaagccccttcgaaaattaatggaaccacagttgttacatctaaagaaagcttcaagcatgatgatgatctaccttcgcctccccccagaacttttatcaaccagttgcctgattggagcatgcttcttgctgctatcacaacaattttcttggccgctgaaaagcagtggatgatgcttgattggaagccaaggcgacctgacatgcttattgacccctttgggataggaaaaattgttcaggatggtcttgtgttccgtgaaaacttttctattagatcatatgagattggcgctgatcgaaccgcatctatagaaacagtaatgaaccatttgcaagaaactgcacttaatcatgttaaaagtgctgggcttcttggtgatggctttggttccacgccagaaatgtgcaaaaagaacttgatatgggtggttactcggatgcaggttgtggtggaacgctatcctacatggggtgacatagttcaagtggacacttgggtttctggatcagggaagaatggtatgcgccgtgattggcttttacgtgactgcaaaactggtgaaatcttgacaagagcttccagtgtttgggtcatgatgaataagctaacacggaggctgtctaaaattccagaagaagtcagacaggagataggatcttattttgtggattctgatccaattctggaagaggataacagaaaactgactaaacttgacgacaacacagcggattatattcgtaccggtttaagtcctaggtggagtgatctagatatcaatcagcatgtcaacaatgtgaagtacattggctggattctggagagtgctccacagccaatcttggagagtcatgagctttcttccatgactttagagtataggagagagtgtggtagggacagtgtgctggattccctgactgctgtatctggggccgacatgggcaatctagctcacagcgggcatgttgagtgcaagcatttgcttcgactggaaaatggtgctgagattgtgaggggcaggactgagtggaggcccaaacctgtgaacaactttggtgttgtgaaccaggttccagcagaaagcacctaa

SEQ ID NO: 109 - protein, 416 aa, Glycine max
MVATAATSSFFPVTSPSPDSGGAGSKLGGGPANLGGLKSKSASSGGLKAKAQAPSKINGTTVVTSKESFKHDDDLPSPPPRTFINQLPDWSMLLAAITTIFLAAEKQWMMLDWKPRRPDMLIDPFGIGKIVQDGLVFRENFSIRSYEIGADRTASIETVMNHLQETALNHVKSAGLLGDGFGSTPEMCKKNLIWVVTRMQVVVERYPTWGDIVQVDTWVSGSGKNGMRRDWLLRDCKTGEILTRASSVWVMMNKLTRRLSKIPEEVRQEIGSYFVDSDPILEEDNRKLTKLDDNTADYIRTGLSPRWSDLDINQHVNNVKYIGWILESAPQPILESHELSSMTLEYRRECGRDSVLDSLTAVSGADMGNLAHSGHVECKHLLRLENGAEIVRGRTEWRPKPVNNFGVVNQVPAEST

SEQ ID NO: 110 - DNA, 1242 bp, Gossypium hirsutum
atggttgctactgctgtgacatcggcgtttttcccagtcacttcttcacctgactcctctgactcgaaaaacaagaagctcggaagcatcaagtcgaagccatcggtttcttctggaagtttgcaagtcaaggcaaatgctcaagcacctccgaaaataaacggcactgtggcgtcgacgactcccgtggaaggttccaagaacgatgacggtgcaagttcccctcctcctaggacgtttatcaaccagttacctgattggagcatgcttcttgctgctatcacaaccattttcttggctgctgagaagcagtggatgatgcttgattggaagccgaggcggcctgacatggtcattgatccgtttggcatagggaagattgttcaggatggtcttgttttcagtcagaacttctcgattagatcatatgagataggcgctgatcaaacagcatccatagagacactaatgaatcatttacaggaaacagctataaatcattgtcgaagtgctggactgcttggagaaggttttggtgcaacacctgagatgtgcaagaagaacctaatatgggttgtcacacggatgcaagttgtggttgatcgctatcctacttggggtgatgttgttcaagtcgacacttgggtcagtgcatcggggaagaatggcatgcgaagagattggcttgtcagcaatagtgaaactggtgaaattttaacacgagccacaagtgtatgggtgatgatgaataaactgactagaaggttatctaaaatcccagaagaggttcgaggggaaatagaacctttttttatgaattcagatcctgttctggctgaggatagccagaaactagtgaaactcgatgacagcacagctgaacacgtgtgcaaaggtttaactcctaaatggagcgacttggatgtcaaccagcatgtcaataatgtgaagtacattggctggatccttgagagtgctccattaccaatcttggagagtcacgagctttccgccttgactctggaatataggagggagtgcgggagggacagcgtgctgcagtcactgaccactgtgtctgattccaatacggaaaatgcagtaaatgttggtgaatttaattgccaacatttgctccgactcgacgatggagctgagattgtgagaggcaggacccgatggaggcctaaacatgccaaaagttccgctaacatggatcaaattaccgcaaaaagggcatag

SEQ ID NO: 111 - protein, 413 aa, Gossypium hirsutum
MVATAVTSAFFPVTSSPDSSDSKNKKLGSIKSKPSVSSGSLQVKANAQAPPKINGTVASTTPVEGSKNDDGASSPPPRTFINQLPDWSMLLAAITTIFLAAEKQWMMLDWKPRRPDMVIDPFGIGKIVQDGLVFSQNFSIRSYEIGADQTASIETLMNHLQETAINHCRSAGLLGEGFGATPEMCKKNLIWVVTRMQVVVDRYPTWGDVVQVDTWVSASGKNGMRRDWLVSNSETGEILTRATSVWVMMNKLTRRLSKIPEEVRGEIEPFFMNSDPVLAEDSQKLVKLDDSTAEHVCKGLTPKWSDLDVNQHVNNVKYIGWILESAPLPILESHELSALTLEYRRECGRDSVLQSLTTVSDSNTENAVNVGEFNCQHLLRLDDGAEIVRGRTRWRPKHAKSSANMDQITAKRA

SEQ ID NO: 112 - DNA, 1293 bp, Helianthus annuus
atggtagctatgagtgctactgcgtcgctgtttccggtttcttccccaaaacctcactctggagccaagacatctgataagcttggaggtgaaccaggtagtgttgctgtgcgcggaatcaagacaaaatctgttaattccggtggtatgaaagttaaggctaacgcacaggctcctactgaggtgaatgggagtagatcacgtatcacgcatggcttcaaaaccgatgattattctacatcacctgccccgagaacctttatcaaccaattgcccgattggagcatgcttcttgctgcaatcacaacaatcttcttggctgcagagaagcaatggatgatgctggaatggaagaccaaacgccccgatatgattgctgatatggatcctttcggtttagggaggattgttcaagatggccttgtattccgtcaaaacttctctattagatcatatgaaataggggctgatcgaactgcatcgatagaaaccctaatgaatcatttacaagaaacggcccttaatcatgtaaagtctgcgggtcttctgggcgatggattcggttcaacaccagaaatgtgcaagaagaatctattttgggtggtgacaaagatgcaggtgatagttgaccgttatccaacttggggtgatgttgttcaagtagatacttgggtagccccaaatgggaaaaatggtatgcgccgtgattggctcgttcgcgattataaaacaggcgagattttaacaagagcctcaagtaactgggttatgatgaataaagagacaaggaggttatcgaaaatcccagatgaagttcgaggtgaaatagagcattactttgtagatgcacctccggttgtggaggatgattctagaaaattatctaaacttgacgaaagcactgctgactatgttcgcgacggtttgattccaagatggagtgatttggatgtcaaccagcatgttaacaatgtgaagtatattggctggatccttgagagtgctccacaagttgtggagaagtacgagcttgctcgcattactctcgagtaccgtagagaatgtaggaaggatagtgtggtgaaatcactgacctcggtattaggtggtggcgacgacgacaatggtggaataggcgattctggccgtgttgattgccaacatgtgctcttgtttgcgggtggyggagatggtactcctggtggcgagattgtgaagggaaggacccagtggcggccgaaatatgagaaacaagatgggagtgttgatcacttctctgctggaaatgtttaa

SEQ ID NO: 113 - protein, 430 aa, Helianthus annuus
MVAMSATASLFPVSSPKPHSGAKTSDKLGGEPGSVAVRGIKTKSVNSGGMKVKANAQAPTEVNGSRSRITHGFKTDDYSTSPAPRTFINQLPDWSMLLAAITTIFLAAEKQWMMLEWKTKRPDMIADMDPFGLGRIVQDGLVFRQNFSIRSYEIGADRTASIETLMNHLQETALNHVKSAGLLGDGFGSTPEMCKKNLFWVVTKMQVIVDRYPTWGDVVQVDTWVAPNGKNGMRRDWLVRDYKTGEILTRASSNWVMMNKETRRLSKIPDEVRGEIEHYFVDAPPVVEDDSRKLSKLDESTADYVRDGLIPRWSDLDVNQHVNNVKYIGWILESAPQVVEKYELARITLEYRRECRKDSVVKSLTSVLGGGDDDNGGIGDSGRVDCQHVLLFAGGGDGTPGGEIVKGRTQWRPKYEKQDGSVDHFSAGNV

SEQ ID NO: 114 - DNA, 1275 bp, Iris tectorum
atggttgcttccgtgtccgcctcggccttcttcccggtcccctcctcctcgtcctcctcttcctcttcgagctctaccgggtccacaaaaccctcgtccatctccctcgggaaagggcccgatgccctcgatgcccggggcctcgtggccaaacccgcatccaattccggcagcttacaagtaaaggtcaatgcccaagccgccaccagggttaatggatccaaggtcgggttgaaaaccgataccaacaagcttgaggacacacccttttttccttcctccgccccgaggactttctacaaccaattgccagactggagcgtctcctttgctgccatcaccaccatcttcttggctgctgagaagcaatggacgcttatcgattggaagccaaggcggcccgacatgctcgccgatgcattcggccttggaaagattattgagaatggacttgtctacaggcagaacttctccataaggtcatatgagattggggcggatcagacggcatctatagagacgttaatgaatcatttacaggaaacggcgttaaaccatgtgaagtgtgccggactcttgggtaatgggtttggttccacgccggagatgagtaaaaagaatttaatatgggtcgtcaccaaaatgcaggtccttgtggagcattatccttcctgggggaatgttattgaagtagatacatgggctgcggtatctggaaagaatggaatgcggcgtgattggcatgttcgggactgccaaaccggtcaaactatcatgagaagctccagcaattgggtgatgatgaacaaggacaccaggaggttgtctaaatttcctgaagaagttagagctgaaatagaaccctacttcatggagcgtgttcctgtcattgatgatgacaacaggaagctccctaagcttgatgatgatactgctgatcatgttcgcaagggtctaactccaagatggagtgacttggatgtcaatcagcatgtgaacaatgtcaagtacattggatggatccttgagagtgctccaatctccatcctggagagtcatgagcttgcaagcatgactcttgagtacaggagggagtgtggaagggacagcatgctgcagtccctcacctcactttctaacgattgcactgatgggctcggcgagcttcccattgaatgtcagcatctactccgctcgagggtgggcctgaatgtgaaaggacgaactgagtggaggcccaagaaacgtgcccccttccctgttgggagcccatga

SEQ ID NO: 115 - protein, 424 aa, Iris tectorum
MVASVSASAFFPVPSSSSSSSSSSSTGSTKPSSISLGKGPDALDARGLVAKPASNSGSLQVKVNAQAATRVNGSKVGLKTDTNKLEDTPFFPSSAPRTFYNQLPDWSVSFAAITTIFLAAEKQWTLIDWKPRRPDMLADAFGLGKIIENGLVYRQNFSIRSYEIGADQTASIETLMNHLQETALNHVKCAGLLGNGFGSTPEMSKKNLIWVVTKMQVLVEHYPSWGNVIEVDTWAAVSGKNGMRRDWHVRDCQTGQTIMRSSSNWVMMNKDTRRLSKFPEEVRAEIEPYFMERVPVIDDDNRKLPKLDDDTADHVRKGLTPRWSDLDVNQHVNNVKYIGWILESAPISILESHELASMTLEYRRECGRDSMLQSLTSLSNDCTDGLGELPIECQHLLRSRVGLNVKGRTEWRPKKRAPFPVGSP

SEQ ID NO: 116 - DNA, 1257 bp, Jatropha curcas
atggttgctactgctgctacttcctcgttcttccctgttcctacttcatctgcagattccaagtccaccaagattggtagtgggtctgcaagtttgggaggaatcaaatcaaaacctgcttcttctgggggcttgcaagtcaaggcaaatgcccaagcccctcccaagataaatggatccacagtaggctatacaacacctgtggacagtgtgaaaaatgagggtgacacgccatcaccgcccccaaggacctttatcaaccaattacctgattggagcatgcttcttgctgctattacaactatattcttggcagcagagaagcagtggatgatgcttgactggaaaccacggcgacctgacatgcttattgacccttttggtctagggagaattgttcaggatggccttgtgttcaggcagaacttctccatccgatcatatgaaattggcgcggatcggacagcatccatagagacattgatgaatcatttacaagaaacagccctcaaccatgttaagactgctggacttcttggtgaggggtttggttcaacaccagagatgagtaaaaggaacctgatatgggtggttactcggatgcaggtcctggtggatcgttatccaacgtggggtgatgttgttgaagtagatacttgggtgagtgcatcaggaaaaaatggcatgcgccgcgattggcttgttcgtgacagtaaaaccggtgaaactctaacaagagcctccagtgtgtgggtaatgatgaataaactgactaggagattatctaaaattcctgaagaggttaggggggaaatagagccttactttttgaattctgatcctattgtggatgaggatggcagaaaactgccaaaacttgatgacaacactgcggattatgtttgcaaaggtttaactcctagatggagtgatttagatgtcaaccaacatgttaacaatgtgaagtacattggctggatccttgagagtgctccgctgccgatcctggagagtcatgagctatcatccattattatggaatataggagggagtgtggaagggatagtgtgcttcagtcgctgactgctgtctctggcaccggcttaggaaatttaggaaatgctggtgaaattgagtgtcagcacttgcttcgactggaggaaggtgctgagatagtaaggggaaggactgcgtggaggccaaagtatcgcagcaactttggaattatgggtcagattccagttgaaagtgcctaa

SEQ ID NO: 117 - protein, 418 aa, Jatropha curcas
MVATAATSSFFPVPTSSADSKSTKIGSGSASLGGIKSKPASSGGLQVKANAQAPPKINGSTVGYTTPVDSVKNEGDTPSPPPRTFINQLPDWSMLLAAITTIFLAAEKQWMMLDWKPRRPDMLIDPFGLGRIVQDGLVFRQNFSIRSYEIGADRTASIETLMNHLQETALNHVKTAGLLGEGFGSTPEMSKRNLIWVVTRMQVLVDRYPTWGDVVEVDTWVSASGKNGMRRDWLVRDSKTGETLTRASSVWVMMNKLTRRLSKIPEEVRGEIEPYFLNSDPIVDEDGRKLPKLDDNTADYVCKGLTPRWSDLDVNQHVNNVKYIGWILESAPLPILESHELSSIIMEYRRECGRDSVLQSLTAVSGTGLGNLGNAGEIECQHLLRLEEGAEIVRGRTAWRPKYRSNFGIMGQIPVESA

SEQ ID NO: 118 - DNA, 1248 bp, Malus domestica
atggttgccactgctgctactgcctcgttctttccggtttcttctcccaactcagactcaagcgccaagaacgccaagctcgggtcagccaatttaggactcaaatcgaagtctgcatctggtggtttgcaggtaaaggcaaatgctcaagccccttcaaagataaatggaactagtgttggtttggcaactgtggaaagtgggaagcatggggatgacatttcatcccctccggcacggactttcattaaccaattacctgattggagtgtgctccttgctgctattaccacaatcttcttggctgcagagaagcaatggacaatgcttgattggaaacccaagcgacctgacatgctcattgacccatttggtctaggacgaattgttcaggatggtcttgtctttcgccagaacttctcaattagatcatatgaaataggtgctgatcgtacggcttcaatagagacgttaatgaatcatttacaggaaacagcacttaatcatgttaagactgctggacttctgggagatggttttggttcaactccagagatgactgtaagaaacctgatatgggtggtaacgaagatgcaggttgtggtagaccgctatcctacttggggtgacgttgttcaagttgacacttgggttagtgcctctgggaagaatggaatgcgtcgtgattggattatccaggatttgaaaactggtcaaattctaacaagagcctccagtgtgtgggtgatgatgaataaagtgacgaggagattatcaaagatgcctgatgcagttcgcggtgaaatagagtccttttttatgaattctcctcctgttgtggaggaagatggcaggaaactgccgaaacttgatgacaaaacagcggacgttgttctctctggtttgactcctagatggagtgatttagatgtcaaccagcatgttaataacgtgaagtacattggctggatccttgagggtgctcccttgccaatcctggagagtcatgagctctcttctttgactctggagtataggagggagtgcgggagggacagtgtgcttcagtctctgactgcagtctcaggtgctgatatcggcaacctgggaagtaatggcacggtggagtgccagcacatgcttcgacttgaggatggggctgagattgtgaggggaaggactgagtggaggcccaaatatgccaacaatcttgggattgtgggtcatcttccagcagaaagcgcatag

SEQ ID NO: 119 - protein, 415 aa, Malus domestica
MVATAATASFFPVSSPNSDSSAKNAKLGSANLGLKSKSASGGLQVKANAQAPSKINGTSVGLATVESGKHGDDISSPPARTFINQLPDWSVLLAAITTIFLAAEKQWTMLDWKPKRPDMLIDPFGLGRIVQDGLVFRQNFSIRSYEIGADRTASIETLMNHLQETALNHVKTAGLLGDGFGSTPEMTVRNLIWVVTKMQVVVDRYPTWGDVVQVDTWVSASGKNGMRRDWIIQDLKTGQILTRASSVWVMMNKVTRRLSKMPDAVRGEIESFFMNSPPVVEEDGRKLPKLDDKTADVVLSGLTPRWSDLDVNQHVNNVKYIGWILEGAPLPILESHELSSLTLEYRRECGRDSVLQSLTAVSGADIGNLGSNGTVECQHMLRLEDGAEIVRGRTEWRPKYANNLGIVGHLPAESA

SEQ ID NO: 120 - DNA, 1284 bp, Oryza sativa
atggctggttctcttgcggcgtctgcattcttccctgtcccagggtcttcccctgcagcttcggctagaagctctaagaacacaaccggtgaattgccagagaatttgagtgtccgcggaatcgtcgcgaagcctaatccgtctccaggggccatgcaagtcaaggcgcaggcgcaagcccttcctaaggttaatggaaccaaggttaacctgaagactacaagcccagacaaggaggatataataccgtacactgctccgaagacattctataaccaattgccagactggagcatgcttcttgcagctgtcacgaccattttcctggcagctgagaagcagtggactctgcttgactggaagccgaagaagcctgacatgctggctgacacattcggctttggtaggatcatccaagacgggctggtgtttaggcaaaacttcttgattcggtcctacgagattggtgctgatcgtacagcttctattgagacattaatgaatcatttacaggaaacagctctgaaccatgtgaaaactgctggtctcttaggtgatggttttggtgctacgccggagatgagcaaacggaacttaatatgggttgtcagcaaaattcagcttcttgttgagcgatacccatcatggggagatatggtccaagttgacacatgggtagctgctgctggcaaaaatggcatgcgtcgagattggcatgttcgggactacaactctggtcaaacaatcttgagggctacaagtgtttgggtgatgatgaataagaacactagaagactttcaaaaatgccagatgaagttagagctgaaataggcccgtatttcaatggccgttctgctatatcagaggagcagggtgaaaagttgcctaagccagggaccacatttgatggcgctgctaccaaacaattcacaagaaaagggcttactccgaagtggagtgaccttgatgtcaaccagcatgtgaacaatgtgaagtatattggttggatacttgagagtgctccaatttcgatactggagaagcacgagcttgcaagcatgaccttggattacaggaaggagtgtggccgtgacagtgtgcttcagtcgcttaccgctgtttcaggtgaatgcgatgatggcaacacagaatcctccatccagtgtgaccatctgcttcagctggagtccggagcagacattgtgaaggctcacacagagtggcgaccgaagcgagctcagggcgaggggaacatgggctttttcccagctgagagtgcatga

SEQ ID NO: 121 - protein, 427 aa, Oryza sativa
MAGSLAASAFFPVPGSSPAASARSSKNTTGELPENLSVRGIVAKPNPSPGAMQVKAQAQALPKVNGTKVNLKTTSPDKEDIIPYTAPKTFYNQLPDWSMLLAAVTTIFLAAEKQWTLLDWKPKKPDMLADTFGFGRIIQDGLVFRQNFLIRSYEIGADRTASIETLMNHLQETALNHVKTAGLLGDGFGATPEMSKRNLIWVVSKIQLLVERYPSWGDMVQVDTWVAAAGKNGMRRDWHVRDYNSGQTILRATSVWVMMNKNTRRLSKMPDEVRAEIGPYFNGRSAISEEQGEKLPKPGTTFDGAATKQFTRKGLTPKWSDLDVNQHVNNVKYIGWILESAPISILEKHELASMTLDYRKECGRDSVLQSLTAVSGECDDGNTESSIQCDHLLQLESGADIVKAHTEWRPKRAQGEGNMGFFPAESA

SEQ ID NO: 122 - DNA, 1326 bp, Picea glauca
atggtagccgccgctgcaacaatgctaatgttttcttcaagctctcagtgcaacacacagaacaagatctcgtcatctgcttcatcagggaagcccacaatgccagttagctctcctgagcgtgttgatgttaagtccaaacccactgcatacaagggactccaagtcaatggaaattcccacggagctactaataagataaatggcactaaggtgaacggaacagcagtggatagcatgaagcataacgttggcctgaaggaagcatccgaggaagaaagcactgctaagagcaggatcaatcagctcccagattggagtatgcttctcgcaactattgctaccattattctggcagccgaaaagcagtggaccaattttgattggaagccaaggaaaacagacgtgtttggtgacgttttcaggctgggcaggtttgtggaagacagtctggttttccggcagaacttcgccataagatcttatgaaattggtgcagacaaaacggcttctattgaaaccttgatgaaccatcttcaggaaactgcccttaatcatgtttggctttctgggctagctggggatggattcggtgctactcttgagatgagccggagaaatctcctatgggttgtggctcgcatgcaaattcaagttgaacgatatccctcatggggtgatgttgtggagatagatacatgggttgggccatcaggtaaaaatggcatgcggcgtgattggcttgttcgagattcgaagacgaatgccatccttacacgagctactagtacctgggtaatgatgaatagaaagacaagaaaactgtccaaaattcctgatgctgtcaaagcagagatacagccttatttcacagaaagaaatgtctttgtggcagaagacaccagaaagttgcataagctggaggatgacactgcccagtacatctgttcggatttaacaccgcggtggagtgatttggatgtgaatcagcatgtcaataatgttaaatatattggttggattttggagagtttacccatctctgttttagagggcaacgaactagctaatataacgttggagtacagacgtgaatgtggaccgacgcatgtactccaatcattgacaagtccacaggctggtgaggtgattgctgcttcagctgcaccattttcacagagaaatgatcctccagacacctggaaacccttgcctgcattgcagtttgcacacttgcttcgattgcaagatgacagatcggaaattctgagggcaaggtcagagtggaggtcaaaggcaaagaacaaccttcacgaccttgcttga

SEQ ID NO: 123 - protein, 441 aa, Picea glauca
MVAAAATMLMFSSSSQCNTQNKISSSASSGKPTMPVSSPERVDVKSKPTAYKGLQVNGNSHGATNKINGTKVNGTAVDSMKHNVGLKEASEEESTAKSRINQLPDWSMLLATIATIILAAEKQWTNFDWKPRKTDVFGDVFRLGRFVEDSLVFRQNFAIRSYEIGADKTASIETLMNHLQETALNHVWLSGLAGDGFGATLEMSRRNLLWVVARMQIQVERYPSWGDVVEIDTWVGPSGKNGMRRDWLVRDSKTNAILTRATSTWVMMNRKTRKLSKIPDAVKAEIQPYFTERNVFVAEDTRKLHKLEDDTAQYICSDLTPRWSDLDVNQHVNNVKYIGWILESLPISVLEGNELANITLEYRRECGPTHVLQSLTSPQAGEVIAASAAPFSQRNDPPDTWKPLPALQFAHLLRLQDDRSEILRARSEWRSKAKNNLHDLA

SEQ ID NO: 124 - DNA, 1266 bp, Populus tomentosiformis
atggttgccacagcagctacttcatcatttttcccagttccttcaccacctggagatgccaagtcctccaaggttggtagtggttctgcaagtttgggaggaatcaaatcgaaatctgcttcctctggagctttgcaggttaaggcaaatgcccaagctcctccgaagataaatggctctccagttggcttgacagcatcagtggaaactgcgaagaaggaggatgttgtctcatcaccggcaccccggacatttatcaaccaattacctgattggagcatgcttcttgctgcaattacaaccatgtttttggcagcagagaagcagtggatgatgcttgattggaaaccaaagcgagctgacatgcttattgatccctttggtattggaagaattgtccaagatggtcttgtcttcagccagaatttctcaattaggtcatatgaaattggtgcagatcgtactgcgtctatagagacgttgatgaaccatttacaagaaacagcacttaatcatgttaagactgctgggcttcttggagatggatttggttcaaccccagagatgtccaaaaggaacctgatatgggtggtaactcgaatgcagattctagtcgatcgttatcctacatggggtgatgttgtccatgtggatacttgggtgagtgcatcaggaaagaatggtatgcgccgtgattggcttgtccgtgatgctaaaactggtgaaactcttacaagagcctccagtttgtgggtgatgatgaataaagtgacaaggaggttatctaaaattcctgaagatgttcgaggtgaaatagagccttattttctgaattctgatcctgttgtgaatgaggacagcacaaaactgccaaaacttgacgacaagacggcggactatatccgcaaaggcctaactcctagatggaatgatttagatgtcaaccagcatgttaacaatgtgaaatacataggctggatccttgagagcgctcctcccccaatcctggagagtcatgagcttgctgccattactttggagtacaggagggagtgtggcagggacagcgtgctgcagtccttgactgctgtatctggcgctggcattggaaatttgggcggtcctggtaaagttgagtgtcaacatttgctgcgacatgaggatggtgctgagatcgtgaggggaaggaccgagtggaggcccaaacatgccaacaattttggcatgatgggtggtcagatgccagctgatgagagcggtgcttaa

SEQ ID NO: 125 - protein, 421 aa, Populus tomentosiformis
MVATAATSSFFPVPSPPGDAKSSKVGSGSASLGGIKSKSASSGALQVKANAQAPPKINGSPVGLTASVETAKKEDVVSSPAPRTFINQLPDWSMLLAAITTMFLAAEKQWMMLDWKPKRADMLIDPFGIGRIVQDGLVFSQNFSIRSYEIGADRTASIETLMNHLQETALNHVKTAGLLGDGFGSTPEMSKRNLIWVVTRMQILVDRYPTWGDVVHVDTWVSASGKNGMRRDWLVRDAKTGETLTRASSLWVMMNKVTRRLSKIPEDVRGEIEPYFLNSDPVVNEDSTKLPKLDDKTADYIRKGLTPRWNDLDVNQHVNNVKYIGWILESAPPPILESHELAAITLEYRRECGRDSVLQSLTAVSGAGIGNLGGPGKVECQHLLRHEDGAEIVRGRTEWRPKHANNFGMMGGQMPADESGA

SEQ ID NO: 126 - DNA, 1260 bp, Ricinus communis
atggttgctactgcggctgctgctacttcctctttctttccagttccttctcaatctgcggatgctaatttcgataaggcacctgcaagcttaggtggaatcaaattaaaatctacctcttgctctcggggtttacaggttaaggcaaatgcgcaagcccctcccaagataaatggatcctcggtaggattcacaacatctgtggaaactgtgaagaatgacggtgacatgccattaccaccaccccctaggacttttatcaaccaattacctgattggagcatgcttcttgctgctattacaactatctttttggctgctgaaaagcagtggatgatgcttgactggaaaccaaggcggcctgacatgcttatcgacccgtttggtataggtagaattgttcaggatggtcttatttttcgccagaacttctccataagatcatatgaaattggtgctgatcgtacagcatccatagagacattaatgaatcatttacaagaaacggccctcaatcatgttaagactgctggacttcttggggatggatttggttcaaccccagagatgagcaaaaggaacctcatatgggtggttactcggatgcaggttctggtggatcgttacccaacatggggtgatgttgttcaagtagatacttgggtgagtaaatcaggaaagaatggcatgcggcgtgattggtgcgtccgtgatagtagaactggtgaaactttaacgagagcatccagcgtgtgggtgatgatgaataaactgactaggaggttatctaaaattcccgaagaagttcgaggagaaatagagccttattttctgaattctgatcctattgtggatgaggatagcagaaaactgccaaagcttgatgatagcaatgcggactatgtccgcaaaggtctaactcctagatggagtgatctagatatcaaccaacatgttaacaatgtgaaatacattggctggattcttgagagtgctccactgccaatactggagagtcatgaactctctgccattactctggagtataggagggagtgcgggagggacagtgtactgcagtctctgactgctgtatccggtaatggtattggaaatttgggaaatgctggtgatattgagtgccagcacttgcttcgacttgaggatggggctgagatagtgaggggaaggaccgagtggaggccaaagtacagcagcaactttggtattatgggtcagattccagtcgaaagtgcttaa

SEQ ID NO: 127 - protein, 419 aa, Ricinus communis
MVATAAAATSSFFPVPSQSADANFDKAPASLGGIKLKSTSCSRGLQVKANAQAPPKINGSSVGFTTSVETVKNDGDMPLPPPPRTFINQLPDWSMLLAAITTIFLAAEKQWMMLDWKPRRPDMLIDPFGIGRIVQDGLIFRQNFSIRSYEIGADRTASIETLMNHLQETALNHVKTAGLLGDGFGSTPEMSKRNLIWVVTRMQVLVDRYPTWGDVVQVDTWVSKSGKNGMRRDWCVRDSRTGETLTRASSVWVMMNKLTRRLSKIPEEVRGEIEPYFLNSDPIVDEDSRKLPKLDDSNADYVRKGLTPRWSDLDINQHVNNVKYIGWILESAPLPILESHELSAITLEYRRECGRDSVLQSLTAVSGNGIGNLGNAGDIECQHLLRLEDGAEIVRGRTEWRPKYSSNFGIMGQIPVESA

SEQ ID NO: 128 - DNA, 1263 bp, Solanum tuberosum
atgatggccactgctgctacttgtgcattcttccctgctgctaatccacctcctgactctggagctaaatcgtctggaaatttaggaggaagtcttcctggaagtatagatacacgggggcttaatgttaagaagccttcttttgggagcctacaagctaaggccaatgcacaagcaccacctaaggtgaatggaacaaaggtaggcgttatggatggcttcaaaaatgacgatgaggtgatttcttcacatcacccaaggacttttatcaaccagttacctgattggagcatgctcctcgccgccatcacgacaatttttttagctgctgagaagcaatggatgatgcttgattggaagcctaagcgtcctgatatgctcgctgatccatttggattaggaaaaattgtgcaggatggctttgttttccgtcaaaatttcagcatcaggtcttatgaaataggggctgataggactgcgtctatagaaacaatgatgaatcatttacaggaaactgctcttaaccatgtcaagagtgctggactcatgcatggtgggttcggatcaactccagagatgtccaagagaaatttgatctgggtcgttactaaaatgcaggttgtggtggaccgttatcctacttggggtgatgttgttcaagtagacacttgggtagctgcatcggggaaaaatggtatgcgcagagattggctcctccgcgatagtaatacaggggatatattgatgagagcttccagccaatgggttatgatgaataaggagacgaggagattatctaaaataccagatgaggctcgggctgaaattgaaggttattttgttgattcacctcctgttattgatgaggacagcaggaagttaccaaaacttgatgagacaacagcagactacactcgaactggtttaactccaagatggagtgatttagatgttaaccagcatgttaataatgtcaagtacattggctggattcttgagagtgcacccatgcaaatactagagggttgtgagcttgctgccatgactttggagtaccgcagggagtgcagaagggacagtgtgcttcagtctcttacctctgtacttgacaaaggagtcggtgacttcaccgactttgggaatgttgagtgtcaacacgtccttcgacttgaaaatggcggagaggttgttaagggacgaactgagtggaggccgaaacttgtcaatggaattgggaccctaggcggattcgacttcgcctga

SEQ ID NO: 129 - protein, 420 aa, Solanum tuberosum
MMATAATCAFFPAANPPPDSGAKSSGNLGGSLPGSIDTRGLNVKKPSFGSLQAKANAQAPPKVNGTKVGVMDGFKNDDEVISSHHPRTFINQLPDWSMLLAAITTIFLAAEKQWMMLDWKPKRPDMLADPFGLGKIVQDGFVFRQNFSIRSYEIGADRTASIETMMNHLQETALNHVKSAGLMHGGFGSTPEMSKRNLIWVVTKMQVVVDRYPTWGDVVQVDTWVAASGKNGMRRDWLLRDSNTGDILMRASSQWVMMNKETRRLSKIPDEARAEIEGYFVDSPPVIDEDSRKLPKLDETTADYTRTGLTPRWSDLDVNQHVNNVKYIGWILESAPMQILEGCELAAMTLEYRRECRRDSVLQSLTSVLDKGVGDFTDFGNVECQHVLRLENGGEVVKGRTEWRPKLVNGIGTLGGFDFA

SEQ ID NO: 130 - DNA, 1254 bp, Tagetes erecta
atggttgctacggctgcaactgcatcgttatttccggtttcttcaccacaacctgactctggtgctaagaattctggcaatcacaaaggcggattgggtagtgttgacttacgtggaattaagtcaaagtcaacgtcttctaatggtttgcaagttaagacgaatgcacaagctcctgcgaaggtgaatgggaccagggtaggtgttatggatggactgaaaattgatgacagttcatcatcgggtgccccaagaacatttattaaccaactgcctgattggagcatgcttcttgctgctattactactattttcttggctgctgaaaagcaatggatgatgctggattggaagactaaacgtccggacatgcttgctgatcttgatccttttggtttcgggcgaattgttgaggatggatttgtatttcgtcaaaacttttcaattagatcatatgaaataggggcggatcgaactgcgtcggttgaaacgttgatgaatcatttgcaggaaacggcccttaatcatgtaaaaaatgctggactcctcggtgatggctttggctcaacacctgaaatgtctaaaaggaatctgttctgggtggtaactaagatgcaagtgctagtagaccgttatccaacttggggtgacgtggttcaagtagatacttgggtagctgcttctgggaaaaatggcatgcgtcgtgattggttgattcgtgattgcaaaacgggtcagatactaacaagagcctcaagtaattgggttatgatgaataaagttacaaggaggttatcaaaaatgcccgatgaagttcgggctgaaattgagccgtattttgttgacacgcctcctgtggttgatgatgatgatagaaaattaccaaaacttgatgagaacactgctgaccatgttcgtaatggtttaactccaaagtggagtgatttggatgtcaatcagcatgtcaacaatgtgaagtatgttggctggattcttgagagtgcaccacagcatgtggtagagaactatgagcttgcaagcctcacccttgagtaccgccgtgagtgtatgaaagacagcgtgctgcagtcactcacttccttgctggcgggtggtgagaaggcggattctgatgatgtggactgtcaacacctgcttcgactagaaggtggcggtgagattgtgaagggaaggaccaaatggaggcccaaatatgtgaaacagattcaagaacatcaatcatttccctactga

SEQ ID NO: 131 - protein, 417 aa, Tagetes erecta
MVATAATASLFPVSSPQPDSGAKNSGNHKGGLGSVDLRGIKSKSTSSNGLQVKTNAQAPAKVNGTRVGVMDGLKIDDSSSSGAPRTFINQLPDWSMLLAAITTIFLAAEKQWMMLDWKTKRPDMLADLDPFGFGRIVEDGFVFRQNFSIRSYEIGADRTASVETLMNHLQETALNHVKNAGLLGDGFGSTPEMSKRNLFWVVTKMQVLVDRYPTWGDVVQVDTWVAASGKNGMRRDWLIRDCKTGQILTRASSNWVMMNKVTRRLSKMPDEVRAEIEPYFVDTPPVVDDDDRKLPKLDENTADHVRNGLTPKWSDLDVNQHVNNVKYVGWILESAPQHVVENYELASLTLEYRRECMKDSVLQSLTSLLAGGEKADSDDVDCQHLLRLEGGGEIVKGRTKWRPKYVKQIQEHQSFPY

SEQ ID NO: 132 - DNA, 1266 bp, Vitis vinifera
atggttgccactgcagccacttctgcattctttgcagttgcttctccatcttctgatccagatgccaaaccttccaccaagccgggggttgggtctgcaattttgaggggaatcaagtcaagaaatgctccttcaggcagtttgcaagttaaggcaaatgcccaagcccctcctaagataaatggtaccacagttggttatacctcctcggcggaaggcgtgaagattgaggatgacatgtcgtcgcctccacctaggactttcatcaaccaattgccagactggagcatgcttcttgctgctattacaaccatcttcttggcagctgagaagcagtggatgatgcttgactggaaaccaaggaggtctgacatgctaatcgacccatttggcttagggaaaattgtccaagatggtcttgttttcaggcaaaacttctcgattagatcatatgaaataggtgctgatcgaaccgcatccatagaaacgttgatgaatcatttacaggaaactgcacttaaccatgttaggactgctggtcttctgggtgatggttttggttcaacgccagagatgagcataaggaacctaatatgggtggtcactcgaatgcaggttgtggtagatcggtaccctacttggggtgatgttgttcaagtggatacttgggtatgtgcatctgggaagaatggcatgcgtcgtgattggataatccgtgattgcaaaactggggaaactctaaccagagcctccagtgtgtgggtgatgatgaataagcagaccaggagattatcaaaaattccagatgcagttcgagctgaaatagagccttattttatggattctgctcctattgtggatgaggatggcagaaaactgcccaaacttgatgacagcactgcggattatatccgcacaggactaactcctagatggagtgatttagatgtcaatcagcatgttaacaatgttaagtacatcggttggatccttgagagtgctccactgccaatcttggagagtcacgagctttcttccatgactctggagtacaggagggagtgtggaagggacagtgtgctgcagtccctcactgctgtctgcggaactggtgttggtaatttgctggattgtggaaatgttgagtgccagcaccttcttcgacttgaggaaggagctgagattgttaagggaaggactgagtggaggccaaagtatgcccacagcatggggggtgtgggccagatcccagcagaaagtgcttga

SEQ ID NO: 133 - protein, 421 aa, Vitis vinifera
MVATAATSAFFAVASPSSDPDAKPSTKPGVGSAILRGIKSRNAPSGSLQVKANAQAPPKINGTTVGYTSSAEGVKIEDDMSSPPPRTFINQLPDWSMLLAAITTIFLAAEKQWMMLDWKPRRSDMLIDPFGLGKIVQDGLVFRQNFSIRSYEIGADRTASIETLMNHLQETALNHVRTAGLLGDGFGSTPEMSIRNLIWVVTRMQVVVDRYPTWGDVVQVDTWVCASGKNGMRRDWIIRDCKTGETLTRASSVWVMMNKQTRRLSKIPDAVRAEIEPYFMDSAPIVDEDGRKLPKLDDSTADYIRTGLTPRWSDLDVNQHVNNVKYIGWILESAPLPILESHELSSMTLEYRRECGRDSVLQSLTAVCGTGVGNLLDCGNVECQHLLRLEEGAEIVKGRTEWRPKYAHSMGGVGQIPAESA

SEQ ID NO: 134 - DNA, 1281 bp, Zea mays
atggctggctcccttgctgcctcagccttcttccctggcccaggggcgtctccagcagcatccgcgaagaacttggctggtgaagtaccggatagtttgagcgtccgtggtattgtcgcaaagcctaatgccaattctgggaacatgcaagtgaaggctcaagcacaaacccttcccaaggttaatggcaccaaggttaacctcaagaatgcaagctcagacacagaggaggcgataccctacactgctcccaagacattctacaaccaactgccagattggagcatgcttcttgcggctgtcactaccatcttcctggcagcagagaagcagtggacactgcttgactggaagccgaagaaacccgacatgcttgttgatacatttggttttggtgggatcatccaggatgggatggtgtttaggcaaaacttcattattcggtcctatgagattggtgccgatcgtactgcttctatagagacattaatgaatcacttacaggaaacagctcttaaccatgtgaagacagctggccttcttggagatggttttggcgccacgccagagatgagcaaacgaaacttgatccacgaggtcagcaaaattcagcttcttgttgagaagtaccccttgtgggaagacacggttcaagtggacacgtgggtagctgccgctgggaaaaatggcatgcgtcgagactggcatgtcctcgactgcaagtctggatgtacgatcttgagagctacaagtgtttgggtgatgatgaataagaacactagaaggttttcaaaaatgccggacgaagtaagggctgagataggcccgtatttcaacgcccgcgcagccataacagatgagcagagcgagaaactggctaagccagggagcactgctggtggcgatgctatgaagcagttcatgagaaaggggctcactcctaggtggtggggtgaccttgatgtcaaccagcacgtgaataacgtcaagtacatcggttggattcttgagagtgctccgatcgcgatcctggagaagcacgagctcgcaagcatgacgctggattacaggaaggagtgcggacgcgacagcgtgctgcagtcgctcaccaccgtcgcgggtgaatgcgtagacggcgacacagactccaccatccagtgcgaccacctgctccagctggaaacaggagccgatattgtgaaggcgcacacggagtggcgcccgaagcgggcgcatggtgaggggacccccatggggggtttcccggcggagagcgcgtga

SEQ ID NO: 135 - protein, 426 aa, Zea mays
MAGSLAASAFFPGPGASPAASAKNLAGEVPDSLSVRGIVAKPNANSGNMQVKAQAQTLPKVNGTKVNLKNASSDTEEAIPYTAPKTFYNQLPDWSMLLAAVTTIFLAAEKQWTLLDWKPKKPDMLVDTFGFGGIIQDGMVFRQNFIIRSYEIGADRTASIETLMNHLQETALNHVKTAGLLGDGFGATPEMSKRNLIHEVSKIQLLVEKYPLWEDTVQVDTWVAAAGKNGMRRDWHVLDCKSGCTILRATSVWVMMNKNTRRFSKMPDEVRAEIGPYFNARAAITDEQSEKLAKPGSTAGGDAMKQFMRKGLTPRWWGDLDVNQHVNNVKYIGWILESAPIAILEKHELASMTLDYRKECGRDSVLQSLTTVAGECVDGDTDSTIQCDHLLQLETGADIVKAHTEWRPKRAHGEGTPMGGFPAESA

SEQ ID NO: 136 - DNA, 1257 bp, Zea mays
atggccgcctccatcgcggcctcgtccttctttccagggtcaccggcgccggccgctcctaagaacggccttggggagcgcccagagagcctggacgtccgcggcgttgcggcgaagccgggagcctcgtctagtgccgtgagggcgagcaagacgcgcgcccacgctgcggtccccaagatgaacggtgggggcaagtccgcggtggcggatggggagcacgaaaccgtaccttcttcggtgccgaagactttctacaaccagcttcccgactggagcatgctccttgcggccatcaccaccatcttcttggccgcagagaagcagtggacgatgcttgactggaagcctaggaggcctgacatgctcactgacacgtttgggtttggccggatcatacatgatgggctcatgttcaggcagaacttctccattaggtcctatgagattggggcagataggacggcatctatagagacgctgatgaaccatttgcaggaaacggcactcaatcatgtgaagaccgctgggctgctaggtgatggatttggctccacaccagagatgagtaaacgaaacttgttctgggtggttagccaaatgcaggccatcatcgagcgttatccatgctggggtgatactgttgaagtagatacatgggttagtgctaatggtaaaaatggaatgcgtagggattggcatatacgtgattctatgacaggccacacaatactgaaggcgacaagtaaatgggttatgatgaacaaactcactaggaagcttgcaagaattccagatgaagtgcggactgaaatagagccatactttgttgggcgttctgctattgttgatgaagacaaccgcaagcttccaaaactgccagagggtcaaagcacttctgcagctaaatatgtgaggacaggcctgactcctcgttgggctgatcttgatataaaccagcatgtcaataatgttaaatacattgcgtggattcttgagagtgcaccgattactatttttgagaatcatgagctggccagcattgtgctggattacaaaagggagtgtgtccgcgatagtgtgctgcagtcacacacctctgtccatgaggattgcaacattgagtctggagaaacaaccttgcactgtgagcatgtgctgagccttgaatcaggtccgaccatagtgaaggcccggaccatgtggaggcctaagggaaccaaggcccaagaaacagcggttccatcttcattctga

SEQ ID NO: 137 - protein, 418 aa, Zea mays
MAASIAASSFFPGSPAPAAPKNGLGERPESLDVRGVAAKPGASSSAVRASKTRAHAAVPKMNGGGKSAVADGEHETVPSSVPKTFYNQLPDWSMLLAAITTIFLAAEKQWTMLDWKPRRPDMLTDTFGFGRIIHDGLMFRQNFSIRSYEIGADRTASIETLMNHLQETALNHVKTAGLLGDGFGSTPEMSKRNLFWVVSQMQAIIERYPCWGDTVEVDTWVSANGKNGMRRDWHIRDSMTGHTILKATSKWVMMNKLTRKLARIPDEVRTEIEPYFVGRSAIVDEDNRKLPKLPEGQSTSAAKYVRTGLTPRWADLDINQHVNNVKYIAWILESAPITIFENHELASIVLDYKRECVRDSVLQSHTSVHEDCNIESGETTLHCEHVLSLESGPTIVKARTMWRPKGTKAQETAVPSSF

SEQ ID NO: 138 - DNA, 1257 bp, Populus trichocarpa
atggttgccgctgcagctgcttcatcatttttcccagttccttcgccatctggagatgccaaggcctccaagtttggtagtgtgtctgcaagtttgggaggaatcaaaacgaaatctgcttcctctggggctttgcaagttaacacaaatgcccaagctcctccaaagataaatggccctccagttggcttgacagcatcagtggaaactctgaagaatgaggatgttgtgtcgtcaccggcacctcggacgttcatcaaccaattacctgattggagcatgcttcttgctgcaattacaaccatgtttttggcagcagagaagcagtggatgatgcttgattggaaaccaaagcgacctgatatgcttattgacccctttggtattgggagaattgtccaagatggtcttgtcttccgccagaatttctcaattaggtcatatgaaattggtgcagatcgtacagcatctatagagacgttgatgaaccatttacaagaaactgcacttaatcatgttaagactgctgggctccttggcgatggatttggtgcaaccccagagatgtccaaaaggaacctgatatgggtggtaactcgtatgcagattctggtagatcgttatcctacatggggtgatgttgttcaagtagatacttgggtgagtgcatcgggaaagaatggcatgcgccgtgattggcttctccgtgatgctaaaactggtgaaacgttgaccagagcctccagtgtgtgggtgatgatgaataaagtgacaaggaggttatccaaaattcctgaagaagttcgaggggaaatagagcctcattttctgacttctgatcctgttgtgaatgaggacagcagaaaacttccaaaaattgatgacaatacagcggactatatctgcgaaagtctaactcctagatggaatgatttagatgtcaaccaacatgttaacaatgtgaagtacataggctggatccttgagagcgctcctccaccaatcatggagagtcatgagcttgctgccattactttggagtacaggagggagtgtggcagggacagcgtgctgcagtccttgactgctgtatctgacactggcattggaaatttaggcagccctggtgaagttgagttccaacacttgctccggtttgaggagggtgctgagattgtgaggggaaggactgagtggagacccaaacatgccgacaattttggtatcatgggtcagatcccagctgtgagcgcttaa

SEQ ID NO: 139 - protein, 418 aa, Populus trichocarpa
MVAAAAASSFFPVPSPSGDAKASKFGSVSASLGGIKTKSASSGALQVNTNAQAPPKINGPPVGLTASVETLKNEDVVSSPAPRTFINQLPDWSMLLAAITTMFLAAEKQWMMLDWKPKRPDMLIDPFGIGRIVQDGLVFRQNFSIRSYEIGADRTASIETLMNHLQETALNHVKTAGLLGDGFGATPEMSKRNLIWVVTRMQILVDRYPTWGDVVQVDTWVSASGKNGMRRDWLLRDAKTGETLTRASSVWVMMNKVTRRLSKIPEEVRGEIEPHFLTSDPVVNEDSRKLPKIDDNTADYICESLTPRWNDLDVNQHVNNVKYIGWILESAPPPIMESHELAAITLEYRRECGRDSVLQSLTAVSDTGIGNLGSPGEVEFQHLLRFEEGAEIVRGRTEWRPKHADNFGIMGQIPAVSA

SEQ ID NO: 140 - protein, 266 aa, Artificial sequence
  IPR002864 Acyl-ACP thioesterase family comprised in SEQ ID NO 2
GLVFRQNFSIRSYEIGADRSASIETVMNHLQETALNHVKTAGLLGDGFGSTPEMFKKNLIWVVTRMQVVVDKYPTWGDVVEVDTWVSQSGKNGMRRDWLVRDCNTGETLTRASSVWVMMNKLTRRLSKIPEEVRGEIEPYFVNSDPVLAEDSRKLTKIDDKTADYVRSGLTPRWSDLDVNQHVNNVKYIGWILESAPVGIMERQKLKSMTLEYRRECGRDSVLQSLTAVTGCDIGNLATAGDVECQHLLRLQDGAEVVRGRTEWSS

SEQ ID NO: 141 - protein, 24 aa, Artificial sequence
  TMpred predicted transmembrane helix
TFINQLPDWSMLLAAITTIFLAAE

SEQ ID NO: 142 - DNA, 52 bp, Artificial sequence
  primer Prm 08145
ggggacaagtttgtacaaaaaagcaggcttaaacaatggtggccacctctgc

SEQ ID NO: 143 - DNA, 50 bp, Artificial sequence
  primer Prm 08146
ggggaccactttgtacaagaaagctgggttttttcttacggtgcagttcc

SEQ ID NO: 144 - DNA, 2194 bp, Oryza sativa; identical to SEQ ID NO: 85

SEQ ID NO: 145 - DNA, 1275 bp, Arabidopsis thaliana
atggatcctgaaggtttcacgagtggcttattccggtggaacccaacgagagcattggttcaagcaccacctccggttccacctccgctgcagcaacagccggtgacaccgcagacggctgcttttgggatgcgacttggtggtttagagggactattcggtccgtacggtatacgtttctacacggcggcgaagatagcggagttaggttttacggcgagcacgcttgtgggtatgaaggacgaggagcttgaagagatgatgaatagtctctctcatatctttcgttgggagcttcttgttggtgaacggtacggtatcaaagctgccgttagagctgaacggagacgattgcaagaagaggaggaagaggaatcttctagacgccgtcatttgctactctccgccgctggtgattccggtactcatcacgctcttgatgctctctcccaagaagatgattggacagggttatctgaggaaccggtgcagcaacaagaccagactgatgcggcggggaataacggcggaggaggaagtggttactgggacgcaggtcaaggaaagatgaagaagcaacagcagcagagacggagaaagaaaccaatgctgacgtcagtggaaaccgacgaagacgtcaacgaaggtgaggatgacgacgggatggataacggcaacggaggtagtggtttggggacagagagacagagggagcatccgtttatcgtaacggagcctggggaagtggcacgtggcaaaaagaacggcttagattatctgttccacttgtacgaacaatgccgtgagttccttcttcaggtccagacaattgctaaagaccgtggcgaaaaatgccccaccaaggtgacgaaccaagtattcaggtacgcgaagaaatcaggagcgagttacataaacaagcctaaaatgcgacactacgttcactgttacgctctccactgcctagacgaagaagcttcaaatactctcagaagagcgtttaaagaacgcggtgagaacgttggctcatggcgtcaggcttgttacaagccacttgtgaacatcgcttgtcgtcatggctgggatatagacgccgtctttaacgctcatcctcgtctctctatttggtatgttccaacaaagctgcgtcagctttgccatttggagcggaacaatgcggttgctgcggctgcggctttagttggcggtattagctgtaccggatcgtcgacgtctggacgtggtggatgcggcggcgacgacttgcgtttctag

SEQ ID NO: 146 - protein, 424 aa, Arabidopsis thaliana
MDPEGFTSGLFRWNPTRALVQAPPPVPPPLQQQPVTPQTAAFGMRLGGLEGLFGPYGIRFYTAAKIAELGFTASTLVGMKDEELEEMMNSLSHIFRWELLVGERYGIKAAVRAERRRLQEEEEEESSRRRHLLLSAAGDSGTHHALDALSQEDDWTGLSEEPVQQQDQTDAAGNNGGGGSGYWDAGQGKMKKQQQQRRRKKPMLTSVETDEDVNEGEDDDGMDNGNGGSGLGTERQREHPFIVTEPGEVARGKKNGLDYLFHLYEQCREFLLQVQTIAKDRGEKCPTKVTNQVFRYAKKSGASYINKPKMRHYVHCYALHCLDEEASNALRRAFKERGENVGSWRQACYKPLVNIACRHGWDIDAVFNAHPRLSIWYVPTKLRQLCHLERNNAVAAAAALVGGISCTGSSTSGRGGCGGDDLRF

SEQ ID NO: 147 - DNA, 55 bp, Artificial sequence
  primer prm4841
ggggacaagtttgtacaaaaaagcaggcttaaacaatggatcctgaaggtttcac

SEQ ID NO: 148 - DNA, 50 bp, Artificial sequence
  primer prm4842
ggggaccactttgtacaagaaagctgggtaaccaaactagaaacgcaagt

SEQ ID NO: 149 - DNA, 2194 bp, Oryza sativa; identical to SEQ ID NO: 85

SEQ ID NO: 150 - DNA, 1179 bp, Oryza sativa; identical to SEQ ID NO: 6

SEQ ID NO: 151 - protein, 420 aa, Arabidopsis thaliana
MDPEGFTSGLFRWNPTRALVQAPPPVPPPLQQQPVTPQTAAFGMRLGGLEGLFGPYGIRFYTAAKIAELGFTASTLVGMKDEELEEMMNSLSHIFRWELLVGERYGIKAAVRAERRRLQEEEEEESSRRRHLLLSAAGDSGTHHALDALSQEGLSEEPVQQQDQTDAAGNNGGGGSGYWDAGQGKMKKQQQQRRRKKPMLTSVETDEDVNEGEDDDGMDNGNGGSGLGTERQREHPFIVTEPGEVARGKKNGLDYLFHLYEQCREFLLQVQTIAKDRGEKCPTKVTNQVFRYAKKSGASYINKPKMRHYVHCYALHCLDEEASNALRRAFKERGENVGSWRQACYKPLVNIACRHGWDIDAVFNAHPRLSIWYVPTKLRQLCHLERNNAVAAAAALVGGISCTGSSTSGRGGCGGDDLRF

SEQ ID NO: 152 - protein, 420 aa, Brassica juncea
MDPEGFTSGLFRWNPTRALVQAPPPVPPPLQQQPVTPQTAAFGMRLGGLEGLFGPYGIRFYTAAKIAELGFTASTLVGMKDEELEEMMNSLSHIFRWELLVGERYGIKAAVRAERRRLQEEEEEESSRRRHLLLSAAGDSGTHHALDALSQEELSEEPVQQQDQTDAAGNNGGGGSGYWDAGQGKMKKQQQQRRRKKPMLTSVETDEDVNEGEDDDGMDNGNGGSGLGTERQREHPFIVTEPGEVARGKKNGLDYLFHLYEQCREFLLQVQTIAKDRGEKCPTKVTNQVFRYAKKSGASYINKPKMRHYVHCYALHCLDEEASNALRRAFKERGENVGSWRQACYKPLVNIACRHGWDIDAVFNAHPRLSIWYVPTKLRQLCHLERNNAVAAAAALVGGISCTGSSTSGRGGCGGDDLRF

SEQ ID NO: 153 - protein, 426 aa, Ionopsidium acaule
MDPEGFTSGLFRWNTTRAMVQHQPPPQVPPPPSQQSPVTPQTAAFGMRLGGLEGLFGPYGIRFYTAAKIAELGFTASTLVGMKDEELEDMMNSLSHIFRWELLVGERYGIKAAVRAERRRLQEEEEDDSSRRRHLLLSAAGDSGTHHALDALSQEDDWTGLSEEPVHQDQTDAAGNGGFGGYLESSVHGKMKKHQPRRRKKPLVLTSVETDDDGNDNEDDDGMDNGNGGIGLGTERQREHPFIVTEPGEVARGKKNGLDYLFHLYEQCREFLLQVQTIAKDRGEKCPTKVTNQVFRYAKKSGASYINKPKMRHYVHCYALHCLDEEASNALRRAFKERGENVGSWRQACYKPLVNIACRHGWDIDAVFNAHPRLSIWYVPTKLRQLCHLERNNAVAAAAALVGGISCTGSSASGRGGCGGDEELRY

SEQ ID NO: 154 - protein, 417 aa, Leavenworthia crassa
MDPEGFTSGLFRWNPTRATVQALPPVPPPLQQQPATVQSAAFGTRLGGLEGLFGVYGIRFYTAAKIAELGFTASTLVGMRDEELEEMMNSLSHIFRWELLVGERYGIKAAVRAERRRLQEEEEEESSRRRHLLLSAAGDSGTHHALDALSQEDDWTGLSEEPVQQIDHLTDAVGNNGGYWEANKGKMKKQQQRRRKKPMLTSVETDDDINEGEDEDGMDNSNGGLGTERQREHPFIVTEPGEVARGKKNGLDYLFHLYEQCREFLLQVQTIAKDRGEKCPTKVTNQVFRYAKKSGASYINKPKMRHYVHCYALHCLDEEASNALRRAFKERGENVGSWRQACYKPLVNIACRHGWDIDAVFNSHPRLSIWYVPTKLRQLCHMERNNEVAAATVLVGGISCTGTSASGHGECGGELHY

SEQ ID NO: 155 - protein, 403 aa, Selenia aurea
MDPEGFTSGLFRWNPTRATVQALAPVPPPLQQQPATAQTAAFGMRLGGLEGLFGAYGIRFYTAAKIAELGFTASTLVGMRDEELEEMMNSLSHIFRWELLVGERYGIKAAVRAERRRLQEEEEEESSRRRHLLLSAAGDSGTHHALDALSQEDDWTGLSEEPVQQQDHQTDAVGNNGGYWDEGKGKMKKQQQRRRMKPLMTSVEPDNDMDECEDEDRMDNGNGGGGGLGMERQREHPFIVTEPGEVARGKKNGLDYLFHLYEQCREFLLQVQLIAKDRGEKCPTKVTNQVFRYAKKSGASYINKPKMRHYVHCYALHCLDEDASNALRRAFKERGENVGSWRQARYKPLVDIACRHGWDIDAVFNAHPRLSIWYVPTKLRQLCHLERNNAVAAAAVLVGGISC

SEQ ID NO: 156 - protein, 430 aa, Arabidopsis lyrata
MDPEGFTSGLFRWNPTRAMVAAPPPVPPQPQQQPATPQTRAFGMRLGGLEGLFGAYGIRFYTAAKIAELGFTASTLVGMKDEELEEMMNSLSHIFRWELLVGERYGIKAAVTAERRRLQEEEEEESSRRRHLLLSAAGDSGTHHALDALSQEDDWTGLSEELDREPVQQQNQTDAAGNNGGGGSGYWEAGQAKMKKQQQQRRRKKPMVTSVETDDDVNEGDDDDGMDNGNGGGGGGLGTERQREHPFIVTEPGEVARGKKNGLDYLFHLYEQCREFLLQVQTIAKDRGEKCPTKVTNQVFRYAKKSGASYINKPKMRHYVHCYALHCLDEDASNALRRAFKERGENVGSWRQACYKPLVNIACRHGWDIDAVFNAHPRLSIWYVPTKLRQLCHLERNNAVAAAAALVGGISCTGSSTSGRGGCGGDDLRF

SEQ ID NO: 157 - protein, 403 aa, Streptanthus glandulosus
SGLFRWNSTRALVQQPPPVPPPQQQPPETPQTVAFGMRLGGLEGLFGAYGIRFYTAAKIAELGFTASTLVGMKDEELEDMMNSLSHIFRWELLVGERYGIKAAVRAERRRLQEVEEEESSRRRHLLLCAAGDSGTHHALDTLSQEDYWTGLSEEPGQQQDQTDAAGNNGGNGGGEGGGYWEAGQAKMKKPQQRRRKKSMVTSVEIDDECNEGEDDDGMDNCNGGGGGLGIERQREHPFIVTEPGEVARGKKNGLDYLFHLYEQCREFLLQVQTIAKDRGEKCPTKGTNQVFRYAKNSGASYINKPKMRHYVHCYALHCLDEEASNALRRAFKERGENVGSWRQACYKPLVNIACRHGWDIDAVFNAHPHLSIWYVPTKLRQLCHLERNNAVAAAAALVGGISC

SEQ ID NO: 158 - protein, 407 aa, Cochlearia officinalis
MDPEGFTNGLFRWNTTRAMIQQQQQLPPPQITPPPQQSPATPQTAAFGMRLGGLEGLFGPYGIRFYTAAKIAELGFTASTLVGMKDEELEDMMNSLSHIFRWELLVGERYGIKAAVRTERRRLQEEEEEESSRRRHFMLSAGGDSGTHHALDALSQEDDWTGLSEEPVHQDQTDAAGNGGFGGYLESGHGKMKKQQQQKRRKKPLVTSVETDDDGNDDDDGMDNGNGGSSGLGTERQREHPFIVTEPGEVARGKKNGLDYLFHLYEQCREFLLQVQTIAKDRGEKCPTKVTNQVFRYAKKSGASYINKPKMRHYVHCYALHCLDEDASNALRRAFKERGENVGSWRQACYKPLVNIACRHGWDIDAVFNAHPRLSIWYVPTKLRQLCHLERNNAVAAASALVGGISC

SEQ ID NO: 159 - protein, 415 aa, Brassica oleracea var. botrytis
MDPEGFTSGLFRWNPTRVMVQAPTPIPPPQQQSPATPQTAAFGMRLGGLEGLFGPYGVRFYTAAKIAELGFTASTLVGMKDEELEDMMNSLSHIFRWELLVGERYGIKAAVRAERRRLQEEEEEESSRRRHLLLSAAGDSGTHLALDALSQEDDWTGLSQEPVQHQDQTDAAGINGGGRGGYWEAGQTTIKKQQQRRRKKRLYVSETDDDGNEGEDDDGMDIVNGSGVGMERQREHPFIVTEPGEVARGKKNGLDYLFHLYEQCREFLLQVQTIAKDRGEKCPTKVTNQVFRYAKKSGANYINKPKMRHYVHCYALHCLDEEASNALRSAFKVRGENVGSWRQACYKPLVDIACRHGWDIDAVFNAHPRLSIWYVPTKLRQLCHLERNNAEAAAATLVGGISCRDRLRLDALGFN

SEQ ID NO: 160 - protein, 389 aa, Idahoa scapigera
MDPDGFANGLFRWKPTRAMVQSPPPVPPPPQQQQTAAAEAFGMRVGGLEGLFRAYGIRFYTSAKIAELGFTASTLLNMKDEELDEMMNSLSHIFRWELLVGERYGIKAAVRAERRRVQEEEEEESSRRRHLLLSAAGDSVAHHALSQEDDWTSLSEEPVQQKDQTDAAGSNGGGVYWGAGQAKMKQKRRKKPTVMMTSVETDDEINECEDDDRMDNGNGGMAIERQREHPFIVTEPGEVARGKKNGLDYLFHLYEQCREFLLQVQTIAKDRGEKCPTKVTNQVFRYAKKSGASYINKPKMRHYVHCYALHCLDENASNALRRSFKERGENVGSWRQACYKPLVDVAFRHGGDIDAVFNAHPRLSIWYVPTKLRQLCHLERNNAGSATAA

SEQ ID NO: 161 - protein, 399 aa, Capsella bursa-pastoris
GLFRWNPMRAMVQAPPPVPPSPQQQQPATPQTAAFGMRLGGLEGLFGAYGIRFYTAAKIAELGFTASTLVGMKDEELEEMMNSLSHIFRWELLVGERYGIKAAVRAERRRLQEEEEESSRRRHLLLSAAGDSGTHHALDALSQEDDWTGLSEEPVQQQDQTDAAGNNGGGGSGYWEAGQAKMKKPQQRRRKKPMVASVETDDDGNEGEDDDGMDNGNGGSGGMGTERQREHPFIVTEPGEVARGKKNGLDYLFHLYEQCREFLLQVIQTIAKDRGEKCPTKVTYQVFRYAKKSGASYINKPKMRHYVHCYALHCLDEDASNALRRSFKERGENVGSWRQACYKPLVNIACRHGWDIDAVFNAHPRLSIWYVPTKLRQLCHLERNNAVAAATALVGGISC

SEQ ID NO: 162 - protein, 393 aa, Barbarea vulgaris
GLFRWNPTRATVQALPPVPPPPQQQPATTQTAAFGMRLGGLEGLFGAYGIRFYTAAKIAELGFTASTLVGMRDEELEEMMNSLSHIFRWELLVGERYGIKAAVRAERRRLQEEEEEESSRRRHLLLSAAGDSGTHHALDALSQEDDWTGLSEEPVQQQDHQTDAAGNNGGNWEAGKGKMKKQQQRRRKKPMMTSVETDDDINEGEDEDGMDNGNGGGGGGGLGTERQREHPFIVTEPGEVARGKKNGLDYLFHLYEQCREFLLQVQTIAKDRGEKCVTNQVFRYAKKSGASYINKPKMRRCVRCCALHCLDEDASSALRRAFKERGGNVGSWRQACCKPLVNIACRHGWDIDAVFNAHPRLSIWYVPTKLRQLCHLERNNAVAAATVLVGGIS

SEQ ID NO: 163 - protein, 412 aa, Petunia hybrida
MDPEAFSASLFKWDPRGAMPPPNRLLEAVAPPQPPPPPLPPPQPLPPAYSIRTRELGGLEEMFQAYGIRYYTAAKITELGFTVNTLLDMKDDELDDMMNSLSQIFRWELLVGERYGIKAAIRAERRRLEEEEGRRRHILSDGGTNVLDALSQEGLSEEPVQQQEREAAGSGGGGTAWEVVAPGGGRMRQRRRKKVVVGRERRGSSMEEDEDTEEGQEDNEDYNINNEGGGGISERQREHPFIVTEPGEVARGKKNGLDYLFHLYEQCRDFLIQVQNIAKERGEKCPTKVTNQVFRFAKKAGASYINKPKMRHYVHCYALHCLDEDASNALRRAFKERGENVGAWRQACYKPLVAIAARQGWDIDAIFNGHPRLSIWYVPTKLRQLCHSERSNAAAAASTSVSGGGVDHLPHF

SEQ ID NO: 164 - protein, 396 aa, Antirhinum majus
MDPDAFLFKWDHRTALPQPNRLLDAVAPPPPPPPQAPSYSMRPRELGGLEELFQAYGIRYYTAAKIAELGFTVNTLLDMRDEELDEMMNSLCQIFRWDLLVGERYGIKAAVRAERRRIDEEEVRRRHLLLGDTTHALDALSQEGLSEEPVQQEKEAMGSGGGGVGGVWEMMGAGGRKAPQRRRKNYKGRSRMASMEEDDDDDDDETEGAEDDENIVSERQREHPFIVTEPGEVARGKKNGLDYLFHLYEQCRDFLIQVQTIAKERGEKCPTKVTNQVFRYAKKAGANYINKPKMRHYVHCYALHCLDEAASNALRRAFKERGENVGAWRQACYKPLVAIAARQGWDIDTIFNAHPRLSIWYVPTKLRQLCHAERSSAAVAATSSITGGGPADHLPF

SEQ ID NO: 165 - protein, 413 aa, Nicotiana tabacum
MDPEAFSASLFKWDPRGAMPPPTRLLEAAVAPPPPPPVLPPPQPLSAAYSIRTRELGGLEELFQAYGIRYYTAAKIAELGFTVNTLLDMKDEELDDMMNSLSQIFRWELLVGERYGIKAAIRAERRRLEEEELRRRSHLLSDGGTNALDALSQEGLSEEPVQQQEREAVGSGGGGTTWEVVAAVGGGRMKQRRRKKVVSTGRERRGRASAEEDEETEEGQEDEWNINDAGGGISERQREHPFIVTEPGEVARGKKNGLDYLFHLYEQCRDFLIQVQNIAKERGEKCPTKVTNQVFRYAKKAGASYINKPKMRHYVHCYALHCLDEEASNALRRAFKERGENVGAWRQACYKPLVAIAARQGWDIDTIFNAHPRLAIWYVPTRLRQLCHSERSNAAAAASSSVSGGVGDHLPHF

SEQ ID NO: 166 - protein, 416 aa, Nicotiana tabacum
MDPEAFSASLFKWDPRGAMPPPTRLLEAAVAPPPPPPALPPPQPLSAAYSIKTRELGGLEELFQAYGIRYYTAAKIAELGFTVNTLLDMKDEELDDMMNSLSQIFRWELLVGERYGIKAAIRAERRRLEEEELRRRGHLLSDGGTNALDALSQEGLSEEPVQQQEREAVGSGGGGTTWEVVAAAGGGRMKQRRRKKVVAAGREKRGGASAEEDEETEEGQEDDWNINDASGGISERQREHPFIVTEPGEVARGKKNGLDYLFHLYEQCRDFLIQVQNIAKERGEKCPTKVTNQVFRYAKKAGASYINKPKMRHYVHCYALHCLDEEASNALRRAFKERGENVGAWRQACYKPLVAIAARQGWDIDTIFNAHPRLAIWYVPTKLRQLCHSERSNAAAAAASSSVSGGGGGGDHLPHF

SEQ ID NO: 167 - protein, 392 aa, Triticum aestivum
MDPNDAFLAAHPFRWDLGPPAPAAVPPPPPPPPPPPALPPANAPRELEDLVVGYGVRASTVARISELGFTASTLLVMTERELDDMTAALAGLFRWDLLIGERFGLRAALRAERGRLMSPGCRHHGYQSGSTIDGASQEVLSNERDGAASGGIGEEDAMRMMASGKKQKNGSAGRKAKKARRKKVNDLRLDMQGDEHEEGGGGRSESTESSAGGGVGGERQREHPFVVTEPGEVARAKKNGLDYLFHLYEQCRLFLLQVQSMAKLHGQKSPTKVTNQVFRYASKVGASYINKPKMRHYVHCYALHCLDEDASDALRRAYKARGENVGAWRQACYAPLVDIAARHGFDIDAVFAAHPRLAIWYVPTRLRQLCHQARSAHDTAAAHAGAMPPPMF

SEQ ID NO: 168 - protein, 392 aa, Triticum aestivum
MDPNDAFLAAHPFRWDLGPPAPAAVPPPPPPPPLPPALPPANAPRELEDLVVGYGVRASTVARISELGFTASTLLVMTESELDDMTAALAGLFRWDLLIGERFGLRAALRAERGRLMSPGCRHHGYQSGSTIDGASQEVLSNERDGAASGGIGEDDAMRMMASGKKQKNGSAARKAKKARRNKVKELRLDMQGDEHEDGGGGRSESTESSAGGVGGERQREHPFVVTEPGEVARAKKNGLDYLFHLYEQRRLFLLQVQSMAKLHGQKSPTKVTNQVFRYASKVGASYINKPKMRHYVHCYALHCLDEDASDALRRAYKARGENVGAWRQACYAPLVDIAARHGFDIDAVFAAHPRLAIWYVPTRLRQLCHQARSAHDAAAAAHAGSMPPPMF

SEQ ID NO: 169 - protein, 400 aa, Lolium temulentum
MDPHDAFLAAHPFRWDLGPPAPAAVPPPPPLPMPQTPALPPANSPRELEDLVAGYGVRGATVARISELGFTASTLLVMTDRELDDMTAALAGLFRWDLLIGERFGLRAALRAERGRLMALHGGRHHGHQSGSTIDGASQEVLSNERDGAASGEDDAGRMMLSGKKLKNGSVARKAKKARRKKVDGLRLDHMQEDEREDGGGRSESTESSAGGGGGVGGERQREHPFVVTEPGEVARAKKNGLDYLFHLYEQCRLFLLQVQSMAKLHGHKSPTKVTNQVFRYASKVGASYINKPKMRHYVHCYALHCLDQEASDALRRAYKARGENVGAWRQACYAPLVDIAAGHGFDVDAVFAAHPRLAIWYVPTRLRQLCHQARSAHEAAAANANANGAMPPPPPPPMF

SEQ ID NO: 170 - protein, 389 aa, Oryza sativa
MDPNDAFSAAHPFRWDLGPPAPAPVPPPPPPPPPPPPANVPRELEELVAGYGVRMSTVARISELGFTASTLLAMTERELDDMMAALAGLFRWDLLLGERFGLRAALRAERGRLMSLGGRHHGHQSGSTVDGASQEVLSDEHDMAGSGGMGDDDNGRRMVTGKKQAKKGSAARKGKKARRKKVDDLRLDMQEDEMDCCDEDGGGGSESTESSAGGGGGERQREHPFVVTEPGEVARAKKNGLDYLFHLYEQCRLFLLQVQSMAKLHGHKSPTKVTNQVFRYAKKVGASYINKPKMRHYVHCYALHCLDEEASDALRRAYKARGENVGAWRQACYAPLVDISARHGFDIDAVFAAHPRLAIWYVPTRLRQLCHQARSSHAAAAAALPPPLF

SEQ ID NO: 171 - protein, 393 aa, Zea mays
DPNDAFSAAHPFRWDLGPPAPAAPAPPPPPPPAPQLLPHAPLLSAPRELEDLVAGYGVRPSTVARISELGFTASTLLGMTERELDDMMAALAGLFRWDVLLGERFGLRAALRAERGRVMSLGGRFHTGSTLDAASQEVLSDERDAAASGGLAEGEAGRRMVTTGKKKGKKGVGARKGKKARRKKELRPLDVLDDENDGDEDGGGGGSDSTESSAGGSGGGERQREHPFVVTEPGEVARAKKNGLDYLFHLYEQCRVFLLQVQSLAKLGGHKSPTKVTNQVFRYAKKCGASYINKPKMRHYVHCYALHCLDEDASNALRRAYKARGENVGAWRQACYAPLVEIAARHGFDIDAVFAAHPRLTIWYVPTRLRQLCHQARGSHAHAAAGLPPPPMF

SEQ ID NO: 172 - protein, 391 aa, Zea mays
MDPNDAFSAAHPFRWDLGPPAHAAPAPAPPPPPLAPLLLPPHAPRELEDLVAGYGVRPSTVARISELGFTASTLLGMTERELDDMMAALAGLFRWDVLLGERFGLRAALRAERGRVMSLGARCFHAGSTLDAASQEALSDERDAAASGGGMAEGEAGRRMVTTTAGKKGKKGVVGTRKGKKARRKKELRPLNVLDDENDGDEYGGGSESTESSAGGSGERQREHPFVVTEPGEVARAKKNGLDYLFHLYEQCRVFLLQVQSIAKLGGHKSPTKVTNQVFRYANKCGASYINKPKMRHYVHCYALHCLDEEASNALRRAYKSRGENVGAWRQACYAPLVEIAARHGFDIDAVFAAHPRLAVWYVPTRLRQLCHQARGSHAHAAAGLPPPPMF

SEQ ID NO: 173 - protein, 456 aa, Ophrys tenthredinifera
MVLATSQQHHQHNPHEVQQHLQPHSTATESSRELEEVFEGYGVRYSTIARIGDLGFTASTLAGMREEEVDDMMAALSHLFRWDLLVGERYGIKAAIRAERRRLEALIFSHVSGAARLSHHQHQMGYLFSSATTGYHLMPDDPRKRHLLLSPDHHSALDALSQEGLSEEPVQLEREAAGSGGEVVGRRDGKGKNQQRQTSAKKKDASSTKSKKKKKKGIEEGDDEEEEVEVWGRGASIENDEDDDGDESQSEQSSAAERQREHPFIVTEPGEVARAKKNGLDYLFNLYEQCHEFLNQVQSVAKERGDKCPTKVTNLVFRYAKKKVGASYINKPKMRHYVHCYALHVLDEDASNSLRRAFKERGENVGAWRLACYKPLVAISASHSFDIDAVFNAHPRLSIWYVPTKLRQLCHLARSSTSQFPLAVPRTTGSSNQRVSSTVHVVEDSAAAHSFRPPMF

SEQ ID NO: 174 - protein, 412 aa, Lycopersicon esculentum
MDPDAFSASLFKWDPRGAMPPPSRLLEPVAPPQPPPSLPPPPPPQPLPTSSYSIRSTRELGGLEELFQAYGIRYYTAAKIAELGFTVNTLLDMKDEELDDMMNSLSQIFRWDLLVGERYGIKAAIRAEWRRLEEEEARRRGHILSDGGTNVLDALSQEGLSEEPVQQQHEREAAGSGGGGTWEVAAGGGGRMKQRRRKKAGRERRGEEDEETEELGEEDEENMNQGGGGGGISERQREHPFIVTEPGEVARGKKNGLDYLFHLYEQCRDFLIQVQTIAKERGEKCPTKVTNQVFRYAKKAGASYINKPKMRHYVHCYALHCLDEDASNALRRAFKERGENVGAWRQACYKPLVAIAARQGWDIDAIFNAHPRLAIWYVPTKLRQLCHSERSNAAAAASSSVSGGVADHLPHF

SEQ ID NO: 175 - protein, 367 aa, Carica papaya
MDPDGFSSSLFKWDPTRGIVQAPVRLLEAVAAAPTQAAYGVRPRELGGLEELFQDYGIRYFTAAKIAELGFTASTLVDMKDEELDEMMNSLSQIFRWELLVGERYGIKAAVRAERRRLDDDDSRRRQTLSTDTTHALDALSQEGLSEEPVQQEKEAAGSGGGTIWEVGPGKKKQRRRKVVGEEEQEEENGGGSERQREHPFIVTEPGEVARGKKNGLDYLFHLYEQCRDFLIQVQNIAKERGEKCPTKVTNQVFRYAKKAGASYINKPKMRHYVHCYALHCLDEKESNALRTAFKERGENVGSWRQACYKPLVAIAARQGWDIDAIFNAHPRLAIWYVPNKLRQLCHAERNNTAIASTSAAAHHLPF

SEQ ID NO: 176 - DNA, 1263 bp, Arabidopsis thaliana
atggatcctgaaggtttcacgagtggcttattccggtggaacccaacgagagcattggttcaagcaccacctccggttccacctccgctgcagcaacagccggtgacaccgcagacggctgcttttgggatgcgacttggtggtttagagggactattcggtccgtacggtatacgtttctacacggcggcgaagatagcggagttaggttttacggcgagcacgcttgtgggtatgaaggacgaggagcttgaagagatgatgaatagtctctctcatatctttcgttgggagcttcttgttggtgaacggtacggtatcaaagctgccgttagagctgaacggagacgattgcaagaagaggaggaagaggaatcttctagacgccgtcatttgctactctccgccgctggtgattccggtactcatcacgctcttgatgctctctcccaagaagggttatctgaggaaccggtgcagcaacaagaccagactgatgcggcggggaataacggcggaggaggaagtggttactgggacgcaggtcaaggaaagatgaagaagcaacagcagcagagacggagaaagaaaccaatgctgacgtcagtggaaaccgacgaagacgtcaacgaaggtgaggatgacgacgggatggataacggcaacggaggtagtggtttggggacagagagacagagggagcatccgtttatcgtaacggagcctggggaagtggcacgtggcaaaaagaacggcttagattatctgttccacttgtacgaacaatgccgtgagttccttcttcaggtccagacaattgctaaagaccgtggcgaaaaatgccccaccaaggtgacgaaccaagtattcaggtacgcgaagaaatcaggagcgagttacataaacaagcctaaaatgcgacactacgttcactgttacgctctccactgcctagacgaagaagcttcaaatgctctcagaagagcgtttaaagaacgcggtgagaacgttggctcatggcgtcaggcttgttacaagccacttgtgaacatcgcttgtcgtcatggctgggatatagacgccgtctttaacgctcatcctcgtctctctatttggtatgttccaacaaagctgcgtcagctttgccatttggagcggaacaatgcggttgctgcggctgcggctttagttggcggtattagctgtaccggatcgtcgacgtctggacgtggtggatgcggcggcgacgacttgcgtttctga

SEQ ID NO: 177 - DNA, 1263 bp, Brassica juncea
atggatcctgaaggtttcacgagtggcttattccggtggaacccaacgagagcattggttcaagcaccacctccggttccacctccgctgcagcaacagccggtgacaccgcagacggctgcttttgggatgcgacttggtggtttagagggactattcggtccatacggtatacgtttctacacggcggcgaagatagcggagttaggctttacggcgagcacgcttgtgggtatgaaggacgaggagcttgaagagatgatgaatagtctctctcatatctttcgttgggagcttcttgttggtgaacggtacggtatcaaagctgccgttagagctgaacggagacgattgcaagaagaggaggaggaggaatcttctagacgccgtcatttgctactctccgccgctggtgattccggtactcatcacgctcttgatgctctctcccaagaagagttatctgaggaaccggtgcagcaacaagaccagactgatgcggcggggaataacggcggaggaggaagtggttactgggacgcaggtcaaggaaagatgaagaagcaacagcagcagagacggagaaagaaaccaatgctgacgtcagtggaaaccgacgaagacgtcaacgaaggtgaggatgacgacgggatggataacggcaacggaggtagtggtttggggacagagagacagagggagcatccgtttatcgtaacggagcctggggaagtggcacgtggcaaaaagaacggcttagattatctgttccacttgtacgaacaatgccgtgagttccttcttcaggtccagacaattgctaaagaccgtggcgaaaaatgccccaccaaggtgacgaaccaagtattcaggtacgcgaagaaatcaggagcgagttacataaacaagcctaaaatgcgacactacgttcactgttacgctctccactgcctagacgaagaagcttcaaatgctctcagaagagcgtttaaagaacgcggtgagaacgttggctcatggcgtcaggcttgttacaagccacttgtgaacatcgcttgtcgtcatggctgggatatagacgccgtctttaacgctcatcctcgcctctctatttggtatgttccaacaaagctgcgtcagctttgccatttggagcggaacaatgcggttgctgcggctgcggctttagttggcggtattagctgtaccggatcgtcgacgtctggacgtggtggatgcggcggcgacgacttgcgtttctag

SEQ ID NO: 178 - DNA, 1281 bp, Ionopsidium acaule
atggaccccgaaggtttcacgagtggcttattccgatggaacacaacaagagcaatggttcaacatcaaccaccaccacaagtccctcctcctccgtcgcagcaatctccggtaacaccacaaacggcggcgtttgggatgagattaggtggtctagaaggtttgttcggtccttacgggatacgtttttacacggcggcgaagatagccgagttaggtttcacggcgagcacgctcgttggtatgaaagacgaagagcttgaagatatgatgaatagtctctctcatatctttcgttgggagcttcttgttggtgaacgttacggtatcaaagctgccgttagagctgaacggaggagattgcaagaagaggaggaggatgattcttctagacgccgtcatttgcttctctccgccgctggtgattccggcactcaccacgctcttgatgctctctctcaagaagatgattggacaggcttatcagaggaaccggtgcatcaagaccaaactgacgcggcgggtaacggcggattcggtggttatttggaatcatcagtacacggaaagatgaagaaacatcaaccaagacgtagaaagaaaccgttggtactgacgtcagttgaaaccgacgatgacggcaacgataacgaggatgacgacgggatggataacggtaacggaggtattgggttagggacggagagacagagagaacatccgtttattgtaactgagcctggggaagtggcacgtggcaaaaagaacggtttggattatcttttccacttgtacgaacaatgccgtgagttccttcttcaggtccagactattgctaaagaccgtggcgaaaaatgccccaccaaggtgacgaaccaagtgtttaggtacgctaaaaaatcaggagcgagttacataaacaaaccaaaaatgcgacactacgtccattgctacgctctccactgcctagacgaagaagcatcaaacgctcttagaagagcgtttaaagaacgcggcgagaacgttggctcgtggcgtcaggcttgttacaagccgctagtgaacatagcctgtcgtcatggctgggacatagacgccgttttcaacgcacatcctcgtctatctatttggtacgttccaactaaactgcgtcagctttgccatttggagcgtaacaacgccgttgctgcggcggctgctttggttggtggtattagctgcaccggctcttctgcgtctggacgcggtggttgcggcggcgacgaggagttacgttactag

SEQ ID NO: 179 - DNA, 1254 bp, Leavenworthia crassa
atggatcctgaaggtttcacgagtggcttattccgatggaacccaacgagagcaacggttcaagcactacctccggttcctcctccactacagcaacagccagcaacagtacagtcagcggcttttgggacgcgacttggtggtttagagggacttttcggtgtttatgggatacgtttttacacggcggcgaagatagccgagttaggttttacggcgagcacgcttgtgggtatgagggatgaggagcttgaggaaatgatgaatagcctctctcatatctttcggtgggagcttcttgttggtgaacggtacggtatcaaagctgccgttagagctgaacggagaagattgcaagaagaagaggaggaggaatcttctagacgacgtcatttgttactctccgccgcaggtgattccggcactcatcacgctcttgatgctctctcccaagaagatgattggacaggtttatcagaggagccggtacagcaaatagatcacctgactgatgcggtggggaataacggtggttattgggaagcaaacaaaggaaagatgaagaagcaacaacaaagaaggagaaagaaaccgatgctgacatcagttgaaacagacgatgacatcaacgaaggtgaggatgaagatggaatggataacagtaacggaggattagggacagagagacaaagggagcatccgtttattgtaacggagcctggggaagtagcacgtggcaaaaagaacggtttagattacctcttccatttgtacgaacaatgtcgtgagttccttcttcaggttcagacaatagctaaagatcgtggcgagaaatgtcccaccaaggtgacgaaccaagtgtttaggtacgcaaaaaaatcaggagcaagttacataaacaagcccaaaatgcgacactacgtccactgttacgctcttcactgcttagacgaagaagcctcaaacgctctccgacgagcgttcaaggaacgcggtgagaacgttggctcttggcgtcaggcttgttacaagccacttgtgaacatcgcttgtcgtcatggttgggatatagacgccgtctttaactctcatcctcgtctctctatttggtatgtcccaaccaagctgcgtcagctctgtcatatggagaggaacaatgaggttgctgcagctacggttttggttggcggtattagctgtacgggaacgtcagcgtctggacacggtgaatgtggaggcgagttacattattag

SEQ ID NO: 180 - DNA, 1210 bp, Selenia aurea
atggatcctgaaggtttcacgagtggcttattccgatggaacccaacgagagcaacggttcaagcactagctccggttcctcctccattgcagcaacaaccagcaacagcacagacggcggcttttgggatgcgacttggtggtttagaaggactctttggtgcttacggaatacgtttttacacggcggcgaagatagcagagttaggttttacggcgagcacgcttgtgggtatgagggacgaggagcttgaggaaatgatgaatagtctctctcatatctttcggtgggagcttcttgttggtgaacggtacggtatcaaagctgccgttagagctgaacgaagaagattgcaggaggaagaggaagaggaatcttctagacgacgtcatttgctactctccgccgcaggtgattccggcactcatcacgctcttgatgctctctcccaagaagatgattggacaggcttatcagaagagccggtgcagcagcaagatcatcagactgatgcggtgggtaataacggcggttactgggatgaaggtaaaggaaagatgaagaagcaacaacaaagaaggaggatgaaaccgttgatgacgtcagtggaacccgacaatgacatggacgagtgtgaggatgaagataggatggataacggtaacggaggaggtggtggattggggatggagagacagagggagcatccgtttattgtaacggagcctggggaagtggcacgtggcaaaaagaacggtttagattacctgttccatttgtacgaacaatgccgtgagttccttcttcaggtccaattaattgccaaagatcgtggcgagaaatgccctaccaaggtgacgaaccaagtgtttaggtacgcgaagaaatcaggagcgagttacataaacaagcctaaaatgagacactacgtccactgttacgctttacactgcttagatgaagacgcctcaaacgctctccgacgagcgttcaaggaacgcggtgagaacgttgggtcatggcgtcaggctcgttacaagccacttgtggatatcgcttgtcgtcatggctgggatatagacgccgtctttaacgctcatcctcgtctctctatttggtatgttcctaccaagctacgtcagctctgccatttggagagaaacaatgcggttgcggctgctgcggttttagttggcggtattagctgta

SEQ ID NO: 181 - DNA, 1293 bp, Arabidopsis lyrata
atggatcctgaaggtttcacgagtggcttattccgatggaacccaacgagagcaatggttgcagcaccacctccggttccacctcagccgcagcaacagccggcaacacctcagacgcgcgcttttgggatgcgacttggtggtttagagggactgttcggagcttacggtatacgtttttacacggcggcgaagatagcggagttaggttttacggcgagcacgcttgtgggtatgaaggacgaggagcttgaggagatgatgaatagtctctctcacatctttcgttgggagcttcttgttggtgaacggtacggtatcaaagctgccgttacagctgaacggagacgattgcaagaagaggaggaggaggaatcttctagacgccgtcatttgctactctccgccgctggtgattccggtactcatcacgctcttgatgctctctcccaagaagatgattggacagggttatctgaggaactggacagggaaccggtgcaacagcaaaaccagacagatgcggctgggaataacggcggaggaggaagtggttactgggaagcaggtcaagcaaagatgaagaagcaacagcagcagagacggagaaagaaaccgatggtgacgtcagtggaaaccgacgatgacgtcaacgaaggtgatgatgacgacgggatggataacggcaacggaggtggtggtggtggattggggacagagagacagagggagcacccgtttatcgtaacggagcctggggaagtggcacgtggcaaaaagaacggtttagattatctgttccacttgtacgaacaatgccgtgagttccttcttcaggtccagacaattgctaaagaccgtggcgaaaaatgtcccaccaaggtgacgaaccaagtgttcaggtacgcgaagaaatcgggagcgagttacataaacaagcccaaaatgcgacactacgtccactgttacgctctccactgcctagacgaagacgcttcaaacgctctccgaagagcgtttaaagaacgcggtgagaacgttggctcgtggcgtcaggcttgttacaagccacttgtgaacattgcttgtcgtcatggctgggatatagacgccgtctttaacgctcatcctcgtctctctatttggtacgttccaactaagctgcgtcagctttgccatttggagcggaacaatgccgtggctgcggccgcggcgttggttggcggtattagctgtaccggatcgtctacgtctggccgtggtggttgcggcggcgacgacttgcgtttctag

SEQ ID NO: 182 - DNA, 1209 bp, Streptanthus glandulosus
agtggcttattccgatggaactcaacgagagcactggttcaacaaccacctccagttcctccaccgcagcagcaaccgccggaaacaccgcagacggtagcgtttggaatgcgactaggtgggttggagggtttgttcggtgcttacggaatacgtttttacacggcggcaaagatagcggagttaggttttacggctagcacgcttgttggcatgaaggacgaggagcttgaggatatgatgaatagcctctctcatatctttcgttgggaacttctcgtcggtgaacggtacggtatcaaagctgccgttagagctgaacggagacgattgcaagaagtggaagaggaggaatcttctagacgccgtcatttgctactctgcgccgcaggtgattcaggcactcatcacgctcttgatactctctcacaagaagattattggacaggattatcagaggagccggggcagcagcaagatcagactgatgcggcgggaaacaacggcggaaacggcggaggagaaggaggaggctattgggaagcagggcaggcgaagatgaagaagccacagcaaagacgtagaaagaaatcgatggtgacgtcagtggaaatcgatgatgaatgcaacgaaggtgaggatgacgatgggatggataactgtaacggaggaggtggtgggttggggatagagagacaaagggagcatccgtttatagtaacggagccaggggaagtggcacgtggcaaaaagaacggtttggattatcttttccacttgtacgaacaatgccgcgagttccttcttcaggtccagacaattgctaaagaccgtggcgaaaaatgccccaccaagggtacgaaccaagttttcaggtacgcaaagaattcgggagcgagttacataaacaagccgaaaatgcgacactacgttcattgttacgcactccactgcctcgacgaagaagcttcaaacgctctccgaagagcgtttaaagaacgcggtgagaacgttgggtcgtggcgtcaggcctgttacaagccacttgtgaacatcgcttgtcgtcatggctgggatatagacgccgtttttaacgctcatcctcacctctccatttggtatgttcccactaagctgcgtcagctctgccatttagagcggaacaatgcggttgctgcggctgcagctttagttggcggtattagctgt

SEQ ID NO: 183 - DNA, 1221 bp, Cochlearia officinalis
atggatcctgaaggtttcacgaatggcttattccgatggaacacaacaagagcaatgattcaacaacaacaacaattaccaccgcctcaaatcactcctccgccgcaacaatcaccggcaacaccacaaacggcggcgtttgggatgagactaggtggtttagaaggtttgttcggtccttacgggatacgtttttacacggcggcgaagatagctgagctaggtttcacggcgagcacgcttgttggtatgaaagacgaagagcttgaagatatgatgaatagtctctcacatatctttcgttgggagcttcttgtcggtgaacgttacggtatcaaagctgccgttagaactgaacggaggagattgcaagaagaggaagaggaggaatcttctagacgccgtcattttatgctctccgccggtggtgattccggcactcaccacgctcttgatgctctctctcaagaagatgattggacaggtttatcagaggaaccggtgcatcaagaccaaactgacgcggcgggtaacggcggattcggtggttatttagaatcaggacacggtaaaatgaagaaacagcaacaacaaaaacgtagaaagaaaccgttagtgacgtcagtggaaacagacgatgacggtaacgatgatgacgacgggatggataacggtaacggagggagtagcgggttgggaacggagagacagagagaacatccgtttatcgtaacggagcctggggaagtggcacgtggcaaaaagaacggtttggattatcttttccacttgtacgaacaatgccgtgagtttcttcttcaggttcagactattgctaaagaccgtggcgaaaaatgtcccaccaaggtgacgaaccaagtgtttaggtacgccaaaaaatcaggagcgagttacataaacaaaccaaaaatgcgacactacgtccattgttacgccctacactgcctagacgaagacgcttcaaacgctctcagaagagcgttcaaagaacgcggcgaaaacgttggctcgtggcggcaggcctgttataaaccgctagtcaacatcgcgtgccgtcacggttgggacatagacgccgttttcaacgcacatccacgtctatctatttggtacgttccgacaaaactgcgtcagctttgccatttggagcgtaacaacgcggttgctgcggcttcggctttagttggcggtattagctgt

SEQ ID NO: 184 - DNA, 1248 bp, Brassica oleracea
atggatcctgaaggtttcacgagtggcttattccgatggaacccaactagagtaatggttcaagcaccaactccgattcctccaccgcagcagcaatcgccagcaacaccgcagaccgcagcgtttggaatgcgactaggtggtttggagggtttgttcggtccttacggtgtacgtttttacacggcggcaaagatagctgagttaggttttacggcgagcacactggtgggcatgaaggacgaggagcttgaggatatgatgaatagcctctctcatatctttcgttgggagcttctcgtcggtgaacggtacggcatcaaagctgccgttagagctgaacggagacgattgcaagaagaggaagaggaggaatcgtctagacgccgtcatttgctactctccgccgcaggtgattccggcactcatcttgctcttgatgctctctcccaagaagatgactggacagggttgtcacaggagccggttcagcaccaagatcagactgatgcggcggggatcaacggcggaggaagaggaggttattgggaagcagggcagacgacaataaaaaagcaacagcagagacgcagaaagaagcgattgtacgtcagtgaaactgatgatgacggcaacgaaggtgaggatgacgacgggatggatattgttaacggaagtggtgtagggatggagagacaaagggagcacccgtttattgtaacggagccaggggaagtagcacgtggcaaaaagaacggtttggattatcttttccacttgtacgaacagtgccgcgagttccttcttcaggtccagaccattgctaaagatcgtggcgaaaaatgccctaccaaggtgacgaaccaggtgttcaggtacgctaagaaatcgggggcaaattacataaataagccaaaaatgcgacactacgttcattgttacgcactccattgcctcgacgaagaagcttcaaacgctctccgaagtgcgtttaaagttcgcggtgagaacgttgggtcgtggcgtcaggcttgttacaagccacttgtggacattgcttgtcgtcatggctgggatatagacgccgtttttaacgctcatcctcgcctttccatttggtatgttcccactaagctgcgtcagctctgccatttggagcggaacaatgcggaagcagcggcagcgactttggttggtggtattagctgcagggatcgcctgcgtctggacgctttggggtttaattag

SEQ ID NO: 185 - DNA, 1167 bp, Idahoa scapigera
atggatcctgatggtttcgcgaatggtttattccgatggaaaccaacgagagcaatggttcaatcaccacctcctgttcctcctccacctcagcaacaacagacggcggctgcagaggcttttgggatgcgagtaggcggtttagaaggtctcttccgtgcttacggtatacgtttttacacgtcggcgaaaatagcggagttaggttttacggcgagcacacttctgaatatgaaggatgaagagcttgatgaaatgatgaatagcctttctcatatctttcggtgggagcttcttgtcggtgaacggtacggtatcaaagctgccgttagagctgaaaggagacgagtgcaagaagaagaggaggaagaatcttctcgacggcgtcatttgttactctccgctgccggggattccgtcgctcatcacgctctctctcaagaagatgactggacaagcttgtcagaggagccggtgcagcaaaaagatcagactgatgcggcggggagtaacggtggaggagtttattggggggcaggtcaagcaaagatgaagcaaaaacggagaaagaaaccgacggtgatgatgacgtcagtggaaacagatgacgaaattaacgaatgtgaggatgacgacaggatggataacggtaacggtggaatggcgatagagagacagagagagcatccgtttattgtaacggagcctggggaagtggcacgtggcaaaaagaacggtttggattatttgtttcatttgtacgaacaatgccgtgagttccttcttcaggttcagacaattgctaaagaccgtggcgaaaaatgccccaccaaggtgacaaaccaagtgttcagatacgcgaagaaatcaggagcgagttacataaataaaccaaaaatgcgacattacgtccactgctacgctttacattgcctagacgaaaacgcttcaaacgctctccgaagatcatttaaggaacgtggcgaaaacgttggatcgtggcgtcaggcttgttacaagccacttgttgacgttgcttttcgtcatggtggggatatagatgctgtctttaacgctcatcctcgcctctctatttggtatgtcccaactaagctgcgtcagctctgccatttggagcggaacaatgcgggttctgcaactgcggct

SEQ ID NO: 186 - DNA, 1199 bp, Capsella bursa-pastoris
gtggcttattccgatggaacccaatgagagcaatggttcaagcaccacctccggttcctccttcgccgcagcagcaacagccggcaacacctcagacggcggctttcgggatgcgacttggtggcttagagggactctttggtgcttacggtatccgtttctacacggcggcgaagatagcggagttgggttttacggccagcacgctcgttggtatgaaggacgaggagcttgaggagatgatgaacagtctctctcacatctttaggtgggagcttctcgttggtgaacggtacggtatcaaagctgccgtaagagctgaacggagacgattgcaagaagaggaggaggaatcttctagacgccgtcatttgctgctctccgccgctggtgattccggtactcatcacgctcttgatgccctctcccaagaagatgactggacagggttatcagaagaaccggtgcagcagcaagaccagacagatgcggcggggaataacggcggaggagggagtggttattgggaagcaggtcaagcaaagatgaagaagccacaacaaaggaggagaaagaaaccgatggtggcgtcagtggaaaccgatgacgacggcaatgaaggcgaggatgatgacgggatggataacggtaacggaggtagtggtgggatggggacggagagacagagggagcatccgtttatcgtaacggagccaggggaagtggcacgtggcaaaaagaacggtttggattatctgttccatttgtacgaacaatgccgtgagttccttcttcaggtcattcagacgatagctaaagaccgtggcgagaaatgccccaccaaggtgacgtaccaagtgtttagatacgcgaagaaatctggggcgagttacataaacaaacccaaaatgcgacactacgtccactgttatgctctccactgtctagacgaagacgcttcgaacgctcttcgaaggtctttcaaagaacgcggtgagaacgttggctcgtggcgtcaggcttgttacaagccacttgtgaacatcgcttgtcgtcatggctgggatattgacgccgtctttaacgcacaccctcgcctctctatttggtatgtccccactaagctacgtcagctttgccatttggagcggaacaatgcggttgctgcggctacggctttagttggcggtattagctgt

SEQ ID NO: 187 - DNA, 1183 bp, Barbarea vulgaris
gtggcttattccgatggaacccaacgagagcaacggttcaagcactacctccggttcctcctccaccacagcaacagccggcaacaacacagacggcggcttttgggatgcgacttggtggtttggagggactgttcggtgcttacgggatacgtttttacacggcggcgaagatagcggagttaggttttacggcgagcacgcttgtgggtatgagggacgaggagcttgaggaaatgatgaatagcctctctcatatctttcggtgggagcttctcgtaggtgagcggtacggtatcaaagctgccgttagagctgaacggagacgattgcaagaagaggaggaggaagaatcttctagacgacgtcatttgctactctccgccgctggtgattccggcactcatcacgctcttgatgctctctcccaagaagatgattggacaggcttatcagaggagccggtgcagcagcaagatcaccagactgatgcggcggggaataacggcggtaattgggaagcaggtaaaggaaagatgaagaagcaacagcagagaaggagaaagaaaccgatgatgacgtcagtggaaacagacgatgacatcaacgaaggtgaggatgaagacgggatggataacggtaacggaggaggaggtggtggtgggttggggacggagagacagagagagcatccgtttattgtaacggagccaggggaagtggcacgtggcaaaaagaacggtttagattacctgttccatttgtacgaacaatgccgtgagttccttcttcaggtccagacaattgctaaagaccgtggcgagaaatgcgtgacgaaccaagtgttcaggtacgcgaagaaatcgggagcgagttacataaacaagcccaaaatgcgacgctgcgtccgctgttgcgctcttcactgcctagacgaggatgcctcgagcgctctccgacgagcgttcaaggaacgcggtgggaacgtaggctcgtggcgtcaggcttgttgcaagccacttgtgaacatcgcttgtcgtcatggctgggacatagatgccgtctttaacgctcatcctcgcctctctatttggtatgtccctaccaagctgcgtcagctctgccatttggaacggaacaatgcggttgctgcagctacggttttagttggcggtattagctg

SEQ ID NO: 188 - DNA, 1239 bp, Petunia hybrida
atggacccagaggctttctcagcaagtttgttcaagtgggacccacgaggtgcaatgccaccaccaaaccggttgttggaagcggtggcaccaccacaaccaccacctcctcctcttccacctccgcagcctctaccaccggcttattccattagaacaagagagctagggggcctagaggaaatgttccaagcttatgggataagatattacactgctgctaagataactgagttaggttttacggtgaatacacttttggacatgaaagatgatgaacttgatgatatgatgaatagcctttcacaaattttcagatgggaactgcttgttggagaaaggtatggtatcaaagctgctattagagctgaacggcggaggcttgaggaggaagaagggcggcgccggcacattctttctgatggtggaactaatgttcttgatgctctctcacaagaagggttatctgaggaaccagtgcagcagcaagagagagaagcagcgggaagcggcggaggagggacggcatgggaagtggtggcgccaggcggtggcagaatgagacaaaggaggaggaagaaggtggtggtggggagggagagaagggggtcatcaatggaggaagatgaagacacggaggagggacaagaagataatgaagattataacattaataatgagggtggtggaggaattagcgagagacaaagggaacatcccttcatagtaactgagcctggggaggtggcgcgtggcaaaaagaatggcttagattacttgttccatctctatgaacaatgcagggatttcttgatccaagttcagaatattgccaaggaacgtggtgaaaaatgccctactaaggtaacaaatcaggtgttcaggttcgcaaagaaggcaggagcaagttacataaacaagccaaaaatgcgacactacgtgcactgctatgcacttcattgccttgatgaggatgcttcaaatgctctaagaagagcattcaaggagagaggagagaatgttggggcatggagacaggcatgttacaaacccctggtagccatagctgctcgacaaggctgggatatcgacgccatttttaatggacatcctcgactatccatttggtatgtgcccaccaagctccgccagctttgccattctgaacgaagcaatgccgctgcagctgcttccacctcagtttctggtggtggtgttgatcatctgcctcatttttag

SEQ ID NO: 189 - DNA, 1191 bp, Antirrhinum majus subsp. majus floricaula
atggatcctgatgcattcttgttcaaatgggaccacagaaccgccctccctcaaccaaacaggctcctcgacgccgtggccccaccgcctcctccgccgcctcaggcgccgtcatactccatgaggccaagagaactcggcggcttagaagaattattccaagcttatggcatcagatactacactgccgctaaaatcgctgaacttggattcactgtgaacacgcttttggacatgagggacgaggagctagacgagatgatgaacagcctttgtcagattttcaggtgggacctacttgtcggagagaggtatgggattaaggcggcggtgagagcggaacgacgtcgtatcgacgaggaggaagtgaggcggaggcatctcttgttgggtgatactacgcatgctcttgatgctctttctcaagaagggttgtcggaggagccggtgcagcaagaaaaggaagcaatgggaagcggcggaggcggtgtaggaggcgtgtgggaaatgatgggggcgggtggtcgaaaagcaccgcagcggcgtaggaagaattacaaagggaggtctagaatggcttcgatggaggaggatgatgatgatgatgacgacgaaaccgaaggggcggaagacgacgaaaatatcgtaagcgagcggcagagggagcatccgtttatcgtgacggagcccggagaggtggcgcgtgggaaaaagaatggtcttgattatttgtttcatttgtacgagcaatgccgcgacttcttgatccaagttcaaactattgctaaggagagaggtgaaaaatgtcccactaaggtgacgaaccaagtgttcaggtacgcaaagaaggctggcgctaactacatcaacaaaccaaaaatgcgccactacgtgcactgctacgccctgcactgccttgatgaggccgcgtccaatgcacttcgtcgggcattcaaggagcgtggtgagaacgtcggtgcatggcgtcaggcatgctacaagcccttggtggccattgcagcaagacaaggatgggatatcgataccatattcaacgctcatccccgtctctcgatctggtatgtccccaccaagcttcgtcagctctgccatgccgagaggagcagtgcggcagttgctgccaccagctccatcaccggaggtgggccggcagatcacttgccgttttag

SEQ ID NO: 190 - DNA, 1242 bp, Nicotiana tabacum
atggacccagaggctttctcagcgagtttgttcaaatgggaccctagaggtgcaatgccaccgccaacccggctgttggaagccgcggtggcgcctcctcctccaccaccagttctgccaccgccgcagcctctatcggcggcctattccattaggacaagggagttaggagggctagaggagttgtttcaagcttacggtatacgttattacactgctgctaaaatagcggagctaggttttacggtgaatactctattggacatgaaagatgaggaacttgatgatatgatgaatagcctttcacagattttcagatgggaactcctcgtcggagaaaggtacggtatcaaagctgcaatcagggcggaacggcggaggcttgaggaggaagaactacggcggcgcagccaccttctgtctgatggtggaactaatgcccttgacgctctctcacaagaagggttgtctgaggaaccagtgcagcagcaagagagagaagcagttggaagcggcggagggggaacgacatgggaagtggtggcggcagttggcggtggaagaatgaaacaaagaaggaggaagaaggtggtgtcgacggggagggagagaaggggaagagcgtcggcggaggaggatgaagaaacggaggaaggtcaagaagatgagtggaatattaacgacgccgggggaggaataagcgagaggcaaagggagcatccttttatcgtgacggagccaggtgaggtggcgcgtgggaaaaagaacggcttggattacttgttccacctctacgagcaatgccgggatttcttgattcaagttcagaatattgccaaggaacgtggtgaaaaatgtcccactaaggtaacaaatcaggtgttcaggtacgcgaagaaggcaggggcaagctacataaataagccaaaaatgcgacactacgtgcattgctacgcacttcattgccttgatgaggaggcctccaatgcgctaagaagagctttcaaggagcgaggagagaatgttggggcatggagacaagcatgttacaagcccctggtagccatagctgctcgacaaggctgggatatcgacaccatctttaatgcacatcctcgactcgccatttggtatgtccccaccaggctccgccagctttgccattctgaacgaagcaacgctgctgctgctgcttctagctcggtttctggtggtgttggtgatcacctgccgcatttctaa

SEQ ID NO: 191 - DNA, 1251 bp, Nicotiana tabacum
atggacccagaggctttctcagcgagtttgttcaagtgggaccctagaggtgcaatgccaccgccaacccggctgttggaagcagcggtggcgcctcctcctcctccgccagctcttccaccgccgcagcctctgtcggcggcttattccattaagacaagggagttaggaggactagaggagttatttcaagcttacggtataagatattacactgctgctaaaatagcggagttaggttttacggtgaacactctattggacatgaaagatgaggaacttgatgatatgatgaatagcctttcacagattttcagatgggaactactcgtcggagaaaggtacggtatcaaagctgcaatcagggcggaacggcggaggcttgaggaggaagaactgcggcggcgtggccaccttctgtctgatggtggaactaatgcccttgacgctctctcacaagaagggttgtctgaggaaccagtgcagcagcaagagagagaagcagtgggaagtggcggagggggaacgacatgggaagtggtggcggcagctggcggtgggagaatgaaacaaaggaggaggaagaaggtggtggcggcggggagggagaaaaggggaggagcgtcggcggaggaggatgaagaaacggaggaaggtcaagaagatgactggaacattaacgacgccagtggaggaataagcgagaggcaaagggagcatccttttatcgtgacggagccaggtgaggtggcgcgtgggaaaaagaacggcttggattacttgttccacctctatgagcaatgccgggatttcttgatccaagttcagaatattgccaaggaacgtggtgaaaaatgccccactaaggtaacaaatcaggtgttcaggtacgcgaagaaggcaggggcaagctacataaacaagccaaaaatgcgacactacgtgcattgctacgcacttcattgccttgacgaggaagcctccaatgcgctaagaagagctttcaaggagcgaggagagaacgtcggggcgtggagacaggcatgttacaaaccccttgtggccatagctgctcgacaaggctgggatatcgacaccatctttaatgcacatcctcgactcgccatttggtatgttcccaccaagctccgccagctttgccactctgaacggagcaatgctgctgctgctgctgcttctagctcggtttctggtggtggtggtggtggtgatcacctccctcatttctaa

SEQ ID NO: 192 - DNA, 1179 bp, Triticum aestivum
atggatcccaacgacgccttcttggccgcgcacccgttcaggtgggacctcggcccgccggctccggcagccgtgcctcctccccctcccccgcctccgcctcctcctgcgctacctccggcgaacgcgccgagggagctggaggacctcgtggtcgggtatggcgtgcgcgcgtccacggtggcgcggatctcggagctcgggttcacggccagcacgctcctcgtcatgacggagcgcgagctcgacgacatgacggccgcgctcgcgggactattccgctgggacctgctcatcggcgagcggttcggccttcgtgccgcgctgcgcgccgagcgcggccgcctcatgtcaccgggctgccgccaccacggataccagtccgggagcaccatcgacggcgcctcacaggaagtgctgtcgaacgagcgcgatggggcggctagcggcggcatcggcgaagaggacgccatgaggatgatggcgtcgggcaagaagcagaagaatgggtccgcagggaggaaggccaagaaggccaggaggaagaaggtgaacgacctgcggctggacatgcagggggacgagcacgaggaaggcgggggcggccggtcggagtcgacggagtcgtcagccggcggaggcgtcggcggggagcggcagcgggagcacccgttcgtggtgacggagcccggcgaggtggcgagggccaagaagaacgggctggactacctgttccatctctacgagcagtgccgcctcttcctgctccaggtgcagtccatggccaagctgcatggccagaagtctccaaccaaggtgacgaaccaggtgttcaggtacgcgagcaaggtgggggcgagctacatcaacaagcccaagatgcggcactacgtgcactgctacgcgctgcactgcctggacgaggacgcctccgacgcgctgcgccgggcgtacaaggcgcgcggcgagaacgtcggggcgtggcggcaggcctgctacgcgccgctggtggacatcgcggcgcgccacggcttcgacatcgacgccgtcttcgccgcgcacccgcggctcgccatctggtacgtgcccaccaggctccgccagctctgccaccaggcccggagcgcccacgacaccgccgccgcgcacgccggcgccatgccgccgcccatgttctag

SEQ ID NO: 193 - DNA, 1179 bp, Triticum aestivum
atggatcccaacgacgccttcttggccgcgcacccgtttaggtgggacctcggcccaccggctccggcagccgtgcctcctcctcctcccccgcctccgcttcctcctgcgctgcctccggcgaacgcgccgagggagctggaggacctcgtggtcgggtatggcgtgcgcgcgtccacggtggcgcggatctcggagctcgggttcacggctagcacgctcctggtcatgaccgagagcgagctcgacgacatgacggccgcgctcgcggggttgttccgctgggacctgctcatcggcgagcggttcggccttcgcgccgcgctgcgcgccgaacgtggccgcctcatgtcaccaggctgccgccaccacggataccagtccggcagcaccatcgacggcgcctcacaggaagtgttgtcgaacgagcgcgatggggcggctagcggcggcatcggcgaagacgacgccatgaggatgatggcgtctggcaagaagcagaagaatgggtccgcagcgaggaaggccaagaaggcgaggaggaacaaggtgaaggagctgcgactggacatgcagggggacgagcacgaggacggcgggggcggccggtcggagtcgacggagtcgtcagccggaggcgtcggcggggagcggcagcgggagcacccgttcgtggtgacggagcccggcgaggtggcgagggcgaagaagaacgggctggactacctgttccatctctacgagcagcgccgcctcttcctgctccaggtgcagtccatggccaagctgcatggccagaagtctccaaccaaggtgacgaaccaggtgttcaggtacgcgagcaaggtgggggcgagctacatcaacaagcccaagatgcggcactacgtgcactgctacgcgctgcactgcctggacgaggacgcctccgacgcgctgcgccgggcgtacaaggcgcgcggcgagaacgtcggcgcctggaggcaggcgtgctacgcgccgctggtggacatcgcggcgcgccacggcttcgacatcgacgccgtcttcgccgcgcacccgcggctcgccatctggtacgtgcccaccaggctccgccagctctgtcaccaggcgcgcagcgcccacgacgccgccgccgccgcacacgccggctccatgccgccgccaatgttctag

SEQ ID NO: 194 - DNA, 1203 bp, Lolium temulentum
atggatccccacgacgccttcctcgccgcgcacccgttccggtgggacctcggcccgccggctccggcggccgtgccccctcctcctccactgcccatgcctcaaactcccgcgctgcctccggcgaactcgccgagggagctggaggatctcgtggccgggtacggcgtgcgcggggccacggttgcgcgaatctccgagctcggcttcacggccagcacgctcctggtcatgacggaccgcgagctggacgacatgacggccgcactcgccggcctgttccgctgggacctgctcatcggcgagcggttcggcctgcgcgccgcgctgcgagcagagcgcggccgcctgatggcactgcatgggggccgacaccacggtcaccagtccggcagcaccatcgacggcgcctcccaagaagtgttgtccaacgaacgggatggggcggcgagcggcgaggacgacgccggcaggatgatgttatcgggcaagaagctgaagaatggatcggtggcgagaaaggccaagaaagcaaggaggaagaaggtggacgggctccggctggaccacatgcaggaggacgagcgcgaggacggcggcggccgctcggagtcaacggagtcgtcggctggcggaggcggcggcgttggaggggagcggcagcgggagcacccgttcgtggtgacggagcccggggaggtggcgagggccaagaagaacgggctggactacctgttccatctctacgagcagtgccgcctcttcctgctccaggtgcagtccatggccaagctgcatggccacaagtctccaaccaaggtgacgaaccaggtgttcaggtacgcgagcaaggtgggggcgagctacatcaacaagcccaagatgcgccactacgtgcactgctacgcgctgcactgcctcgaccaggaggcctccgacgcgctgcgccgcgcgtacaaggcccgcggcgagaacgtcggcgcctggaggcaggcatgctacgcgccgctcgtcgacatcgccgccggccacggcttcgacgtcgacgccgtcttcgccgcgcacccgcgactcgccatctggtacgtgcccaccaggctccgccagctctgccaccaggcaaggagcgcgcacgaagccgccgccgccaacgccaacgccaacggggccatgccgccgccgccgccgccgcccatgttctag

SEQ ID NO: 195 - DNA, 1170 bp, Oryza sativa
atggatcccaacgatgccttctcggccgcgcacccgttccggtgggacctcggcccgccggcgccggcgcccgtgccaccaccgccgccaccaccgccgccgccgccgccggctaacgtgcccagggagctggaggagctggtggcagggtacggcgtgcggatgtcgacggtggcgcggatctcggagctcgggttcacggcgagcacgctcctggccatgacggagcgcgagctcgacgacatgatggccgcgctcgccgggctgttccgctgggacctgctcctcggcgagcggttcggcctccgcgccgcgctgcgagccgagcgcggccgcctgatgtcgctcggcggccgccaccatgggcaccagtccgggagcaccgtggacggcgcctcccaggaagtgttgtccgacgagcatgacatggcggggagcggcggcatgggcgacgacgacaacggcaggaggatggtgaccggcaagaagcaggcgaagaagggatccgcggcgaggaagggcaagaaggcgaggaggaagaaggtggacgacctaaggctggacatgcaggaggacgagatggactgctgcgacgaggacggcggcggcgggtcggagtcgacggagtcgtcggccggcggcggcggcggggagcggcagagggagcatcctttcgtggtgacggagcccggcgaggtggcgagggccaagaagaacgggctggactacctgttccatctgtacgagcagtgccgcctcttcctgctgcaggtgcaatccatggctaagctgcatggacacaagtccccaaccaaggtgacgaaccaggtgttccggtacgcgaagaaggtcggggcgagctacatcaacaagcccaagatgcggcactacgtgcactgctacgcgctgcactgcctggacgaggaggcgtcggacgcgctgcggcgcgcctacaaggcccgcggcgagaacgtgggggcgtggaggcaggcctgctacgcgccgctcgtcgacatctccgcgcgccacggattcgacatcgacgccgtcttcgccgcgcacccgcgcctcgccatctggtacgtgcccaccagactccgccagctctgccaccaggcgcggagcagccacgccgccgccgccgccgcgctcccgccgcccttgttctaa

SEQ ID NO: 196 - DNA, 1182 bp, Zea mays
gatcccaacgacgccttctcggcggcgcacccgttccggtgggacctgggcccgccggcccccgccgcgcccgcgcctccgcccccaccgccgcccgcgccgcagctgctgccccacgcgccgctgctgagcgcgccgagggagctggaggacctggtggccggctacggcgtgcgcccgtccacggtggcgcggatctcggagctcgggttcacggccagcacgctcctcggcatgacggagcgcgagctcgacgacatgatggccgcgctcgcggggctgttccgctgggacgtgctcctcggcgagcgcttcggcctccgcgccgcgctgcgggccgagcgcgggcgtgtcatgtccctcggcggccgcttccacaccgggagcacattggacgccgcgtcacaagaagtgctgtccgacgagcgcgacgccgcggccagcggcggcttagcggaaggcgaggccggcaggaggatggtgacgaccggcaagaagaagggcaagaaaggggttggcgcgaggaagggcaagaaggcgaggaggaagaaggagctgaggccgttggacgtgctggacgacgagaacgacggagacgaggacggcggcggcggcgggtcagactcgacggagtcttccgctggcggctccggcggcggggagaggcagcgggagcaccccttcgtggtcacagagcccggcgaggtggccagggccaagaagaacgggcttgactacctcttccatctgtacgagcagtgccgcgtcttcctgctgcaggtgcagtcccttgctaagctgggcggccacaagtcccctacaaaggtgaccaaccaggtgttccggtacgccaagaagtgcggcgcgagctacatcaacaagcccaagatgcggcactacgtgcactgctacgcgctgcactgcctggacgaggatgcctccaacgcgctgcgccgggcgtacaaggcccgtggcgagaacgtcggtgcctggaggcaggcctgctacgcgccgctcgtcgagatcgccgcgcgccacggcttcgacatcgacgccgtcttcgccgcgcacccgcgcctcaccatctggtacgtgcccaccaggttgcgccagctctgccaccaggcacgggggagccacgcccacgccgccgccggcctccccccgcccccgatgttctag

SEQ ID NO: 197 - DNA, 1176 bp, Zea mays
atggatcccaacgacgccttctcggcggcgcacccgttccggtgggacctcggcccgccggcgcacgccgcgcccgcgcccgcgcctccgcctccgccgctagcaccgctgctgctgccgcctcacgcgccgcgggagctggaggacctggtggccggctacggcgtgcgcccgtccacggtggcgcggatctcggagctcgggttcacggcgagcacgctcctcggcatgacggagcgcgagctggacgacatgatggccgcgctcgcggggctgttccgctgggacgtgctcctcggcgagcgcttcggcctccgcgccgcgctgcgcgccgagcgcggccgcgtcatgtccctcggcgcccgctgcttccacgccgggagcaccttggatgccgcgtcacaagaagcgctgtccgacgagcgcgacgccgcggccagcggcggcggcatggcagaaggcgaggccggcaggaggatggtgacgacgaccgccggcaagaagggcaagaaaggggtcgttggcacgaggaagggcaagaaggcgaggaggaagaaggagctgaggccgctgaacgtgctggacgacgagaacgacggggacgagtacggcggcgggtcggagtcgaccgagtcgtccgcgggaggctccggggagaggcagcgggagcacccgttcgtggtcaccgagcccggcgaggtggcgagggccaagaagaacgggctcgactacctcttccacctgtacgagcagtgccgcgtcttcctgctccaggtgcagtccatcgctaagctgggcggccacaaatcccctaccaaggtgaccaaccaggtgttccggtacgcgaacaagtgcggggcgagctacatcaacaagcccaagatgcggcactacgtgcactgctacgcgctgcactgcctggacgaggaggcctccaacgcgctgcgccgggcgtacaagtcccgcggcgagaacgtgggcgcctggaggcaggcctgctacgcgccgctcgtcgagatcgccgcgcgccacggcttcgacattgacgccgtcttcgccgcgcacccgcgcctcgccgtctggtacgtgcccaccaggctgcgccagctctgccaccaggcgcgggggagccacgcccacgctgccgccggactcccgccgcccccgatgttctag

SEQ ID NO: 198 - DNA, 1371 bp, Ophrys tenthredinifera
atggtgctggccacatcgcagcaacaccaccagcataaccctcacgaagtccagcagcacctgcagccgcattcgacggcaacagagtcgtcgcgggagctagaggaggtgttcgaggggtacggagttcggtactcgacgattgctcggattggggatctgggcttcacagcgagcacgctggcaggtatgagggaggaggaggtggacgatatgatggccgcactgtcgcatctcttccggtgggatcttcttgtcggcgaacgatatgggatcaaagcggcaattagggcagagcgacgccgtcttgaagcgctcattttttctcatgtctccggcgcagcccgcctaagccatcatcaacatcaaatgggatacctcttttcgtctgccaccacaggctaccacttaatgcctgatgatccacgcaagaggcaccttctcctctcccccgatcaccacagcgctctcgacgcactttcccaagaaggactctctgaggagccagtgcagctggagagggaggcggctggcagcggtggcgaagtggtaggcaggagagatggaaaggggaagaaccaacaacggcaaacctcggcaaagaagaaggatgcctcctctacgaagagcaagaaaaagaagaagaaagggatcgaagaaggagacgatgaagaagaggaggtcgaagtgtgggggcgcggggcaagcattgagaatgatgaggatgacgacggggatgagtcgcaatcagagcaaagcagcgctgcagagcggcagagggagcacccgttcatcgtgacggagccaggtgaggtggcgcgagctaagaagaacgggctcgattacctcttcaatctttacgaacaatgccatgaatttctgaaccaggtccagtccgtggcgaaggagcgcggggacaagtgcccaactaaggtgacgaacctggtattccgatatgcgaagaagaaagtgggagcaagctacatcaataagccgaagatgaggcactacgtgcactgctacgcgctccacgtgctagacgaggatgcgtccaactccctgaggcgggcgttcaaggaacgcggggagaacgttggcgcctggcgacttgcctgctacaagcccttggtggccatctccgcctcccacagcttcgacatagacgccgttttcaacgcgcatccccgcctctccatctggtacgtccccactaagctacgccagctctgccacctcgcccgcagttccacctctcagttcccgctggccgttcccagaactacaggcagttcgaaccaacgcgtctcatccaccgtccacgttgttgaagactcagctgcggcacactccttccgtccgcccatgttctaa

SEQ ID NO: 199 - DNA, 1239 bp, Lycopersicon esculentum
atggacccagatgctttctcggcgagtttgttcaagtgggacccaagaggtgcaatgccaccaccaagccggttattagaaccggtggcgccaccacaacctcctccatctctaccaccaccaccacctcctcagccgctcccaacatcatcctactccatacggagtacgagggagctcggaggactagaggagttatttcaagcgtacggcatacgctactacaccgccgctaagatagcggagttagggttcactgtgaacacgttattggacatgaaagatgaagaacttgatgatatgatgaatagcctctcgcagatatttcggtgggacctactcgtcggagagaggtacggtatcaaagcggcgattagagctgaatggcggaggctggaggaggaggaagcacggcgccgcggacacattttgtccgacggtggaacgaatgtccttgacgctctatcacaagaaggattatcggaggaaccagtgcagcagcagcacgagagagaagcggcaggaagtggtggtggaggtacatgggaagtggctgccggtggtggtggtaggatgaaacaaaggaggaggaagaaggcggggagggagagaagaggggaagaggatgaggaaacggaggaattaggagaagaagatgaagaaaatatgaaccaaggaggtggaggtggaggaataagcgagagacaaagggagcatccgtttatcgtgacggagcctggtgaagtagcacgtggcaaaaagaacggcttggattatctgttccatctctacgaacaatgccgtgatttcttgatccaagttcagactattgctaaggaacgaggtgaaaaatgccctacgaaggtgacgaatcaggtgttcaggtacgcgaagaaggcaggggcaagctacataaacaagcccaaaatgagacattatgtgcattgctatgcacttcactgccttgatgaggatgcttccaatgctctgagaagagctttcaaggagcggggagagaatgttggggcatggagacaggcgtgttacaagccattggtggctatagcggctcgacaaggctgggatatcgatgcaatcttcaatgcacatcctcgactagccatttggtatgtccccaccaagctccgacagctgtgccattctgaaagaagcaacgcagctgcagctgcttctagctccgtctctggtggtgttgctgatcacctgccacatttctaa

SEQ ID NO: 200 - DNA, 1104 bp, Carica papaya
atggatccagacggcttttcttccagcttgttcaagtgggacccaacgaggggaatagtgcaggcgccagtcaggttgctggaggcggtagctgcggcgcctacgcaggcggcgtacggagtgaggccgagggagctgggtggtctagaggagctttttcaagattacggcatcaggtacttcaccgctgcgaagatcgccgagctgggtttcacggctagcacgctggtggatatgaaggatgaggaactggacgagatgatgaacagcttgagccagatttttaggtgggagcttctggtgggagagaggtatgggattaaggctgctgttcgcgctgaaaggaggcggcttgacgacgacgattccagaagaagacagaccctctctactgacactacccacgctctcgatgctctctcccaggaagggttatcagaggagccggtgcagcaggagaaggaggcggcggggagcgggggaggtacgatatgggaggttgggccggggaagaaaaagcagcggcggagaaaggtggtgggtgaggaggagcaggaggaggaaaacggtggtggaagcgagagacagcgcgagcaccctttcatcgtgacagagcctggggaggtggcacgtggcaaaaaaaatggccttgattatctcttccacttgtacgagcagtgtcgtgacttcttgatccaagtccagaacatcgccaaggagcgaggagaaaagtgtcccacgaaggtgacgaaccaggtgtttagatatgcaaagaaagctggggcgagttatataaacaagccaaaaatgcgacactatgtgcactgctatgctttacactgtcttgacgagaaggaatcaaatgcgttgaggacagcatttaaggagagaggagaaaatgtagggtcgtggagacaggcgtgttataagcctcttgtcgccattgcagcacgccaaggttgggacattgatgccattttcaatgcacatcctcgtcttgccatttggtatgtccccaacaagcttcgccaactttgccatgccgagcgcaataatactgccattgcttctacctccgcggctgctcatcatcttccattctaa

(19) United States
(12) Patent Application Publication
(10) Pub. No.: US 2011/0099669 A1
(43) Pub. Date: Apr. 28, 2011

(54) PLANTS HAVING ENHANCED YIELD-RELATED TRAITS AND A METHOD FOR MAKING THE SAME

(75) Inventors: Ana Isabel Sanz Molinero, Gentbrugge (BE); Yves Hatzfeld, Lille (FR); Valerie Frankard, Waterloo (BE); Christophe Reuzeau, Tocan Saint Apre (FR)

(73) Assignee: BASF Plant Science GmbH, Ludwigshafen (DE)

(21) Appl. No.: 12/999,804

(22) PCT Filed: Jun. 10, 2009
(86) PCT No.: PCT/EP09/57190

§ 371 Date: Dec. 17, 2010

Related U.S. Application Data
(60) Provisional application No. 61/074,868, filed on Jun. 23, 2008.
(60) Provisional application No. 61/074,712, filed on Jun. 23, 2008.
(60) Provisional application No. 61/075,784, filed on Jun. 26, 2008.
(60) Provisional application No. 61/075,850, filed on Jun. 26, 2008.

(30) Foreign Application Priority Data
Jun. 23, 2008 (EP) 08158760.2
Jun. 26, 2008 (EP) 08159081.2
Jun. 26, 2008 (EP) 08159085.3
Jun. 30, 2008 (EP) 08158684.4

(51) Int. Cl. A01H 1/00; A01H 5/00; C12N 15/82; C12N 9/00; C07H 21/04

(52) U.S. Cl. 800/290; 800/298; 435/320.1; 800/320; 435/183; 536/23.2

(57) ABSTRACT

The present invention relates generally to the field of molecular biology and concerns a method for improving various plant growth characteristics by modulating expression in a plant of a nucleic acid sequence encoding a GS1 (Glutamine Synthase 1). The present invention also concerns plants having modulated expression of a nucleic acid sequence encoding a GS1, which plants have improved growth characteristics relative to corresponding wild type plants or other control plants. The invention also provides constructs useful in the methods of the invention. Furthermore, the present invention relates generally to the field of molecular biology and concerns a method for enhancing various plant yield-related traits by modulating expression in a plant of a nucleic acid sequence encoding a PEAMT (Phosphoethanolamine N-methyltransferase) polypeptide. The present invention also concerns plants having modulated expression of a nucleic acid sequence encoding a PEAMT, which plants have enhanced yield-related traits relative to corresponding wild type plants or other control plants. The invention also provides hitherto unknown PEAMT-encoding nucleic acid sequences, and constructs comprising the same, useful in performing the methods of the invention. Yet furthermore, the present invention relates generally to the field of molecular biology and concerns a method for increasing various plant seed yield-related traits by increasing expression in a plant of a nucleic acid sequence encoding a fatty acyl-acyl carrier protein (ACP) thioesterase B (FATB) polypeptide. The present invention also concerns plants having increased expression of a nucleic acid sequence encoding a FATB polypeptide, which plants have increased seed yield-related traits relative to control plants. The invention additionally relates to nucleic acid sequences, nucleic acid sequence constructs, vectors and plants containing said nucleic acid sequences. Even furthermore, the present invention relates generally to the field of molecular biology and concerns a method for improving various plant growth characteristics by modulating expression in a plant of a nucleic acid sequence encoding a LFY-like (LEAFY-like). The present invention also concerns plants having modulated expression of a nucleic acid sequence encoding a LFY-like, which plants have improved growth characteristics relative to corresponding wild type plants or other control plants. The invention also provides constructs useful in the methods of the invention.

The present invention relates generally to the field of molecular biology and concerns a method for improving various plant growth characteristics by modulating expression in a plant of a nucleic acid sequence encoding a GS1 (Glutamine Synthase 1). The present invention also concerns plants having modulated expression of a nucleic acid sequence encoding a GS1, which plants have improved growth characteristics relative to corresponding wild type plants or other control plants. The invention also provides constructs useful in the methods of the invention.

Furthermore, the present invention relates generally to the field of molecular biology and concerns a method for enhancing various plant yield-related traits by modulating expression in a plant of a nucleic acid sequence encoding a PEAMT (Phosphoethanolamine N-methyltransferase) polypeptide. The present invention also concerns plants having modulated expression of a nucleic acid sequence encoding a PEAMT, which plants have enhanced yield-related traits relative to corresponding wild type plants or other control plants. The invention also provides hitherto unknown PEAMT-encoding nucleic acid sequences, and constructs comprising the same, useful in performing the methods of the invention.

Yet furthermore, the present invention relates generally to the field of molecular biology and concerns a method for increasing various plant seed yield-related traits by increasing expression in a plant of a nucleic acid sequence encoding a fatty acyl-acyl carrier protein (ACP) thioesterase B (FATB) polypeptide. The present invention also concerns plants having increased expression of a nucleic acid sequence encoding a FATB polypeptide, which plants have increased seed yield-related traits relative to control plants. The invention additionally relates to nucleic acid sequences, nucleic acid sequence constructs, vectors and plants containing said nucleic acid sequences.

Even furthermore, the present invention relates generally to the field of molecular biology and concerns a method for improving various plant growth characteristics by modulating expression in a plant of a nucleic acid sequence encoding a LFY-like (LEAFY-like). The present invention also concerns plants having modulated expression of a nucleic acid sequence encoding a LFY-like, which plants have improved growth characteristics relative to corresponding wild type plants or other control plants. The invention also provides constructs useful in the methods of the invention.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the above-mentioned factors may therefore contribute to increasing crop yield.

Seed yield is a particularly important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

Plant biomass is yield for forage crops like alfalfa, silage corn and hay. Many proxies for yield have been used in grain crops. Chief amongst these are estimates of plant size. Plant size can be measured in many ways depending on species and developmental stage, but include total plant dry weight, above-ground dry weight, above-ground fresh weight, leaf area, stem volume, plant height, rosette diameter, leaf length, root length, root mass, tiller number and leaf number. Many species maintain a conservative ratio between the size of different parts of the plant at a given developmental stage. These allometric relationships are used to extrapolate from one of these measures of size to another (e.g. Tittonell et al 2005 Agric Ecosys & Environ 105: 213). Plant size at an early developmental stage will typically correlate with plant size later in development. A larger plant with a greater leaf area can typically absorb more light and carbon dioxide than a smaller plant and therefore will likely gain a greater weight during the same period (Fasoula & Tollenaar 2005 Maydica 50:39). This is in addition to the potential continuation of the micro-environmental or genetic advantage that the plant had to achieve the larger size initially. There is a strong genetic component to plant size and growth rate (e.g. ter Steege et al 2005 Plant Physiology 139:1078), and so for a range of diverse genotypes plant size under one environmental condition is likely to correlate with size under another (Hittalmani et al 2003 Theoretical Applied Genetics 107:679). In this way a standard environment is used as a proxy for the diverse and dynamic environments encountered at different locations and times by crops in the field.

Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. For example, poor early vigour has been a limitation to the introduction of maize (Zea mays L.) hybrids based on Corn Belt germplasm in the European Atlantic.

Harvest index, the ratio of seed yield to aboveground dry weight, is relatively stable under many environmental conditions and so a robust correlation between plant size and grain yield can often be obtained (e.g. Rebetzke et al 2002 Crop Science 42:739). These processes are intrinsically linked because the majority of grain biomass is dependent on current or stored photosynthetic productivity by the leaves and stem of the plant (Gardener et al 1985 Physiology of Crop Plants. Iowa State University Press, pp 68-73). Therefore, selecting for plant size, even at early stages of development, has been used as an indicator for future potential yield (e.g. Tittonell et al 2005 Agric Ecosys & Environ 105: 213). When testing for the impact of genetic differences on stress tolerance, the ability to standardize soil properties, temperature, water and nutrient availability and light intensity is an intrinsic advantage of greenhouse or plant growth chamber environments compared to the field. However, artificial limitations on yield due to poor pollination due to the absence of wind or insects, or insufficient space for mature root or canopy growth, can restrict the use of these controlled environments for testing yield differences. Therefore, measurements of plant size in early development, under standardized conditions in a growth chamber or greenhouse, are standard practices to provide indication of potential genetic yield advantages.

A further important trait is that of improved abiotic stress tolerance. Abiotic stress is a primary cause of crop loss worldwide, reducing average yields for most major crop plants by more than 50% (Wang et al., Planta (2003) 218: 1-14). Abiotic stresses may be caused by drought, salinity, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible.

Crop yield may therefore be increased by optimising one of the above-mentioned factors.

Depending on the end use, the modification of certain yield traits may be favoured over others. For example for applications such as forage or wood production, or bio-fuel resource, an increase in the vegetative parts of a plant may be desirable, and for applications such as flour, starch or oil production, an increase in seed parameters may be particularly desirable. Even amongst the seed parameters, some may be favoured over others, depending on the application. Various mechanisms may contribute to increasing seed yield, whether that is in the form of increased seed size or increased seed number.

One approach to increasing yield (seed yield and/or biomass) in plants may be through modification of the inherent growth mechanisms of a plant, such as the cell cycle or various signalling pathways involved in plant growth or in defense mechanisms.

Concerning GS1 polypeptides, it has now been found that various growth characteristics may be improved in plants by modulating expression in a plant of a nucleic acid encoding a GS1 (Glutamine Synthase 1) in a plant.

Concerning PEAMT polypeptides, it has now been found that various yield-related traits may be improved in plants by modulating expression in a plant of a nucleic acid sequence encoding a PEAMT (Phosphoethanolamine N-methyltransferase) in a plant.

Concerning FATB polypeptides, it has now been found that various seed yield-related traits may be increased in plants relative to control plants, by increasing expression in a plant of a nucleic acid sequence encoding a fatty acyl-acyl carrier protein (ACP) thioesterase B (FATB) polypeptide. The increased seed yield-related traits comprise one or more of: increased total seed yield per plant, increased total number of seeds, increased number of filled seeds, increased seed fill rate, and increased harvest index.

Concerning LFY-like polypeptides, it has now been found that various growth characteristics may be improved in plants by modulating expression in a plant of a nucleic acid sequence encoding a LFY-like (LEAFY-like) in a plant.

### BACKGROUND

### Glutamine Synthase (GS1)

Glutamine synthase catalyses the formation of glutamine from glutamate and NH₃, it is the last step of the nitrate assimilation pathway. Based on sequence comparison, glutamine synthases are grouped in two families, cytosolic (GS1) and chloroplastic (GS2) isoforms. GS1 glutamine synthases form a small gene family, where GS2 seems to occur as a single copy gene and both GS1 and GS2 occur in plants and algae. Many reports describe that glutamine synthases from higher plants have a direct impact on plant growth under conditions of nitrogen limitation (Oliveira et al. Plant Physiol. 129, 1170-1180, 2002; Fuentes et al. J. Exp. Bot. 52, 1071-1081, 2001; Migge et al. Planta 210, 252-260, 2000; Martin et al. Plant Cell 18, 3252-3274). However, so far no data are available on the effect of algal-type glutamine synthases on plant growth, in particular under conditions of reduced nitrogen availability.

### Phosphoethanolamine N-methyltransferase (PEAMT)

Phosphoethanolamine N-methyltransferase (PEAMT), also called S-adenosyl-L-methionine:ethanolamine-phosphate N-methyltransferase is involved in choline biosynthesis in plants. PEAMT functions in the methylation steps required to convert phosphoethanolamine to phosphocholine (Nuccio et al. 2000. J Biol Chem. 275(19):14095-101). Accordingly a PEAMT enzyme catalyzes one or more of the following reactions:
1) N-dimethylethanolamine phosphate+S-adenosyl-L-methionine<=>phosphoryl-choline+S-adenosyl-homocysteine
2) N-methylethanolamine phosphate+S-adenosyl-L-methionine<=>N-dimethylethanolamine phosphate+S-adenosyl-homocysteine
3) phosphoryl-ethanolamine+S-adenosyl-L-methionine<=>S-adenosyl-homocysteine+N-methylethanolamine phosphate.

The Enzyme Commission numbers assigned by IUPAC-IUBMB (International Union of Biochemistry and Molecular Biology) to PEAMT is EC2.1.1.103. The PEAMT enzyme belongs a class of methyltransferases (Mtases) which are dependent on S-adenosyl-L-methionine (SAM). Methyl transfer from the ubiquitous SAM to nitrogen, oxygen or carbon atoms is frequently employed in diverse organisms ranging from bacteria to plants and mammals. Structural analysis shows that PEAMT proteins belongs to a class of Mtases comprising methyltransferase domains that form the Rossman-like alpha-beta fold (Yang et al. 2004 J. Mol. Biol. 340, 695-706). In addition Phosphatidylethanolamine transferases typically comprise a ubiE/COQ5 methyltransferase domain (Pfam reference PF01209). This domain is also present in a number of methyltransferases involved in ubiquinone/menaquinone, biotin and sterol biosynthesis.

Phospholipids are important structural components of cellular membranes and in addition they play a relevant role in metabolism of essential compounds such as fatty acids. In humans Choline, a B vitamin-like molecule, is an essential nutrient naturally produced and participates in building cell membranes and move fats and nutrients between cells.

Phosphocholine is the major phospholipid in almost every plant tissue. In non-photosynthetic tissue, phosphoethanolamine is the second most prevalent phospholipid, whereas in green tissue the levels of phosphocholine are similar to those of phosphatidylglycerol (Dykes et al. 1976. Biochem J. 158(3): 575-581).

Tobacco plants overexpressing a gene encoding a PEAMT enzyme had reportedly increased the levels of phosphocholine and free Choline without affecting phosphatidylcholine content or growth (McNeil et al. 2001. PNAS. 2001, vol. 98, no. 17 10001-10005).

### Fatty Acyl-Acyl Carrier Protein (ACP) Thioesterase B (FATB)

Plants contain a considerable variety of membrane and storage lipids, and in each lipid, a number of different fatty acids is found. Fatty acids differ by their chain length and the number of double bonds. All plant cells synthesize de novo fatty acids from acetyl-CoA by a common pathway localized in plastids, unlike in other organisms. Fatty acids are either utilized in this organelle or transported to supply diverse cytoplasmic biosynthetic pathways and cellular processes. Production of fatty acids for transport depends on the activity of fatty acyl-acyl carrier protein (ACP) thioesterases (FATs; also called acyl-ACP TE) that release free fatty acids and ACP. Their activity represents the terminal step in the plastidial fatty acid biosynthesis pathway. The resulting free fatty acids can enter the cytosol where they are esterified to coenzyme A and further metabolized into membrane lipids and/or storage triacylglycerols.

FATs play an essential role in determining the amount and composition of fatty acids entering the storage lipid pool. Two classes of FATs have been described in plants, based on amino acid sequence comparisons and substrate specificity: the FATA class and the FATB class (Voelker et al. (1997) Plant Physiol 114:669-677). Substrate specificity of these isoforms determines the chain length and level of saturated fatty acids in plants. The highest activity of FATA is with oleoly-ACP, an unsaturated acyl-ACP, with very low activities towards other acyl-ACPs. FATB has highest activity with saturated acyl-ACPs.

FATA and FATB are nuclear-encoded, plastid-targeted golubular proteins that are functional as dimers. In addition, FATB polypeptides comprise a helical transmembrane anchor. FATB activity is encoded by at least two genes in Arabidopsis (Bonaventure et al. (2003) Plant Cell 15: 1020-1033), and by at least four genes in Oryza sativa.

Transgenic Arabidopsis plants (Doermann et al. (2000) Plant Physiol 123: 637-643) and transgenic canola plants (Jones et al. (1995) Plant Cell 7: 359-371) expressing a gene encoding a FATB under the control of a seed-specific promoter, displayed modified seed oil composition.

International patent application WO 2008/006171 describes methods for genetically modifying rice plants such that rice oil, rice bran and rice seeds produced therefrom have altered levels of oleic oil, palmitic acid and/or linoleic acid, by modulation of FAD2 and/or FATB gene expression.

### Leafy-Like (LFY-Like)

Leafy is a transcription factor necessary for floral induction and flower development, and is involved in the specification of floral meristem identity: LFY expression is regulated and restricted to small groups of cells flanking the shoot apical meristem wherein its high level expression marks the alteration of fate from a leaf primordium to a floral primordium (Weigel et al., Cell 69, 843-859, 1992). The protein sequence is highly conserved and in many plant species the protein is encoded by a single gene, in a few species also paralogues are present. In corn, 2 copies of the gene are present (zfl1 and zfl2). Double mutants show a normal development during vegetative growth, but floral development is disturbed (Bomblies et al., Development 130, 2385-2395, 2003). Also in Arabidopsis, loss-of-function mutants of LFY show deficiencies in floral development with a partial transformation of flowers into inflorescence shoots (Weigel et al., 1992). Leafy is also reported to play a role in the timing of flowering.

### SUMMARY

### Glutamine Synthase (GS1)

Surprisingly, it has now been found that modulating expression of a nucleic acid sequence encoding an algal-type GS1 polypeptide gives plants having enhanced yield-related traits, in particular increased seed yield relative to control plants.

According one embodiment, there is provided a method for improving yield related traits of a plant relative to control plants, comprising modulating expression of a nucleic acid sequence encoding a GS1 polypeptide in a plant.

### Phosphoethanolamine N-methyltransferase (PEAMT)

Surprisingly, it has now been found that modulating expression of a nucleic acid sequence encoding a PEAMT polypeptide gives plants having enhanced yield-related traits, relative to control plants.

According to one embodiment, there is provided a method for enhancing yield-related traits of a plant relative to control plants, comprising modulating expression of a nucleic acid sequence encoding a PEAMT polypeptide in a plant.

### Fatty Acyl-Acyl Carrier Protein (ACP) Thioesterase B (FATB)

Surprisingly, it has now been found that increasing expression in a plant of a nucleic acid sequence encoding a FATB polypeptide as defined herein, gives plants having increased seed yield-related traits relative to control plants.

According to one embodiment, there is provided a method for increasing seed yield-related traits in plants relative to control plants, comprising increasing expression in a plant of a nucleic acid sequence encoding a FATB polypeptide as defined herein. The increased seed yield-related traits comprise one or more of: increased total seed yield per plant, increased total number of seeds, increased number of filled seeds, increased seed fill rate, and increased harvest index.

### Leafy-Like (LFY-Like)

Surprisingly, it has now been found that modulating expression of a nucleic acid sequence encoding a LFY-like polypeptide gives plants having enhanced yield-related traits, in particular increased seed yield relative to control plants.

According one embodiment, there is provided a method for improving yield related traits of a plant relative to control plants, comprising modulating expression of a nucleic acid sequence encoding a LFY-like polypeptide in a plant. The improved yield related traits comprised increased seed yield and were obtained without change of flowering time compared to control plants.

### DEFINITIONS

### Polypeptide(s)/Protein(s)

The terms “polypeptide” and “protein” are used interchangeably herein and refer to amino acids in a polymeric form of any length, linked together by peptide bonds.

### Polynucleotide(s)/Nucleic Acid Sequence(s)/Nucleic Acid Sequence(s)/Nucleotide Sequence(s)

The terms “polynucleotide(s)”, “nucleic acid sequence(s)”, “nucleotide sequence(s)”, “nucleic acid sequence(s)”, “nucleic acid sequence molecule” are used interchangeably herein and refer to nucleotides, either ribonucleotides or deoxyribonucleotides or a combination of both, in a polymeric unbranched form of any length.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant” as used herein refers not only to whole plants, but also to plant parts, including seeds and seed parts.

### Homologue(s)

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds) and Table 1 below).

TABLE 1
Examples of conserved amino acid substitutions

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

### Derivatives

“Derivatives” include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

### Orthologue(s)/Paralogue(s)

Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

### Domain

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

Motif/Consensus sequence/Signature

The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

### Hybridisation

The term “hybridisation” as defined herein is a process wherein substantially homologous complementary nucleotide sequences anneal to each other. The hybridisation process can occur entirely in solution, i.e. both complementary nucleic acid sequences are in solution. The hybridisation process can also occur with one of the complementary nucleic acid sequences immobilised to a matrix such as magnetic beads, Sepharose beads or any other resin. The hybridisation process can furthermore occur with one of the complementary nucleic acid sequences immobilised to a solid support such as a nitro-cellulose or nylon membrane or immobilised by e.g. photolithography to, for example, a siliceous glass support (the latter known as nucleic acid sequence arrays or microarrays or as nucleic acid sequence chips). In order to allow hybridisation to occur, the nucleic acid sequence molecules are generally thermally or chemically denatured to melt a double strand into two single strands and/or to remove hairpins or other secondary structures from single stranded nucleic acid sequences.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acid sequences may deviate in sequence and still encode a substantially identical polypeptide, due to the degeneracy of the genetic code. Therefore medium stringency hybridisation conditions may sometimes be needed to identify such nucleic acid sequence molecules.

The Tm is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid sequence strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

1) DNA-DNA hybrids (Meinkoth and Wahl, Anal. Biochem., 138: 267-284, 1984):

Tₘ=81.5° C.+16.6×log₁₀[Na⁺]^{a}+0.41×%[G/C^{b}]−500×[L^{c}]⁻¹−0.61×% formamide

2) DNA-RNA or RNA-RNA hybrids:

Tₘ=79.8+18.5(log₁₀[Na⁺]^{a})+0.58(% G/C^{b})+11.8(% G/C^{b})²−820/L^{c}

3) oligo-DNA or oligo-RNA^{d} hybrids:

For <20 nucleotides: Tₘ=2(lₙ)

For 20-35 nucleotides: Tₘ=22+1.46(lₙ)
^{a} or for other monovalent cation, but only accurate in the 0.01-0.4 M range.
^{b} only accurate for % GC in the 30% to 75% range.
^{c} L=length of duplex in base pairs.
^{d} oligo, oligonucleotide; lₙ, =effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid sequence hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid sequence. When nucleic acid sequences of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3^{rd} Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Gene Shuffling/Directed Evolution

Gene shuffling or directed evolution consists of iterations of DNA shuffling followed by appropriate screening and/or selection to generate variants of nucleic acid sequences or portions thereof encoding proteins having a modified biological activity (Castle et al., (2004) Science 304(5674): 1151-4; U.S. Pat. Nos. 5,811,238 and 6,395,547).

### Regulatory Element/Control Sequence/Promoter

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid sequence control sequence located upstream from the transcriptional start of a gene and which is involved in recognising and binding of RNA polymerase and other proteins, thereby directing transcription of an operably linked nucleic acid sequence. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid sequence molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid sequence molecule must, as described above, be linked operably to or comprise a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid sequence used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter, in particular at a level that is in all instances below that obtained when under the control of a 35S CaMV promoter.

### Operably Linked

The term “operably linked” as used herein refers to a functional linkage between the promoter sequence and the gene of interest, such that the promoter sequence is able to initiate transcription of the gene of interest.

### Constitutive Promoter

A “constitutive promoter” refers to a promoter that is transcriptionally active during most, but not necessarily all, phases of growth and development and under most environmental conditions, in at least one cell, tissue or organ. Table 2a below gives examples of constitutive promoters.

TABLE 2a
Examples of constitutive promoters

| Gene Source | Reference |
|---|---|
| Actin | McElroy et al, Plant Cell, 2: 163-171, 1990 |
| HMGP | WO 2004/070039 |
| CAMV 35S | Odell et al, Nature, 313: 810-812, 1985 |
| CaMV 19S | Nilsson et al., Physiol. Plant. 100: 456-462, 1997 |
| GOS2 | de Pater et al, Plant J Nov; 2(6): 837-44, 1992, WO 2004/065596 |
| Ubiquitin | Christensen et al, Plant Mol. Biol. 18: 675-689, 1992 |
| Rice cyclophilin | Buchholz et al, Plant Mol Biol. 25(5): 837-43, 1994 |
| Maize H3 histone | Lepetit et al, Mol. Gen. Genet. 231: 276-285, 1992 |
| Alfalfa H3 histone | Wu et al. Plant Mol. Biol. 11: 641-649, 1988 |
| Actin 2 | An et al, Plant J. 10(1); 107-121, 1996 |
| 34S FMV | Sanger et al., Plant. Mol. Biol., 14, 1990: 433-443 |
| Rubisco small subunit | U.S. Pat. No. 4,962,028 |
| OCS | Leisner (1988) Proc Natl Acad Sci USA 85(5): 2553 |
| SAD1 | Jain et al., Crop Science, 39 (6), 1999: 1696 |
| SAD2 | Jain et al., Crop Science, 39 (6), 1999: 1696 |
| nos | Shaw et al. (1984) Nucleic acid sequences Res. 12(20): 7831-7846 |
| V-ATPase | WO 01/14572 |
| Super promoter | WO 95/14098 |
| G-box proteins | WO 94/12015 |

### Ubiquitous Promoter

A ubiquitous promoter is active in substantially all tissues or cells of an organism.

### Developmentally-Regulated Promoter

A developmentally-regulated promoter is active during certain developmental stages or in parts of the plant that undergo developmental changes.

### Inducible Promoter

An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

### Organ-Specific/Tissue-Specific Promoter

An organ-specific or tissue-specific promoter is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

Examples of root-specific promoters are listed in Table 2b below:

TABLE 2b
Examples of root-specific promoters

| Gene Source | Reference |
|---|---|
| RCc3 | Plant Mol Biol. 1995 January; 27(2): 237-48 |
| Arabidopsis PHT1 | Kovama et al., 2005; Mudge et al. (2002, Plant J. 31: 341) |
| Medicago phosphate transporter | Xiao et al., 2006 |
| Arabidopsis Pyk10 | Nitz et al. (2001) Plant Sci 161 (2): 337-346 |
| root-expressible genes | Tingey et al., EMBO J. 6: 1, 1987. |
| tobacco auxin-inducible gene | Van der Zaal et al., Plant Mol. Biol. 16, 983, 1991. |
| β-tubulin | Oppenheimer, et al., Gene 63: 87, 1988. |
| tobacco root-specific genes | Conkling, et al., Plant Physiol. 93: 1203, 1990. |
| B. napus G1-3b gene | U.S. Pat. No. 5,401,836 |
| SbPRP1 | Suzuki et al., Plant Mol. Biol. 21: 109-119, 1993. |
| LRX1 | Baumberger et al. 2001, Genes & Dev. 15: 1128 |
| BTG-26 Brassica napus | US 20050044585 |
| LeAMT1 (tomato) | Lauter et al. (1996, PNAS 3: 8139) |
| The LeNRT1-1 (tomato) | Lauter et al. (1996, PNAS 3: 8139) |
| class I patatin gene (potato) | Liu et al., Plant Mol. Biol. 153: 386-395, 1991. |
| KDC1 (Daucus carota) | Downey et al. (2000, J. Biol. Chem. 275: 39420) |
| TobRB7 gene | W Song (1997) PhD Thesis, North Carolina State |
| | University, Raleigh, NC USA |
| OsRAB5a (rice) | Wang et al. 2002, Plant Sci. 163: 273 |
| ALF5 (Arabidopsis) | Diener et al. (2001, Plant Cell 13: 1625) |
| NRT2; 1Np (N. plumbaginifolia) | Quesada et al. (1997, Plant Mol. Biol. 34: 265) |

A seed-specific promoter is transcriptionally active predominantly in seed tissue, but not necessarily exclusively in seed tissue (in cases of leaky expression). The seed-specific promoter may be active during seed development and/or during germination. The seed specific promoter may be endosperm/aleurone/embryo specific. Examples of seed-specific promoters (endosperm/aleurone/embryo specific) are shown in Table 2c to Table 2f below. Further examples of seed-specific promoters are given in Qing Qu and Takaiwa (Plant Biotechnol. J. 2, 113-125, 2004), which disclosure is incorporated by reference herein as if fully set forth.

TABLE 2c
Examples of seed-specific promoters

| Gene source | Reference |
|---|---|
| seed-specific genes | Simon et al., Plant Mol. Biol. 5: 191, 1985; |
| | Scofield et al., J. Biol. Chem. 262: 12202, 1987.; |
| | Baszczynski et al., Plant Mol. Biol. 14: 633, 1990. |
| Brazil Nut albumin | Pearson et al., Plant Mol. Biol. 18: 235-245, 1992. |
| legumin | Ellis et al., Plant Mol. Biol. 10: 203-214, 1988. |
| glutelin (rice) | Takaiwa et al., Mol. Gen. Genet. 208: 15-22, 1986; |
| | Takaiwa et al., FEBS Letts. 221: 43-47, 1987. |
| zein | Matzke et al Plant Mol Biol, 14(3): 323-32 1990 |
| napA | Stalberg et al, Planta 199: 515-519, 1996. |
| wheat LMW and HMW glutenin-1 | Mol Gen Genet 216: 81-90, 1989; NAR 17: 461-2, 1989 |
| wheat SPA | Albani et al, Plant Cell, 9: 171-184, 1997 |
| wheat α, β, γ-gliadins | EMBO J. 3: 1409-15, 1984 |
| barley Itr1 promoter | Diaz et al. (1995) Mol Gen Genet 248(5): 592-8 |
| barley B1, C, D, hordein | Theor Appl Gen 98: 1253-62, 1999; Plant J 4: 343-55, 1993; |
| | Mol Gen Genet 250: 750-60, 1996 |
| barley DOF | Mena et al, The Plant Journal, 116(1): 53-62, 1998 |
| blz2 | EP99106056.7 |
| synthetic promoter | Vicente-Carbajosa et al., Plant J. 13: 629-640, 1998. |
| rice prolamin NRP33 | Wu et al, Plant Cell Physiology 39(8) 885-889, 1998 |
| rice a-globulin Glb-1 | Wu et al, Plant Cell Physiology 39(8) 885-889, 1998 |
| rice OSH1 | Sato et al, Proc. Natl. Acad. Sci. USA, 93: 8117-8122, |
| | 1996 |
| rice α-globulin REB/OHP-1 | Nakase et al. Plant Mol. Biol. 33: 513-522, 1997 |
| rice ADP-glucose pyrophos- | Trans Res 6: 157-68, 1997 |
| phorylase | |
| maize ESR gene family | Plant J 12: 235-46, 1997 |
| sorghum α-kafirin | DeRose et al., Plant Mol. Biol 32: 1029-35, 1996 |
| KNOX | Postma-Haarsma et al, Plant Mol. Biol. 39: 257-71, 1999 |
| rice oleosin | Wu et al, J. Biochem. 123: 386, 1998 |
| sunflower oleosin | Cummins et al., Plant Mol. Biol. 19: 873-876, 1992 |
| PRO0117, putative rice 40S | WO 2004/070039 |
| ribosomal protein | |
| PRO0136, rice alanine | unpublished |
| aminotransferase | |
| PRO0147, trypsin inhibitor ITR1 | unpublished |
| (barley) | |
| PRO0151, rice WSI18 | WO 2004/070039 |
| PRO0175, rice RAB21 | WO 2004/070039 |
| PRO005 | WO 2004/070039 |
| PRO0095 | WO 2004/070039 |
| α-amylase (Amy32b) | Lanahan et al, Plant Cell 4: 203-211, 1992; Skriver et al, |
| | Proc Natl Acad Sci USA 88: 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

TABLE 2d
examples of endosperm-specific promoters

| Gene source | Reference |
|---|---|
| glutelin (rice) | Takaiwa et al. (1986) Mol Gen Genet 208: 15-22; |
| | Takaiwa et al. (1987) FEBS Letts. 221: 43-47 |
| zein | Matzke et al., (1990) Plant Mol Biol 14(3): 323-32 |
| wheat LMW and HMW glutenin-1 | Colot et al. (1989) Mol Gen Genet 216: 81-90, |
| | Anderson et al. (1989) NAR 17: 461-2 |
| wheat SPA | Albani et al. (1997) Plant Cell 9: 171-184 |
| wheat gliadins | Rafalski et al. (1984) EMBO 3: 1409-15 |
| barley Itr1 promoter | Diaz et al. (1995) Mol Gen Genet 248(5): 592-8 |
| barley B1, C, D, hordein | Cho et al. (1999) Theor Appl Genet 98: 1253-62; |
| | Muller et al. (1993) Plant J 4: 343-55; |
| | Sorenson et al. (1996) Mol Gen Genet 250: 750-60 |
| barley DOF | Mena et al, (1998) Plant J 116(1): 53-62 |
| blz2 | Onate et al. (1999) J Biol Chem 274(14): 9175-82 |
| synthetic promoter | Vicente-Carbajosa et al. (1998) Plant J 13: 629-640 |
| rice prolamin NRP33 | Wu et al, (1998) Plant Cell Physiol 39(8) 885-889 |
| rice globulin Glb-1 | Wu et al. (1998) Plant Cell Physiol 39(8) 885-889 |
| rice globulin REB/OHP-1 | Nakase et al. (1997) Plant Molec Biol 33: 513-522 |
| rice ADP-glucose pyrophosphorylase | Russell et al. (1997) Trans Res 6: 157-68 |
| maize ESR gene family | Opsahl-Ferstad et al. (1997) Plant J 12: 235-46 |
| sorghum kafirin | DeRose et al. (1996) Plant Mol Biol 32: 1029-35 |

TABLE 2e
Examples of embryo specific promoters:

| Gene source | Reference |
|---|---|
| rice OSH1 | Sato et al, Proc. Natl. Acad. Sci. USA, 93: 8117-8122, |
| | 1996 |
| KNOX | Postma-Haarsma et al, Plant Mol. Biol. 39: 257-71, 1999 |
| PRO0151 | WO 2004/070039 |
| PRO0175 | WO 2004/070039 |
| PRO005 | WO 2004/070039 |
| PRO0095 | WO 2004/070039 |

TABLE 2f
Examples of aleurone-specific promoters:

| Gene source | Reference |
|---|---|
| α-amylase | Lanahan et al, Plant Cell 4: 203-211, 1992; |
| (Amy32b) | Skriver et al, Proc Natl Acad Sci USA 88: |
| | 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

A green tissue-specific promoter as defined herein is a promoter that is transcriptionally active predominantly in green tissue, substantially to the exclusion of any other parts of a plant, whilst still allowing for any leaky expression in these other plant parts.

Examples of green tissue-specific promoters which may be used to perform the methods of the invention are shown in Table 2g below.

TABLE 2g
Examples of green tissue-specific promoters

| Gene | Expression | Reference |
|---|---|---|
| Maize Orthophosphate dikinase | Leaf specific | Fukavama et al., 2001 |
| Maize Phosphoenolpyruvate | Leaf specific | Kausch et al., 2001 |
| carboxylase | | |
| Rice Phosphoenolpyruvate | Leaf specific | Liu et al., 2003 |
| carboxylase | | |
| Rice small subunit Rubisco | Leaf specific | Nomura et al., 2000 |
| rice beta expansin EXBP9 | Shoot specific | WO 2004/070039 |
| Pigeonpea small subunit Rubisco | Leaf specific | Panguluri et al., 2005 |
| Pea RBCS3A | Leaf specific | |

Another example of a tissue-specific promoter is a meristem-specific promoter, which is transcriptionally active predominantly in meristematic tissue, substantially to the exclusion of any other parts of a plant, whilst still allowing for any leaky expression in these other plant parts. Examples of green meristem-specific promoters which may be used to perform the methods of the invention are shown in Table 2h below.

TABLE 2h
Examples of meristem-specific promoters

| Gene source | Expression pattern | Reference |
|---|---|---|
| rice OSH1 | Shoot apical meristem, | Sato et al. (1996) |
| | from embryo globular | Proc. Natl. Acad. Sci. |
| | stage to seedling stage | USA, 93: 8117-8122 |
| Rice metallothionein | Meristem specific | BAD87835.1 |
| WAK1 & WAK 2 | Shoot and root apical | Wagner & Kohorn |
| | meristems, and in ex- | (2001) Plant Cell |
| | panding leaves and sepals | 13(2): 303-318 |

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Modulation

The term “modulation” means in relation to expression or gene expression, a process in which the expression level is changed by said gene expression in comparison to the control plant, the expression level may be increased or decreased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to increased yield and/or increased growth of the plants.

### Expression

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

### Increased Expression/Overexpression

The term “increased expression” or “overexpression” as used herein means any form of expression that is additional to the original wild-type expression level.

Methods for increasing expression of genes or gene products are well documented in the art and include, for example, overexpression driven by appropriate promoters, the use of transcription enhancers or translation enhancers. Isolated nucleic acid sequences which serve as promoter or enhancer elements may be introduced in an appropriate position (typically upstream) of a non-heterologous form of a polynucleotide so as to upregulate expression of a nucleic acid sequence encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994).

### Endogenous Gene

Reference herein to an “endogenous” gene not only refers to the gene in question as found in a plant in its natural form (i.e., without there being any human intervention), but also refers to that same gene (or a substantially homologous nucleic acid sequence/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

### Decreased Expression

Reference herein to “decreased expression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants. Methods for decreasing expression are known in the art and the skilled person would readily be able to adapt the known methods for silencing so as to achieve reduction of expression of an endogenous gene in a whole plant or in parts thereof through the use of an appropriate promoter, for example.

For the reduction or substantial elimination of expression an endogenous gene in a plant, a sufficient length of substantially contiguous nucleotides of a nucleic acid sequence is required. In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from the nucleic acid sequence encoding the protein of interest (target gene), or from any nucleic acid sequence capable of encoding an orthologue, paralogue or homologue of the protein of interest. Preferably, the stretch of substantially contiguous nucleotides is capable of forming hydrogen bonds with the target gene (either sense or antisense strand), more preferably, the stretch of substantially contiguous nucleotides has, in increasing order of preference, 50%, 60%, 70%, 80%, 85%, 90%, 95%, 96%, 97%, 98%, 99%, 100% sequence identity to the target gene (either sense or antisense strand). A nucleic acid sequence encoding a (functional) polypeptide is not a requirement for the various methods discussed herein for the reduction or substantial elimination of expression of an endogenous gene.

Examples of various methods for the reduction or substantial elimination of expression in a plant of an endogenous gene, or for lowering levels and/or activity of a protein, are known to the skilled in the art. A skilled person would readily be able to adapt the known methods for silencing, so as to achieve reduction of expression of an endogenous gene in a whole plant or in parts thereof through the use of an appropriate promoter, for example.

This reduction or substantial elimination of expression may be achieved using routine tools and techniques. A preferred method for the reduction or substantial elimination of endogenous gene expression is by introducing and expressing in a plant a genetic construct into which the nucleic acid sequence (in this case a stretch of substantially contiguous nucleotides derived from the gene of interest, or from any nucleic acid sequence capable of encoding an orthologue, paralogue or homologue of any one of the protein of interest) is cloned as an inverted repeat (in part or completely), separated by a spacer (non-coding DNA).

In such a preferred method, expression of the endogenous gene is reduced or substantially eliminated through RNA-mediated silencing using an inverted repeat of a nucleic acid sequence or a part thereof (in this case a stretch of substantially contiguous nucleotides derived from the gene of interest, or from any nucleic acid sequence capable of encoding an orthologue, paralogue or homologue of the protein of interest), preferably capable of forming a hairpin structure. The inverted repeat is cloned in an expression vector comprising control sequences. A non-coding DNA nucleic acid sequence (a spacer, for example a matrix attachment region fragment (MAR), an intron, a polylinker, etc.) is located between the two inverted nucleic acid sequences forming the inverted repeat. After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into an RNA-induced silencing complex (RISC). The RISC further cleaves the mRNA transcripts, thereby substantially reducing the number of mRNA transcripts to be translated into polypeptides. For further general details see for example, Grierson et al. (1998) WO 98/53083; Waterhouse et al. (1999) WO 99/53050).

Performance of the methods of the invention does not rely on introducing and expressing in a plant a genetic construct into which the nucleic acid sequence is cloned as an inverted repeat, but any one or more of several well-known “gene silencing” methods may be used to achieve the same effects.

One such method for the reduction of endogenous gene expression is RNA-mediated silencing of gene expression (downregulation). Silencing in this case is triggered in a plant by a double stranded RNA sequence (dsRNA) that is substantially similar to the target endogenous gene. This dsRNA is further processed by the plant into about 20 to about 26 nucleotides called short interfering RNAs (siRNAs). The siRNAs are incorporated into an RNA-induced silencing complex (RISC) that cleaves the mRNA transcript of the endogenous target gene, thereby substantially reducing the number of mRNA transcripts to be translated into a polypeptide. Preferably, the double stranded RNA sequence corresponds to a target gene.

Another example of an RNA silencing method involves the introduction of nucleic acid sequences or parts thereof (in this case a stretch of substantially contiguous nucleotides derived from the gene of interest, or from any nucleic acid sequence capable of encoding an orthologue, paralogue or homologue of the protein of interest) in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

Another example of an RNA silencing method involves the use of antisense nucleic acid sequences. An “antisense” nucleic acid sequence comprises a nucleotide sequence that is complementary to a “sense” nucleic acid sequence encoding a protein, i.e. complementary to the coding strand of a double-stranded cDNA molecule or complementary to an mRNA transcript sequence. The antisense nucleic acid sequence is preferably complementary to the endogenous gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire nucleic acid sequence (in this case a stretch of substantially contiguous nucleotides derived from the gene of interest, or from any nucleic acid sequence capable of encoding an orthologue, paralogue or homologue of the protein of interest), but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine substituted nucleotides may be used. Examples of modified nucleotides that may be used to generate the antisense nucleic acid sequences are well known in the art. Known nucleotide modifications include methylation, cyclization and ‘caps’ and substitution of one or more of the naturally occurring nucleotides with an analogue such as inosine. Other modifications of nucleotides are well known in the art.

The antisense nucleic acid sequence can be produced biologically using an expression vector into which a nucleic acid sequence has been subcloned in an antisense orientation (i.e., RNA transcribed from the inserted nucleic acid sequence will be of an antisense orientation to a target nucleic acid sequence of interest). Preferably, production of antisense nucleic acid sequences in plants occurs by means of a stably integrated nucleic acid sequence construct comprising a promoter, an operably linked antisense oligonucleotide, and a terminator.

The nucleic acid sequence molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using the vectors described herein.

According to a further aspect, the antisense nucleic acid sequence is an a-anomeric nucleic acid sequence. An a-anomeric nucleic acid sequence forms specific double-stranded hybrids with complementary RNA in which, contrary to the usual b-units, the strands run parallel to each other (Gaultier et al. (1987) Nucl Ac Res 15: 6625-6641). The antisense nucleic acid sequence may also comprise a 2′-o-methylribonucleotide (Inoue et al. (1987) Nucl Ac Res 15, 6131-6148) or a chimeric RNA-DNA analogue (Inoue et al. (1987) FEBS Lett. 215, 327-330).

The reduction or substantial elimination of endogenous gene expression may also be performed using ribozymes. Ribozymes are catalytic RNA molecules with ribonuclease activity that are capable of cleaving a single-stranded nucleic acid sequence, such as an mRNA, to which they have a complementary region. Thus, ribozymes (e.g., hammerhead ribozymes (described in Haselhoff and Gerlach (1988) Nature 334, 585-591) can be used to catalytically cleave mRNA transcripts encoding a polypeptide, thereby substantially reducing the number of mRNA transcripts to be translated into a polypeptide. A ribozyme having specificity for a nucleic acid sequence can be designed (see for example: Cech et al. U.S. Pat. No. 4,987,071; and Cech et al. U.S. Pat. No. 5,116,742). Alternatively, mRNA transcripts corresponding to a nucleic acid sequence can be used to select a catalytic RNA having a specific ribonuclease activity from a pool of RNA molecules (Bartel and Szostak (1993) Science 261, 1411-1418). The use of ribozymes for gene silencing in plants is known in the art (e.g., Atkins et al. (1994) WO 94/00012; Lenne et al. (1995) WO 95/03404; Lutziger et al. (2000) WO 00/00619; Prinsen et al. (1997) WO 97/13865 and Scott et al. (1997) WO 97/38116).

Gene silencing may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682).

Gene silencing may also occur if there is a mutation on an endogenous gene and/or a mutation on an isolated gene/nucleic acid sequence subsequently introduced into a plant. The reduction or substantial elimination may be caused by a non-functional polypeptide. For example, the polypeptide may bind to various interacting proteins; one or more mutation(s) and/or truncation(s) may therefore provide for a polypeptide that is still able to bind interacting proteins (such as receptor proteins) but that cannot exhibit its normal function (such as signalling ligand).

A further approach to gene silencing is by targeting nucleic acid sequences complementary to the regulatory region of the gene (e.g., the promoter and/or enhancers) to form triple helical structures that prevent transcription of the gene in target cells. See Helene, C., Anticancer Drug Res. 6, 569-84, 1991; Helene et al., Ann. N.Y. Acad. Sci. 660, 27-36 1992; and Maher, L. J. Bioassays 14, 807-15, 1992.

Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

Alternatively, a screening program may be set up to identify in a plant population natural variants of a gene, which variants encode polypeptides with reduced activity. Such natural variants may also be used for example, to perform homologous recombination.

Artificial and/or natural microRNAs (miRNAs) may be used to knock out gene expression and/or mRNA translation. Endogenous miRNAs are single stranded small RNAs of typically 19-24 nucleotides long. They function primarily to regulate gene expression and/or mRNA translation. Most plant microRNAs (miRNAs) have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. mRNAs serve as the specificity components of RISC, since they base-pair to target nucleic acid sequences, mostly mRNAs, in the cytoplasm. Subsequent regulatory events include target mRNA cleavage and destruction and/or translational inhibition. Effects of miRNA overexpression are thus often reflected in decreased mRNA levels of target genes.

Artificial microRNAs (amiRNAs), which are typically 21 nucleotides in length, can be genetically engineered specifically to negatively regulate gene expression of single or multiple genes of interest. Determinants of plant microRNA target selection are well known in the art. Empirical parameters for target recognition have been defined and can be used to aid in the design of specific amiRNAs, (Schwab et al., Dev. Cell 8, 517-527, 2005). Convenient tools for design and generation of amiRNAs and their precursors are also available to the public (Schwab et al., Plant Cell 18, 1121-1133, 2006).

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid sequence to be introduced.

Described above are examples of various methods for the reduction or substantial elimination of expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

### Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid sequence construct of the invention. These marker genes enable the identification of a successful transfer of the nucleic acid sequence molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acid sequences into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid sequence molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid sequence can be identified for example by selection (for example, cells which have integrated the selectable marker survive whereas the other cells die). The marker genes may be removed or excised from the transgenic cell once they are no longer needed. Techniques for marker gene removal are known in the art, useful techniques are described above in the definitions section.

Since the marker genes, particularly genes for resistance to antibiotics and herbicides, are no longer required or are undesired in the transgenic host cell once the nucleic acid sequences have been introduced successfully, the process according to the invention for introducing the nucleic acid sequences advantageously employs techniques which enable the removal or excision of these marker genes. One such a method is what is known as co-transformation. The co-transformation method employs two vectors simultaneously for the transformation, one vector bearing the nucleic acid sequence according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid sequence (known as the Ac/Ds technology). The transformants can be crossed with a transposase source or the transformants are transformed with a nucleic acid sequence construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transgenic/Transgene/Recombinant

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acid sequences used in the method of the invention are not at their natural locus in the genome of said plant, it being possible for the nucleic acid sequences to be expressed homologously or heterologously. However, as mentioned, transgenic also means that, while the nucleic acid sequences according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acid sequences according to the invention at an unnatural locus in the genome, i.e. homologous or, preferably, heterologous expression of the nucleic acid sequences takes place. Preferred transgenic plants are mentioned herein.

### Transformation

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acid sequences or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet 208:274-289; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

### T-DNA Activation Tagging

T-DNA activation tagging (Hayashi et al. Science (1992) 1350-1353), involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### Tilling

The term “TILLING” is an abbreviation of “Targeted Induced Local Lesions In Genomes” and refers to a mutagenesis technology useful to generate and/or identify nucleic acid sequences encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet. 5(2): 145-50).

### Homologous Recombination

Homologous recombination allows introduction in a genome of a selected nucleic acid sequence at a defined selected position. Homologous recombination is a standard technology used routinely in biological sciences for lower organisms such as yeast or the moss Physcomitrella. Methods for performing homologous recombination in plants have been described not only for model plants (Offringa et al. (1990) EMBO J 9(10): 3077-84) but also for crop plants, for example rice (Terada et al. (2002) Nat Biotech 20(10): 1030-4; Iida and Terada (2004) Curr Opin Biotech 15(2): 132-8), and approaches exist that are generally applicable regardless of the target organism (Miller et al, Nature Biotechnol. 25, 778-785, 2007).

### Yield

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increase/Improve/Enhance

The terms “increase”, “improve” or “enhance” are interchangeable and shall mean in the sense of the application at least a 3%, 4%, 5%, 6%, 7%, 8, %, 9% or 10%, preferably at least 15% or 20%, more preferably 25%, 30%, 35% or 40% more yield and/or growth in comparison to control plants as defined herein.

### Seed Yield

Increased seed yield may manifest itself as one or more of the following: a) an increase in seed biomass (total seed weight) which may be on an individual seed basis and/or per plant and/or per square meter; b) increased number of flowers per plant; c) increased number of (filled) seeds; d) increased seed filling rate (which is expressed as the ratio between the number of filled seeds divided by the total number of seeds); e) increased harvest index, which is expressed as a ratio of the yield of harvestable parts, such as seeds, divided by the total biomass; and f) increased thousand kernel weight (TKW), and g) increased number of primary panicles, which is extrapolated from the number of filled seeds counted and their total weight. An increased TKW may result from an increased seed size and/or seed weight, and may also result from an increase in embryo and/or endosperm size.

An increase in seed yield may also be manifested as an increase in seed size and/or seed volume. Furthermore, an increase in seed yield may also manifest itself as an increase in seed area and/or seed length and/or seed width and/or seed perimeter. Increased seed yield may also result in modified architecture, or may occur because of modified architecture.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid sequence of interest. The term “plant” also encompasses plant cells, suspension cultures, callus tissue, embryos, meristematic regions, gametophytes, sporophytes, pollen and microspores, again wherein each of the aforementioned comprises the gene/nucleic acid sequence of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocaffis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticale sp., Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

### DETAILED DESCRIPTION OF THE INVENTION

Surprisingly, it has now been found that modulating expression in a plant of a nucleic acid sequence encoding a GS1 polypeptide gives plants having enhanced yield-related traits relative to control plants. According to a first embodiment, the present invention provides a method for enhancing yield-related traits in plants relative to control plants, comprising modulating expression in a plant of a nucleic acid sequence encoding a GS1 polypeptide.

Furthermore, surprisingly, it has now been found that modulating expression in a plant of a nucleic acid sequence encoding a PEAMT polypeptide gives plants having enhanced yield-related traits relative to control plants. According to a first embodiment, the present invention provides a method for enhancing yield-related traits in plants relative to control plants, comprising modulating expression in a plant of a nucleic acid sequence encoding a PEAMT polypeptide.

Furthermore, surprisingly, it has now been found that increasing expression in a plant of a nucleic acid sequence encoding a FATB polypeptide as defined herein, gives plants having increased seed yield-related traits relative to control plants. According to a first embodiment, the present invention provides a method for increasing seed yield-related traits in plants relative to control plants, comprising increasing expression in a plant of a nucleic acid sequence encoding a FATB polypeptide.

Furthermore, surprisingly, it has now been found that modulating expression in a plant of a nucleic acid sequence encoding a LFY-like polypeptide gives plants having enhanced yield-related traits relative to control plants. According to a first embodiment, the present invention provides a method for enhancing yield-related traits in plants relative to control plants, comprising modulating expression in a plant of a nucleic acid sequence encoding a LFY-like polypeptide.

A preferred method for modulating (preferably, increasing) expression of a nucleic acid sequence encoding a GS1 polypeptide, or a PEAMT polypeptide, or a FATB polypeptide, or a LFY-like polypeptide is by introducing and expressing in a plant a nucleic acid sequence encoding a GS1 polypeptide, or a PEAMT polypeptide, or a FATB polypeptide, or a LFY-like polypeptide.

Concerning GS1 polypeptides, any reference hereinafter to a “protein useful in the methods of the invention” is taken to mean a GS1 polypeptide as defined herein. Any reference hereinafter to a “nucleic acid sequence useful in the methods of the invention” is taken to mean a nucleic acid sequence capable of encoding such a GS1 polypeptide. The nucleic acid sequence to be introduced into a plant (and therefore useful in performing the methods of the invention) is any nucleic acid sequence encoding the type of protein which will now be described, hereafter also named “GS1 nucleic acid sequence” or “GS1 gene”.

A “GS1 polypeptide” as defined herein for the purpose of the present invention refers to any Glutamine Synthase 1 (GS1) that clusters together with GS1 proteins of algal origin (to form an algal-type clade) in a phylogenetic tree such as the one displayed in FIG. 3. Preferably the GS1 is of algal origin. Glutamine synthase (Enzyme Catalogue number EC 6.3.1.2) catalyses the following reaction:

ATP+L-Glutamate+NH₃⇄μL-Glutamine+ADP+Phosphate

Preferably, the GS1 protein comprises Gln-synt_C domain (Pfam accession PF00120) and a Gln-synt_N domain (Pfam accession PF03951). Further preferably, the GS1 protein useful in the methods of the present invention comprises at least one, preferably at least two, more preferably all three of the following conserved sequences in which maximally 4, preferably 3 or less, more preferably 2 or less, most preferably 1 or no mismatches are present:

Motif 1 (SEQ ID NO: 3):
GY(Y/L/F)(E/T)DRRP(A/S/P)(A/S)(N/D)(V/L/A/M)D
(P/A)Y
Preferably Motif 1 is GY (Y/L/F)(E/T)DRRP(A/P)
(A/S)(N/D)(V/L/A)D(P/A)Y
Motif 2 (SEQ ID NO: 4):
DP(I/F)RG(A/E/D/S/G/L/V)(P/N/D)(H/N)(V/I)(L/I)V
(L/I/M)(C/T/A)
Preferably, motif 2 is DP(I/F)RG(A/E/G)(P/N/D)
(H/N)(V/I)LV(L/M)(C/A)
Motif 3 (SEQ ID NO: 5):
G(A/L/M/G/C)H(T/S/I/V/F)(N/K)(F/Y/V)S(T/S/N)
Preferably Motif 3 is G(A/M/G/C)H(T/I/V/F)(N/K)
(F/Y)S(T/N)

Alternatively, the homologue of a GS1 protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 2, provided that the homologous protein comprises the conserved motifs as outlined above. The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered.

Preferably, the polypeptide sequence which when used in the construction of a phylogenetic tree, such as the one depicted in FIGS. 3a and 3b, clusters with the algal-type clade (the group of algal GS1 polypeptides comprising the amino acid sequence represented by SEQ ID NO: 2) rather than with the plant chloroplastic or plant cytosolic glutamine synthase group.

Concerning PEAMT polypeptides, any reference hereinafter to a “protein (or polypeptide) useful in the methods of the invention” is taken to mean a PEAMT polypeptide as defined herein. Any reference hereinafter to a “nucleic acid sequence useful in the methods of the invention” is taken to mean a nucleic acid sequence capable of encoding such a PEAMT polypeptide. The nucleic acid sequence to be introduced into a plant (and therefore useful in performing the methods of the invention) is any nucleic acid sequence encoding the type of protein which will now be described, hereafter also named “PEAMT nucleic acid sequence” or “PEAMT gene”.

A “PEAMT polypeptide” as defined herein refers to any polypeptide having phosphoethanolamine N-methyltransferase activity.

Tools and techniques for measuring Phosphoethanolamine N-methyltransferase activity are well known in the art. For example in vivo activity of PEAMT polynucleotide and the polypeptide encoded thereof can be analyzed by complementation in Schizosaccharommyces pombe (Nuccio et al; 2000). PEAMT activity may also be determined in vitro as described by (Nuccio et al; 2000).

A “PEAMT polypeptide comprises two IPR013216, Methyltransferase type 11 domains (Interpro accession number: IPR013216; pfam accession number: PF08241) and optionally a ubiE/COQ5 methyltransferase domain (Ubie_methyltran (pfam accession number: PF01209).

A Methyltransferase type 11 domain and method to identify the presence of such domain in a polypeptide are well known in the art. Examples of proteins comprising two Methyltransferase type 11 domains are set forth in Table A2. The Methyltransferase type 11 domains as present in SEQ ID NO: 58 are given in SEQ ID NO: 86 and 87. The Example section teaches methods to identify the presence of Methyltransferase type 11 and ubiE/COQ5 methyltransferase in the PEAMT polypeptide represented by SEQ ID NO: 58.

SEQ ID NO: 58 comprises two Methyltransferase
type 11 domains represented by
SEQ ID NO: 86
(PPYEGKSVLELGAGIGRFTGELAQKAGEVIALDIIESAIQKNESVNG
HYKNIKFMCADVTSPDLKIKDGSIDLIFSNWLLMYLSDKEVELMAERM
IGWVKPGGYIFFRES)
and
SEQ ID NO: 87
(DLKPGQKVLDVGCGIGGGDFYMAENFDVHVVGIDLSVNMISFALERA
IGLKCSVEFEVADCTTKTYPDNSFDVIYSRDTILHIQDKPALFRTFFK
WLKPGGKVLITDY).
Additionally, SEQ ID NO: 58 comprises a ubiE/COQ5
methyltransferase domain represented by
SEQ ID NO: 88
(ERVFGEGYVSTGGFETTKEFVAKMDLKPGQKVLDVGCGIGGGDFYMA
ENFDVHVVGIDLSVNMISFALERAIGLKCSVEFEVADCTTKTYPDNSF
DVIYSRDTILHIQDKPALFRTFFKWLKPGGKVLITDYCRSAETPSPEF
AEYIKQRGYDLHDVQAYGQMLKDAGFDDVIAEDRTDQ)

A “PEAMT polypeptide” useful in the methods of the invention may additionally comprise one or more of the following motifs:

| 1. Motif 4: | |
|---|---|
| IFFRESCFHQSGD; | (SEQ ID NO: 89) |
| 2. Motif 5: | |
| EYIKQR; | (SEQ ID NO: 90) |
| 3. Motif 6: | |
| WGLFIA; | (SEQ ID NO: 91) |

Motifs 4 to 6 are located in the C-terminal half of the PEAMT polypeptide represented by SEQ ID NO: 58 at amino acid positions 138-150, 383-388 and 467-472 respectively.

Preferably, the PEAMT protein useful in the methods of the invention comprises a motif having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of Motifs 1 to 3.

More preferably, the PEAMT protein useful in the methods of the invention comprises a a conserved domain having in increasing order of preference at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of SEQ ID NO: 86 to 88 or to any of the amino acid domains set forth in Table C2 of the Example section.

A “PEAMT or a homologue thereof” as defined herein refers to any polypeptide having in increasing order of preference at least 50%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 58.

Alternatively, the homologue of a PEAMT protein comprises a conserved amino acid domain having in increasing order of preference at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid motifs set forth in Table C2.

The sequence identity is determined using an alignment algorithms, such as the Needleman Wunsch algorithm in the program GAP (GCG Wisconsin Package, Accelrys), preferably with default parameters or BLAST. Compared to overall sequence identity, the sequence identity will generally be higher when only conserved domains or motifs are considered.

Preferably, the polypeptide sequence which when used in the construction of a phylogenetic tree, such as the one depicted in FIG. 6, clusters with the group I of PEAMT polypeptides comprising the amino acid sequence represented by SEQ ID NO: 58 rather than with any other group.

Furthermore, the invention also provides hitherto unknown a nucleic acid sequence encoding a FATB polypeptide and a FATB polypeptide.

According to one embodiment of the present invention, there is therefore provided an isolated nucleic acid sequence comprising:
(i) a nucleic acid sequence as represented by SEQ ID NO: 130;
(ii) the complement of a nucleic acid sequence as represented by SEQ ID NO: 130;
(iii) a nucleic acid sequence encoding FATB polypeptide having, in increasing order of preference, at least 70%, 75%, 80%, 85%, 90%, 95%, 96%, 97%, 98%, 99% or more amino acid sequence identity to the polypeptide sequence as represented by SEQ ID NO: 131.

According to a further embodiment of the present invention, there is also provided an isolated polypeptide comprising:
(i) a polypeptide sequence represented by SEQ ID NO: 131;
(ii) a polypeptide sequence having, in increasing order of preference, at least 70%, 75%, 80%, 85%, 90%, 95%, 96%, 97%, 98%, 99% or more sequence identity to the polypeptide sequence as represented by SEQ ID NO: 131;
(iii) derivatives of any of the polypeptide sequences given in (i) or (ii) above.

A preferred method for increasing expression in a plant of a nucleic acid sequence encoding a FATB polypeptide is by introducing and expressing in a plant a nucleic acid sequence encoding a FATB polypeptide.

Concerning FATB polypeptides, any reference hereinafter to a “protein useful in the methods of the invention” is taken to mean a FATB polypeptide as defined herein. Any reference hereinafter to a “nucleic acid sequence useful in the methods of the invention” is taken to mean a nucleic acid sequence capable of encoding such a FATB polypeptide. The nucleic acid sequence to be introduced into a plant (and therefore useful in performing the methods of the invention) is any nucleic acid sequence encoding the type of polypeptide, which will now be described, hereafter also named “FATB nucleic acid sequence” or “FATB gene”.

A “FATB polypeptide” as defined herein refers to any polypeptide comprising (i) a plastidic transit peptide; (ii) at least one transmembrane helix; (iii) and an acyl-ACP thioesterase family domain with an InterPro accession IPR002864;

Alternatively or additionally, a “FATB polypeptide” as defined herein refers to any polypeptide sequence having (i) a plastidic transit peptide; (ii) in increasing order of preference at least 50%, 55%, 60%, 65%, 70%, 75%, 80%, 85%, 90%, 95%, 98%, 99% or more amino acid sequence identity to a transmembrane helix as represented by SEQ ID NO: 141; and having in increasing order of preference at least 50%, 55%, 60%, 65%, 70%, 75%, 80%, 85%, 90%, 95%, 98%, 99% or more amino acid sequence identity to an acyl-ACP thioesterase family domain as represented by SEQ ID NO: 140.

Alternatively or additionally, a “FATB polypeptide” as defined herein refers to any polypeptide having in increasing order of preference at least 50%, 55%, 60%, 65%, 70%, 75%, 80%, 85%, 90%, 95%, 98%, 99% or more amino acid sequence identity to a FATB polypeptide as represented by SEQ ID NO: 93 or to any of the polypeptide sequences given in Table A3 herein.

Alternatively or additionally, a “FATB polypeptide” as defined herein refers to any polypeptide sequence which when used in the construction of a FATs (FATA and FATB together) phylogenetic tree, such as the one depicted in FIG. 10, clusters with the clade of FATB polypeptides comprising the polypeptide sequence as represented by SEQ ID NO: 93 (shown by an arrow in FIG. 10) rather than with the Glade of FATA polypeptides.

Alternatively or additionally, an “FATB polypeptide” is a polypeptide with enzymatic activity consisting in hydrolyzing acyl-ACP thioester bonds, preferentially from saturated acyl-ACPs (with chain lengths that vary between 8 and 18 carbons), releasing free fatty acids and acyl carrier protein (ACP).

Concerning LFY-like polypeptides, any reference hereinafter to a “protein useful in the methods of the invention” is taken to mean a LFY-like polypeptide as defined herein. Any reference hereinafter to a “nucleic acid sequence useful in the methods of the invention” is taken to mean a nucleic acid sequence capable of encoding such a LFY-like polypeptide. The nucleic acid sequence to be introduced into a plant (and therefore useful in performing the methods of the invention) is any nucleic acid sequence encoding the type of protein which will now be described, hereafter also named “LFY-like nucleic acid sequence” or “LFY-like gene”.

A “LFY-like polypeptide” as defined herein refers to any transcription factor comprising a FLO_LFY domain (InterPro accession IPR002910; Pfam accession PF01698). The FLO_LFY domain represents the major part of the protein sequence (see FIG. 14) and is highly conserved (FIG. 15).

Preferably, the LFY-like protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 146, provided that the homologous protein comprises the conserved FLO_LFY motif as outlined above. The overall sequence identity is determined using a global alignment algorithm, such as the Needleman Wunsch algorithm in the program GAP (GCG Wisconsin Package, Accelrys), preferably with default parameters. Compared to overall sequence identity, the sequence identity will generally be higher when only conserved domains or motifs (such as the FLO_LFY domain) are considered.

Preferably, the polypeptide sequence which when used in the construction of a phylogenetic tree, such as the one depicted in FIG. 16, clusters with the group of LFY-like polypeptides.

The terms “domain”, “signature” and “motif” are defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic acid sequences Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic acid sequences Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic acid sequences Res. 31:3784-3788 (2003)). Domains or motifs may also be identified using routine techniques, such as by sequence alignment.

Concerning FATB polypeptides, analysis of the polypeptide sequence of SEQ ID NO: 93 is presented below in Example 4 herein. For example, a FATB polypeptide as represented by SEQ ID NO: 93 comprises an acyl-ACP thioesterase family domain with an InterPro accession IPR002864. An alignment of the polypeptides of Table A3 herein, is shown in FIG. 13. Such alignments are useful for identifying the most conserved domains or motifs between the FATB polypeptides, such as the TMpred predicted transmembrane helix (see Example 5 herein) as represented by SEQ ID NO: 141 (comprised in SEQ ID NO: 93).

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid sequence or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

Concerning FATB polypeptides, example 3 herein describes in Table B3 the percentage identity between the FATB polypeptide as represented by SEQ ID NO: 93 and the FATB polypeptides listed in Table A2, which can be as low as 53% amino acid sequence identity.

The task of protein subcellular localisation prediction is important and well studied. Knowing a protein's localisation helps elucidate its function. Experimental methods for protein localization range from immunolocalization to tagging of proteins using green fluorescent protein (GFP) or beta-glucuronidase (GUS). Such methods are accurate although labor-intensive compared with computational methods. Recently much progress has been made in computational prediction of protein localisation from sequence data. Among algorithms well known to a person skilled in the art are available at the ExPASy Proteomics tools hosted by the Swiss Institute for Bioinformatics, for example, PSort, TargetP, ChloroP, LocTree, Predotar, LipoP, MITOPROT, PATS, PTS1, SignalP, TMHMM, and others. The identification of subcellular localisation of the polypeptide of the invention is shown in Example 5. In particular SEQ ID NO: 2 of the present invention is assigned to the plastidic (chloroplastic) compartment of plant cells. In addition to a transit peptide, FATB polypeptides further comprise a predicted transmembrane helix (see Example 5 herein) for anchoring to a chloroplast membrane.

Methods for targeting to plastids are well known in the art and include the use of transit peptides. Table 3 below shows examples of transit peptides which can be used to target any FATB polypeptide to a plastid, which FATB polypeptide is not, in its natural form, normally targeted to a plastid, or which FATB polypeptide in its natural form is targeted to a plastid by virtue of a different transit peptide (for example, its natural transit peptide). Cloning a nucleic acid sequence encoding a transit peptide upstream and in-frame of a nucleic acid sequence encoding a polypeptide (for example, a FATB polypeptide lacking its own transit peptide), involves standard molecular techniques that are well-known in the art.

TABLE 3
Examples of transit peptide sequences useful in
targeting polypeptides to plastids

| NCBI | | | |
|---|---|---|---|
| Accession | | | |
| Number/SEQ | | | |
| ID NO | Source Organism | Protein Function | Transit Peptide Sequence |
| SEQ ID NO: | Chlamydomonas | Ferredoxin | MAMAMRSTFAARVGAKPAVRGARPASRMSCMA |
| P07839 | | | |
| SEQ ID NO: | Chlamydomonas | Rubisco activase | MQVTMKSSAVSGQRVGGARVATRSVRRAQLQV |
| AAR23425 | | | |
| SEQ ID NO: | Arabidopsis thaliana | Aspartate amino | MASLMLSLGSTSLLPREINKDKLKLGTSASNPFLKAK |
| CAA56932 | | transferase | SFSRVTMTVAVKPSR |
| SEQ ID NO: | Arabidopsis thaliana | Acyl carrier protein1 | MATQFSASVSLQTSCLATTRISFQKPALISNHGKTNL |
| CAA31991 | | | SFNLRRSIPSRRLSVSC |
| SEQ ID NO: | Arabidopsis thaliana | Acyl carrier protein2 | MASIAASASISLQARPRQLAIAASQVKSFSNGRRSSL |
| CAB63798 | | | SFNLRQLPTRLTVSCAAKPETVDKVCAVVRKQL |
| SEQ ID NO: | Arabidopsis thaliana | Acyl carrier protein3 | MASIATSASTSLQARPRQLVIGAKQVKSFSYGSRSNL |
| CAB63799 | | | SFNLRQLPTRLTVYCAAKPETVDKVCAVVRKQLSLKE |

The FATB polypeptide is targeted and active in the chloroplast, i.e., the FATB polypeptide is capable of hydrolyzing acyl-ACP thioester bonds, preferentially from saturated acyl-ACPs (with chain lengths that vary between 8 and 18 carbons), releasing free fatty acids and acyl carrier protein (ACP). Assays for testing these activities are well known in the art. Further details are provided in Example 6.

Furthermore, GS1 polypeptides (at least in their native form) typically have glutamine synthase activity. Tools and techniques for measuring glutamine synthase activity are well known in the art (see for example Martin et al. Anal. Biochem. 125, 24-29, 1982 and Example 6).

In addition, PEAMT polypeptides, when expressed in rice according to the methods of the present invention as outlined in the Example section, give plants having increased yield related traits, in particular one or more of increased green biomass, early vigour, total seed weight, number of flowers per panicle, seed filing rate, thousand kernel weight and harvest index.

Furthermore, LFY-like polypeptides (at least in their native form) typically have DNA-binding activity. Tools and techniques for measuring DNA-binding activity are well known in the art. An example of characterisation of DNA binding properties of a protein is provided by Xue (Plant J. 41, 638-649, 2005).

In addition, LFY-like polypeptides, when expressed in rice according to the methods of the present invention as outlined in Examples 7 and 8, give plants having increased yield related traits, in particular increased seed yield.

Concerning GS1 polypeptides, the present invention is illustrated by transforming plants with the nucleic acid sequence represented by SEQ ID NO: 1, encoding the polypeptide sequence of SEQ ID NO: 2. However, performance of the invention is not restricted to these sequences; the methods of the invention may advantageously be performed using any GS1-encoding nucleic acid sequence or GS1 polypeptide as defined herein.

Examples of nucleic acid sequences encoding GS1 polypeptides are given in Table A1 of Example 1 herein. Such nucleic acid sequences are useful in performing the methods of the invention. The amino acid sequences given in Table A1 of Example 1 are example sequences of orthologues and paralogues of the GS1 polypeptide represented by SEQ ID NO: 2, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A1 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1 or SEQ ID NO: 2, the second BLAST would therefore be against Chlamydomonas sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning PEAMT polypeptides, the present invention is illustrated by transforming plants with the nucleic acid sequence represented by SEQ ID NO: 57, encoding the polypeptide sequence of SEQ ID NO: 58. However, performance of the invention is not restricted to these sequences; the methods of the invention may advantageously be performed using any PEAMT-encoding nucleic acid sequence or PEAMT polypeptide as defined herein.

Examples of nucleic acid sequences encoding PEAMT polypeptides are given in Table A2 of the Examples section herein. Such nucleic acid sequences are useful in performing the methods of the invention. The amino acid sequences given in Table A of the Examples section are example sequences of orthologues and paralogues of the PEAMT polypeptide represented by SEQ ID NO: 58, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A2 of the Examples section) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 57 or SEQ ID NO: 58, the second BLAST would therefore be against Arabidopsis thaliana sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning FATB polypeptides, the present invention is illustrated by transforming plants with the nucleic acid sequence represented by SEQ ID NO: 92, encoding the FATB polypeptide sequence of SEQ ID NO: 93. However, performance of the invention is not restricted to these sequences; the methods of the invention may advantageously be performed using any nucleic acid sequence encoding a FATB polypeptide as defined herein.

Examples of nucleic acid sequences encoding FATB polypeptides are given in Table A3 of Example 1 herein. Such nucleic acid sequences are useful in performing the methods of the invention. The polypeptide sequences given in Table A3 of Example 1 are example sequences of orthologues and paralogues of the FATB polypeptide represented by SEQ ID NO: 93, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A3 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 92 or SEQ ID NO: 93, the second BLAST would therefore be against Arabidopsis thaliana sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning LFY-like polypeptides, the present invention is illustrated by transforming plants with the nucleic acid sequence represented by SEQ ID NO: 145, encoding the polypeptide sequence of SEQ ID NO: 146. However, performance of the invention is not restricted to these sequences; the methods of the invention may advantageously be performed using any LFY-like-encoding nucleic acid sequence or LFY-like polypeptide as defined herein.

Examples of nucleic acid sequences encoding LFY-like polypeptides are given in Table A4 of Example 1 herein. Such nucleic acid sequences are useful in performing the methods of the invention. The amino acid sequences given in Table A4 of Example 1 are example sequences of orthologues and paralogues of the LFY-like polypeptide represented by SEQ ID NO: 146, the terms “orthologues” and “paralogues” being as defined herein. Further orthologues and paralogues may readily be identified by performing a so-called reciprocal blast search.

Typically, this involves a first BLAST involving BLASTing a query sequence (for example using any of the sequences listed in Table A4 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 145 or SEQ ID NO: 146, the second BLAST would therefore be against Arabidopsis sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid sequence (or polypeptide) sequences over a particular length. In the case of large families, ClustalW may be used, followed by a neighbour joining tree, to help visualize clustering of related genes and to identify orthologues and paralogues.

Furthermore, the invention also provides hitherto unknown GS1-encoding nucleic acid sequences and GS1 polypeptides.

According to a further embodiment of the present invention, there is also provided an isolated polypeptide selected from:
(i) an amino acid sequence represented by SEQ ID NO: 53 or SEQ ID NO: 54;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 53 or SEQ ID NO: 54,
(iii) derivatives of any of the amino acid sequences given in (i) or (ii) above.

The inventions also provides nucleic acid sequences encoding the unknown GS1 polypeptides as disclosed above and nucleic acid sequences hybridising thereto, preferably under stringent conditions.

Nucleic acid sequence variants may also be useful in practising the methods of the invention. Examples of such variants include nucleic acid sequences encoding homologues and derivatives of any one of the amino acid sequences given in Table A1 to A4 of the Examples section, the terms “homologue” and “derivative” being as defined herein. Also useful in the methods of the invention are nucleic acid sequences encoding homologues and derivatives of orthologues or paralogues of any one of the amino acid sequences given in Table A1 to A4 of the Examples section. Homologues and derivatives useful in the methods of the present invention have substantially the same biological and functional activity as the unmodified protein from which they are derived.

Further nucleic acid sequence variants useful in practising the methods of the invention include portions of nucleic acid sequences encoding GS1 polypeptides, or PEAMT polypeptides, or FATB polypeptides, or LFY-like polypeptides, nucleic acid sequences hybridising to nucleic acid sequences encoding GS1 polypeptides, or PEAMT polypeptides, or FATB polypeptides, or LFY-like polypeptides, splice variants of nucleic acid sequences encoding GS1 polypeptides, or PEAMT polypeptides, or FATB polypeptides, or LFY-like polypeptides, allelic variants of nucleic acid sequences encoding GS1 polypeptides, or PEAMT polypeptides, or FATB polypeptides, or LFY-like polypeptides, and variants of nucleic acid sequences encoding GS1 polypeptides, or PEAMT polypeptides, or FATB polypeptides, or LFY-like polypeptides, obtained by gene shuffling. The terms hybridising sequence, splice variant, allelic variant and gene shuffling are as described herein.

Nucleic acid sequences encoding GS1 polypeptides, or PEAMT polypeptides, or FATB polypeptides, or LFY-like polypeptides, need not be full-length nucleic acid sequences, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table A1 to A4 of the Examples section, or a portion of a nucleic acid sequence encoding an orthologue, paralogue or homologue of any of the amino acid sequences given in Table A1 to A4 of the Examples section.

A portion of a nucleic acid sequence may be prepared, for example, by making one or more deletions to the nucleic acid sequence. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Concerning GS1 polypeptides, portions useful in the methods of the invention, encode a GS1 polypeptide as defined herein, and have substantially the same biological activity as the amino acid sequences given in Table A1 of Example 1. Preferably, the portion is a portion of any one of the nucleic acid sequences given in Table A1 of Example 1, or is a portion of a nucleic acid sequence encoding an orthologue or paralogue of any one of the amino acid sequences given in Table A1 of Example 1. Preferably the portion is at least 500, 550, 600, 650, 700, 750, 800, 850, 900, 950, 1000, 1050, 1100, 1150 consecutive nucleotides in length, the consecutive nucleotides being of any one of the nucleic acid sequences given in Table A1 of Example 1, or of a nucleic acid sequence encoding an orthologue or paralogue of any one of the amino acid sequences given in Table A1 of Example 1. Most preferably the portion is a portion of the nucleic acid sequence of SEQ ID NO: 1. Preferably, the portion encodes a fragment of an amino acid sequence which, when used in the construction of a phylogenetic tree, such as the one depicted in FIGS. 3a and 3b, clusters with the algal-type clade (the group of algal GS1 polypeptides comprising the amino acid sequence represented by SEQ ID NO: 2) rather than with the plant chloroplastic or plant cytosolic glutamine synthase group.

Concerning PEAMT polypeptides, portions useful in the methods of the invention, encode a PEAMT polypeptide as defined herein, and have substantially the same biological activity as the amino acid sequences given in Table A2 of the Examples section. Preferably, the portion is a portion of any one of the nucleic acid sequences given in Table A2 of the Examples section, or is a portion of a nucleic acid sequence encoding an orthologue or paralogue of any one of the amino acid sequences given in Table A2 of the Examples section. Preferably the portion is at least 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, consecutive nucleotides in length, the consecutive nucleotides being of any one of the nucleic acid sequences given in Table A2 of the Examples section, or of a nucleic acid sequence encoding an orthologue or paralogue of any one of the amino acid sequences given in Table A2 of the Examples section. Most preferably the portion is a portion of the nucleic acid sequence of SEQ ID NO: 57. Preferably, the portion encodes a fragment of an amino acid sequence which, when used in the construction of a phylogenetic tree, such as the one depicted in FIG. 6, clusters with the group I of PEAMT polypeptides comprising the amino acid sequence represented by SEQ ID NO: 58 rather than with any other group.

Concerning FATB polypeptides, portions useful in the methods of the invention, encode a FATB polypeptide as defined herein, and have substantially the same biological activity as the polypeptide sequences given in Table A3 of Example 1. Preferably, the portion is a portion of any one of the nucleic acid sequences given in Table A3 of Example 1, or is a portion of a nucleic acid sequence encoding an orthologue or paralogue of any one of the polypeptide sequences given in Table A3 of Example 1. Preferably the portion is, in increasing order of preference at least 600, 650, 700, 750, 800, 850, 900, 950, 1000, 1050, 1100, 1150, 1200 or more consecutive nucleotides in length, the consecutive nucleotides being of any one of the nucleic acid sequences given in Table A3 of Example 1, or of a nucleic acid sequence encoding an orthologue or paralogue of any one of the polypeptide sequences given in Table A3 of Example 1. Preferably, the portion is a portion of a nucleic sequence encoding a polypeptide sequence having in increasing order of preference at least 50%, 55%, 60%, 65%, 70%, 75%, 80%, 85%, 90%, 95%, 98%, 99% or more amino acid sequence identity to the FATB polypeptide as represented by SEQ ID NO: 93 or to any of the polypeptide sequences given in Table A herein. Most preferably, the portion is a portion of the nucleic acid sequence of SEQ ID NO: 92.

Concerning LFY-like polypeptide, portions useful in the methods of the invention, encode a LFY-like polypeptide as defined herein, and have substantially the same biological activity as the amino acid sequences given in Table A4 of Example 1. Preferably, the portion is a portion of any one of the nucleic acid sequences given in Table A4 of Example 1, or is a portion of a nucleic acid sequence encoding an orthologue or paralogue of any one of the amino acid sequences given in Table A4 of Example 1. Preferably the portion is at least 600, 650, 700, 750, 800, 850, 900, 950, 1000, 1050, 1100, 1150, 1200, 1250, 1300, 1350 consecutive nucleotides in length, the consecutive nucleotides being of any one of the nucleic acid sequences given in Table A4 of Example 1, or of a nucleic acid sequence encoding an orthologue or paralogue of any one of the amino acid sequences given in Table A4 of Example 1. Most preferably the portion is a portion of the nucleic acid sequence of SEQ ID NO: 145. Preferably, the portion encodes a fragment of an amino acid sequence which, when used in the construction of a phylogenetic tree, such as the one depicted in FIG. 16, clusters with the group of LFY-like polypeptides.

Another nucleic acid sequence variant useful in the methods of the invention is a nucleic acid sequence capable of hybridising, under reduced stringency conditions, preferably under stringent conditions, with a nucleic acid sequence encoding a GS1 polypeptide, or a PEAMT polypeptide, or a FATB polypeptide, a LFY-like polypeptide, as defined herein, or with a portion as defined herein.

According to the present invention, there is provided a method for enhancing yield-related traits in plants, comprising introducing and expressing in a plant a nucleic acid sequence capable of hybridizing to any one of the nucleic acid sequences given in Table A1 to A4 of Example 1, or comprising introducing and expressing in a plant a nucleic acid sequence capable of hybridising to a nucleic acid sequence encoding an orthologue, paralogue or homologue of any of the nucleic acid sequences given in Table A1 to A4 of Example 1.

Concerning GS1 polypeptides, hybridising sequences useful in the methods of the invention encode a GS1 polypeptide as defined herein, having substantially the same biological activity as the amino acid sequences given in Table A1 of Example 1. Preferably, the hybridising sequence is capable of hybridising to the complement of any one of the nucleic acid sequences given in Table A1 of Example 1, or to a portion of any of these sequences, a portion being as defined above, or the hybridising sequence is capable of hybridising to the complement of a nucleic acid sequence encoding an orthologue or paralogue of any one of the amino acid sequences given in Table A1 of Example 1. Most preferably, the hybridising sequence is capable of hybridising to the complement of a nucleic acid sequence as represented by SEQ ID NO: 1 or to a portion thereof.

Concerning GS1 polypeptides, preferably, the hybridising sequence encodes a polypeptide with an amino acid sequence which, when full-length and used in the construction of a phylogenetic tree, such as the one depicted in FIGS. 3a and 3b, clusters with the algal-type clade (the group of algal GS1 polypeptides comprising the amino acid sequence represented by SEQ ID NO: 2) rather than with the plant chloroplastic or plant cytosolic glutamine synthase group.

Concerning PEAMT polypeptides, hybridising sequences useful in the methods of the invention encode a PEAMT polypeptide as defined herein, having substantially the same biological activity as the amino acid sequences given in Table A2 of the Examples section. Preferably, the hybridising sequence is capable of hybridising to the complement of any one of the nucleic acid sequences given in Table A2 of the Examples section, or to a portion of any of these sequences, a portion being as defined above, or the hybridising sequence is capable of hybridising to the complement of a nucleic acid sequence encoding an orthologue or paralogue of any one of the amino acid sequences given in Table A2 of the Examples section. Most preferably, the hybridising sequence is capable of hybridising to the complement of a nucleic acid sequence as represented by SEQ ID NO: 57 or to a portion thereof.

Concerning PEAMT polypeptides, preferably, the hybridising sequence encodes a polypeptide with an amino acid sequence which, when full-length and used in the construction of a phylogenetic tree, such as the one depicted in FIG. 6, clusters with the group I of PEAMT polypeptides comprising the amino acid sequence represented by SEQ ID NO: 58 rather than with any other group.

Concerning FATB polypeptides, hybridising sequences useful in the methods of the invention encode a FATB polypeptide as defined herein, and have substantially the same biological activity as the polypeptide sequences given in Table A3 of Example 1. Preferably, the hybridising sequence is capable of hybridising to any one of the nucleic acid sequences given in Table A3 of Example 1, or to a complement thereof, or to a portion of any of these sequences, a portion being as defined above, or wherein the hybridising sequence is capable of hybridising to a nucleic acid sequence encoding an orthologue or paralogue of any one of the polypeptide sequences given in Table A3 of Example 1, or to a complement thereof.

Concerning FATB polypeptides, preferably, the hybridising sequence is capable of hybridising to a nucleic acid sequence encoding a polypeptide sequence having in increasing order of preference at least 50%, 55%, 60%, 65%, 70%, 75%, 80%, 85%, 90%, 95%, 98%, 99% or more amino acid sequence identity to the FATB polypeptide as represented by SEQ ID NO: 93 or to any of the polypeptide sequences given in Table A3 of Example 1 herein. Most preferably, the hybridising sequence is capable of hybridising to a nucleic acid sequence as represented by SEQ ID NO: 92 or to a portion thereof.

Concerning LFY-like polypeptides, hybridising sequences useful in the methods of the invention encode a LFY-like polypeptide as defined herein, having substantially the same biological activity as the amino acid sequences given in Table A4 of Example 1. Preferably, the hybridising sequence is capable of hybridising to the complement of any one of the nucleic acid sequences given in Table A4 of Example 1, or to a portion of any of these sequences, a portion being as defined above, or the hybridising sequence is capable of hybridising to the complement of a nucleic acid sequence encoding an orthologue or paralogue of any one of the amino acid sequences given in Table A4 of Example 1. Most preferably, the hybridising sequence is capable of hybridising to the complement of a nucleic acid sequence as represented by SEQ ID NO: 145 or to a portion thereof.

Concerning LFY-like polypeptides, preferably, the hybridising sequence encodes a polypeptide with an amino acid sequence which, when full-length and used in the construction of a phylogenetic tree, such as the one depicted in FIG. 16, clusters with the group of LFY-like polypeptides.

Another nucleic acid sequence variant useful in the methods of the invention is a splice variant encoding a GS1 polypeptide, or a PEAMT polypeptide, or a FATB polypeptide, or a LFY-like polypeptide, as defined hereinabove, a splice variant being as defined herein.

Concerning GS1 polypeptides, or PEAMT polypeptides, or LFY-like polypeptides, according to the present invention, there is provided a method for enhancing yield-related traits in plants, comprising introducing and expressing in a plant a splice variant of any one of the nucleic acid sequences given in Table A1, or A2, or A4 of Example 1, or a splice variant of a nucleic acid sequence encoding an orthologue, paralogue or homologue of any of the amino acid sequences given in Table A1, or A2, or A4 of Example 1.

Concerning FATB polypeptides, according to the present invention, there is provided a method for increasing seed yield-related traits, comprising introducing and expressing in a plant, a splice variant of any one of the nucleic acid sequences given in Table A3 of Example 1, or a splice variant of a nucleic acid sequence encoding an orthologue, paralogue or homologue of any of the polypeptide sequences given in Table A3 of Example 1, having substantially the same biological activity as the polypeptide sequence as represented by SEQ ID NO: 93 and any of the polypeptide sequences depicted in Table A3 of Example 1.

Concerning GS1 polypeptides, preferred splice variants are splice variants of a nucleic acid sequence represented by SEQ ID NO: 1, or a splice variant of a nucleic acid sequence encoding an orthologue or paralogue of SEQ ID NO: 2. Preferably, the amino acid sequence encoded by the splice variant, when used in the construction of a phylogenetic tree, such as the one depicted in FIGS. 3a and 3b, clusters with the algal-type clade (the group of algal GS1 polypeptides comprising the amino acid sequence represented by SEQ ID NO: 2) rather than with the plant chloroplastic or plant cytosolic glutamine synthase group.

Concerning PEAMT polypeptides, preferred splice variants are splice variants of a nucleic acid sequence represented by SEQ ID NO: 57, or a splice variant of a nucleic acid sequence encoding an orthologue or paralogue of SEQ ID NO: 58. Preferably, the amino acid sequence encoded by the splice variant, when used in the construction of a phylogenetic tree, such as the one depicted in FIG. 6, clusters with the group I of PEAMT polypeptides comprising the amino acid sequence represented by SEQ ID NO: 58 rather than with any other group.

Concerning FATB polypeptides; preferred splice variants are splice variants of a nucleic acid sequence represented by SEQ ID NO: 92, or a splice variant of a nucleic acid sequence encoding an orthologue or paralogue of SEQ ID NO: 93. Preferably, the splice variant is a splice variant of a nucleic acid sequence encoding a polypeptide sequence having in increasing order of preference at least 50%, 55%, 60%, 65%, 70%, 75%, 80%, 85%, 90%, 95%, 98%, 99% or more amino acid sequence identity to the FATB polypeptide as represented by SEQ ID NO: 93 or to any of the polypeptide sequences given in Table A3 herein.

Concerning LFY-like polypeptides, preferred splice variants are splice variants of a nucleic acid sequence represented by SEQ ID NO: 145, or a splice variant of a nucleic acid sequence encoding an orthologue or paralogue of SEQ ID NO: 146. Preferably, the amino acid sequence encoded by the splice variant, when used in the construction of a phylogenetic tree, such as the one depicted in FIG. 16, clusters with the group of LFY-like polypeptides.

Another nucleic acid sequence variant useful in performing the methods of the invention is an allelic variant of a nucleic acid sequence encoding a GS1 polypeptide, or a PEAMT polypeptide, or a FATB polypeptide, or a LFY-like polypeptide, as defined hereinabove, an allelic variant being as defined herein.

According to the present invention, there is provided a method for enhancing yield-related traits in plants, comprising introducing and expressing in a plant an allelic variant of any one of the nucleic acid sequences given in Table A1 to A4 of Example 1, or comprising introducing and expressing in a plant an allelic variant of a nucleic acid sequence encoding an orthologue, paralogue or homologue of any of the amino acid sequences given in Table A1 to A4 of Example 1.

Concerning GS1 polypeptides, the polypeptides encoded by allelic variants useful in the methods of the present invention have substantially the same biological activity as the GS1 polypeptide of SEQ ID NO: 2 and any of the amino acids depicted in Table A1 of Example 1. Allelic variants exist in nature, and encompassed within the methods of the present invention is the use of these natural alleles. Preferably, the allelic variant is an allelic variant of SEQ ID NO: 1 or an allelic variant of a nucleic acid sequence encoding an orthologue or paralogue of SEQ ID NO: 2. Preferably, the amino acid sequence encoded by the allelic variant, when used in the construction of a phylogenetic tree, such as the one depicted in FIGS. 3a and 3b, clusters with the algal-type clade (the group of algal GS1 polypeptides comprising the amino acid sequence represented by SEQ ID NO: 2) rather than with the plant chloroplastic or plant cytosolic glutamine synthase group.

Concerning PEAMT polypeptides, the polypeptides encoded by allelic variants useful in the methods of the present invention have substantially the same biological activity as the PEAMT polypeptide of SEQ ID NO: 58 and any of the amino acids depicted in Table A2 of the Examples section. Allelic variants exist in nature, and encompassed within the methods of the present invention is the use of these natural alleles. Preferably, the allelic variant is an allelic variant of SEQ ID NO: 57 or an allelic variant of a nucleic acid sequence encoding an orthologue or paralogue of SEQ ID NO: 58. Preferably, the amino acid sequence encoded by the allelic variant, when used in the construction of a phylogenetic tree, such as the one depicted in FIG. 6, clusters with the group I of PEAMT polypeptides comprising the amino acid sequence represented by SEQ ID NO: 58 rather than with any other group.

Concerning FATB polypeptides, the allelic variants useful in the methods of the present invention have substantially the same biological activity as the FATB polypeptide of SEQ ID NO: 93 and any of the polypeptide sequences depicted in Table A3 of Example 1. Allelic variants exist in nature, and encompassed within the methods of the present invention is the use of these natural alleles. Preferably, the allelic variant is an allelic variant of SEQ ID NO: 92 or an allelic variant of a nucleic acid sequence encoding an orthologue or paralogue of SEQ ID NO: 93. Preferably, the allelic variant is an allelic variant of a polypeptide sequence having in increasing order of preference at least 50%, 55%, 60%, 65%, 70%, 75%, 80%, 85%, 90%, 95%, 98%, 99% or more amino acid sequence identity to the FATB polypeptide as represented by SEQ ID NO: 93 or to any of the polypeptide sequences given in Table A3 of Example 1 herein.

Concerning LFY-like polypeptides, the polypeptides encoded by allelic variants useful in the methods of the present invention have substantially the same biological activity as the LFY-like polypeptide of SEQ ID NO: 146 and any of the amino acids depicted in Table A4 of Example 1. Allelic variants exist in nature, and encompassed within the methods of the present invention is the use of these natural alleles. Preferably, the allelic variant is an allelic variant of SEQ ID NO: 145 or an allelic variant of a nucleic acid sequence encoding an orthologue or paralogue of SEQ ID NO: 146. Preferably, the amino acid sequence encoded by the allelic variant, when used in the construction of a phylogenetic tree, such as the one depicted in FIG. 16, clusters with the group of LFY-like polypeptides.

Gene shuffling or directed evolution may also be used to generate variants of nucleic acid sequences encoding GS1 polypeptides, or PEAMT polypeptides, or FATB polypeptides, or LFY-like polypeptides, as defined above; the term “gene shuffling” being as defined herein.

According to the present invention, there is provided a method for enhancing yield-related traits in plants, comprising introducing and expressing in a plant a variant of any one of the nucleic acid sequences given in Table A1 to A4 of Example 1, or comprising introducing and expressing in a plant a variant of a nucleic acid sequence encoding an orthologue, paralogue or homologue of any of the amino acid sequences given in Table A1 to A4 of Example 1, which variant nucleic acid sequence is obtained by gene shuffling.

Concerning GS1 polypeptides, preferably, the amino acid sequence encoded by the variant nucleic acid sequence obtained by gene shuffling, when used in the construction of a phylogenetic tree such as the one depicted in FIGS. 3a and 3b, clusters with the algal-type clade (the group of algal GS1 polypeptides comprising the amino acid sequence represented by SEQ ID NO: 2) rather than with the plant chloroplastic or plant cytosolic glutamine synthase group.

Concerning PEAMT polypeptides, preferably, the amino acid sequence encoded by the variant nucleic acid sequence obtained by gene shuffling, when used in the construction of a phylogenetic tree such as the one depicted in FIG. 6, clusters with the group I of PEAMT polypeptides comprising the amino acid sequence represented by SEQ ID NO: 58 rather than with any other group.

Concerning FATB polypeptides, preferably, the variant nucleic acid sequence obtained by gene shuffling encodes a polypeptide sequence having in increasing order of preference at least 50%, 55%, 60%, 65%, 70%, 75%, 80%, 85%, 90%, 95%, 98%, 99% or more amino acid sequence identity to the FATB polypeptide as represented by SEQ ID NO: 93 or to any of the polypeptide sequences given in Table A3 herein.

Concerning LFY-like polypeptides, preferably, the amino acid sequence encoded by the variant nucleic acid sequence obtained by gene shuffling, when used in the construction of a phylogenetic tree, such as the one depicted in FIG. 16, clusters with the group of LFY-like polypeptides.

Furthermore, nucleic acid sequence variants may also be obtained by site-directed mutagenesis. Several methods are available to achieve site-directed mutagenesis, the most common being PCR based methods (Current Protocols in Molecular Biology. Wiley Eds.).

Nucleic acid sequences encoding GS1 polypeptides may be derived from any natural or artificial source. The nucleic acid sequence may be modified from its native form in composition and/or genomic environment through deliberate human manipulation. Preferably the GS1 polypeptide-encoding nucleic acid sequence is from the division of the Chlorophyta, further preferably from the class of the Chlorophyceae, more preferably from the family Chlamydomonadaceae, most preferably the nucleic acid sequence is from Chlamydomonas reinhardtii.

Nucleic acid sequences encoding PEAMT polypeptides may be derived from any natural or artificial source. The nucleic acid sequence may be modified from its native form in composition and/or genomic environment through deliberate human manipulation. Preferably the PEAMT polypeptide-encoding nucleic acid sequence is from a plant, further preferably from a dicotyledonous plant, more preferably from the family Brasicaceae, most preferably the nucleic acid sequence is from Arabidopsis thaliana.

Advantageously, the present invention provides hitherto unknown PEAMT nucleic acid sequence and polypeptide sequences.

According to a further embodiment of the present invention, there is provided an isolated PEAMT nucleic acid sequence molecule comprising at least 98% sequence identity to SEQ ID NO: 57.

Additionally an isolated polypeptide comprising at least 99% sequence identity to SEQ ID NO: 58, is provided.

Nucleic acid sequences encoding FATB polypeptides, or LFY-like polypeptides may be derived from any natural or artificial source. The nucleic acid sequence may be modified from its native form in composition and/or genomic environment through deliberate human manipulation. The nucleic acid sequence encoding a FATB polypeptide or a LFY-like polypeptide, is from a plant, further preferably from a dicotyledonous plant, more preferably from the family Brassicaceae, most preferably the nucleic acid sequence is from Arabidopsis thaliana.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants.

Taking corn as an example, a yield increase may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per square meter, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially seed yield of plants, relative to control plants, which method comprises modulating expression in a plant of a nucleic acid sequence encoding a GS1 polypeptide, or a PEAMT polypeptide, or a FATB polypeptide, or a LFY-like polypeptide, as defined herein.

The present invention provides a method for increasing yield, especially seed yield of plants, relative to control plants, which method comprises modulating expression in a plant of a nucleic acid sequence encoding a GS1 polypeptide, or a PEAMT polypeptide, or a LFY-like polypeptide, as defined herein.

The present invention also provides a method for increasing seed yield-related traits of plants relative to control plants, which method comprises increasing expression in a plant of a nucleic acid sequence encoding a FATB polypeptide as defined herein.

Since the transgenic plants according to the present invention have increased yield and/or increased seed yield-related traits, it is likely that these plants exhibit an increased growth rate (during at least part of their life cycle), relative to the growth rate of control plants at a corresponding stage in their life cycle. However, concerning LFY-like polypeptides, no earlier induction of flowering time was observed.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as speed of germination, early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per square meter (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating and/or increasing expression in a plant of a nucleic acid sequence encoding a GS1 polypeptide, or a PEAMT polypeptide, or a FATB polypeptide, or a LFY-like polypeptide, as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi, nematodes and insects.

Increased seed yield-related traits occur whether the plant is under non-stress conditions or whether the plant is exposed to various stresses compared to control plants grown under comparable conditions. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi, nematodes, and insects. The term “non-stress” conditions as used herein are those environmental conditions that allow optimal growth of plants. Persons skilled in the art are aware of normal soil conditions and climatic conditions for a given location.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 90%, 87%, 85%, 83%, 80%, 77% or 75% of the average production of such plant in a given environment. Average production may be calculated on harvest and/or season basis. Persons skilled in the art are aware of average yield productions of a crop.

Concerning GS1 polypeptides performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises modulating expression in a plant of a nucleic acid sequence encoding a GS1 polypeptide.

Concerning PEAMT polypeptides, performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises modulating expression in a plant of a nucleic acid sequence encoding a PEAMT polypeptide.

Concerning FATB polypeptides, performance of the methods of the invention gives plants grown under non-stress conditions or under mild stress conditions having increased seed yield-related traits, relative to control plants grown under comparable conditions. Therefore, according to the present invention, there is provided a method for increasing seed yield-related traits in plants grown under non-stress conditions or under mild stress conditions, which method comprises increasing expression in a plant of a nucleic acid sequence encoding a FATB polypeptide.

Concerning LFY-like polypeptides, performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises modulating expression in a plant of a nucleic acid sequence encoding a LFY-like polypeptide.

Concerning GS1 polypeptides performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises modulating expression in a plant of a nucleic acid sequence encoding a GS1 polypeptide. Nutrient deficiency may result from a lack of nutrients such as nitrogen, phosphates and other phosphorous-containing compounds, potassium, calcium, cadmium, magnesium, manganese, iron and boron, amongst others. In a particular embodiment of the present invention, there is provided a method for increasing yield in plants grown under conditions of nitrogen deficiency, which method comprises modulating expression in a plant of a nucleic acid sequence encoding a GS1 polypeptide.

Concerning GS1 polypeptides performance of the methods of the invention gives plants grown under conditions of salt stress, increased yield relative to control plants grown under comparable conditions. Therefore, according to the present invention, there is provided a method for increasing yield in plants grown under conditions of salt stress, which method comprises modulating expression in a plant of a nucleic acid sequence encoding a GS1 polypeptide. The term salt stress is not restricted to common salt (NaCl), but may be any one or more of: NaCl, KCl, LiCl, MgCl₂, CaCl₂, amongst others.

Concerning PEAMT polypeptides, performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises modulating expression in a plant of a nucleic acid sequence encoding a PEAMT polypeptide. Nutrient deficiency may result from a lack of nutrients such as nitrogen, phosphates and other phosphorous-containing compounds, potassium, calcium, cadmium, magnesium, manganese, iron and boron, amongst others.

Concerning FATB polypeptides, performance of the methods according to the present invention results in plants grown under abiotic stress conditions having increased seed yield-related traits relative to control plants grown under comparable stress conditions. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. Since diverse environmental stresses activate similar pathways, the exemplification of the present invention with drought stress should not be seen as a limitation to drought stress, but more as a screen to indicate the involvement of FATB polypeptides as defined above, in increasing seed yield-related traits relative to control plants grown in comparable stress conditions, in abiotic stresses in general.

The term “abiotic stress” as defined herein is taken to mean any one or more of: water stress (due to drought or excess water), anaerobic stress, salt stress, temperature stress (due to hot, cold or freezing temperatures), chemical toxicity stress and oxidative stress. According to one aspect of the invention, the abiotic stress is an osmotic stress, selected from water stress, salt stress, oxidative stress and ionic stress. Preferably, the water stress is drought stress. The term salt stress is not restricted to common salt (NaCl), but may be any stress caused by one or more of: NaCl, KCl, LiCl, MgCl₂, CaCl₂, amongst others.

Concerning FATB polypeptides, performance of the methods of the invention gives plants having increased seed yield-related traits, under abiotic stress conditions relative to control plants grown in comparable stress conditions. Therefore, according to the present invention, there is provided a method for increasing seed yield-related traits, in plants grown under abiotic stress conditions, which method comprises increasing expression in a plant of a nucleic acid sequence encoding a FATB polypeptide. According to one aspect of the invention, the abiotic stress is an osmotic stress, selected from one or more of the following: water stress, salt stress, oxidative stress and ionic stress.

Another example of abiotic environmental stress is the reduced availability of one or more nutrients that need to be assimilated by the plants for growth and development. Because of the strong influence of nutrition utilization efficiency on plant yield and product quality, a huge amount of fertilizer is poured onto fields to optimize plant growth and quality. Productivity of plants ordinarily is limited by three primary nutrients, phosphorous, potassium and nitrogen, which is usually the rate-limiting element in plant growth of these three. Therefore the major nutritional element required for plant growth is nitrogen (N). It is a constituent of numerous important compounds found in living cells, including amino acids, proteins (enzymes), nucleic acid sequences, and chlorophyll. 1.5% to 2% of plant dry matter is nitrogen and approximately 16% of total plant protein. Thus, nitrogen availability is a major limiting factor for crop plant growth and production (Frink et al. (1999) Proc Natl Acad Sci USA 96(4): 1175-1180), and has as well a major impact on protein accumulation and amino acid composition. Therefore, of great interest are crop plants with increased seed yield-related traits, when grown under nitrogen-limiting conditions.

Concerning FATB polypeptides, performance of the methods of the invention gives plants grown under conditions of reduced nutrient availability, particularly under conditions of reduced nitrogen availablity, having increased seed yield-related traits relative to control plants grown under comparable conditions. Therefore, according to the present invention, there is provided a method for increasing seed yield-related traits in plants grown under conditions of reduced nutrient availablity, preferably reduced nitrogen availability, which method comprises increasing expression in a plant of a nucleic acid sequence encoding a FATB polypeptide. Reduced nutrient availability may result from a deficiency or excess of nutrients such as nitrogen, phosphates and other phosphorous-containing compounds, potassium, calcium, cadmium, magnesium, manganese, iron and boron, amongst others. Preferably, reduced nutrient availablity is reduced nitrogen availability.

Concerning LFY-like polypeptides, performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises modulating expression in a plant of a nucleic acid sequence encoding a LFY-like polypeptide. Nutrient deficiency may result from a lack of nutrients such as nitrogen, phosphates and other phosphorous-containing compounds, potassium, calcium, cadmium, magnesium, manganese, iron and boron, amongst others.

The present invention encompasses plants or parts thereof (including seeds) or cells obtainable by the methods according to the present invention. The plants or parts or cells thereof comprise a nucleic acid sequence transgene encoding a GS1 polypeptide, or a PEAMT polypeptide, or a FATB polypeptide, or a LFY-like polypeptide, as defined above.

The invention also provides genetic constructs and vectors to facilitate introduction and/or expression in plants of nucleic acid sequences encoding GS1 polypeptides, or PEAMT polypeptides, or FATB polypeptides, or LFY-like polypeptides, as defined herein. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid sequence encoding a GS1 polypeptide, or a PEAMT polypeptide, or a FATB polypeptide, or a LFY-like polypeptide, as defined above;
(b) one or more control sequences capable of driving expression of the nucleic acid sequence of (a); and optionally
(c) a transcription termination sequence.

Preferably, the nucleic acid sequence encoding a GS1 polypeptide, or a PEAMT polypeptide, or a FATB polypeptide, or a LFY-like polypeptide, is as defined above. The term “control sequence” and “termination sequence” are as defined herein.

Plants are transformed with a vector comprising any of the nucleic acid sequences described above. The skilled artisan is well aware of the genetic elements that must be present on the vector in order to successfully transform, select and propagate host cells containing the sequence of interest. The sequence of interest is operably linked to one or more control sequences (at least to a promoter).

Concerning FATB, preferably, one of the control sequences of a construct is a constitutive promoter isolated from a plant genome. An example of a plant constitutive promoter is a GOS2 promoter, preferably a rice GOS2 promoter, more preferably a GOS2 promoter as represented by SEQ ID NO: 144.

Concerning GS1, advantageously, any type of promoter, whether natural or synthetic, may be used to drive expression of the nucleic acid sequence, but preferably the promoter is of plant origin. A promoter capable of driving expression in shoots, and in particular in green tissue, is particularly useful in the methods. See the “Definitions” section herein for definitions of the various promoter types.

Concerning PEAMT, advantageously, any type of promoter, whether natural or synthetic, may be used to drive expression of the nucleic acid sequence, but preferably the promoter is of plant origin. A constitutive promoter is particularly useful in the methods. Preferably the constitutive promoter is also a ubiquitous promoter of medium strength. See the “Definitions” section herein for definitions of the various promoter types.

Concerning FATB, advantageously, any type of promoter, whether natural or synthetic, may be used to increase expression of the nucleic acid sequence. A constitutive promoter is particularly useful in the methods, preferably a constitutive promoter isolated from a plant genome. The plant constitutive promoter drives expression of a coding sequence at a level that is in all instances below that obtained under the control of a 35S CaMV viral promoter.

Also concerning FATB, organ-specific promoters, for example for preferred expression in leaves, stems, tubers, meristems, are useful in performing the methods of the invention. Developmentally-regulated promoters are also useful in performing the methods of the invention See the “Definitions” section herein for definitions of the various promoter types.

Concerning LFY-like, advantageously, any type of promoter, whether natural or synthetic, may be used to drive expression of the nucleic acid sequence, but preferably the promoter is of plant origin. A constitutive promoter is particularly useful in the methods. Preferably the constitutive promoter is also a ubiquitous promoter of medium strength. See the “Definitions” section herein for definitions of the various promoter types. Also useful in the methods of the invention is a shoot-specific (or green-tissue specific) promoter.

Concerning GS1 polypeptides, It should be clear that the applicability of the present invention is not restricted to the GS1 polypeptide-encoding nucleic acid sequence represented by SEQ ID NO: 1, nor is the applicability of the invention restricted to expression of a GS1 polypeptide-encoding nucleic acid sequence when driven by a shoot-specific promoter.

The shoot-specific promoter preferentially, drives expression in green tissue, further preferably the shoot-specific promoter is isolated from a plant, such as a protochlorophyllide reductase promoter (pPCR), more preferably the protochlorophyllide reductase promoter is from rice. Further preferably the protochlorophyllide reductase promoter is represented by a nucleic acid sequence substantially similar to SEQ ID NO: 6, most preferably the constitutive promoter is as represented by SEQ ID NO: 6. See the “Definitions” section herein for further examples of green-tissue specific promoters.

Concerning GS1 polypeptides, optionally, one or more terminator sequences may be used in the construct introduced into a plant. Preferably, the construct comprises an expression cassette comprising a protochlorophyllide reductase promoter, substantially similar to SEQ ID NO: 6, and the nucleic acid encoding the GS1 polypeptide.

Concerning PEAMT polypeptides, it should be clear that the applicability of the present invention is not restricted to the PEAMT polypeptide-encoding nucleic acid sequence represented by SEQ ID NO: 57, nor is the applicability of the invention restricted to expression of a PEAMT polypeptide-encoding nucleic acid sequence when driven by a constitutive promoter.

The constitutive promoter is preferably a medium strength promoter, more preferably selected from a plant derived promoter, such as a GOS2 promoter, more preferably is the promoter GOS2 promoter from rice. Further preferably the constitutive promoter is represented by a nucleic acid sequence substantially similar to SEQ ID NO: 85, most preferably the constitutive promoter is as represented by SEQ ID NO: 85. See the “Definitions” section herein for further examples of constitutive promoters.

Concerning PEAMT polypeptides, optionally, one or more terminator sequences may be used in the construct introduced into a plant. Preferably, the construct comprises an expression cassette comprising a GOS2 promoter, substantially similar to SEQ ID NO: 85, and the nucleic acid encoding the PEAMT polypeptide.

Concerning FATB polypeptides, it should be clear that the applicability of the present invention is not restricted to a nucleic acid sequence encoding the FATB polypeptide, as represented by SEQ ID NO: 92, nor is the applicability of the invention restricted to expression of a FATB polypeptide-encoding nucleic acid sequence when driven by a constitutive promoter.

Optionally, one or more terminator sequences may be used in the construct introduced into a plant. Additional regulatory elements may include transcriptional as well as translational increasers. Those skilled in the art will be aware of terminator and increaser sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, increaser, silencer, intron sequences, 3′UTR and/or 5′UTR regions) may be protein and/or RNA stabilizing elements. Such sequences would be known or may readily be obtained by a person skilled in the art.

Concerning LFY-like polypeptides, it should be clear that the applicability of the present invention is not restricted to the LFY-like polypeptide-encoding nucleic acid represented by SEQ ID NO: 145, nor is the applicability of the invention restricted to expression of a LFY-like polypeptide-encoding nucleic acid when driven by a constitutive promoter, or when driven by a shoot-specific promoter.

The constitutive promoter is preferably a medium strength promoter, such as a GOS2 promoter, preferably the promoter is a GOS2 promoter from rice. Further preferably the constitutive promoter is represented by a nucleic acid sequence substantially similar to SEQ ID NO: 149, most preferably the constitutive promoter is as represented by SEQ ID NO: 149. See Table 2a in the “Definitions” section herein for further examples of constitutive promoters.

Concerning LFY-like polypeptides, according to another preferred feature of the invention, the nucleic acid encoding a LFY-like polypeptide is operably linked to a shoot-specific (or green-tissue specific) promoter. The shoot-specific promoter is preferably a protochlorophyllid reductase promoter, more preferably the protochlorophyllid reductase promoter is from rice, further preferably the protochlorophyllid reductase promoter is represented by a nucleic acid sequence substantially similar to SEQ ID NO: 150, most preferably the promoter is as represented by SEQ ID NO: 150. Examples of other shoot-specific promoters which may also be used to perform the methods of the invention are shown in Table 2b in the “Definitions” section above.

Concerning LFY-like polypeptides, optionally, one or more terminator sequences may be used in the construct introduced into a plant. Preferably, the construct comprises an expression cassette comprising the GOS2 promoter, or the protochlorophyllid reductase promoter, operably linked to the nucleic acid encoding the LFY-like polypeptide.

Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

It is known that upon stable or transient integration of nucleic acid sequences into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid sequence molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid sequence can be identified for example by selection (for example, cells which have integrated the selectable marker survive whereas the other cells die). The marker genes may be removed or excised from the transgenic cell once they are no longer needed. Techniques for marker gene removal are known in the art, useful techniques are described above in the definitions section.

The invention also provides a method for the production of transgenic plants having enhanced yield-related traits relative to control plants, comprising introduction and expression in a plant of any nucleic acid encoding a GS1 polypeptide, or a PEAMT polypeptide, or a LFY-like polypeptide, as defined hereinabove.

More specifically, the present invention provides a method for the production of transgenic plants having enhanced yield-related traits, particularly increased (seed) yield, which method comprises:
(i) introducing and expressing in a plant or plant cell a GS1 polypeptide-encoding, or a PEAMT polypeptide-encoding, or a LFY-like polypeptide-encoding nucleic acid sequence; and
(ii) cultivating the plant cell under conditions promoting plant growth and development.

The nucleic acid of (i) may be any of the nucleic acids capable of encoding a GS1 polypeptide, or a PEAMT polypeptide, or a LFY-like polypeptide, as defined herein.

The invention also provides a method for the production of transgenic plants having increased seed yield-related traits relative to control plants, comprising introduction and expression in a plant of any nucleic acid sequence encoding a FATB polypeptide as defined hereinabove.

More specifically, the present invention provides a method for the production of transgenic plants having increased seed yield-related traits relative to control plants, which method comprises:
(i) introducing and expressing in a plant, plant part, or plant cell a nucleic acid sequence encoding a FATB polypeptide; and
(ii) cultivating the plant cell, plant part or plant under conditions promoting plant growth and development.

The nucleic acid sequence of (i) may be any of the nucleic acid sequences capable of encoding a FATB polypeptide as defined herein.

The nucleic acid sequence may be introduced directly into a plant cell or into the plant itself (including introduction into a tissue, organ or any other part of a plant). According to a preferred feature of the present invention, the nucleic acid sequence is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid sequence encoding a GS1 polypeptide, or a PEAMT polypeptide, or a LFY-like polypeptide, as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

Furthermore, the invention also includes host cells containing an isolated nucleic acid sequence encoding a FATB polypeptide as defined hereinabove, opereably linked to a constitutive promoter. Preferred host cells according to the invention are plant cells. Host plants for the nucleic acid sequences or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, linseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum, emmer, spelt, secale, einkorn, teff, milo and oats.

The invention also extends to harvestable parts of a plant such as, but not limited to seeds, leaves, fruits, flowers, stems, roots, rhizomes, tubers and bulbs, which harvestable parts comprise a recombinant nucleic acid sequence encoding a GS1 polypeptide, or a PEAMT polypeptide, or a LFY-like polypeptide. The invention furthermore relates to products derived, preferably directly derived, from a harvestable part of such a plant, such as dry pellets or powders, oil, fat and fatty acids, starch or proteins.

Furthermore, the invention also extends to harvestable parts of a plant comprising an isolated nucleic acid sequence encoding a FATB (as defined hereinabove) operably linked to a constitutive promoter, such as, but not limited to seeds, leaves, fruits, flowers, stems, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids sequences or genes, or gene products, are well documented in the art and examples are provided in the definitions section.

As mentioned above, a preferred method for modulating expression of a nucleic acid sequence encoding a GS1 polypeptide, or a PEAMT polypeptide, or a FATB polypeptide, or a LFY-like polypeptide, is by introducing and expressing in a plant a nucleic acid encoding a GS1 polypeptide, or a PEAMT polypeptide, or a FATB polypeptide, or a LFY-like polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acid sequences encoding GS1 polypeptides, or PEAMT polypeptides, or LFY-like polypeptides, as described herein and use of these GS1 polypeptides, or PEAMT polypeptides, or LFY-like polypeptides, in enhancing any of the aforementioned yield-related traits in plants.

Furthermore, the present invention also encompasses use of nucleic acid sequences encoding FATB polypeptides as described herein and use of these FATB polypeptides in increasing any of the aforementioned seed yield-related traits in plants, under normal growth conditions, under abiotic stress growth (preferably osmotic stress growth conditions) conditions, and under growth conditions of reduced nutrient availability, preferably under conditions of reduced nitrogen availability.

Concerning GS1 polypeptides, nucleic acid sequences encoding GS1 polypeptides, or PEAMT polypeptides, or LFY-like polypeptides, described herein, or the GS1 polypeptides themselves, may find use in breeding programmes in which a DNA marker is identified which may be genetically linked to gene encoding a GS1 polypeptide, or a PEAMT polypeptide, or a LFY-like polypeptide. The nucleic acids/genes, or the GS1 polypeptides themselves, or the PEAMT polypeptides themselves, or the LFY-like polypeptides, may be used to define a molecular marker. This DNA or protein marker may then be used in breeding programmes to select plants having enhanced yield-related traits as defined hereinabove in the methods of the invention.

Concerning FATB polypeptides, nucleic acid sequences encoding FATB polypeptides described herein, or the FATB polypeptides themselves, may find use in breeding programmes in which a DNA marker is identified that may be genetically linked to a FATB polypeptide-encoding gene. The genes/nucleic acid sequences, or the FATB polypeptides themselves may be used to define a molecular marker. This DNA or protein marker may then be used in breeding programmes to select plants having increased seed yield-related traits, as defined hereinabove in the methods of the invention.

Allelic variants of a gene/nucleic acid sequence encoding a GS1 polypeptide, or a PEAMT polypeptide, or a FATB polypeptide, or a LFY-like polypeptide, may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acid sequences encoding GS1 polypeptides, or PEAMT polypeptides, or FATB polypeptides, or LFY-like polypeptides, may also be used as probes for genetically and physically mapping the genes that they are a part of, and as markers for traits linked to those genes. Such information may be useful in plant breeding in order to develop lines with desired phenotypes. Such use of nucleic acid sequences encoding GS1 polypeptides, or PEAMT polypeptides, or FATB polypeptides, or LFY-like polypeptides, requires only a nucleic acid sequence of at least 15 nucleotides in length. The nucleic acid sequences encoding GS1 polypeptides, or PEAMT polypeptides, or FATB polypeptides, or LFY-like polypeptides, may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the GS1-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the nucleic acid sequence encoding GS1 polypeptides, or PEAMT polypeptides, or FATB polypeptides, or LFY-like polypeptides, in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid sequence probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid sequence probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid sequence amplification-based methods for genetic and physical mapping may be carried out using the nucleic acid sequences. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic acid sequence Res. 18:3671), Radiation Hybrid Mapping (Walter et al. (1997) Nat. Genet. 7:22-28) and Happy Mapping (Dear and Cook (1989) Nucleic acid sequence Res. 17:6795-6807). For these methods, the sequence of a nucleic acid sequence is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related or enhanced seed-yield related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### Items

1. A method for enhancing yield-related traits in plants relative to control plants, comprising modulating expression in a plant of a nucleic acid encoding an algal-type cytoplasmic glutamine synthase (GS1) polypeptide, wherein said algal-type GS1 polypeptide comprises a Gln-synt_C domain (Pfam accession PF00120) and a Gln-synt_N domain (Pfam accession PF03951).
2. Method according to item 1, wherein said GS1 polypeptide comprises one or more of the following motifs:
   (a) Motif 1, SEQ ID NO: 3;
   (b) Motif 2, SEQ ID NO: 4;
   (c) Motif 3, SEQ ID NO: 5,
   in which motifs maximally 2 mismatches are allowed.
3. Method according to item 1 or 2, wherein said modulated expression is effected by introducing and expressing in a plant a nucleic acid encoding an algal-type GS1 polypeptide.
4. Method according to any of items 1 to 3, wherein said nucleic acid encoding a GS1 polypeptide encodes any one of the proteins listed in Table A1 or is a portion of such a nucleic acid, or a nucleic acid capable of hybridising with such a nucleic acid.
5. Method according to any of items 1 to 4, wherein said nucleic acid sequence encodes an orthologue or paralogue of any of the proteins given in Table A1.
6. Method according to any of items 1 to 5, wherein said enhanced yield-related traits comprise increased yield, preferably increased biomass and/or increased seed yield relative to control plants.
7. Method according to any one of items 1 to 6, wherein said enhanced yield-related traits are obtained under conditions of nutrient deficiency.
8. Method according to any one of items 3 to 7, wherein said nucleic acid is operably linked to a shoot-specific promoter, preferably to a protochlorophyllide reductase promoter, most preferably to a protochlorophyllide reductase promoter from rice.
9. Method according to any of items 1 to 8, wherein said nucleic acid encoding a GS1 polypeptide is of plant origin, preferably from a alga, further preferably from the class of Chlorophyceae, more preferably from the family Chlamydomonadaceae, most preferably from Chlamydomonas reinhardtii.
10. Plant or part thereof, including seeds, obtainable by a method according to any of items 1 to 9, wherein said plant or part thereof comprises a recombinant nucleic acid encoding a GS1 polypeptide.
11. Construct comprising:
   (i) nucleic acid encoding a GS1 polypeptide as defined in items 1 or 2;
   (ii) one or more control sequences capable of driving expression of the nucleic acid sequence of (a); and optionally
   (iii) a transcription termination sequence.
12. Construct according to item 11, wherein one of said control sequences is a shoot-specific promoter, preferably a protochlorophyllide reductase promoter, most preferably a protochlorophyllide reductase promoter from rice.
13. Use of a construct according to item 11 or 12 in a method for making plants having increased yield, particularly increased biomass and/or increased seed yield relative to control plants.
14. Plant, plant part or plant cell transformed with a construct according to item 11 or 12.
15. Method for the production of a transgenic plant having increased yield, particularly increased biomass and/or increased seed yield relative to control plants, comprising:
   (i) introducing and expressing in a plant a nucleic acid encoding a GS1 polypeptide as defined in item 1 or 2; and
   (ii) cultivating the plant cell under conditions promoting plant growth and development.
16. Transgenic plant having increased yield, particularly increased biomass and/or increased seed yield, relative to control plants, resulting from modulated expression of a nucleic acid encoding a GS1 polypeptide as defined in item 1 or 2, or a transgenic plant cell derived from said transgenic plant.
17. Transgenic plant according to item 10, 14 or 16, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
18. Harvestable parts of a plant according to item 17, wherein said harvestable parts are preferably shoot biomass and/or seeds.
19. Products derived from a plant according to item 17 and/or from harvestable parts of a plant according to item 18.
20. Use of a nucleic acid encoding a GS1 polypeptide in increasing yield, particularly in increasing seed yield and/or shoot biomass in plants, relative to control plants.
21. An isolated polypeptide selected from:
   (i) an amino acid sequence represented by SEQ ID NO: 53 or 54;
   (ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 53 or 54,
   (iii) derivatives of any of the amino acid sequences given in (i) or (ii) above.
22. An isolated nucleic acid encoding a polypeptide as defined in item 22, or a nucleic acid hybridising thereto.
23. A method for enhancing yield-related traits in plants relative to that of control plants, comprising modulating expression in a plant of a nucleic acid encoding a PEAMT polypeptide or a homologue thereof comprising a protein domain having in increasing order of preference at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to anyone of the protein domains set forth in Table C2.
24. Method according to item 23, wherein the nucleic acid encodes a PEAMT polypeptide or a homologue thereof having in increasing order of preference at least 50%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid sequence represented by SEQ ID NO: 58.
25. Method according to item 23 or 24, wherein said nucleic acid encoding a PEAMT polypeptide or a homologue thereof is a portion of the nucleic acid represented by SEQ ID NO: 57, or is a portion of a nucleic acid encoding an orthologue or paralogue of the amino acid sequence of SEQ ID NO: 58, wherein the portion is at least 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, consecutive nucleotides in length, the consecutive nucleotides being of SEQ ID NO: 57, or of a nucleic acid encoding an orthologue or paralogue of the amino acid sequence of SEQ ID NO: 58.
26. Method according to any one of items 23 to 25, wherein the nucleic acid encoding a PEAMT polypeptide or a homologue thereof is capable of hybridising to the nucleic acid represented by SEQ ID NO: 1 or is capable of hybridising to a nucleic acid encoding an orthologue, paralogue or homologue of SEQ ID NO: 58.
27. Method according to any one of items 23 to 26, wherein said nucleic acid encoding a PEAMT polypeptide or a homologue thereof encodes an orthologue or paralogue of the sequence represented by SEQ ID NO: 58.
28. Method according to any one of items 23 to 27, wherein said modulated expression is effected by introducing and expressing in a plant a nucleic acid encoding a PEAMT polypeptide or a homologue thereof.
29. Method according to any one of items 23 to 28, wherein said enhanced yield-related traits comprising increased yield, preferably increased biomass and/or increased seed yield relative to control plants is obtained under non-stress conditions.
30. Method according to any one of items 23 to 29, wherein said enhanced yield-related traits comprising increased yield, preferably increased biomass and/or increased seed yield relative to control plants is obtained under conditions of drought stress.
31. Method according to item 28, 29 or 30 wherein said nucleic acid is operably linked to a constitutive promoter, preferably to a GOS2 promoter, most preferably to a GOS2 promoter from rice.
32. Method according to any one of items 23 to 31, wherein said nucleic acid encoding a PEAMT polypeptide is of plant origin, preferably from a dicotyledonous plant, further preferably from the family Brassicaceae, more preferably from the genus Arabidopsis, most preferably from Arabidopsis thaliana.
33. Plant or part thereof, including seeds, obtainable by a method according to any preceding item, wherein said plant or part thereof comprises a recombinant nucleic acid encoding a PEAMT polypeptide or a homologue thereof.
34. An isolated nucleic acid molecule comprising at least 98% sequence identity to SEQ ID NO: 57.
35. An isolated polypeptide comprising at least 99% sequence identity to SEQ ID NO: 58.
36. Construct comprising:
   (i) A nucleic acid encoding a PEAMT polypeptide or a homologue thereof as defined in any of items 23 to 27 and items 34 and 35;
   (ii) one or more control sequences capable of driving expression of the nucleic acid sequence of (a); and optionally
   (iii) a transcription termination sequence.
37. Construct according to item 36, wherein one of said control sequences is a constitutive promoter, preferably a GOS2 promoter, most preferably a GOS2 promoter from rice.
38. Use of a construct according to item 36 or 37 in a method for making plants having an altered yield-related traits relative to control plants.
39. Plant, plant part or plant cell transformed with a construct according to item 36 or 37.
40. Method for the production of a transgenic plant having an enhanced yield-related traits relative to control plants, comprising:
   (i) introducing and expressing in a plant a nucleic acid encoding a PEAMT polypeptide or a homologue thereof as defined in any one of items 23 to 27 and items 34 and 35; and
   (ii) cultivating the plant cell under conditions promoting plant growth and development.
41. Transgenic plant having enhanced yield-related traits relative to control plants, resulting from modulated expression of a nucleic acid encoding a PEAMT polypeptide or a homologue thereof as defined in any one of items 23 to 27 and items 34 and 35.
42. Transgenic plant according to item 33, 39 or 41, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
43. Products derived from a plant according to item 42.
44. Use of a nucleic acid encoding a PEAMT polypeptide or a homologue thereof in altering yield-related traits of plants relative to control plants.
45. A method for increasing seed yield-related traits in plants relative to control plants, comprising increasing expression in a plant of a nucleic acid sequence encoding a fatty acyl-acyl carrier protein (ACP) thioesterase B (FATB) polypeptide, which FATB polypeptide comprises (i) a plastidic transit peptide; (ii) at least one transmembrane helix; (iii) and an acyl-ACP thioesterase family domain with an InterPro accession IPR002864, and optionally selecting for plants having increased seed yield-related traits.
46. Method according to item 45, wherein said FATB polypeptide has (i) a plastidic transit peptide; (ii) in increasing order of preference at least 50%, 55%, 60%, 65%, 70%, 75%, 80%, 85%, 90%, 95%, 98%, 99% or more amino acid sequence identity to a transmembrane helix as represented by SEQ ID NO: 141; and having in increasing order of preference at least 50%, 55%, 60%, 65%, 70%, 75%, 80%, 85%, 90%, 95%, 98%, 99% or more amino acid sequence identity to an acyl-ACP thioesterase family domain as represented by SEQ ID NO: 140.
47. Method according to item 45 or 46, wherein said FATB polypeptide has in increasing order of preference at least 50%, 55%, 60%, 65%, 70%, 75%, 80%, 85%, 90%, 95%, 98%, 99% or more amino acid sequence identity to the FATB polypeptide as represented by SEQ ID NO: 93 or to any of the polypeptide sequences given in Table A3 herein.
48. Method according to any of item 45 to 47, wherein said FATB polypeptide is any polypeptide sequence which when used in the construction of a FATs phylogenetic tree, such as the one depicted in FIG. 10, clusters with the clade of FATB polypeptides comprising the polypeptide sequence as represented by SEQ ID NO: 93 rather than with the clade of FATA polypeptides.
49. Method according to any of item 45 to 48, wherein said FATB polypeptide is a polypeptide with enzymatic activity consisting in hydrolyzing acyl-ACP thioester bonds, preferentially from saturated acyl-ACPs (with chain lengths that vary between 8 and 18 carbons), releasing free fatty acids and acyl carrier protein (ACP).
50. Method according to any of item 45 to 49, wherein said nucleic acid sequence encoding a FATB polypeptide is represented by any one of the nucleic acid sequence SEQ ID NOs given in Table A3 or a portion thereof, or a sequence capable of hybridising with any one of the nucleic acid sequences SEQ ID NOs given in Table A3, or to a complement thereof.
51. Method according to any preceding item, wherein said nucleic acid sequence encodes an orthologue or paralogue of any of the polypeptide sequence SEQ ID NOs given in Table A3.
52. Method according to any preceding item, wherein said increased expression is effected by any one or more of: T-DNA activation tagging, TILLING, or homologous recombination.
53. Method according to any preceding item, wherein said increased expression is effected by introducing and expressing in a plant a nucleic acid sequence encoding a FATB polypeptide.
54. Method according to any preceding item, wherein said increased yield-related trait is one or more of: increased total seed yield per plant, increased total number of seeds, increased number of filled seeds, increased seed fill rate, and increased harvest index.
55. Method according to any preceding item, wherein said nucleic acid sequence is operably linked to a constitutive promoter.
56. Method according to item 55, wherein said constitutive promoter is a GOS2 promoter, preferably a rice GOS2 promoter, more preferably a GOS2 promoter as represented by SEQ ID NO: 144.
57. Method according to any preceding item, wherein said nucleic acid sequence encoding a FATB polypeptide is from a plant, further preferably from a dicotyledonous plant, more preferably from the family Brassicaceae, most preferably the nucleic acid sequence is from Arabidopsis thaliana.
58. Plants, parts thereof (including seeds), or plant cells obtainable by a method according to any preceding item, wherein said plant, part or cell thereof comprises an isolated nucleic acid transgene encoding a FATB polypeptide, operably linked to a constitutive promoter.
59. An isolated nucleic acid sequence comprising:
   (i) a nucleic acid sequence as represented by SEQ ID NO: 130;
   (ii) the complement of a nucleic acid sequence as represented by SEQ ID NO: 130;
   (iii) a nucleic acid sequence encoding FATB polypeptide having, in increasing order of preference, at least 70%, 75%, 80%, 85%, 90%, 95%, 96%, 97%, 98%, 99% or more amino acid sequence identity to the polypeptide sequence as represented by SEQ ID NO: 131.
60. An isolated polypeptide comprising:
   (i) a polypeptide sequence represented by SEQ ID NO: 131;
   (ii) a polypeptide sequence having, in increasing order of preference, at least 70%, 75%, 80%, 85%, 90%, 95%, 96%, 97%, 98%, 99% or more sequence identity to the polypeptide sequence as represented by SEQ ID NO: 131;
   (iii) derivatives of any of the polypeptide sequences given in (i) or (ii) above.
61. Construct comprising:
   (a) a nucleic acid sequence encoding a FATB polypeptide as defined in any one of items 45 to 51;
   (b) one or more control sequences capable of driving expression of the nucleic acid sequence of (a); and optionally
   (c) a transcription termination sequence.
62. Construct according to item 61, wherein said control sequence is a constitutive promoter.
63. Construct according to item 60, wherein said constitutive promoter is a GOS2 promoter, preferably a rice GOS2 promoter, more preferably a GOS2 promoter as represented by SEQ ID NO: 144.
64. Use of a construct according to any one of items 61 to 63, in a method for making plants having increased seed yield-related traits relative to control plants, which increased seed yield-related traits are one or more of: increased total seed yield per plant, increased total number of seeds, increased number of filled seeds, increased seed fill rate, and increased harvest index.
65. Plant, plant part or plant cell transformed with a construct according to any one of items 61 to 63.
66. Method for the production of transgenic plants having increased seed yield-related traits relative to control plants, comprising:
   (i) introducing and expressing in a plant, plant part, or plant cell, a nucleic acid sequence encoding a FATB polypeptide as defined in any one of items 45 to 51; and
   (ii) cultivating the plant cell, plant part, or plant under conditions promoting plant growth and development.
67. Transgenic plant having increased seed yield-related traits relative to control plants, resulting from increased expression of a nucleic acid sequence encoding a FATB polypeptide as defined in any one of items 45 to 51, operably linked to a constitutive promoter, or a transgenic plant cell or transgenic plant part derived from said transgenic plant.
68. Transgenic plant according to item 58, 65 or 67, wherein said plant is a crop plant or a monocot or a cereal, such as rice, maize, wheat, barley, millet, rye, triticale, sorghum and oats, or a transgenic plant cell derived from said transgenic plant.
69. Harvestable parts comprising an isolated nucleic acid sequence encoding a FATB polypeptide of a plant according to item 68, wherein said harvestable parts are preferably seeds.
70. Products derived from a plant according to item 68 and/or from harvestable parts of a plant according to item 69.
71. Use of a nucleic acid sequence encoding a FATB polypeptide as defined in any one of items 45 to 51 in increasing seed yield-related traits, comprising one or more of increased increased total seed yield per plant, increased total number of seeds, increased number of filled seeds, increased seed fill rate, and increased harvest index.
72. A method for enhancing yield-related traits in plants relative to control plants, comprising modulating expression in a plant of a nucleic acid encoding a LFY-like polypeptide, wherein said LFY-like polypeptide comprises a FLO_LFY domain.
73. Method according to item 72, wherein said LFY-like polypeptide has at least 50% sequence identity to SEQ ID NO: 146.
74. Method according to item 72 or 73, wherein said modulated expression is effected by introducing and expressing in a plant a nucleic acid encoding a LFY-like polypeptide.
75. Method according to any one of items 72 to 74, wherein said nucleic acid encoding a LFY-like polypeptide encodes any one of the proteins listed in Table A4 or is a portion of such a nucleic acid, or a nucleic acid capable of hybridising with such a nucleic acid.
76. Method according to any one of items 72 to 75, wherein said nucleic acid sequence encodes an orthologue or paralogue of any of the proteins given in Table A4.
77. Method according to any one of items 72 to 76, wherein said enhanced yield-related traits comprise increased yield, preferably increased seed yield relative to control plants.
78. Method according to any one of items 72 to 77, wherein said enhanced yield-related traits are obtained under non-stress conditions.
79. Method according to any one of items 74 to 78, wherein said nucleic acid is operably linked to a constitutive promoter, preferably to a GOS2 promoter, most preferably to a GOS2 promoter from rice.
80. Method according to any one of items 72 to 79, wherein said nucleic acid encoding a LFY-like polypeptide is of plant origin, preferably from a dicotyledonous plant, further preferably from the family Brassicaceae, more preferably from the genus Arabidopsis, most preferably from Arabidopsis thaliana.
81. Plant or part thereof, including seeds, obtainable by a method according to any preceding item, wherein said plant or part thereof comprises a recombinant nucleic acid encoding a LFY-like polypeptide.
82. Construct comprising:
   (i) nucleic acid encoding a LFY-like polypeptide as defined in items 72 or 73;
   (ii) one or more control sequences capable of driving expression of the nucleic acid sequence of (a); and optionally
   (iii) a transcription termination sequence.
83. Construct according to item 82, wherein one of said control sequences is a constitutive promoter, preferably a GOS2 promoter, most preferably a GOS2 promoter from rice.
84. Use of a construct according to item 82 or 83 in a method for making plants having increased yield, particularly increased seed yield relative to control plants.
85. Plant, plant part or plant cell transformed with a construct according to item 82 or 83.
86. Method for the production of a transgenic plant having increased yield, particularly increased seed yield relative to control plants, comprising:
   (i) introducing and expressing in a plant a nucleic acid encoding a LFY-like polypeptide as defined in item 72 or 73; and
   (ii) cultivating the plant cell under conditions promoting plant growth and development.
87. Transgenic plant having increased yield, particularly increased seed yield, relative to control plants, resulting from modulated expression of a nucleic acid encoding a LFY-like polypeptide as defined in item 72 or 73, or a transgenic plant cell derived from said transgenic plant.
88. Transgenic plant according to item 81, 85 or 87, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
89. Harvestable parts of a plant according to item 88, wherein said harvestable parts are preferably seeds.
90. Products derived from a plant according to item 88 and/or from harvestable parts of a plant according to item 89.
91. Use of a nucleic acid encoding a LFY-like polypeptide in increasing yield, particularly in increasing seed yield in plants, relative to control plants.

### DESCRIPTION OF FIGURES

The present invention will now be described with reference to the following figures in which:

FIG. 1 represents the domain structure of SEQ ID NO: 2 with the Gln-synt_N domain (PF03951) shown in bold underlined, the Gln-synt_C domain (PF00120) shown in italics underlined and the conserved motifs 1 to 3 by the dashed line.

FIG. 2 represents a multiple alignment of algal GS1 protein sequences.

FIG. 3 shows phylogenetic trees of GS1 proteins. Panel a gives an overview of GS1 (cytosolic) and GS2 (chloroplastic) proteins in a circular phylogram. Panel b shows the sequences grouping in the algal group, with a few sequences of the cytosolic and cytoplasmic outgroups. The numbers in the tree of panel b correspond to the following SEQ ID NOs: (1) SEQ ID NO: 21, (2) SEQ ID NO: 26, (3) SEQ ID NO: 27, (4) SEQ ID NO: 10, (5) SEQ ID NO: 11, (6) SEQ ID NO: 15, (7) SEQ ID NO: 24, (8) SEQ ID NO: 25, (9) SEQ ID NO: 12, (10) SEQ ID NO: 2, (11) SEQ ID NO: 16, (12) SEQ ID NO: 13, (13) SEQ ID NO: 28, (14) SEQ ID NO: 14, (15) SEQ ID NO: 9, (16) SEQ ID NO: 17, (17) SEQ ID NO: 19, (18) SEQ ID NO: 22, (19) SEQ ID NO: 30, (20) SEQ ID NO: 18, (21) SEQ ID NO: 20, (22) SEQ ID NO: 23, (23) SEQ ID NO: 29.

FIG. 4 represents the binary vector for increased expression in Oryza sativa of a GS1-encoding nucleic acid under the control of a rice protochlorophyllide reductase promoter (pPCR).

FIG. 5 represents a multiple alignment of the amino acid sequences of the PEAMT polypeptides of Table A2.

FIG. 6 represents a phylogenetic tree of the amino acid sequences of the PEAMT polypeptides of Table A2.

FIG. 7 represents the binary vector for increased expression in Oryza sativa of the Arath_PEAMT_{—}1 encoding nucleic acid under the control of a rice GOS2 promoter (pGOS2)

FIG. 8 schematically represents the general pathway for synthesis of various fatty acids (triacylglycerols; TAGs, synthesized via the Kennedy pathway) and steps normally involved for the production of seed storage lipids. The FATB polypeptides useful in performing the methods of the invention are shown with an arrow. According to Marillia et al. (2000) Developments in Plant Genetics and Breeding. Volume 5, 2000, Pages 182-188.

FIG. 9 represents a cartoon of a FATB polypeptide as represented by SEQ ID NO: 93, which comprises the following features: (i) a plastidic transit peptide; (ii) at least one transmembrane helix; (iii) and an acyl-ACP thioesterase family domain with an InterPro accession IPR002864.

FIG. 10 shows a phylogenetic tree of FATs polypeptides from various source organisms, according to Mayer et al. (2007) BMC Plant Biology 2007. FATA polypeptides and FATBA polypeptides belong to very clearly distinct clades. The FATB clade of polypeptides useful in performing the methods of the invention has been circled, the arrow points to the Arabidopsis thaliana FATB polypeptide as represented by SEQ ID NO: 93.

FIG. 11 represents the graphical output of the algorithm TMpred for SEQ ID NO: 93. From the algorithm prediction using SEQ ID NO: 93, a transmembrane helix is predicted between the transit peptide (located at the N-terminus of the polypeptide) and the acyl-ACP thioesterase family domain with an InterPro accession IPR002864 (located at the C-terminus of the polypeptide).

FIG. 12 shows the binary vector for increased expression in Oryza sativa plants of a nucleic acid sequence encoding a FATB polypeptide under the control of a constitutive promoter from rice.

FIG. 13 shows an AlignX (from Vector NTI 10.3, Invitrogen Corporation) multiple sequence alignment of the FATB polypeptides from Table A3. The N-terminal plastidic transit peptide as predicted by TargetP has been boxed in SEQ ID NO: 93 (Arath_FATB), and the predicted transmembrane helix (typical of FATB polypeptides only) as predicted by TMpred has been boxed across FATB polypeptides useful for performing the methods of the invention. The conserved IPR002864 of the acyl-ACP thioesterase family is marked by X under the consensus sequence. The three highly conserved catalytic residues have been boxed across the alignment.

FIG. 14 represents the LFY-like protein sequence of SEQ ID NO: 146, with the FLO_LFY domain shown in bold.

FIG. 15 represents a ClustalW 2.0.3 multiple alignment of various LFY-like proteins. The asterisks indicate absolutely conserved amino acids, the colons show highly conserved amino acid residues and the dots indicate conserved amino acids.

FIG. 16 shows a phylogenetic tree created from the alignment of FIG. 15 with the Neighbour Joining algorithm and 1000 bootstrap repetitions. The bootstrap values are shown.

FIG. 17 represents the binary vector for increased expression in Oryza sativa of a LFY-like-encoding nucleic acid under the control of a rice GOS2 promoter (pGOS2)

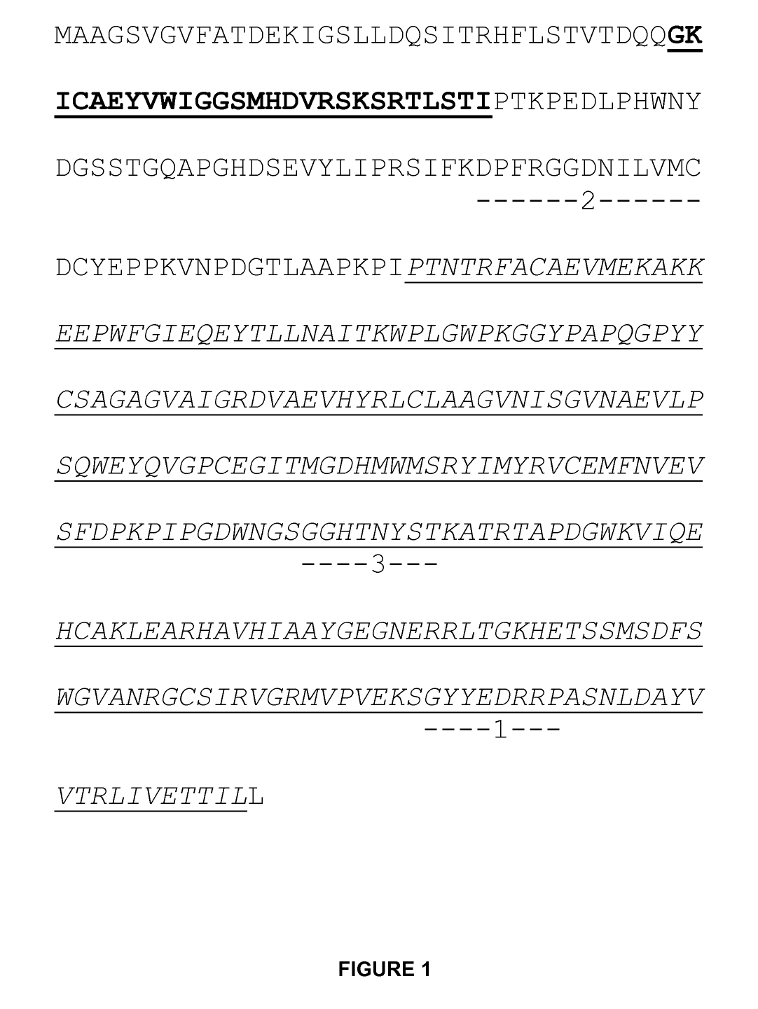

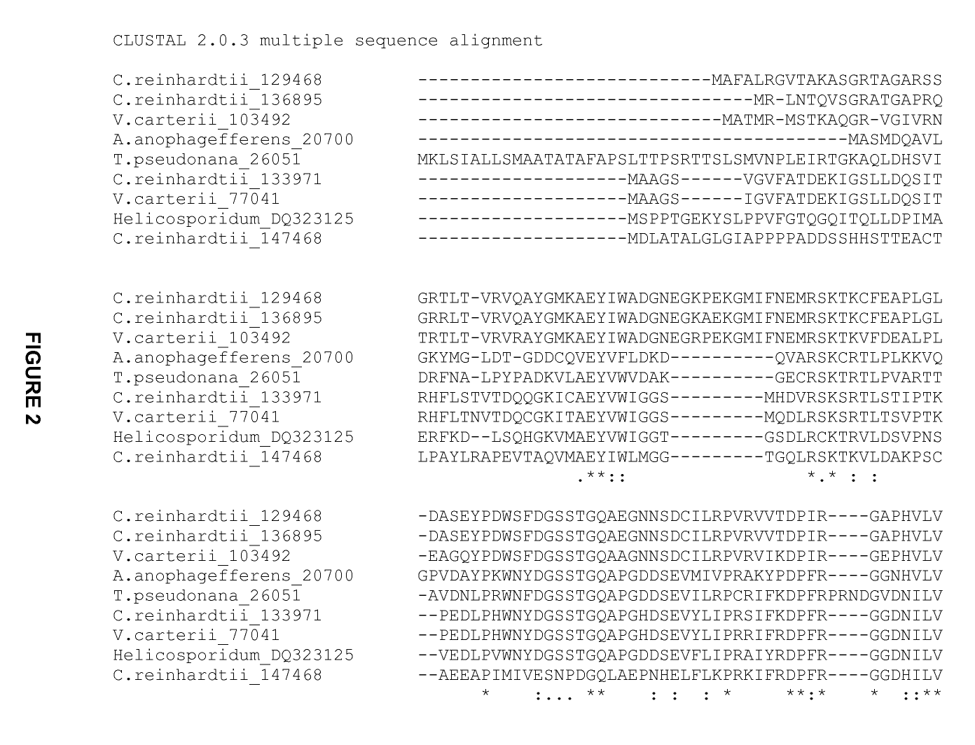

### EXAMPLES

The present invention will now be described with reference to the following examples, which are by way of illustration alone. The following examples are not intended to completely define or otherwise limit the scope of the invention.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Identification of Sequences Useful in the Invention

### 1.1 Glutamine Synthase (GS1)

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

Table A1 provides a list of nucleic acid sequences related to the nucleic acid sequence used in the methods of the present invention.

TABLE A1
Examples of algal-type GS1 polypeptides:

| | Nucleic acid | Protein |
|---|---|---|
| Plant Source | SEQ ID NO: | SEQ ID NO: |
| Chlamydomonas reinhardtii 133971 | 1 | 2 |
| Aureococcus anophagefferens_20700 | 31 | 9 |
| Chlamydomonas reinhardtii_129468 | 32 | 10 |
| Chlamydomonas reinhardtii_136895 | 33 | 11 |
| Chlamydomonas reinhardtii_147468 | 34 | 12 |
| Helicosporidum sp. DQ323125 | 35 | 13 |
| Thalassiosira pseudonana_26051 | 36 | 14 |
| Volvox carterii_103492 | 37 | 15 |
| Volvox carterii_77041 | 38 | 16 |
| Hordeum vulgare_TA45411_4513 | 43 | 21 |
| Physcomitrella patens_122526 | 46 | 24 |
| Physcomitrella patens_146278 | 47 | 25 |
| Pinus taeda_TA26121_3352 | 48 | 26 |
| Pinus taeda_TA8958_3352 | 49 | 27 |
| Phaedactylum tricornutum_51092 | 50 | 28 |
| Hordeum vulgare_7728 | 53 | 55 |
| Hordeum vulgare_7958 | 54 | 56 |

In some instances, related sequences have tentatively been assembled and publicly disclosed by research institutions, such as The Institute for Genomic Research (TIGR). The Eukaryotic Gene Orthologs (EGO) database may be used to identify such related sequences, either by keyword search or by using the BLAST algorithm with the nucleic acid or polypeptide sequence of interest. Preferably the algal-type GS1 polypeptide is of algal origin (such as the proteins exemplified by SEQ ID NO: 2, and SEQ ID NO: 9 to 16).

### 1.2. Phosphoethanolamine N-methyltransferase (PEAMT)

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters were adjusted to modify the stringency of the search, for example the cut-off threshold for the E-value was increased to show less stringent matches. This way, short nearly exact matches may be identified.

Table A2 provides a list of nucleic acid sequences and thereof encoded polypeptides related to the nucleic acid sequence used in the methods of the present invention.

TABLE A2
Examples of PEAMT polypeptides:

| | | Nucleic acid | Protein |
|---|---|---|---|
| Name | Plant Source | SEQ ID NO: | SEQ ID NO: |
| Arath_PEAMT_1 | Arabidopsis thaliana | 57 | 58 |
| AT1G48600_1 | Arabidopsis thaliana | 59 | 60 |
| AT1G73600_1 | Arabidopsis thaliana | 61 | 62 |
| AT3gG18000 | Arabidopsis thaliana | 63 | 64 |
| Os01g50030 | Oryza sativa | 65 | 66 |
| Os05g47540_1 | Oryza sativa | 67 | 68 |
| Os05g47540_2 | Oryza sativa | 69 | 70 |
| Os05g47540_3 | Oryza sativa | 71 | 72 |
| PtPEAMT1 | Populus trichocarpa | 73 | 74 |
| PtPEAMT2 | Populus trichocarpa | 75 | 76 |
| ZmPEAMTa | Zea Mays | 77 | 78 |
| ZmPEAMTb | Zea Mays | 79 | 80 |
| ZmPEAMTc | Zea Mays | 81 | 82 |

### 1.3. Fatty Acyl-Acyl Carrier Protein (ACP) Thioesterase B (FATB)

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid sequence or polypeptide sequences to sequence databases and by calculating the statistical significance of matches. For example, the polypeptide encoded by the nucleic acid sequence of the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid sequence (or polypeptide) sequences over a particular length. In some instances, the default parameters may be adjusted to modify the stringency of the search. For example the E-value may be increased to show less stringent matches. This way, short nearly exact matches may be identified.

Table A3 provides a list of nucleic acid sequences related to the nucleic acid sequence used in the methods of the present invention.

TABLE A3
Examples of FATB polypeptide sequences, and encoding nucleic acid sequences:

| | | Public database | Nucleic acid | Polypeptide |
|---|---|---|---|---|
| Name | Source organism | accession number | SEQ ID NO: | SEQ ID NO: |
| Arath_FATB | Arabidopsis thaliana | NM_100724.2 | 92 | 93 |
| Aqufo_FATB | Aquilegia formosa × | TA8354_338618 | 94 | 95 |
| | Aquilegia pubescens | | | |
| Arahy_FATB | Arachis hypogaea | EF117305.1 | 96 | 97 |
| Braju_FATB | Brassica juncea | DQ856315.1 | 98 | 99 |
| Brasy_FATB | Brachypodium sylvaticum | EF059989 | 100 | 101 |
| Citsi_FATB | Citrus sinensis | TA12334_2711 | 102 | 103 |
| Elagu_FATB | Elaeis guineensis | AF147879 | 104 | 105 |
| Garma_FATB | Garcinia mangostana | U92878 | 106 | 107 |
| Glyma_FATB | Glycine max | BE211486.1 | 108 | 109 |
| | | CX703472.1 | | |
| Goshi_FATB | Gossypium hirsutum | AF034266 | 110 | 111 |
| Helan_FATB | Helianthus annuus | AF036565 | 112 | 113 |
| Irite_FATB | Iris tectorum | AF213480 | 114 | 115 |
| Jatcu_FATB | Jatropha curcas | EU106891.1 | 116 | 117 |
| Maldo_FATB | Madus domestica | TA26272_3750 | 118 | 119 |
| Orysa_FATB | Oryza sativa | NM_001063311 | 120 | 121 |
| Picgl_FATB | Picea glauca | TA16055_3330 | 122 | 123 |
| Popto_FATB | Populus tomentosa | DQ321500.1 | 124 | 125 |
| Ricco_FATB | Ricinus communis | EU000562.1 | 126 | 127 |
| Soltu_FATB | Solanum tuberosum | TA28470_4113 | 128 | 129 |
| Tager_FATB | Tagetes erecta | Proprietary | 130 | 131 |
| Vitvi_FATB | Vitis vinifera | GSVIVT00016807001 | 132 | 133 |
| | | (Genoscope) | | |
| Zeama_FATB | Zea mays | EE033552.2, | 134 | 135 |
| | | BQ577487.1, | | |
| | | AW066432.1 | | |
| Zeama_FATB II | Zea mays | DV029251.1, | 136 | 137 |
| | | CF010081.1 | | |
| Poptr_FATB | Populus trichocarpa | Poptr_FATB | 138 | 139 |

In some instances, related sequences have tentatively been assembled and publicly disclosed by research institutions, such as The Institute for Genomic Research (TIGR; beginning with TA). The Eukaryotic Gene Orthologs (EGO) database may be used to identify such related sequences, either by keyword search or by using the BLAST algorithm with the nucleic acid sequence or polypeptide sequence of interest. On other instances, special nucleic acid sequence databases have been created for particular organisms, such as by the Joint Genome Institute.

### 1.4. Leafy-Like (LFY-Like)

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

Table A4 provides a list of nucleic acid sequences related to the nucleic acid sequence used in the methods of the present invention.

TABLE A4
Examples of LFY-like polypeptides:

| | Nucleic acid | Protein |
|---|---|---|
| Plant Source | SEQ ID NO: | SEQ ID NO: |
| Arabidopsis thaliana | 145 | 146 |
| Arabidopsis thaliana | 176 | 151 |
| Brassica juncea | 177 | 152 |
| Ionopsidium acaule | 178 | 153 |
| Leavenworthia crassa | 179 | 154 |
| Selenia aurea | 180 | 155 |
| Arabidopsis lyrata | 181 | 156 |
| Streptanthus glandulosus | 182 | 157 |
| Cochlearia officinalis | 183 | 158 |
| Brassica oleracea var. botrytis | 184 | 159 |
| Idahoa scapigera | 185 | 160 |
| Capsella bursa-pastoris | 186 | 161 |
| Barbarea vulgaris | 187 | 162 |
| Petunia hybrida | 188 | 163 |
| Antirhinum majus | 189 | 164 |
| Nicotiana tabacum | 190 | 165 |
| Nicotiana tabacum | 191 | 166 |
| Triticum aestivum | 192 | 167 |
| Triticum aestivum | 193 | 168 |
| Lolium temulentum | 194 | 169 |
| Oryza sativa | 195 | 170 |
| Zea mays | 196 | 171 |
| Zea mays | 197 | 172 |
| Ophrys tenthredinifera | 198 | 173 |
| Lycopersicon esculentum | 199 | 174 |
| Carica papaya | 200 | 175 |

In some instances, related sequences have tentatively been assembled and publicly disclosed by research institutions, such as The Institute for Genomic Research (TIGR). The Eukaryotic Gene Orthologs (EGO) database may be used to identify such related sequences, either by keyword search or by using the BLAST algorithm with the nucleic acid or polypeptide sequence of interest.

### Example 2

### Alignment of Sequences Useful in the Invention

### 2.1 Glutamine Synthase (GS1)

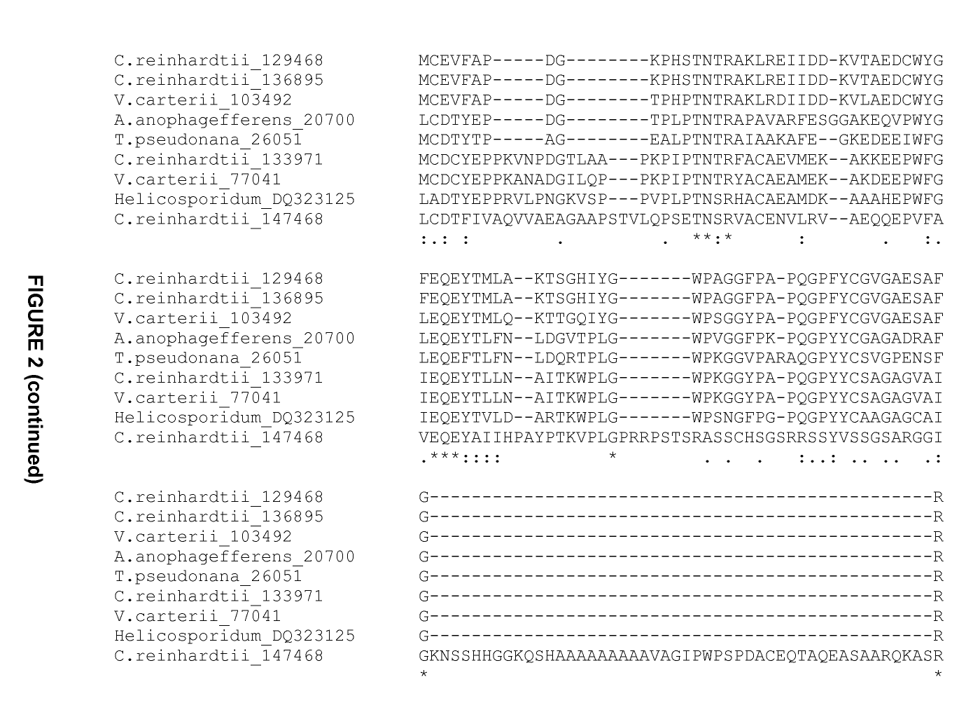

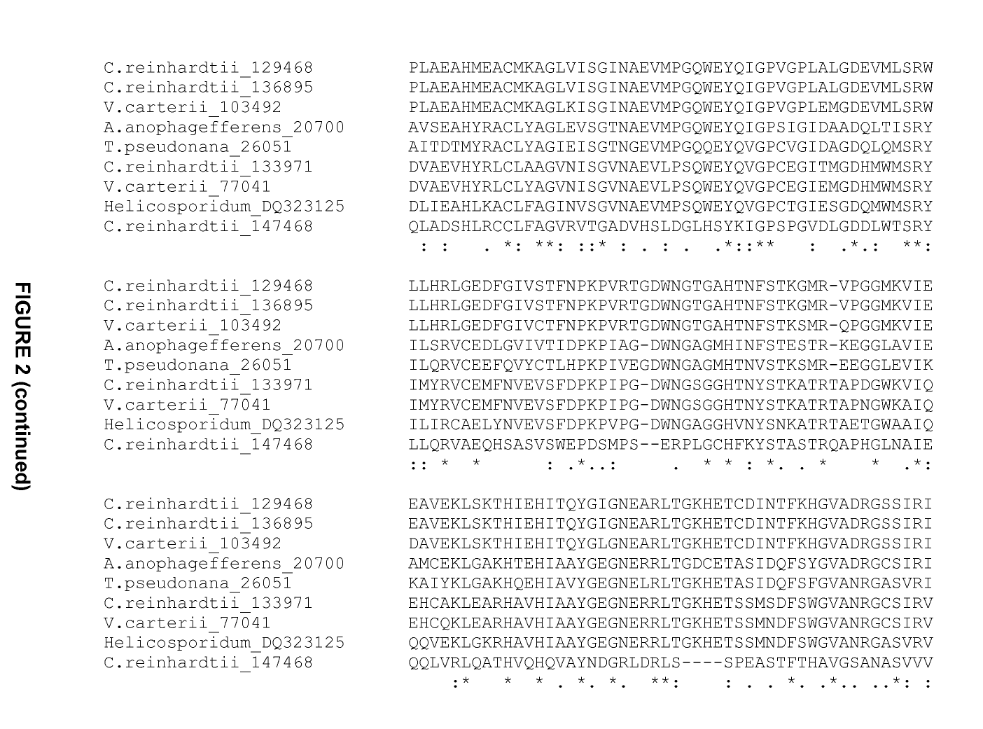

Alignment of polypeptide sequences was performed using the ClustalW 2 algorithm of progressive alignment (Larkin et al., Bioinformatics 23, 2947-2948, 2007). Default values are for the gap open penalty of 10, for the gap extension penalty of 0.2 and the selected weight matrix is Gonnet (if polypeptides are aligned). Minor manual editing may be done to further optimise the alignment. Sequence conservation among GS1 polypeptides is essentially throughout the complete sequence and corresponds to the fact that the Gln-synt_C domain and the Gln-synt_N domain largely span the complete protein sequence. The GS1 polypeptides are aligned in FIG. 2.

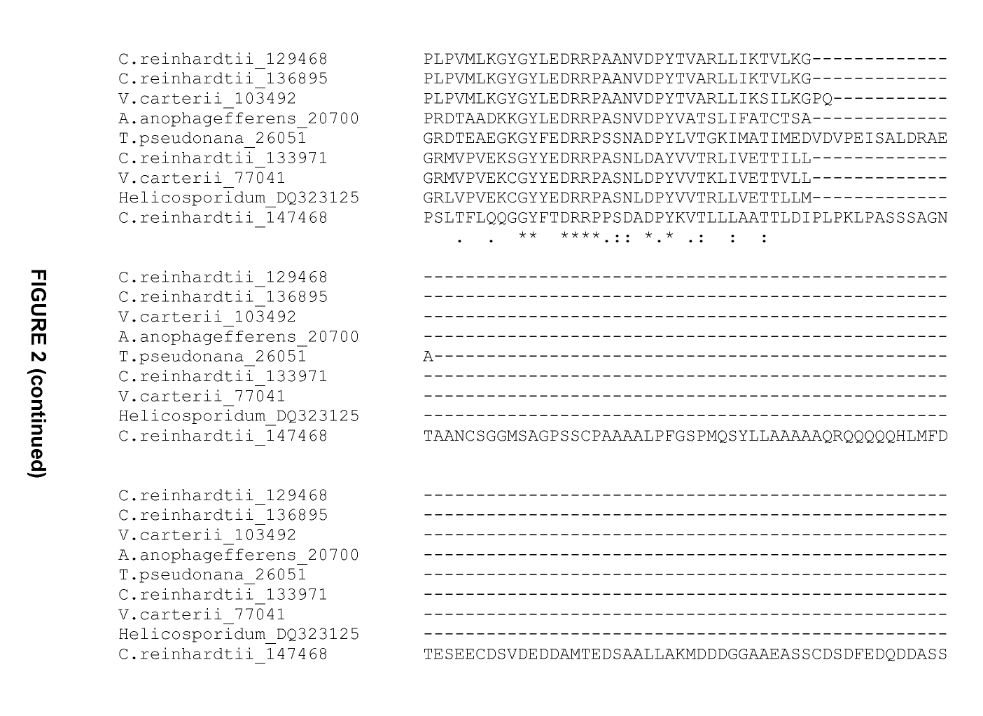

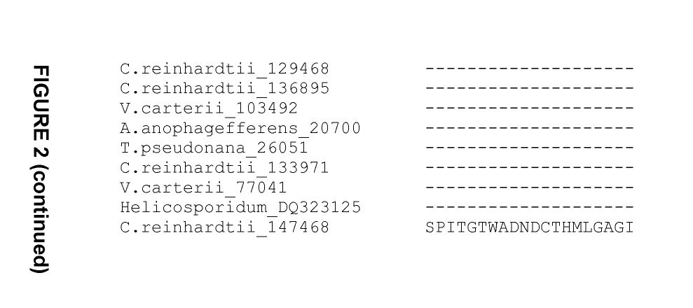

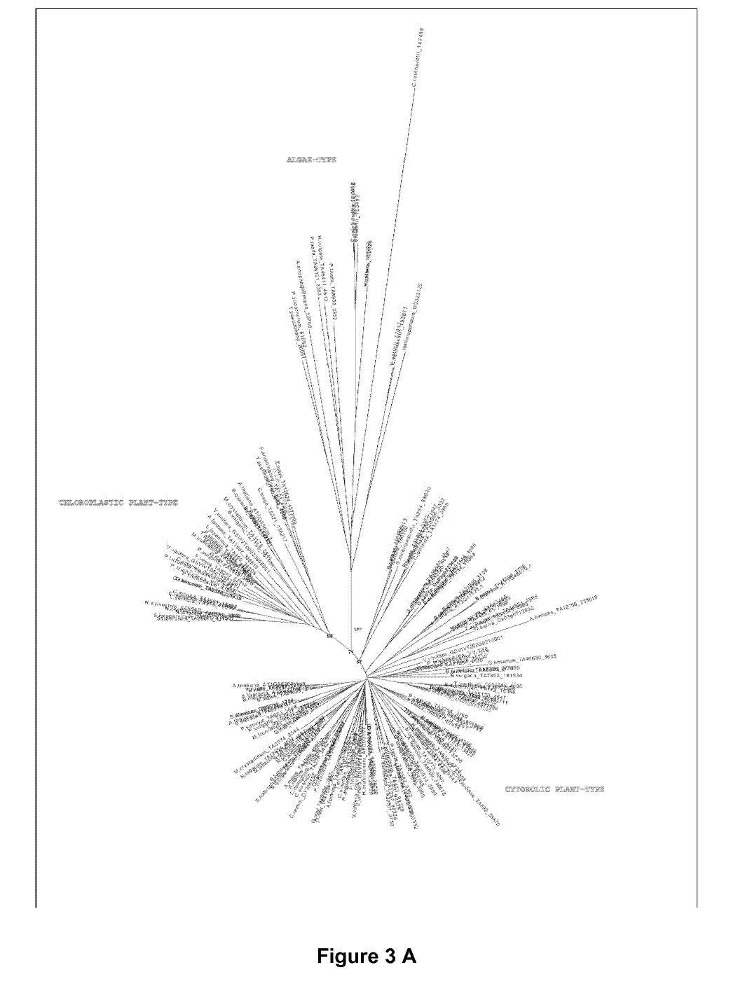

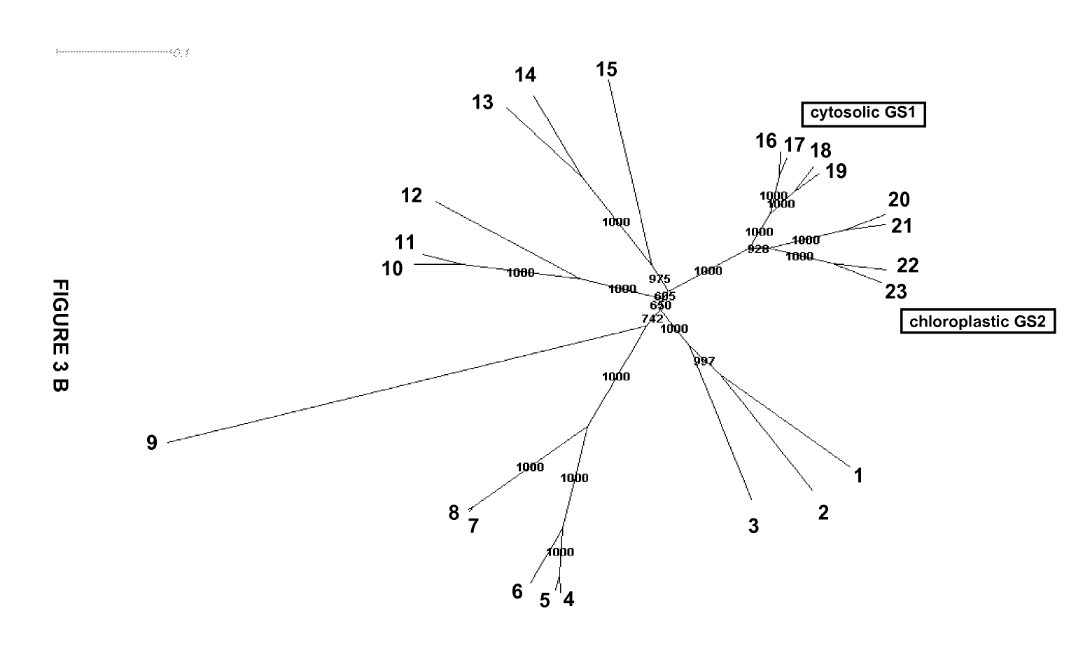

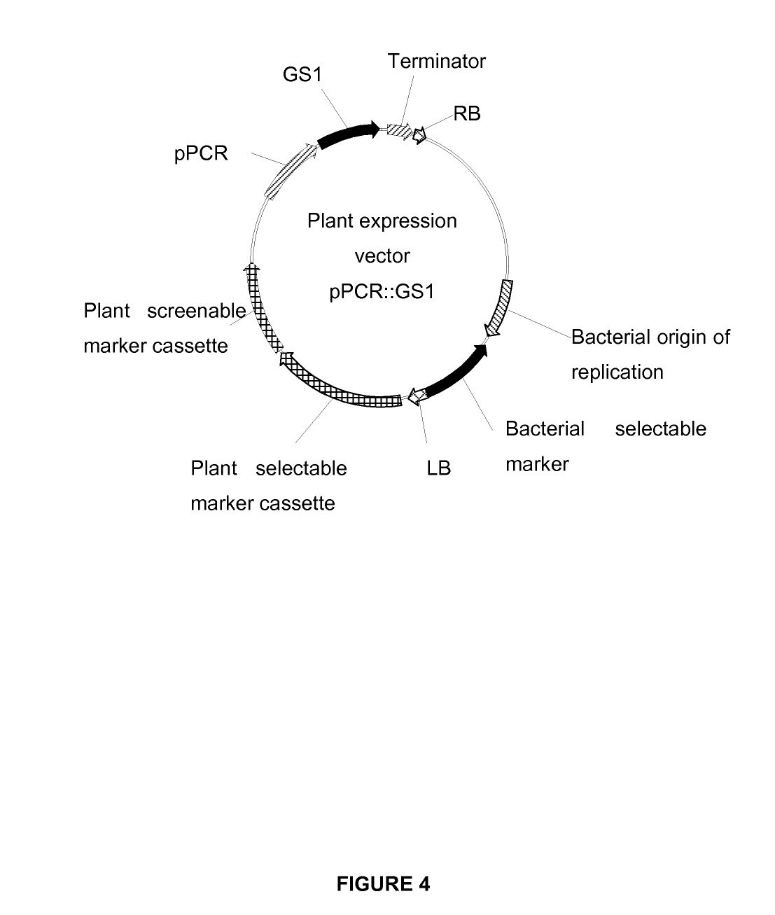

A phylogenetic tree of GS1 polypeptides (FIG. 3) was constructed from alignment using a large number of plant glutamine synthase protein sequences (panel a). From this tree, it can clearly be seen that the algal glutamine synthase proteins form a distinct group (the algal-type clade) compared to other glutamine synthase proteins of plant origin. Panel b shows the same algal-type clade of glutamine synthase proteins but with a limited set of outgroup proteins.

The proteins shown in panel a were aligned using MUSCLE (Edgar (2004), Nucleic Acids Research 32(5): 1792-97). A Neighbour-Joining tree was calculated using QuickTree (Howe et al. (2002), Bioinformatics 18(11): 1546-7). Support of the major branching is indicated for 100 bootstrap repetitions. A circular phylogram was drawn using Dendroscope (Huson et al. (2007), BMC Bioinformatics 8(1):460). The tree clearly shows that the algal GS1 proteins form a distinct group. The sequences shown in panel b were aligned using ClustalW 2 (protein weight matrix: Gonnet series, Gap opening penalty 10, Gap extension penalty 0.2) and a tree was calculated using the Neighbour Joining algorithm with 1000 bootstrap repetitions. Dendroscope was used for drawing the circular phylogram.

### 2.2. Phosphoethanolamine N-methyltransferase (PEAMT)

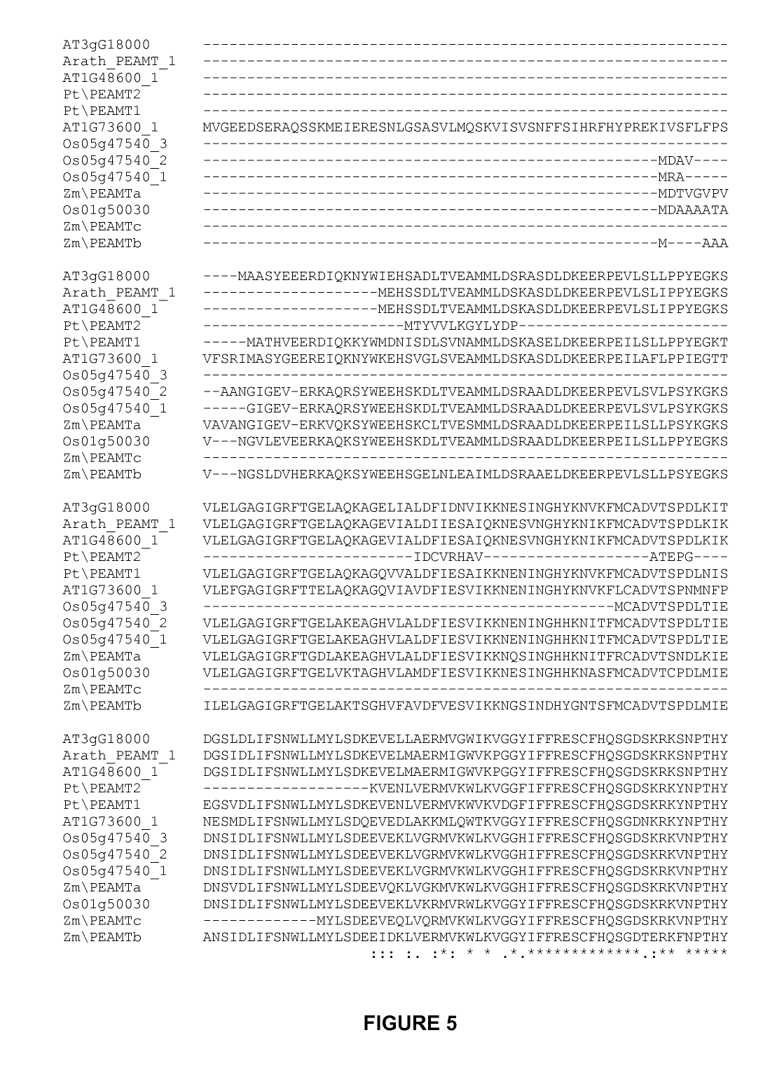

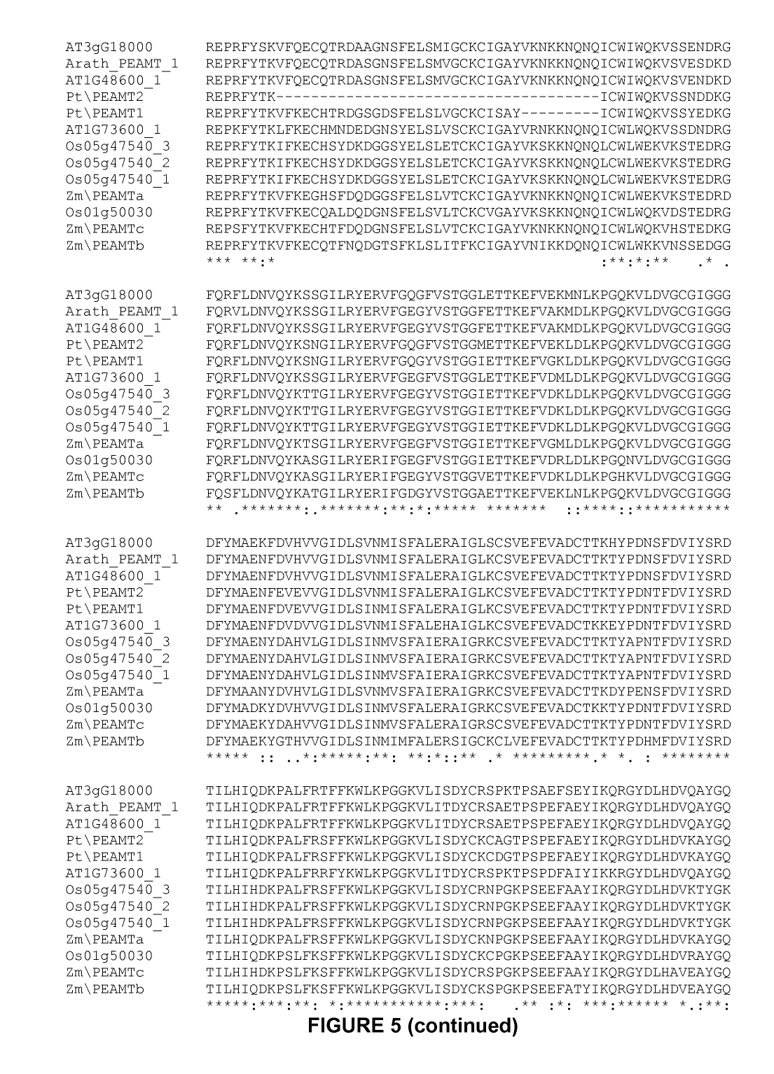

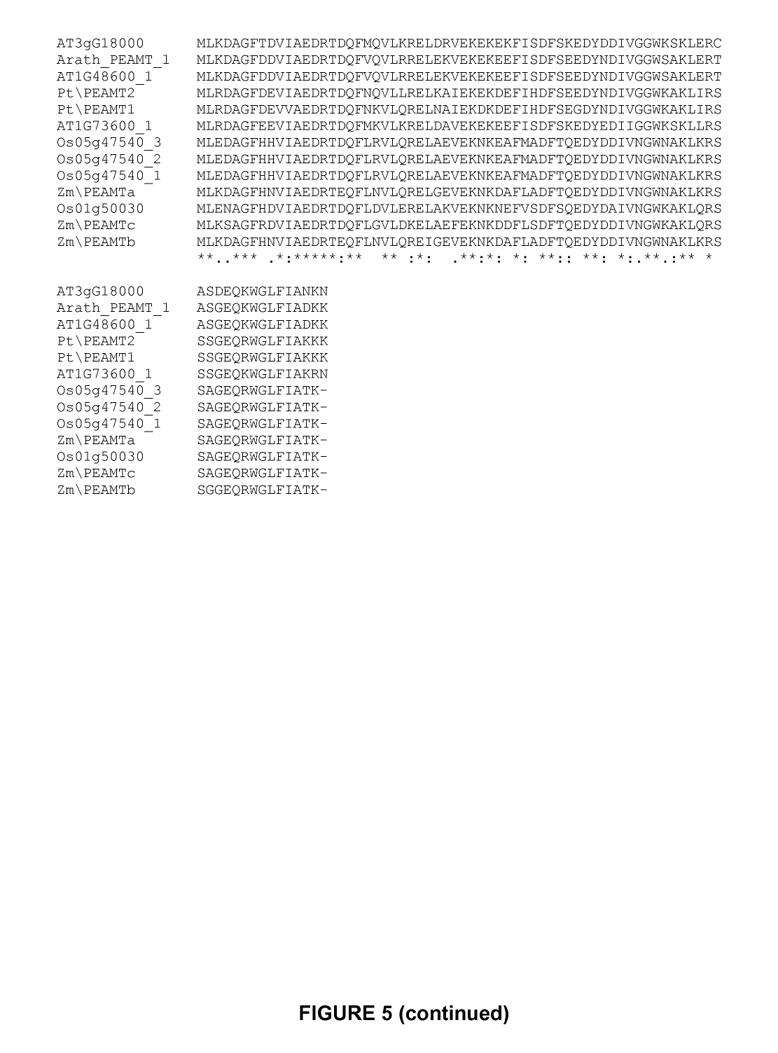

Alignment of polypeptide sequences was performed Clustal W algorithm of progressive alignment (Thompson et al. (1997) Nucleic Acids Res 25:4876-4882; Chema et al. (2003). Nucleic Acids Res 31:3497-3500). Default values are for the gap open penalty of 10, for the gap extension penalty of 0.1 and the selected weight matrix is Blosum 62 (if polypeptides are aligned). Sequence conservation among PEAMT polypeptides is essentially in the C-terminal halt of the polypeptides, the N-terminal domain usually being more variable in sequence length and composition. The PEAMT polypeptides are aligned in FIG. 5. Amino acid residues at positions labelled with * or : are highly conserved in PEAMT proteins.

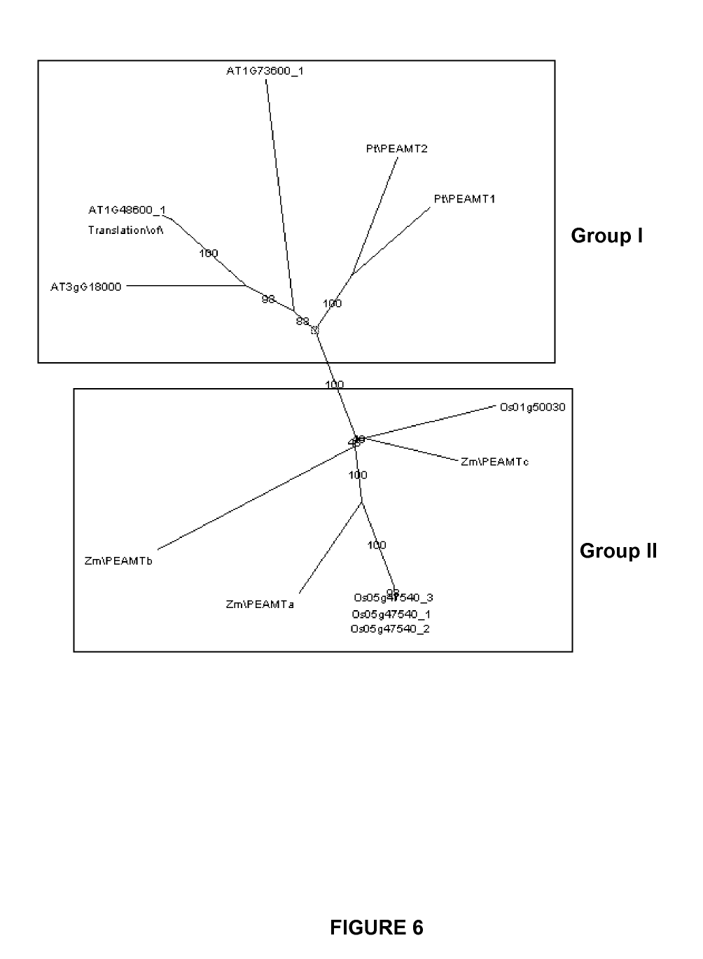

A phylogenetic tree of PEAMT polypeptides (FIG. 6) was constructed using a neighbour-joining clustering algorithm as provided in the Clustal W programme.

### 2.3. Fatty Acyl-Acyl Carrier Protein (ACP) Thioesterase B (FATB)

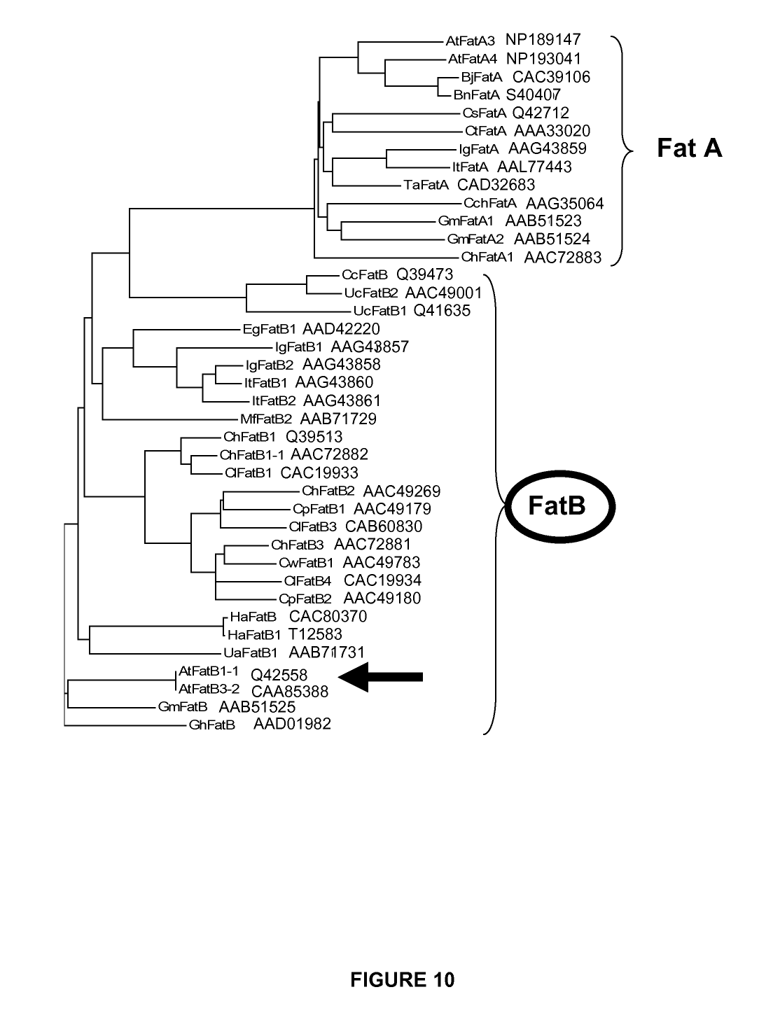

Multiple sequence alignment of all the FATB polypeptide sequences in Table A was performed using the AlignX algorithm (from Vector NTI 10.3, Invitrogen Corporation). Results of the alignment are shown in FIG. 10 of the present application. The N-terminal plastidic transit peptide as predicted by TargetP (Example 5 herein) has been boxed in SEQ ID NO: 93 (Arath_FATB), and the predicted transmembrane helix (typical of FATB polypeptides only) as predicted by TMpred (Example 5 herein) has been boxed across FATB polypeptides useful for performing the methods of the invention. The conserved IPR002864 of the acyl-ACP thioesterase family is marked by X under the consensus sequence. The three highly conserved catalytic residues have been boxed across the alignment.

### 2.4. Leafy-Like (LFY-Like)

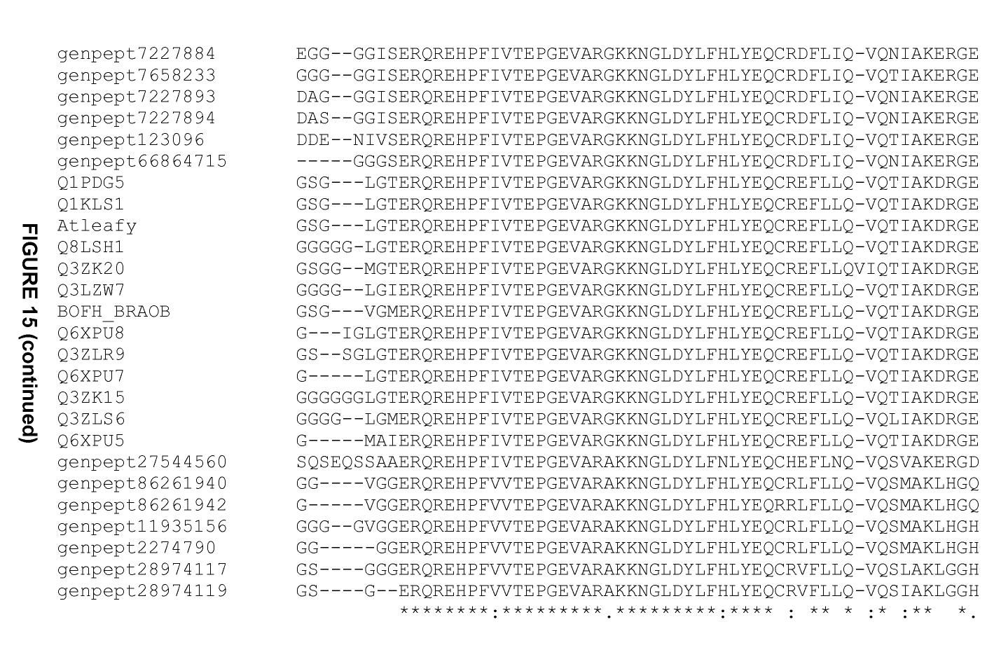

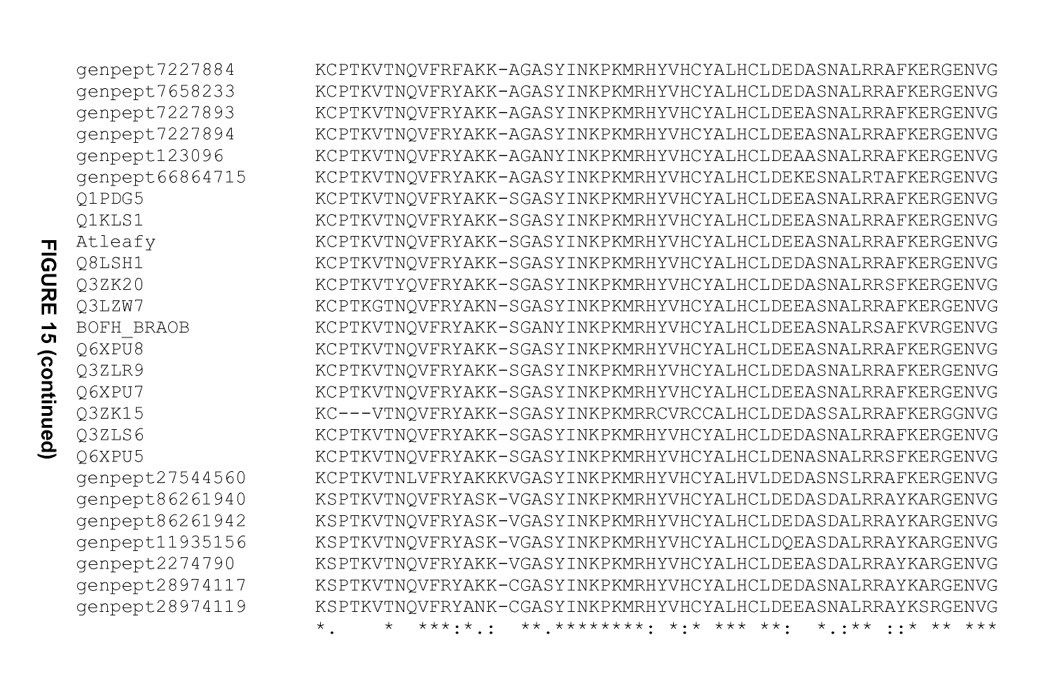

Alignment of polypeptide sequences was performed using ClustalW 2.0.3 (Thompson et al. (1997) Nucleic Acids Res 25:4876-4882; Chema et al. (2003). Nucleic Acids Res 31:3497-3500) with standard setting (slow alignment, similarity matrix: Gonnet, gap opening penalty 10, gap extension penalty: 0.2). Sequence conservation among LFY-like polypeptides is essentially over the whole length of the polypeptides, the N-terminus and the C-terminus usually being more variable in sequence length and composition. The LFY-like polypeptides are aligned in FIG. 15.

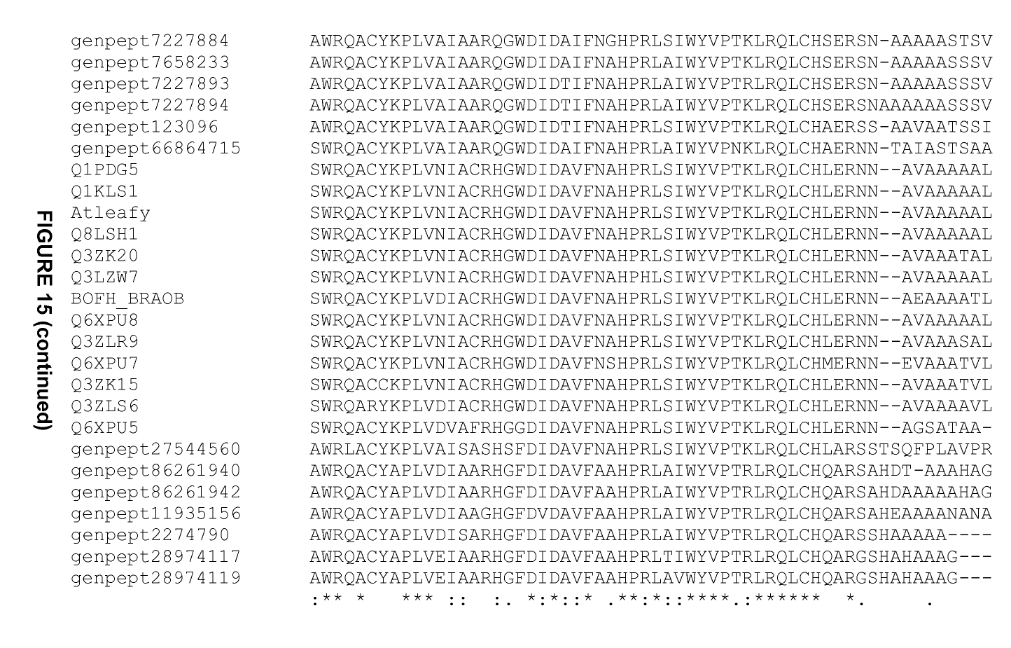

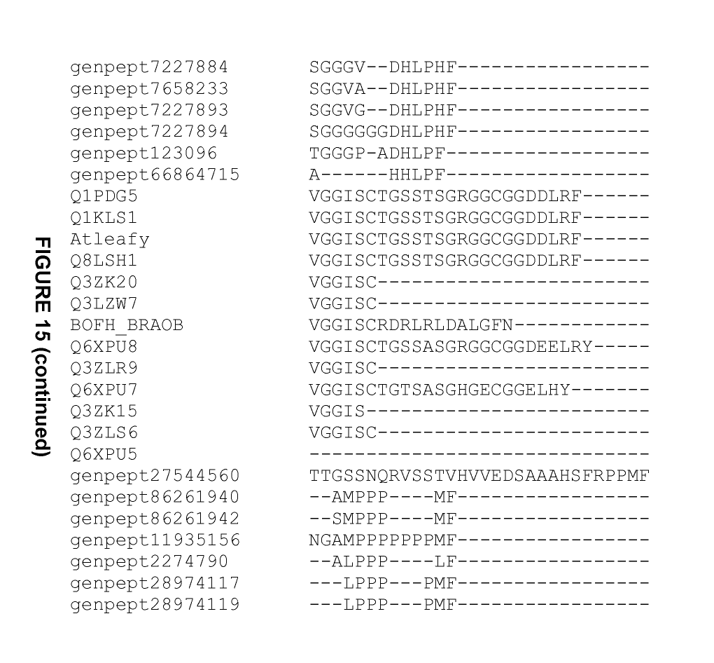

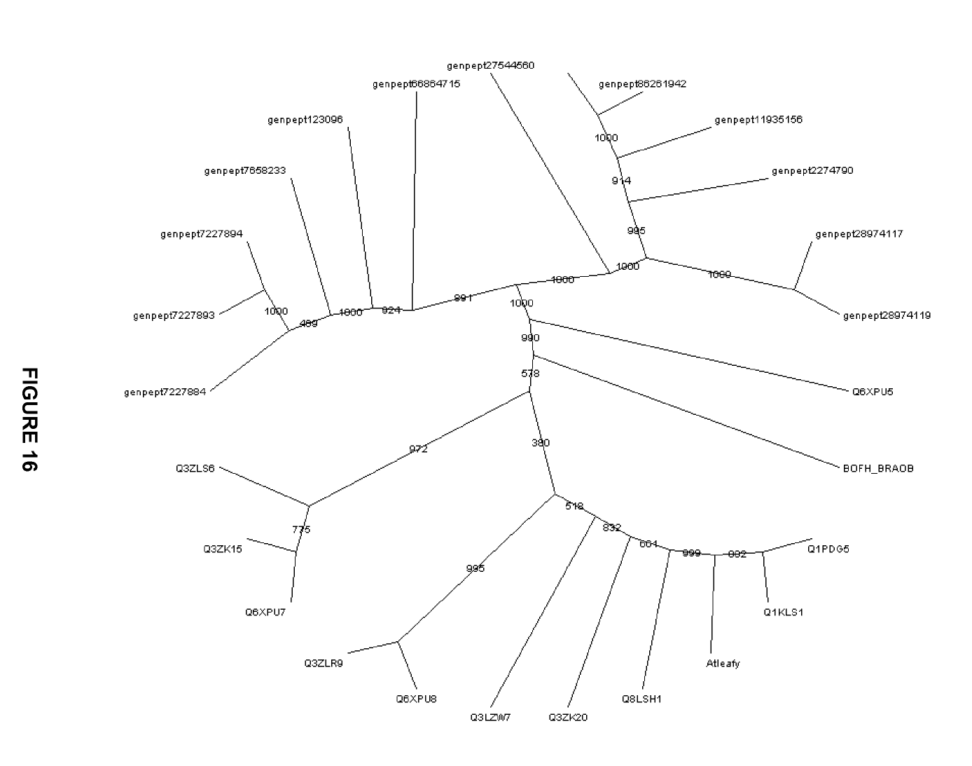

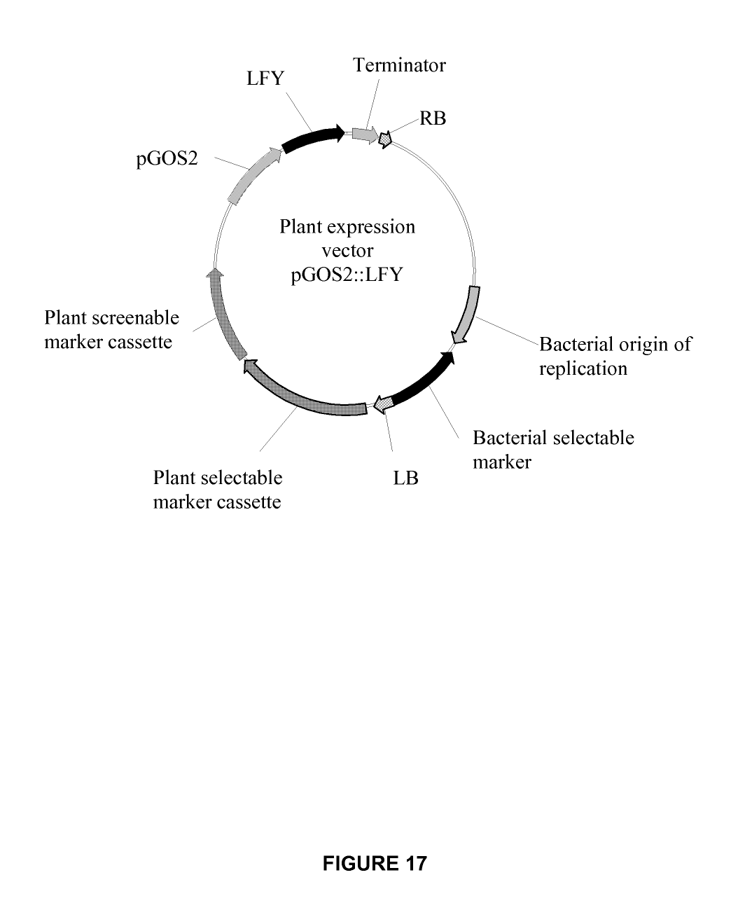

A phylogenetic tree of LFY-like polypeptides (FIG. 16) was constructed using a neighbour-joining clustering algorithm as provided in ClustalW 2.0.3, with 1000 bootstrap repetitions.

### Example 3

### Calculation of Global Percentage Identity Between Polypeptide Sequences Useful in the Invention

### 3.1 Glutamine Synthase (GS1)

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table B1 for the global similarity and identity over the full length of the polypeptide sequences. Percentage identity is given above the diagonal in bold and percentage similarity is given below the diagonal (normal face).

The percentage identity between the algal GS1 polypeptide sequences useful in performing the methods of the invention can be as low as 23% amino acid identity compared to SEQ ID NO: 2 (C.reinhardtii_{—}133971). It should be noted that the algal-type GS1 polypeptides from higher plants (such as SEQ ID NO: 21, 24, 25, 26, 27, and 28) have at least 41% sequence identity when analysed with MatGAT as described above.

TABLE B1
MatGAT results for global similarity and identity over the full length of the GS1
polypeptide sequences.

| | 1 | 2 | 3 | 4 | 5 | 6 | 7 | 8 | 9 |
|---|---|---|---|---|---|---|---|---|---|
| 1. C.reinhardtii_129468 | | 43.7 | 95.3 | 20.5 | 86.6 | 43.9 | 45.6 | 41.7 | 40.0 |
| 2. C.reinhardtii_133971 | 62.3 | | 42.1 | 23.0 | 43.7 | 92.1 | 52.1 | 68.3 | 48.5 |
| 3. C.reinhardtii_136895 | 95.8 | 61.3 | | 20.1 | 86.3 | 42.9 | 46.2 | 42.2 | 39.8 |
| 4. C.reinhardtii_147468 | 31.5 | 36.6 | 31.2 | | 21.0 | 23.0 | 20.7 | 26.1 | 22.1 |
| 5. V.carterii_103492 | 92.4 | 63.9 | 91.3 | 33.6 | | 43.4 | 46.3 | 42.3 | 41.5 |
| 6. V.carterii_77041 | 62.3 | 95.3 | 61.3 | 37.1 | 63.9 | | 52.2 | 70.4 | 49.0 |
| 7. A.anophagefferens_20700 | 57.4 | 64.9 | 58.4 | 30.8 | 59.9 | 65.4 | | 49.6 | 52.3 |
| 8. Helicosporidum_DQ323125 | 60.1 | 79.8 | 59.6 | 37.1 | 60.1 | 81.1 | 62.7 | | 46.3 |
| 9. T.pseudonana_26051 | 56.0 | 60.1 | 55.0 | 34.8 | 57.2 | 61.1 | 63.5 | 59.9 | |

### 3.2. Phosphoethanolamine N-methyltransferase (PEAMT)

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table B2 for the global similarity and identity over the full length of the polypeptide sequences. Percentage identity is given below the diagonal in bold and percentage similarity is given above the diagonal (normal face).

The percentage identity between the PEAMT polypeptide sequences useful in performing the methods of the invention can be as low as 60.2% amino acid identity compared to SEQ ID NO: 58.

TABLE B2
MatGAT results for global similarity and identity over the full length of the PEAMT polypeptide sequences.

| Polypeptide name | 1 | 2 | 3 | 4 | 5 | 6 | 7 | 8 | 9 | 10 | 11 | 12 | 13 |
|---|---|---|---|---|---|---|---|---|---|---|---|---|---|
| 1. AT3gG18000 | | 86.2 | 60.9 | 76.0 | 76.6 | 77.6 | 72.3 | 86.8 | 58.7 | 74.0 | 75.5 | 56.6 | 79.6 |
| 2. Arath_PEAMT_1 | 93.1 | | 63.2 | 74.4 | 75.0 | 75.0 | 68.5 | 99.4 | 60.2 | 70.8 | 73.5 | 59.2 | 80.0 |
| 3. Os05g47540_3 | 70.7 | 73.3 | | 78.2 | 78.8 | 66.9 | 53.5 | 63.4 | 80.9 | 64.6 | 70.1 | 68.0 | 62.2 |
| 4. Os05g47540_2 | 88.7 | 86.7 | 78.2 | | 99.0 | 85.8 | 66.3 | 74.8 | 63.2 | 80.6 | 89.2 | 53.8 | 75.8 |
| 5. Os05g47540_1 | 89.4 | 87.4 | 78.8 | 99.0 | | 85.0 | 66.2 | 75.4 | 63.7 | 80.2 | 88.4 | 54.1 | 76.2 |
| 6. Os01g50030 | 88.6 | 85.2 | 73.1 | 93.6 | 92.8 | | 67.6 | 75.4 | 64.3 | 81.4 | 84.1 | 54.8 | 76.0 |
| 7. AT1G73600_1 | 81.8 | 78.6 | 62.2 | 79.1 | 78.6 | 80.0 | | 69.0 | 50.8 | 62.0 | 66.9 | 49.9 | 69.5 |
| 8. AT1G48600_1 | 93.5 | 99.6 | 73.5 | 87.1 | 87.8 | 85.6 | 78.9 | | 60.4 | 71.2 | 73.9 | 59.4 | 80.4 |
| 9. Zm\PEAMTc | 66.8 | 68.0 | 88.1 | 68.9 | 69.5 | 69.5 | 58.6 | 68.2 | | 61.4 | 62.1 | 68.6 | 58.4 |
| 10. Zm\PEAMTb | 86.3 | 84.0 | 72.5 | 91.9 | 91.5 | 91.6 | 76.9 | 84.4 | 67.5 | | 80.3 | 52.8 | 73.0 |
| 11. Zm\PEAMTa | 87.6 | 85.6 | 74.3 | 94.8 | 94.0 | 92.4 | 80.7 | 86.0 | 67.5 | 89.6 | | 54.2 | 74.5 |
| 12. Pt\PEAMT2 | 63.1 | 65.7 | 76.0 | 60.2 | 61.3 | 61.5 | 57.1 | 66.1 | 81.2 | 60.0 | 60.1 | | 65.1 |
| 13. Pt\PEAMT1 | 91.0 | 90.2 | 69.6 | 85.7 | 86.2 | 86.2 | 79.3 | 90.6 | 65.7 | 83.6 | 84.4 | 68.0 | |

### 3.3. Fatty Acyl-Acyl Carrier Protein (ACP) Thioesterase B (FATB)

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table B3 for the global similarity and identity over the full length of the polypeptide sequences (excluding the partial polypeptide sequences).

The percentage identity between the full length polypeptide sequences useful in performing the methods of the invention can be as low as 53% amino acid identity compared to SEQ ID NO: 93.

TABLE B3
MatGAT results for global similarity and identity over the full length of the FATB polypeptide sequences of Table A3.

| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
|---|---|---|---|---|---|---|---|---|---|---|---|---|---|---|---|---|---|---|---|---|---|---|---|---|
| 1. Aqufo_FATB | | 64 | 63 | 61 | 57 | 67 | 64 | 65 | 65 | 62 | 59 | 58 | 68 | 66 | 59 | 51 | 68 | 66 | 63 | 63 | 69 | 56 | 56 | 34 |
| 2. Arahy_FATB | 80 | | 75 | 72 | 60 | 75 | 67 | 80 | 88 | 74 | 68 | 63 | 80 | 79 | 63 | 53 | 78 | 79 | 71 | 71 | 79 | 60 | 61 | 35 |
| 3. Arath_FATB | 78 | 86 | | 89 | 59 | 73 | 66 | 72 | 75 | 71 | 65 | 63 | 76 | 74 | 60 | 53 | 75 | 76 | 69 | 67 | 74 | 59 | 58 | 36 |
| 4. Braju_FATB | 76 | 83 | 93 | | 56 | 70 | 64 | 72 | 71 | 68 | 64 | 62 | 73 | 71 | 59 | 53 | 72 | 73 | 66 | 64 | 71 | 56 | 57 | 35 |
| 5. Brasy_FATB | 72 | 74 | 73 | 72 | | 60 | 69 | 60 | 60 | 58 | 56 | 62 | 62 | 61 | 86 | 50 | 63 | 61 | 61 | 60 | 62 | 81 | 64 | 31 |
| 6. Citsi_FATB | 79 | 86 | 81 | 80 | 74 | | 67 | 71 | 76 | 76 | 65 | 64 | 79 | 79 | 62 | 52 | 78 | 78 | 70 | 69 | 79 | 60 | 58 | 34 |
| 7. Elagu_FATB | 76 | 80 | 78 | 76 | 81 | 79 | | 64 | 66 | 64 | 60 | 71 | 71 | 67 | 71 | 54 | 68 | 68 | 64 | 64 | 70 | 67 | 65 | 35 |
| 8. Garma_FATB | 79 | 88 | 83 | 82 | 73 | 85 | 78 | | 78 | 71 | 68 | 62 | 80 | 76 | 62 | 52 | 79 | 79 | 71 | 70 | 76 | 59 | 60 | 37 |
| 9. Glyma_FATB | 78 | 93 | 85 | 80 | 72 | 87 | 79 | 89 | | 74 | 69 | 63 | 80 | 79 | 63 | 52 | 77 | 78 | 71 | 70 | 79 | 59 | 59 | 37 |
| 10. Goshi_FATB | 77 | 86 | 81 | 80 | 72 | 84 | 77 | 82 | 86 | | 65 | 61 | 79 | 74 | 59 | 52 | 76 | 77 | 67 | 66 | 75 | 56 | 56 | 34 |
| 11. Helan_FATB | 73 | 81 | 77 | 75 | 73 | 79 | 76 | 80 | 82 | 80 | | 59 | 67 | 69 | 58 | 51 | 67 | 67 | 70 | 75 | 68 | 56 | 58 | 34 |
| 12. Irite_FATB | 74 | 77 | 76 | 75 | 78 | 78 | 85 | 77 | 77 | 75 | 76 | | 68 | 64 | 64 | 52 | 64 | 64 | 64 | 64 | 65 | 61 | 59 | 33 |
| 13. Jatcu_FATB | 81 | 89 | 85 | 83 | 74 | 89 | 82 | 88 | 89 | 88 | 80 | 80 | | 80 | 65 | 56 | 84 | 89 | 71 | 70 | 84 | 62 | 63 | 36 |
| 14. Maldo_FATB | 81 | 88 | 84 | 82 | 73 | 87 | 78 | 87 | 89 | 84 | 81 | 77 | 90 | | 64 | 55 | 80 | 79 | 71 | 72 | 81 | 60 | 59 | 35 |
| 15. Orysa_FATB | 73 | 76 | 74 | 73 | 92 | 77 | 82 | 75 | 75 | 75 | 74 | 79 | 77 | 76 | | 50 | 65 | 63 | 63 | 61 | 64 | 85 | 65 | 33 |
| 16. Picgl_FATB | 66 | 67 | 67 | 68 | 66 | 67 | 67 | 66 | 66 | 68 | 65 | 69 | 69 | 69 | 66 | | 55 | 54 | 52 | 53 | 55 | 48 | 49 | 34 |
| 17. Popto_FATB | 78 | 87 | 84 | 81 | 76 | 88 | 80 | 86 | 87 | 86 | 80 | 78 | 91 | 88 | 78 | 67 | | 83 | 70 | 70 | 80 | 62 | 61 | 35 |
| 18. Ricco_FATB | 79 | 87 | 84 | 82 | 74 | 87 | 79 | 88 | 89 | 85 | 79 | 79 | 94 | 88 | 76 | 69 | 90 | | 69 | 69 | 80 | 61 | 63 | 37 |
| 19. Soltu_FATB | 77 | 82 | 80 | 77 | 74 | 82 | 79 | 80 | 82 | 79 | 81 | 76 | 81 | 82 | 76 | 67 | 82 | 83 | | 75 | 74 | 61 | 59 | 33 |
| 20. Tager_FATB | 77 | 84 | 82 | 78 | 73 | 82 | 79 | 82 | 84 | 83 | 84 | 80 | 83 | 84 | 74 | 68 | 82 | 82 | 84 | | 72 | 58 | 60 | 33 |
| 21. Vitvi_FATB | 80 | 87 | 84 | 80 | 75 | 88 | 80 | 85 | 87 | 85 | 80 | 79 | 90 | 90 | 78 | 68 | 90 | 88 | 83 | 83 | | 63 | 60 | 34 |
| 22. Zeama_FATB | 70 | 74 | 73 | 70 | 89 | 74 | 79 | 73 | 74 | 72 | 71 | 77 | 74 | 73 | 90 | 64 | 75 | 75 | 74 | 71 | 75 | | 64 | 31 |
| 23. Zeama_FATB\II | 72 | 75 | 73 | 70 | 78 | 73 | 78 | 73 | 73 | 74 | 71 | 76 | 75 | 73 | 78 | 62 | 73 | 76 | 73 | 75 | 74 | 77 | | 33 |
| 24. Arath_FATA | 51 | 51 | 53 | 52 | 49 | 52 | 52 | 56 | 54 | 53 | 50 | 50 | 53 | 54 | 50 | 49 | 51 | 53 | 52 | 51 | 51 | 47 | 49 | |

### 3.4. Leafy-Like (LFY-Like)

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum 62
First Gap: 12
Extending gap: 2

Results of the software analysis are shown in Table B4 for the global similarity and identity over the full length of the polypeptide sequences. Percentage identity is given above the diagonal and percentage similarity is given below the diagonal.

The percentage identity between the LFY-like polypeptide sequences useful in performing the methods of the invention can be as low as 50% amino acid identity compared to SEQ ID NO: 146.

TABLE B4
MatGAT results for global similarity and identity over the full length of the LFY-like
polypeptide sequences.

| | 1 | 2 | 3 | 4 | 5 | 6 | 7 | 8 | 9 | 10 | 11 | 12 | 13 |
|---|---|---|---|---|---|---|---|---|---|---|---|---|---|
| 1. Atleafy | | 99.1 | 98.8 | 90.4 | 90.8 | 87.8 | 94.9 | 85.8 | 86.0 | 87.5 | 79.8 | 88.8 | 85.0 |
| 2. Q1PDG5 | 99.1 | | 99.8 | 89.5 | 89.9 | 86.9 | 94.0 | 85.1 | 85.1 | 86.6 | 78.7 | 87.8 | 84.1 |
| 3. Q1KLS1 | 99.1 | 99.8 | | 89.3 | 89.6 | 86.6 | 93.5 | 84.8 | 84.9 | 86.4 | 78.7 | 87.6 | 83.9 |
| 4. Q6XPU8 | 94.1 | 93.2 | 93.2 | | 87.1 | 83.2 | 87.8 | 82.3 | 87.9 | 83.9 | 76.1 | 84.2 | 81.0 |
| 5. Q6XPU7 | 93.9 | 93.8 | 93.8 | 90.6 | | 88.3 | 87.9 | 81.8 | 82.9 | 83.4 | 78.1 | 84.7 | 86.5 |
| 6. Q3ZLS6 | 90.3 | 90.2 | 90.2 | 86.6 | 91.6 | | 85.8 | 85.2 | 86.1 | 84.2 | 81.0 | 87.8 | 89.9 |
| 7. Q8LSH1 | 96.5 | 95.6 | 95.1 | 92.1 | 91.4 | 88.8 | | 84.8 | 85.1 | 84.7 | 78.7 | 88.4 | 84.7 |
| 8. Q3LZW7 | 88.0 | 88.1 | 88.1 | 85.7 | 86.1 | 90.1 | 87.0 | | 83.9 | 82.7 | 79.3 | 90.6 | 86.2 |
| 9. Q3ZLR9 | 90.8 | 90.7 | 90.7 | 91.8 | 89.2 | 90.7 | 88.4 | 88.9 | | 83.5 | 78.9 | 87.2 | 84.3 |
| 10. BOFH_BRAOB | 90.6 | 90.5 | 90.5 | 87.3 | 88.0 | 88.4 | 88.4 | 86.7 | 89.9 | | 76.1 | 85.0 | 80.8 |
| 11. Q6XPU5 | 85.1 | 84.3 | 84.3 | 82.2 | 84.7 | 88.3 | 84.2 | 85.9 | 85.0 | 82.9 | | 82.2 | 78.9 |
| 12. Q3ZK20 | 91.0 | 91.0 | 91.0 | 87.1 | 89.0 | 91.8 | 89.8 | 92.6 | 90.9 | 88.7 | 88.5 | | 90.1 |
| 13. Q3ZK15 | 88.0 | 87.9 | 87.9 | 84.5 | 89.0 | 92.1 | 87.0 | 88.8 | 88.7 | 85.3 | 87.3 | 92.7 | |
| 14. genpept7227884 | 78.8 | 79.5 | 79.3 | 76.3 | 76.0 | 77.4 | 77.4 | 76.2 | 78.4 | 77.3 | 76.7 | 77.2 | 74.0 |
| 15. genpept123096 | 73.8 | 74.5 | 74.3 | 73.5 | 74.6 | 77.4 | 74.4 | 76.7 | 75.9 | 74.0 | 77.8 | 78.4 | 75.0 |
| 16. genpept7227893 | 77.8 | 78.6 | 78.3 | 76.1 | 76.7 | 77.2 | 76.3 | 76.3 | 78.2 | 78.6 | 75.3 | 78.0 | 74.3 |
| 17. genpept7227894 | 80.2 | 81.0 | 80.7 | 77.2 | 77.2 | 77.4 | 77.2 | 75.2 | 77.6 | 79.1 | 74.8 | 77.6 | 74.5 |
| 18. genpept86261940 | 62.5 | 61.9 | 61.7 | 62.2 | 63.8 | 65.5 | 61.9 | 62.8 | 65.4 | 63.4 | 65.8 | 65.2 | 64.9 |
| 19. genpept86261942 | 63.2 | 61.9 | 61.7 | 62.9 | 64.0 | 64.0 | 62.1 | 64.3 | 65.1 | 63.4 | 65.1 | 65.7 | 63.9 |
| 20. genpept11935156 | 63.7 | 64.0 | 64.0 | 63.6 | 62.8 | 63.8 | 62.8 | 64.5 | 67.1 | 64.6 | 63.3 | 66.3 | 62.3 |
| 21. genpept2274790 | 63.9 | 64.5 | 64.5 | 63.6 | 64.5 | 65.5 | 62.1 | 66.7 | 67.3 | 63.1 | 66.8 | 64.9 | 63.6 |
| 22. genpept28974117 | 65.8 | 66.4 | 66.4 | 64.1 | 65.0 | 64.3 | 63.7 | 64.5 | 66.3 | 63.6 | 64.6 | 64.9 | 65.1 |
| 23. genpept28974119 | 62.5 | 63.1 | 63.8 | 62.2 | 62.4 | 65.0 | 61.9 | 65.5 | 65.4 | 62.9 | 66.2 | 64.2 | 63.9 |
| 24. genpept27544560 | 62.9 | 62.9 | 62.7 | 61.6 | 62.9 | 61.6 | 60.7 | 61.0 | 61.8 | 63.2 | 58.8 | 60.7 | 60.1 |
| 25. genpept7658233 | 77.6 | 78.3 | 78.1 | 76.8 | 76.5 | 77.4 | 76.0 | 77.4 | 79.1 | 78.3 | 77.2 | 77.9 | 75.7 |
| 26. genpept66864715 | 73.6 | 74.3 | 74.0 | 73.2 | 74.6 | 76.7 | 71.9 | 73.7 | 76.4 | 74.2 | 76.1 | 75.9 | 73.8 |
| | 14 | 15 | 16 | 17 | 18 | 19 | 20 | 21 | 22 | 23 | 24 | 25 | 26 |
| 1. Atleafy | 65.5 | 65.0 | 65.8 | 67.3 | 50.3 | 50.7 | 51.3 | 51.5 | 51.9 | 51.3 | 49.5 | 64.8 | 65.8 |
| 2. Q1PDG5 | 66.1 | 65.6 | 66.4 | 67.9 | 49.8 | 49.5 | 51.7 | 52.5 | 52.4 | 52.0 | 49.9 | 65.4 | 66.4 |
| 3. Q1KLS1 | 65.8 | 65.3 | 66.2 | 67.7 | 49.5 | 49.3 | 51.7 | 52.5 | 52.4 | 51.5 | 49.7 | 65.1 | 66.2 |
| 4. Q6XPU8 | 63.9 | 63.7 | 64.0 | 64.7 | 50.2 | 50.1 | 51.4 | 51.2 | 50.5 | 51.2 | 49.5 | 63.9 | 63.6 |
| 5. Q6XPU7 | 62.8 | 65.0 | 64.5 | 64.3 | 50.6 | 50.5 | 50.8 | 52.0 | 50.0 | 50.2 | 50.0 | 63.5 | 66.7 |
| 6. Q3ZLS6 | 64.5 | 66.0 | 66.0 | 65.0 | 51.8 | 52.2 | 51.4 | 52.4 | 52.6 | 53.2 | 49.0 | 65.4 | 67.0 |
| 7. Q8LSH1 | 65.8 | 64.1 | 64.7 | 65.7 | 50.7 | 51.2 | 50.5 | 50.8 | 51.5 | 50.9 | 48.6 | 64.2 | 64.4 |
| 8. Q3LZW7 | 63.7 | 64.0 | 64.5 | 64.3 | 50.1 | 50.4 | 50.2 | 52.4 | 52.0 | 52.5 | 49.5 | 64.8 | 64.0 |
| 9. Q3ZLR9 | 65.4 | 64.6 | 64.6 | 64.6 | 51.4 | 51.9 | 51.2 | 52.6 | 53.4 | 51.8 | 49.8 | 65.3 | 66.2 |
| 10. BOFH_BRAOB | 64.1 | 64.1 | 64.3 | 64.8 | 51.3 | 51.2 | 51.9 | 51.6 | 50.9 | 52.0 | 49.3 | 64.1 | 64.4 |
| 11. Q6XPU5 | 63.1 | 64.6 | 64.1 | 64.3 | 52.8 | 52.4 | 51.8 | 53.1 | 52.4 | 53.1 | 47.6 | 65.4 | 65.6 |
| 12. Q3ZK20 | 65.0 | 65.5 | 64.5 | 64.5 | 51.2 | 51.6 | 50.5 | 51.8 | 51.6 | 51.2 | 49.5 | 65.6 | 65.7 |
| 13. Q3ZK15 | 62.1 | 63.2 | 63.2 | 61.7 | 50.7 | 50.0 | 48.2 | 49.4 | 50.5 | 49.5 | 48.3 | 63.6 | 64.4 |
| 14. genpept7227884 | | 76.2 | 89.9 | 89.3 | 55.4 | 55.4 | 55.0 | 55.5 | 55.7 | 55.2 | 49.5 | 89.3 | 72.6 |
| 15. genpept123096 | 84.7 | | 76.2 | 76.3 | 54.5 | 55.4 | 55.4 | 56.0 | 54.2 | 56.3 | 50.2 | 76.0 | 73.8 |
| 16. genpept7227893 | 93.9 | 84.5 | | 96.4 | 55.7 | 56.1 | 55.1 | 56.9 | 54.6 | 54.7 | 50.5 | 89.1 | 73.2 |
| 17. genpept7227894 | 93.3 | 83.4 | 97.6 | | 55.9 | 55.2 | 54.1 | 56.5 | 54.2 | 54.4 | 50.3 | 88.0 | 72.4 |
| 18. genpept86261940 | 68.4 | 66.2 | 67.1 | 67.5 | | 96.7 | 87.3 | 86.4 | 80.0 | 78.7 | 48.9 | 56.5 | 53.4 |
| 19. genpept86261942 | 68.0 | 66.9 | 67.6 | 67.1 | 98.0 | | 88.1 | 85.9 | 79.9 | 78.2 | 48.6 | 55.0 | 52.9 |
| 20. genpept11935156 | 69.2 | 67.5 | 67.8 | 66.6 | 91.8 | 92.0 | | 83.1 | 76.4 | 74.6 | 47.1 | 55.8 | 52.3 |
| 21. genpept2274790 | 69.2 | 67.7 | 68.8 | 68.0 | 91.3 | 90.6 | 88.8 | | 82.5 | 80.4 | 50.0 | 56.8 | 54.9 |
| 22. genpept28974117 | 69.2 | 65.4 | 66.8 | 66.3 | 87.0 | 86.5 | 85.3 | 89.6 | | 91.2 | 48.2 | 55.5 | 52.5 |
| 23. genpept28974119 | 68.4 | 68.4 | 67.6 | 66.8 | 85.7 | 85.7 | 84.0 | 87.5 | 94.4 | | 48.5 | 55.6 | 54.2 |
| 24. genpept27544560 | 62.5 | 63.6 | 64.0 | 63.6 | 58.8 | 60.3 | 58.8 | 61.2 | 58.8 | 59.2 | | 50.1 | 50.5 |
| 25. genpept7658233 | 93.9 | 84.5 | 93.7 | 93.3 | 67.7 | 67.2 | 68.4 | 69.4 | 68.2 | 69.2 | 62.9 | | 73.4 |
| 26. genpept66864715 | 80.1 | 81.8 | 80.1 | 79.3 | 65.6 | 65.8 | 65.8 | 67.9 | 63.9 | 67.0 | 62.5 | 80.1 | |

### Example 4

### Identification of Domains Comprised in Polypeptide Sequences Useful in the Invention

### 4.1. Glutamine Synthase (GS1)

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The results of the InterPro scan of the polypeptide sequence as represented by SEQ ID NO: 2 are presented in Table C1.

TABLE C1
InterPro scan results (major accession numbers) of the polypeptide sequence as represented by SEQ ID NO: 2.

| | Accession | | Amino acid coordinates |
|---|---|---|---|
| Database | number | Accession name | on SEQ ID NO 2 |
| InterPro | IPR008146 | Glutamine synthetase, catalytic region | |
| PRODOM | PD001057 | Gln_synt_C | 153-370 |
| PFAM | PF00120 | Gln-synt_C | 132-381 |
| PROSITE | PS00181 | GLNA_ATP | 264-280 |
| InterPro | IPR008147 | Glutamine synthetase, beta-Grasp | |
| PFAM | PF03951 | Gln-synt_N | 36-116 |
| PROSITE | PS00180 | GLNA_1 | 74-91 |
| InterPro | IPR014746 | NGlutamine synthetase/guanido kinase, catalytic region | |
| GENE3D | G3DSA: 3.30.590.10 | no description | 135-376 |
| PANTHER | PTHR20852 | GLUTAMINE SYNTHETASE | 42-381 |
| PANTHER | PTHR20852: SF14 | GLUTAMINE SYNTHETASE (GLUTAMATE-AMMONIA | 42-381 |
| | | LIGASE) (GS) | |

### 4.2. Phosphoethanolamine N-methyltransferase (PEAMT)

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The results of the InterPro scan of the polypeptide sequence as represented by SEQ ID NO: 58 are presented in Table C2.

TABLE C2
InterPro scan results (major accession numbers) of the
polypeptide sequence as represented by SEQ ID NO: 58.

| | Accession | | SEQ | Amino acid coordinates |
|---|---|---|---|---|
| Database | number | Accession name | ID NO: | on SEQ ID NO 58 |
| Interpro | IPR013216 | Methyltransferase type 11 | 86 | 34-143 |
| Interpro | IPR013216 | Methyltransferase type 11 | 87 | 263-370 |
| Interpro | IPR001601 | Generic methyltransferase | | 104-144 |
| Interpro | IPR001601 | Generic methyltransferase | | 333-371 |
| Interpro | IPR004033 | UbiE/COQ5 methyltransferase | 88 | 239-418 |

### 4.3. Fatty Acyl-Acyl Carrier Protein (ACP) Thioesterase B (FATB)

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, Panther, ProDom and Pfam, Smart and TIGRFAMs. Interpro is hosted at the European Bioinformatics Institute in the United Kingdom.

The results of the InterPro scan of the polypeptide sequence as represented by SEQ ID NO: 93 are presented in Table C3.

TABLE C3
InterPro scan results of the polypeptide sequence as represented by SEQ ID NO: 93

| InterPro accession | Integrated database | Integrated database | Integrated database |
|---|---|---|---|
| number and name | name | accession number | accession name |
| IPR002864 Acyl- | Pfam | PF01643 | Acyl-ACP_TE |
| ACP thioesterase | | | |
| family | | | |
| No IPR integrated | G3DSA: 3.10.129.10 | CATH | G3DSA: 3.10.129.10 |
| No IPR integrated | SSF54637 | Superfamily | SSF54637 Thioesterase/thiol ester |
| | | | dehydrase-isomerase |

### 4.4. Leafy-Like (LFY-Like)

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

The results of the InterPro scan of the polypeptide sequence as represented by SEQ ID NO: 146 are presented in Table C4.

TABLE C4
InterPro scan results (major accession numbers) of the
polypeptide sequence as represented by SEQ ID NO: 146.

| | Accession | | Amino acid coordinates |
|---|---|---|---|
| Database | number | Accession name | on SEQ ID NO 146 |
| InterPro | IPR002910 | Floricaula/leafy | |
| | | protein | |
| HMMPfam | PF01698 | FLO_LFY | T[1-395] 0.0 |

### Example 5

### Topology Prediction of the Polypeptide Sequences Useful in the Invention

### 5.1. Glutamine Synthase (GS1)

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

SEQ ID NO: 2 was analysed with TargetP 1.1. The “plant” organism group was selected, no cutoffs defined, and the predicted length of the transit peptide requested. The subcellular localization of the polypeptide sequence as represented by SEQ ID NO: 2 may be the cytoplasm or nucleus, no transit peptide is predicted (predicted localisation: Other: probability 0.737, reliability class 3). Predictions from other algorithms gave similar results:

Psort: peroxisome 0.503; cytoplasm 0.450
PA-SUB: cytoplasm, certainity 100%
PTS1: not targeted to peroxisome

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark
PLOC (Park and Kanehisa, Bioinformatics, 19, 1656-1663, 2003).

### 5.2. Fatty Acyl-Acyl Carrier Protein (ACP) Thioesterase B (FATB)

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

### TargetP v1.1 Prediction Results:

Number of query sequences: 1
Cleavage site predictions included.
Using PLANT networks.

| Name | Length | cTP | mTP | SP | other | Loc | RC | TP length |
|---|---|---|---|---|---|---|---|---|
| Sequence | 412 | 0.957 | 0.010 | 0.089 | 0.144 | C | 1 | 49 |

The subcellular localization of the polypeptide sequence as represented by SEQ ID NO: 93 is the chloroplast, and the predicted length of the transit peptide is of 49 amino acids starting from the N-terminus (not as reliable as the prediction of the subcellular localization itself, may vary in length by a few amino acids).

Many algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark

A transmembrane domain usually denotes a single transmembrane alpha helix of a transmembrane protein. It is called “domain” because an alpha-helix in membrane can be folded independently on the rest of the protein. More broadly, a transmembrane domain is any three-dimensional protein structure which is thermodynamically stable in membrane. This may be a single alpha helix, a stable complex of several transmembrane alpha helices, a transmembrane beta barrel, a beta-helix of gramicidin A, or any other structure.

The TMpred program makes a prediction of membrane-spanning regions and their orientation. The algorithm is based on the statistical analysis of TMbase, a database of naturally occurring transmembrane proteins. The prediction is made using a combination of several weight-matrices for scoring (K. Hofmann & W. Stoffel (1993) TMbase—A database of membrane spanning proteins segments. Biol. Chem. Hoppe-Seyler 374,166). TMpred is part of the European Molecular Biology network (EMBnet.ch) services and is maintained at the server of the Swiss Institute of Bioinformatics.

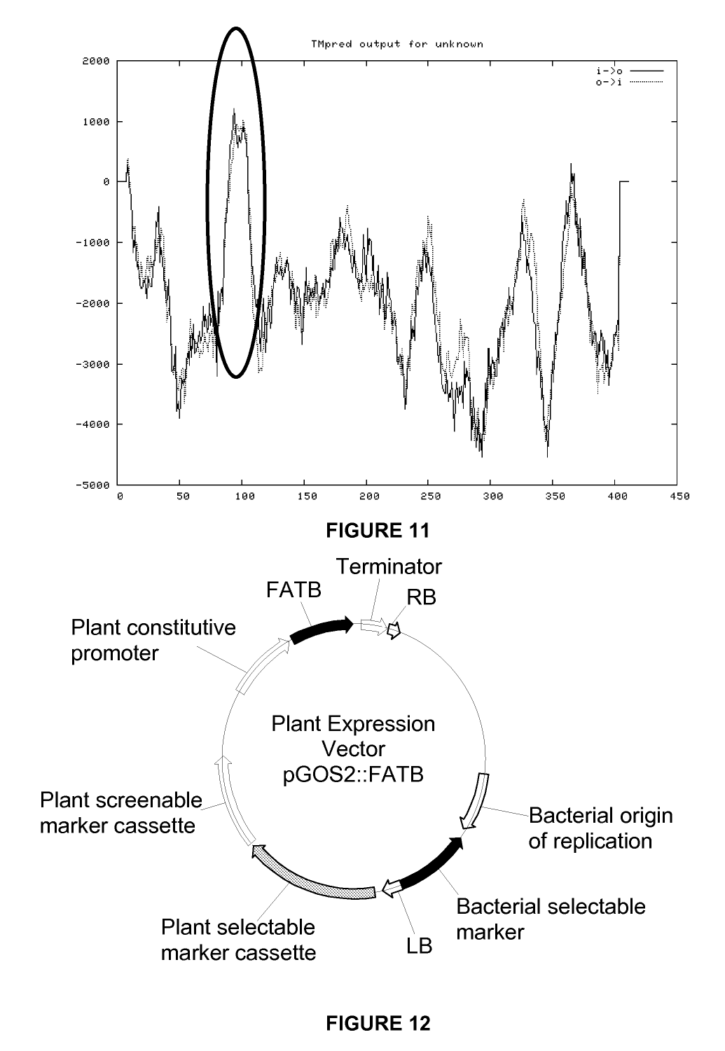

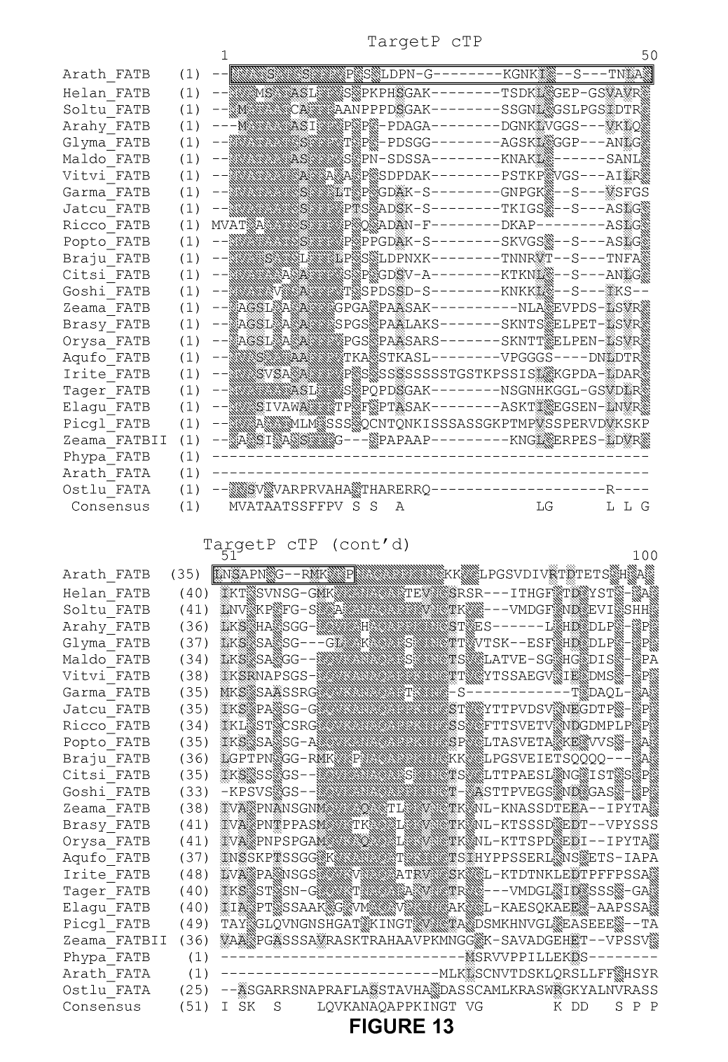

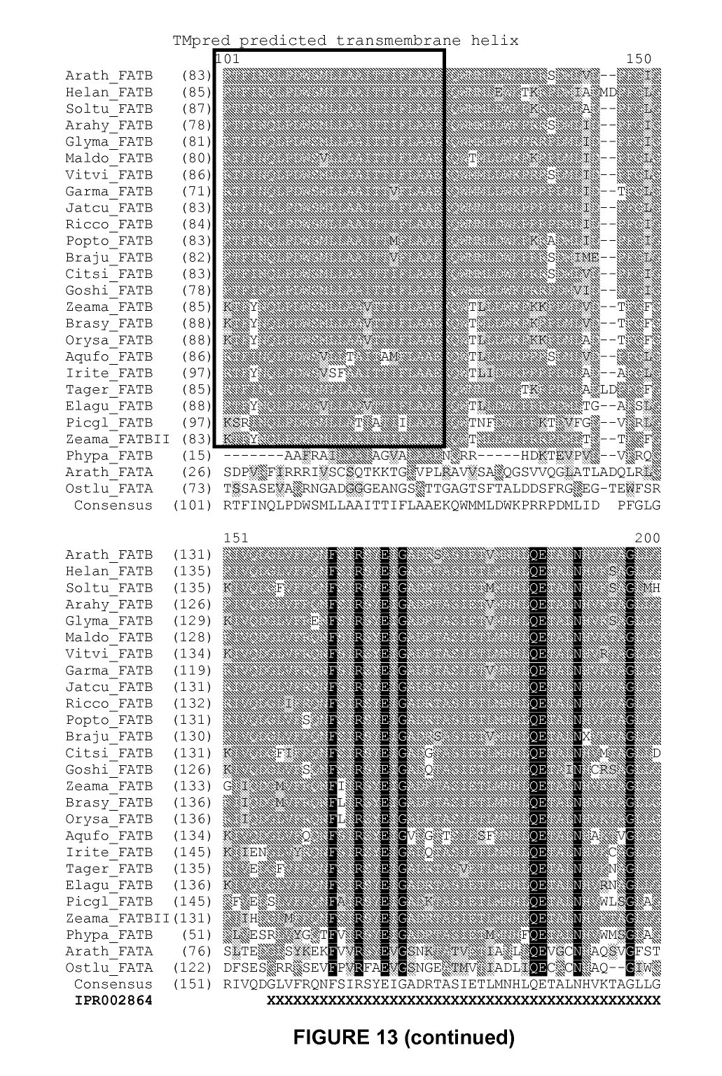

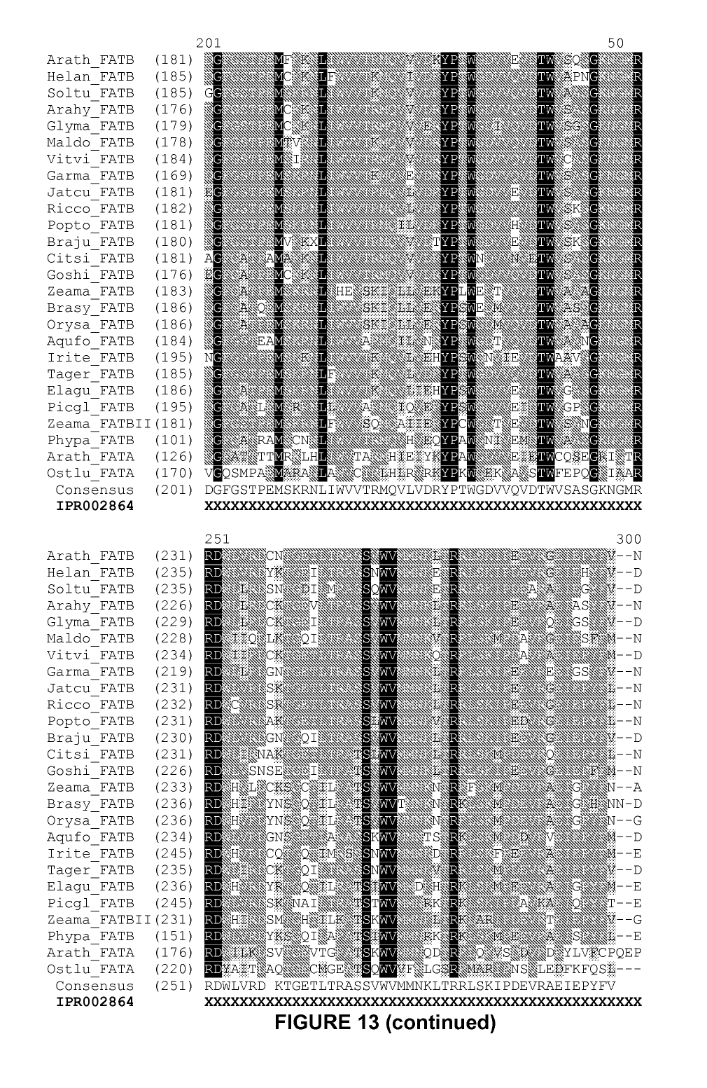

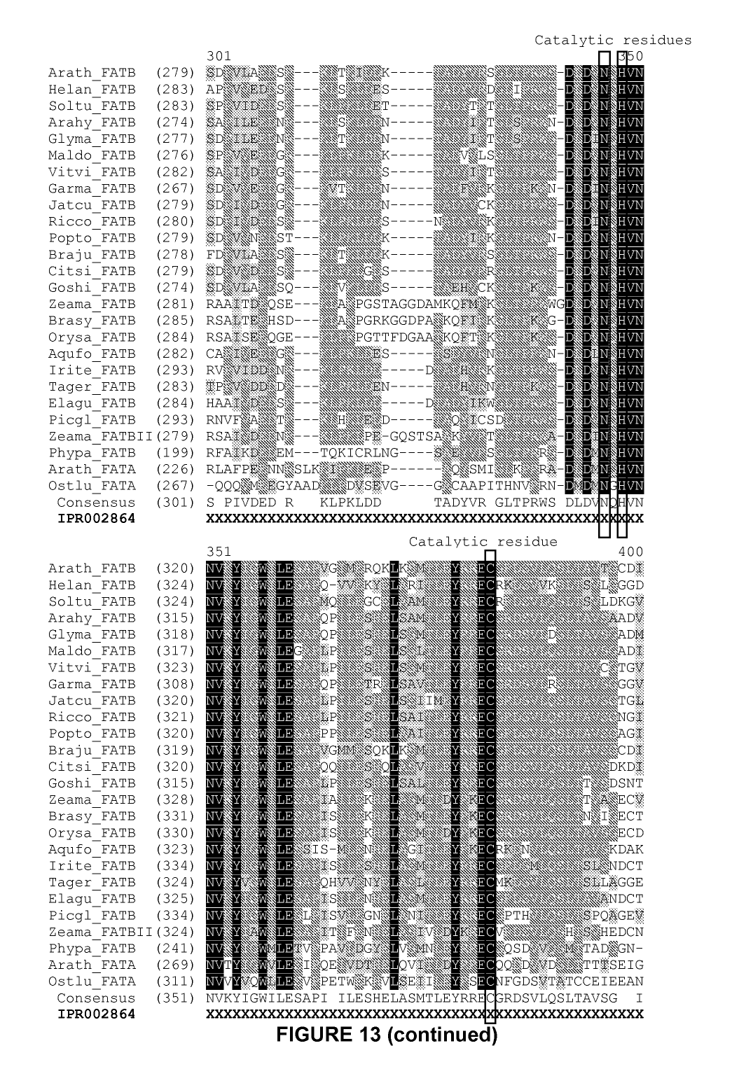

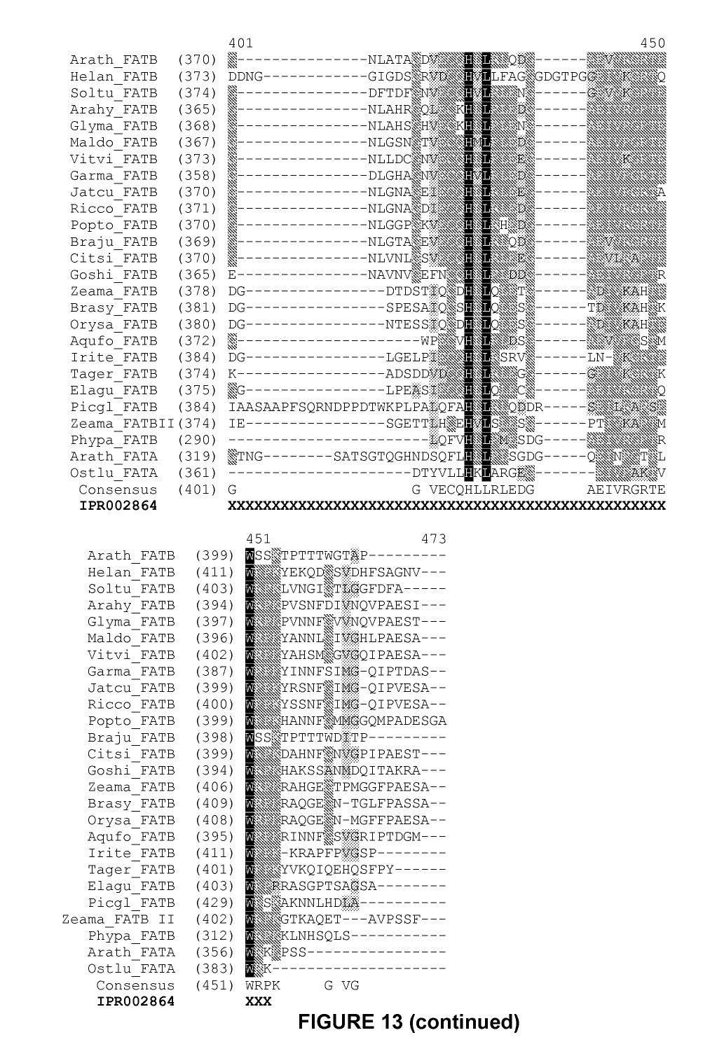

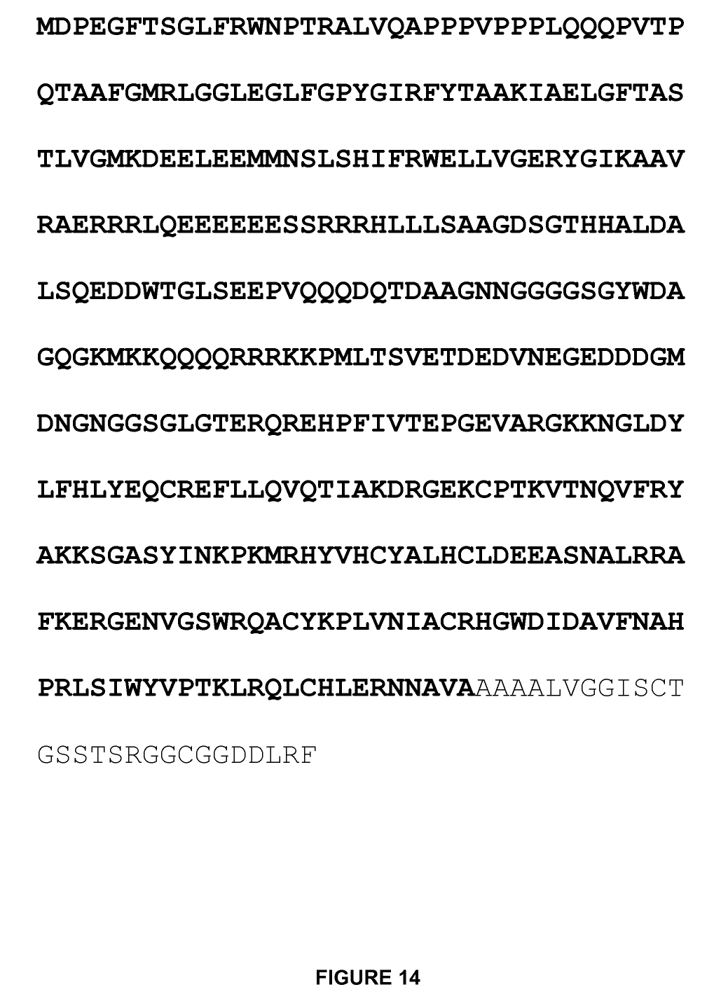

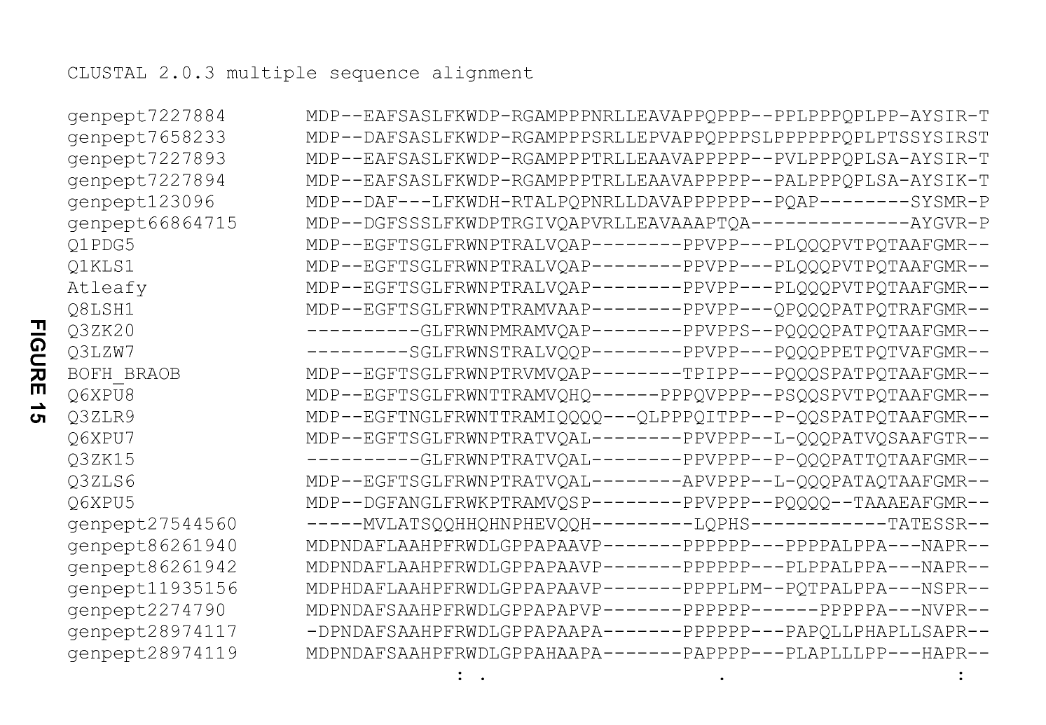

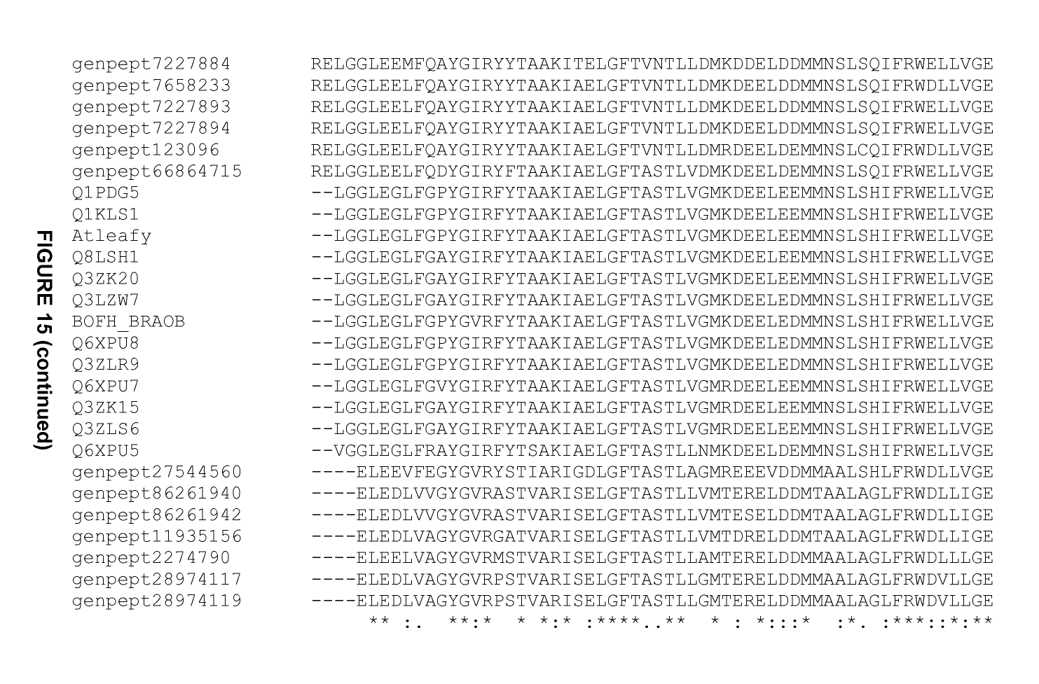

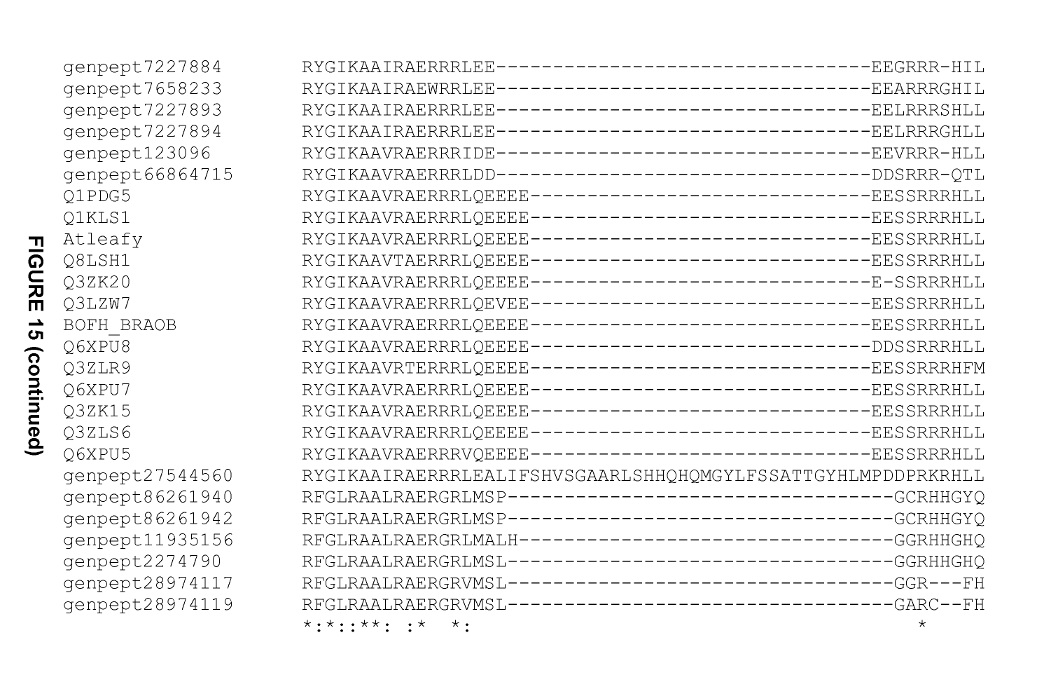

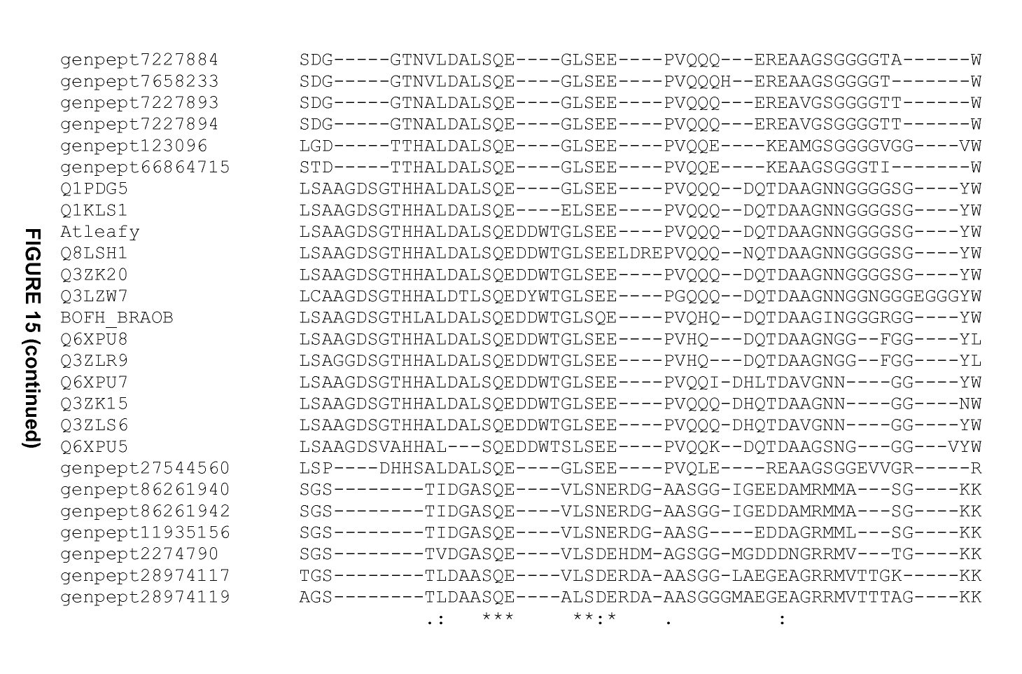

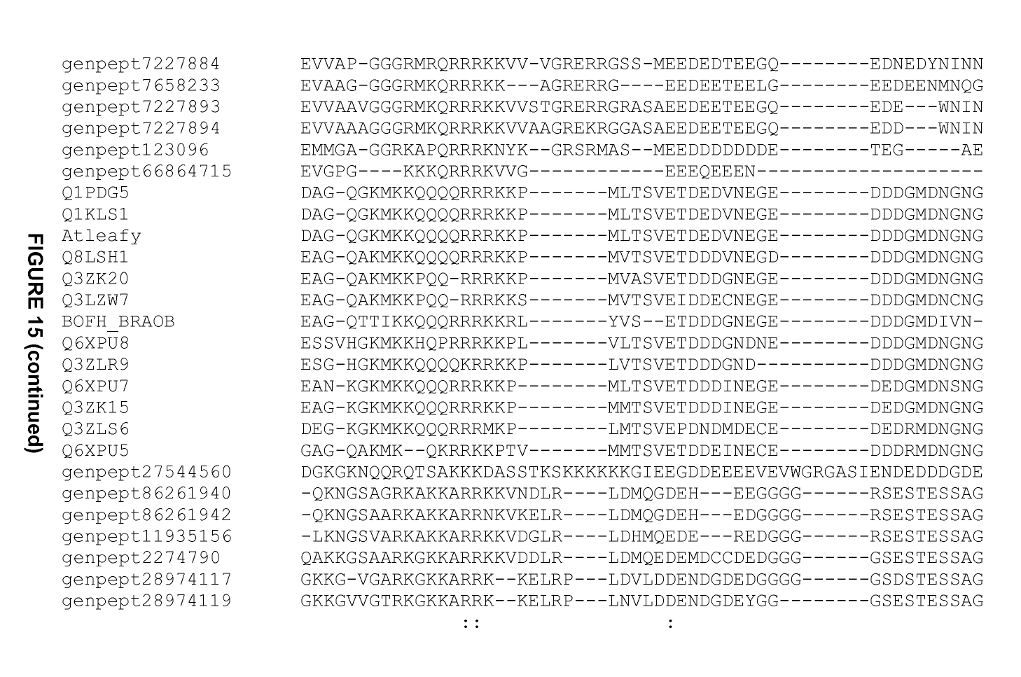

### TMpred Output (See FIG. 11 for Graphical Output):

| | # | from AA | To AA | length | Total score |
|---|---|---|---|---|---|
| Strongly preferred | 1 | 84 | 107 | 24 | 1214 |
| model | | | | | |
| Alternative model | 1 | 89 | 113 | 25 | 1018 |

### 5.3. Leafy-Like (LFY-Like)

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 146 are presented Table D. The “plant” organism group has been selected, no cutoffs defined, and the predicted length of the transit peptide requested. The subcellular localization of the polypeptide sequence as represented by SEQ ID NO: 146 may be the mitochondrion, though the reliability of the prediction is low.

TABLE D
TargetP 1.1 analysis of Atleafy as represented by SEQ ID NO:
146, wherein Len is length of the protein, cTP: probability for a
Chloroplastic transit peptide, mTP: probability for a Mitochondrial
transit peptide, SP: probability for a Secretory pathway signal
peptide, other: probability for a Other subcellular targeting, Loc:
Predicted Location, RC: Reliability class, TPlen: Predicted transit
peptide length:

| Name | Len | cTP | mTP | SP | other | Loc | RC | TPlen |
|---|---|---|---|---|---|---|---|---|
| Atleafy | 424 | 0.181 | 0.432 | 0.015 | 0.404 | M | 5 | 61 |

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark

### Example 6

### Assay Related to the Polypeptide Sequences Useful in the Invention

### 6.1. Glutamine Synthase (GS1)

Assay for glutamine synthase as commercialised by Sigma-Aldrich (modified from Kingdon, H. S., Hubbard, J. S., and Stadtman, E. R. (1968) Biochemistry 7, 2136-2142):

### Principle:

ADP, generated by GS1 upon synthesis of glutamine, is used with phosphor(enol)pyruvate and pyruvate kinase to generate pyruvate and ATP. Pyruvate is converted by L-Lactic Dehydrogenase into L-Lactate with oxidation of β-NADH to β-NAD. The oxidation of NADH is followed spectrophotometrically at 340 nm at 37° C. with a light path of 1 cm in a buffer with pH 7.1.

### Reagents:

### A. 100 mM Imidazole HCl Buffer, pH 7.1 at 37° C.

(Prepare 200 ml in deionized water using Imidazole, Sigma Prod. No. I-0250. Adjust to pH 7.1 at 37° C. with 1 M HCl.)

### B. 3 M Sodium Glutamate Solution (Glu)

(Prepare 10 ml in deionized water using L-Glutamic Acid, Monosodium Salt, Sigma Prod. No. G-1626.)

### C. 250 mM Adenosine 5′-Triphosphate Solution (ATP)

(Prepare 5 ml in deionized water using Adenosine 5′-Triphosphate, Disodium Salt, Sigma Prod. No. A-5394. PREPARE FRESH.)

### D. 33 mM Phospho(enol)pyruvate Solution (PEP)

(Prepare 10 ml in deionized water using Phospho(enol)pyruvate, Trisodium Salt, Hydrate, Sigma Prod. No. P-7002. PREPARE FRESH.)

### E. 900 mM Magnesium Chloride Solution (MgCl₂)

(Prepare 10 ml in deionized water using Magnesium Chloride, Hexahydrate, Sigma Prod. No. M-0250.)

### F. 1 M Potassium Chloride Solution (KCl)

(Prepare 5 ml in deionized water using Potassium Chloride, Sigma Prod. No. P-4504.)

### G. 1.2 M Ammonium Chloride Solution (NH4Cl)

(Prepare 5 ml in deionized water using Ammonium Chloride, Sigma Prod. No. A-4514.)

### H. 12.8 mM β-Nicotinamide Adenine Dinucleotide Solution, Reduced Form (β-NADH)

(Dissolve the contents of one 10 mg vial of β-Nicotinamide Adenine Dinucleotide, Reduced Form, Disodium Salt, Sigma Stock No. 340-110 in the appropriate volume of Reagent A. PREPARE FRESH.)

### I. PK/LDH Enzymes Solution (PK/LDH)

(Use PK/LDH Enzymes Solution in 50% Glycerol, Sigma Prod. No. P-0294; contains approximately 700 units/ml pyruvate kinase and 1,000 units/ml lactic dehydrogenase. L-Lactic Dehydrogenase Unit Definition: One unit will reduce 1.0 μmole of pyruvate to L-lactate per minute at pH 7.5 at 37° C. Pyruvate Kinase Unit Definition: One unit will convert 1.0 μmole of phospho(enol)pyruvate to pyruvate per minute at pH 7.6 at 37° C.)

### J. Glutamine Synthetase Enzyme Solution

(Immediately before use, prepare a solution containing 4-8 units/ml of Glutamine Synthetase in cold deionized water).

### Procedure:

Prepare a Reaction Cocktail by pipetting (in milliliters) the following reagents into a suitable container:

| | |
|---|---|
| Deionized Water | 20.60 |
| Reagent A (Buffer) | 17.20 |
| Reagent B (Glu) | 1.80 |
| Reagent C (ATP) | 1.80 |
| Reagent E (MgCl₂) | 3.55 |
| Reagent F (KCl) | 0.90 |
| Reagent G (NH₄Cl) | 1.80 |

Mix by stirring and adjust to pH 7.1 at 37° C. with 0.1 N HCl or 0.1 N NaOH, if necessary. Pipette (in milliliters) the following reagents into suitable cuvettes:

| | Test | Blank |
|---|---|---|
| Reaction Cocktail | 2.70 | 2.70 |
| Reagent D (PEP) | 0.10 | 0.10 |
| Reagent H (β-NADH) | 0.06 | 0.06 |

Mix by inversion and equilibrate to 37° C. Monitor the A₃₄₀nm until constant, using a suitably thermostatted spectrophotometer. Then add:

| | | |
|---|---|---|
| Reagent I (PK/LDH) | 0.04 | 0.04 |

Mix by inversion and equilbrate to 37° C. Monitor the A₃₄₀nm until constant, using a suitably thermostatted spectrophotometer. Then add:

| | | |
|---|---|---|
| Deionized water | — | 0.10 |
| Reagent J (Enzyme Solution) | 0.10 | — |

Immediately mix by inversion and record the decrease in A₃₄₀nm for approximately 10 minutes. Obtain the ΔA₃₄₀nm/min using the maximum linear rate for both the Test and Blank.

### Calculations:

$$Units \text{/} ml enzyme = \frac{( \begin{matrix}Δ A 340 nm \text{/} min Test - \\ Δ A 340 nm \text{/} min Blank\end{matrix} ) ( 3 ) ( 15 )}{( 6.22 ) ( 0.1 )}$$

3=Total volume (in milliliters) of assay
15=Conversion factor to 15 minutes (Unit Definition)
6.22=Millimolar extinction coefficient of β-NADH at 340 nm
0.1=Volume (in milliliter) of enzyme used

$$Units \text{/} mg solid = \frac{units \text{/} ml enzyme}{mg solid \text{/} ml enzyme}$$

$$Units \text{/} mg protein = \frac{units \text{/} ml enzyme}{mg protein \text{/} ml enzyme}$$

### Unit Definition:

One unit will convert 1.0 pmole of L-glutamate to L-glutamine in 15 minutes at pH 7.1 at 37° C.

### Final Assay Concentrations:

In a 3.00 ml reaction mix, the final concentrations are 34.1 mM imidazole, 102 mM sodium glutamate, 8.5 mM adenosine 5′-triphosphate, 1.1 mM phosphoenolpyruvate, 60 mM magnesium chloride, 18.9 mM potassium chloride, 45 mM ammonium chloride, 0.25 mM β-nicotinamide adenine dinucleotide, 28 units pyruvate kinase, 40 units L-lactic dehydrogenase and 0.4-0.8 units glutamine synthetase.

### 6.2. Fatty Acyl-Acyl Carrier Protein (ACP) Thioesterase B (FATB)

Polypeptides useful in performing the methods of the invention typically display thioesterase enzymatic activity. Many assays exist to measure such activity, for example, the FATB polypeptide can be expressed in an E. coli strain deficient in free fatty acid uptake from the medium. Thus, when a FATB polypeptide is functioning in this system, the free fatty acid product of the thioesterase reaction accumulates in the medium. By measuring the free fatty acids in the medium, the enzymatic activity of the polypeptide can be identified (Mayer & Shanklin (2005) J Biol Chem 280: 3621). Thioesterase assays related to FATB polypeptide enzymatic activity can also performed, as described in Voelker et al. (1992; Science 257: 72-74).

A person skilled in the art is well aware of such experimental procedures to measure FATB polypeptide enzymatic activity, including the activity of a FATB polypeptide as represented by SEQ ID NO: 93.

### Example 7

### Cloning of the Nucleic Acid Sequence Used in the Methods of the Invention

### 7.1. Glutamine Synthase (GS1)

The nucleic acid sequence used in the methods of the invention was amplified by PCR using as template a custom-made Chlamydomonas reinhardtii cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm08458 (SEQ ID NO: 7; sense, start codon in bold): 5′-ggggacaagtttgtacaaaaaagcaggcttaaacaatggccgcgggatctgtt-3′ and prm08459 (SEQ ID NO: 8, reverse, complementary): 5′-ggggaccactttgtacaagaaagctgggtgctgctcctgcgcttacagaa-3′, which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pGS1. Plasmid pDONR201 was purchased from Invitrogen, as part of the Gateway® technology.

The entry clone comprising SEQ ID NO: 1 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice protochlorophyllide reductase promoter promoter (pPCR, SEQ ID NO: 6) for shoot specific expression was located upstream of this Gateway cassette.

After the LR recombination step, the resulting expression vector pPCR::GS1 (FIG. 3) was transformed into Agrobacterium strain LBA4044 according to methods well known in the art.

### 7.2. Phosphoethanolamine N-methyltransferase (PEAMT)

The nucleic acid sequence used in the methods of the invention was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were primer: 5′-ggggacaagtttgtacaaaaaagcaggcttaaacaatggagcattctagtgatttg-3′ (SEQ ID NO: 83; sense) and primer 5′-ggggaccactttgtacaagaaagctgggtcagagttttgggataaaaaca-3′ (SEQ ID NO: 84; reverse, complementary): which include the AttB sites for Gateway recombination. The amplified PCR fragment was purified also using standard methods. The first step of the Gateway procedure, the BP reaction, was then performed, during which the PCR fragment recombined in vivo with the pDONR201 plasmid to produce, according to the Gateway terminology, an “entry clone”, pArath_PEAMT_{—}1. Plasmid pDONR201 was purchased from Invitrogen, as part of the Gateway® technology.

The entry clone comprising SEQ ID NO: 57 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 85) for constitutive expression was located upstream of this Gateway cassette.

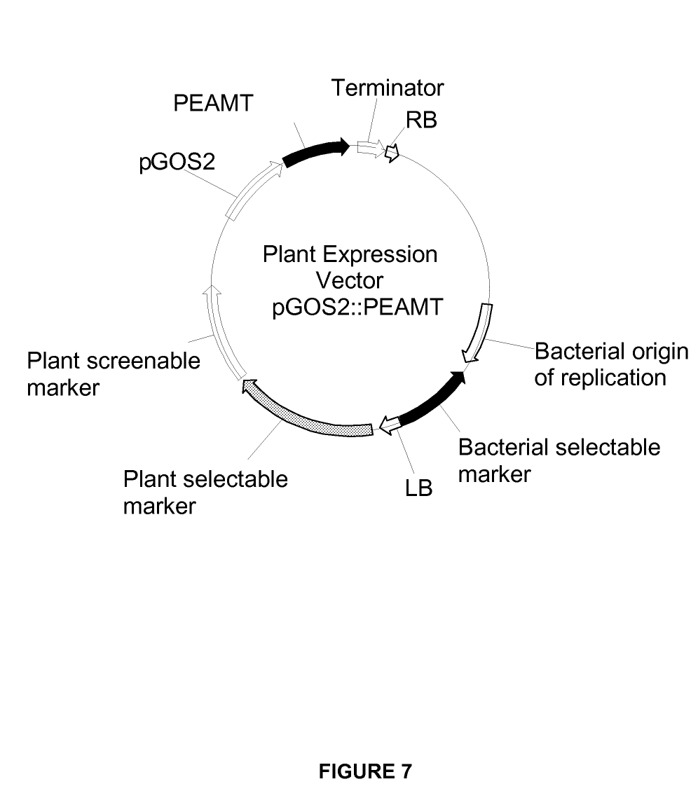

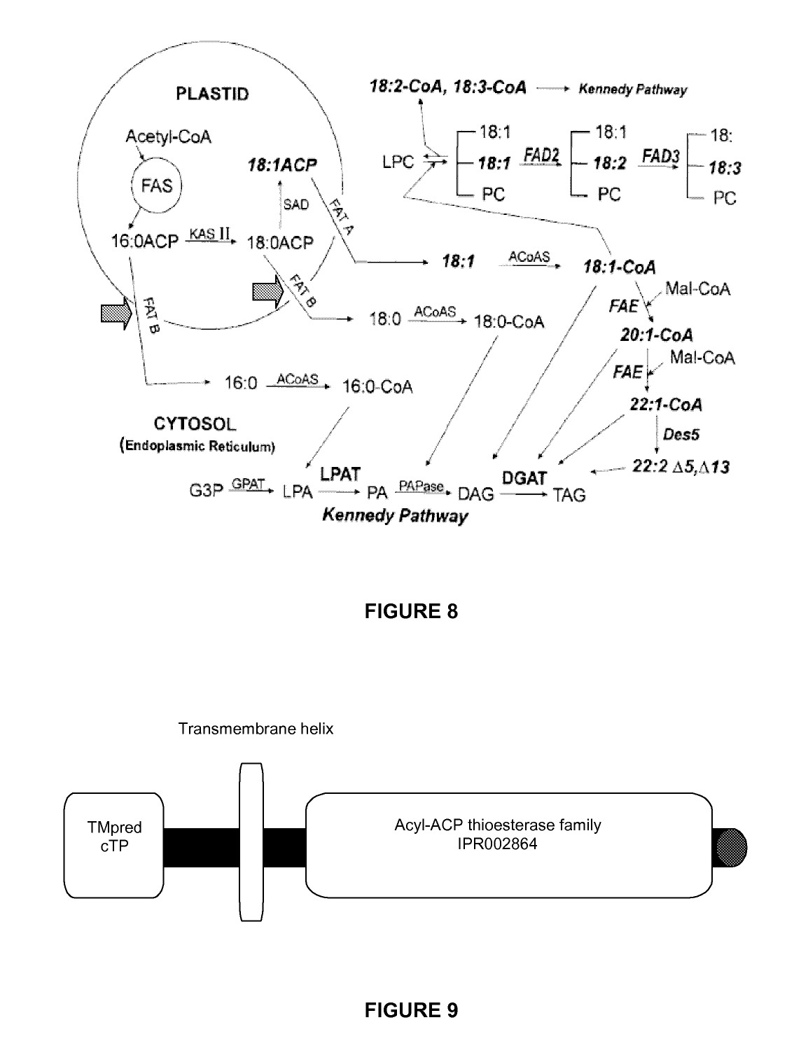

After the LR recombination step, the resulting expression vector pGOS2::Arath_PEAMT_{—}1 (FIG. 7) was transformed into Agrobacterium strain LBA4044 according to methods well known in the art.

### 7.3. Fatty Acyl-Acyl Carrier Protein (ACP) Thioesterase B (FATB)

Unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

The Arabidopsis thaliana nucleic acid sequence encoding a FATB polypeptide sequence as represented by SEQ ID NO: 93 was amplified by PCR using as template a cDNA bank constructed using RNA from Arabidopsis plants at different developmental stages. The following primers, which include the AttB sites for Gateway recombination, were used for PCR amplification: prm08145: 5′-ggggacaagtttgtacaaaaaagcaggcttaaacaatggtggccacctctgc-3′ (SEQ ID NO: 142, sense) and prm08146: 5′-ggggaccactttgtacaagaaagctgggttttttcttacggtgcagttcc-3′ (SEQ ID NO: 143, reverse, complementary). PCR was performed using Hifi Taq DNA polymerase in standard conditions. A PCR fragment of the expected length (including attB sites) was amplified and purified also using standard methods. The first step of the Gateway procedure, the BP reaction, was then performed, during which the PCR fragment recombined in vivo with the pDONR201 plasmid to produce, according to the Gateway terminology, an “entry clone”. Plasmid pDONR201 was purchased from Invitrogen, as part of the Gateway® technology.

The entry clone comprising SEQ ID NO: 92 was subsequently used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 144) for constitutive expression was located upstream of this Gateway cassette.

After the LR recombination step, the resulting expression vector pGOS2::FATB (FIG. 12) for constitutive expression, was transformed into Agrobacterium strain LBA4044 according to methods well known in the art.

### 7.4. Leafy-Like (LFY-Like)

The nucleic acid sequence used in the methods of the invention was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm4841 (SEQ ID NO: 147; sense, start codon in bold): 5′-ggggacaagtttgtacaaaaaagcaggcttaaacaatggatcctgaaggtttcac-3′ and prm4842 (SEQ ID NO: 148; reverse, complementary): 5′-ggggaccactttgtacaagaaagctgggtaaccaaactagaaacgcaagt-3′, which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pLFY-like. Plasmid pDONR201 was purchased from Invitrogen, as part of the Gateway® technology.

The entry clone comprising SEQ ID NO: 145 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 5 for constitutive expression was located upstream of this Gateway cassette. In an alternative embodiment, a shoot-specific promoter was used (PCR, protochlorophyllid reductase promoter, SEQ ID NO: 150)

After the LR recombination step, the resulting expression vector pGOS2::LFY-like (FIG. 16) or pPCR::LFY-like, was transformed into Agrobacterium strain LBA4044 according to methods well known in the art.

### Example 8

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges 1996, Chan et al. 1993, Hiei et al. 1994).

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown D C W and A Atanassov (1985. Plant Cell Tissue Organ Culture 4: 111-112). Alternatively, the RA3 variety (University of Wisconsin) has been selected for use in tissue culture (Walker et al., 1978 Am J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton is transformed using Agrobacterium tumefaciens according to the method described in U.S. Pat. No. 5,159,135. Cotton seeds are surface sterilised in 3% sodium hypochlorite solution during 20 minutes and washed in distilled water with 500 μg/ml cefotaxime. The seeds are then transferred to SH-medium with 50 μg/ml benomyl for germination. Hypocotyls of 4 to 6 days old seedlings are removed, cut into 0.5 cm pieces and are placed on 0.8% agar. An Agrobacterium suspension (approx. 108 cells per ml, diluted from an overnight culture transformed with the gene of interest and suitable selection markers) is used for inoculation of the hypocotyl explants. After 3 days at room temperature and lighting, the tissues are transferred to a solid medium (1.6 g/l Gelrite) with Murashige and Skoog salts with B5 vitamins (Gamborg et al., Exp. Cell Res. 50:151-158 (1968)), 0.1 mg/l 2,4-D, 0.1 mg/l 6-furfurylaminopurine and 750 μg/ml MgCL2, and with 50 to 100 μg/ml cefotaxime and 400-500 μg/ml carbenicillin to kill residual bacteria. Individual cell lines are isolated after two to three months (with subcultures every four to six weeks) and are further cultivated on selective medium for tissue amplification (30° C., 16 hr photoperiod). Transformed tissues are subsequently further cultivated on non-selective medium during 2 to 3 months to give rise to somatic embryos. Healthy looking embryos of at least 4 mm length are transferred to tubes with SH medium in fine vermiculite, supplemented with 0.1 mg/l indole acetic acid, 6 furfurylaminopurine and gibberellic acid. The embryos are cultivated at 30° C. with a photoperiod of 16 hrs, and plantlets at the 2 to 3 leaf stage are transferred to pots with vermiculite and nutrients. The plants are hardened and subsequently moved to the greenhouse for further cultivation.

### Example 9

### Phenotypic Evaluation Procedure

### 9.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Plants grown under non-stress conditions were watered at regular intervals to ensure that water and nutrients were not limiting and to satisfy plant needs to complete growth and development.

Four events were further evaluated following the same evaluation procedure as for the T2 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from T2 seeds are grown in potting soil under normal conditions until they approach the heading stage. They are then transferred to a “dry” section where irrigation is withheld. Humidity probes are inserted in randomly chosen pots to monitor the soil water content (SWC). When SWC goes below certain thresholds, the plants are automatically re-watered continuously until a normal level is reached again. The plants are then re-transferred again to normal conditions. The rest of the cultivation (plant maturation, seed harvest) is the same as for plants not grown under abiotic stress conditions. Growth and yield parameters are recorded as detailed for growth under normal conditions.

### Nitrogen Use Efficiency Screen

Rice plants from T2 seeds were grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters were recorded as detailed for growth under normal conditions.

### Salt Stress Screen

Plants are grown on a substrate made of coco fibers and argex (3 to 1 ratio). A normal nutrient solution is used during the first two weeks after transplanting the plantlets in the greenhouse. After the first two weeks, 25 mM of salt (NaCl) is added to the nutrient solution, until the plants are harvested. Seed-related parameters are then measured.

### 9.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment-event-segregants). P values were obtained by comparing likelihood ratio test to chi square distributions.

### 9.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Early vigour was determined by counting the total number of pixels from aboveground plant parts discriminated from the background. This value was averaged for the pictures taken on the same time point from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. The Harvest Index (HI) in the present invention is defined as the ratio between the total seed yield and the above ground area (mm²), multiplied by a factor 10⁶. The seed fill rate as defined in the present invention is the proportion (expressed as a %) of the number of filled seeds over the total number of seeds (or florets).

### Example 10

### Results of the Phenotypic Evaluation of the Transgenic Plants

### 10.1 Glutamine Synthase (GS1)

Rice plants from T2 seeds were grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters were recorded as detailed for growth under normal conditions.

The results of the evaluation of transgenic rice plants expressing a GS1 nucleic acid under conditions of nutrient deficiency are presented below in Table E1. An increase of more than 5% was observed for total seed yield, number of filled seeds, fill rate, total number of seeds, and harvest index. These increases were confirmed in a subsequent experiment.

TABLE E1

| | 1^{st} experiment | | Confirmation experiment | |
|---|---|---|---|---|
| parameter | % increase | p-value | % increase | p-value |
| total seed yield | 17 | 0.011 | 18 | 0.000 |
| number of filled seeds | 16 | 0.014 | 18 | 0.000 |
| fill rate | 7 | 0.043 | 10 | 0.308 |
| total number of seeds | 26 | 0.117 | 15 | 0.000 |
| harvest index | 12 | 0.019 | 14 | 0.021 |

In addition, an increase was found for biomass (2 positive lines out of 4, overall increase 13%) and for early vigour (3 positive lines out of 4, overall increase 28%).

### 10.2. Phosphoethanolamine N-methyltransferase (PEAMT)

The results of the evaluation of transgenic rice plants expressing the Arath_PEAMT_{—}1 nucleic acid under non-stress conditions are presented below. An increase of at least 5% was observed for the total seed yield, seed fill rate, number of flowers per panicle and harvest index (Table E2).

TABLE E2
Results phenotypic evaluation under non-stress conditions.

| | % increase in transgenic |
|---|---|
| Parameter | plant versus control plant |
| Total Seed Yield | 12 |
| Flowers Per Panicle | 5.1 |
| See Fill Rate | 12 |
| Harvest Index | 3.4 |

Plants from T2 seeds were grown in potting soil under normal conditions until they approached the heading stage. They were then transferred to a “dry” section where irrigation was withheld. Humidity probes were inserted in randomly chosen pots to monitor the soil water content (SWC). When SWC went below certain thresholds, the plants were automatically re-watered continuously until a normal level was reached again. The plants were then re-transferred again to normal conditions. The rest of the cultivation (plant maturation, seed harvest) was the same as for plants not grown under abiotic stress conditions. Growth and yield parameters were recorded as detailed for growth under normal conditions.

The results of the evaluation of transgenic rice plants expressing a PEAMT nucleic acid under drought-stress conditions are presented hereunder. An increase was observed for total seed weight, number of filled seeds, fill rate, harvest index and thousand-kernel weight (Table E3). An increase of at least 5% was observed for aboveground area (AreaMax; green biomass), emergence vigour (early vigour), and of 2.5% for thousand kernel weight.

TABLE E3
Results phenotypic evaluation under drought screen.

| | % increase in transgenic |
|---|---|
| Parameter | plant versus control plant |
| Aboveground Area | 5.4 |
| Emergence Vigour | 15 |
| Thousand Kernel Weight | 3 |

### 10.3. Fatty Acyl-Acyl Carrier Protein (ACP) Thioesterase B (FATB)

The results of the evaluation of T1 and T2 generation transgenic rice plants expressing the nucleic acid sequence encoding a FATB polypeptide as represented by SEQ ID NO: 93, under the control of a GOS2 constitutive promoter, and grown under normal growth conditions, are presented below.

There was a significant increase in the early vigor, in the aboveground biomass, in the total seed yield per plant, in the total number of seeds, in the number of filled seeds, in the seed filling rate, and in the harvest index of the transgenic plants compared to corresponding nullizygotes (controls), as shown in Table E4

TABLE E4
Results of the evaluation of T1 and T2 generation transgenic
rice plants expressing the nucleic acid sequence encoding a
FATB polypeptide as represented by SEQ ID NO: 93, under the
control of a GOS2 promoter for constitutive expression.

| | overall average % | overall average % |
|---|---|---|
| | increase in 6 events | increase in 4 events |
| Trait | in the T1 generation | in the T2 generation |
| Total seed yield per plant | 17% | 9% |
| Total number of seeds | 1% | 8% |
| Total number of filled seeds | 17% | 10% |
| Seed filling rate | 14% | 2% |
| Harvest index | 17% | 6% |

### 10.4. Leafy-Like (LFY-Like)

Transgenic rice plants expressing a LFY-like nucleic acid under non-stress conditions showed increased seed yield. The plants expressing Atleafy under control of the constitutive promoter or the shoot specific promoter gave an increase in one or more of the following parameters: fillrate, harvest index, thousand kernel weight, flowers per panicle.

1. A method for enhancing yield-related traits in plants relative to control plants, comprising modulating expression in a plant of a nucleic acid encoding an algal-type cytoplasmic glutamine synthase (GS1) polypeptide, wherein said algal-type GS1 polypeptide comprises a Gln-synt_C domain (Pfam accession PF00120) and a Gln-synt_N domain (Pfam accession PF03951).

2. The method of claim 1, wherein said GS1 polypeptide comprises one or more of the following motifs:
(i) Motif 1, SEQ ID NO: 3;
(ii) Motif 2, SEQ ID NO: 4;
(iii) Motif 3, SEQ ID NO: 5,
in which motifs maximally 2 mismatches are allowed.

3. The method of claim 1, wherein said modulated expression is effected by introducing and expressing in a plant a nucleic acid encoding an algal-type GS1 polypeptide.

4. The method of claim 1, wherein said nucleic acid encoding a GS1 polypeptide encodes any one of the proteins listed in Table A or is a portion of such a nucleic acid, or a nucleic acid capable of hybridising with such a nucleic acid.

5. The method of claim 1, wherein said nucleic acid sequence encodes an orthologue or paralogue of any of the proteins given in Table A.

6. The method of claim 1, wherein said enhanced yield-related traits comprise increased yield relative to control plants.

7. The method of claim 1, wherein said enhanced yield-related traits are obtained under conditions of nutrient deficiency.

8. The method of claim 1, wherein said nucleic acid is operably linked to a shoot-specific promoter, to a protochlorophyllide reductase promoter, or to a protochlorophyllide reductase promoter from rice.

9. The method of claim 1, wherein said nucleic acid encoding a GS1 polypeptide is of plant origin.

10. A plant or part thereof, including seeds, obtained by the method of claim 1, wherein said plant or part thereof comprises a recombinant nucleic acid encoding a GS1 polypeptide.

11. A construct comprising:
(i) a nucleic acid encoding an algal-type cytoplasmic glutamine synthase (GS1) polypeptide, wherein said algal-type GS1 polypeptide comprises a Gln-synt_C domain (Pfam accession PF00120) and a Gln-synt_N domain (Pfam accession PF03951);
(ii) one or more control sequences capable of driving expression of the nucleic acid sequence of (a); and optionally
(iii) a transcription termination sequence.

12. The construct of claim 11, wherein one of said control sequences is a shoot-specific promoter, a protochlorophyllide reductase promoter, or a protochlorophyllide reductase promoter from rice.

13. A method for making plants having increased yield, increased biomass and/or increased seed yield relative to control plants, comprising introducing into a plant, plant part or plant cell the construct of claim 11.

14. A plant, plant part or plant cell transformed with the construct claim 11.

15. A method for the production of a transgenic plant having increased yield, increased biomass and/or increased seed yield relative to control plants, comprising:
(i) introducing and expressing in a plant a nucleic acid encoding a GS1 polypeptide as defined in claim 1; and
(ii) cultivating the plant cell under conditions promoting plant growth and development.

16. A transgenic plant having increased yield, increased biomass and/or increased seed yield, relative to control plants, resulting from modulated expression of a nucleic acid encoding a GS1 polypeptide as defined in claim 1, or a transgenic plant cell derived from said transgenic plant.

17. The transgenic plant according to claim 14, or a transgenic plant cell derived thereof, wherein said plant is a crop plant or a monocot or a cereal.

18. Harvestable parts of a plant according to claim 17, wherein said harvestable parts are preferably shoot biomass and/or seeds.

19. Products derived from a plant according to claim 17 and/or from harvestable parts thereof.

20. (canceled)

21. An isolated polypeptide selected from:
(i) an amino acid sequence represented by SEQ ID NO: 53 or 54;
(ii) an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence represented by SEQ ID NO: 53 or 54,
(iii) derivatives of any of the amino acid sequences given in (i) or (ii) above.

22. An isolated nucleic acid encoding a polypeptide as defined in claim 22, or a nucleic acid hybridising thereto.
